(19) United States
(12) Patent Application Publication
(10) Pub. No.: US 2002/0081584 A1
(43) Pub. Date: Jun. 27, 2002

(54) GENES, PROTEINS AND BIALLELIC MARKERS RELATED TO CENTRAL NERVOUS SYSTEM DISEASE

(76) Inventors: MARTA BLUMENFELD, PARIS (FR); LYDIE BOUGUELERET, VANVES (FR); ILYA CHUMAKOV, VAUX-LE-PENIL (FR); LAURENT ESSIOUX, PARIS (FR); DANIEL COHEN, NEUILLY-SUR-SEINE (FR)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/416,384

(22) Filed: Oct. 12, 1999

Related U.S. Application Data
(60) Provisional application No. 60/103,955, filed on Oct. 13, 1998.
(60) Provisional application No. 60/106,457, filed on Oct. 30, 1998.

(51) Int. Cl. C12Q 1/68; C07H 21/02; C07H 21/04; C12P 19/34

(52) U.S. Cl. 435/6; 435/91.1; 435/91.2; 536/23.1; 536/24.3

(57) ABSTRACT

The invention concerns genes, polymorphisms and polypeptides related to central nervous systems disease. Included are the G713 gene, the G713 protein and G713 biallelic markers, as well as biallelic markers located on the human chromosome 13q31-q33 locus, and the association established between these biallelic markers and schizophrenia. The invention also provides means to determine the predisposition of individuals to schizophrenia as well as means for the diagnosis of this disease and for the prognosis and detection of an eventual treatment response to therapeutic agents acting against schizophrenia

### RELATED APPLICATIONS

This application claims priority under 35 U.S.C. §119(e) to U.S. Provisional Application Ser. Nos. 60/103,955, filed Oct. 12, 1998, and 60/106,457, filed Oct. 30, 1998.

### FIELD OF THE INVENTION

The present invention a novel gene, the G713 gene, located on the 13q33 locus of chromosome 13, and expressed in human brain, the coding sequence of which comprises repeated CAG triplet nucleotide repeats, as well as with single nucleotide polymorphisms, also termed biallelic markers, that are harbored by the G713 gene. The G713 gene represents a candidate gene for central nervous system disorders, including schizophrenia and bipolar disorder.

The invention also concerns biallelic markers located on the human chromosome 13q31-q33 locus and the association established between these markers and predisposition to schizophrenia. The invention provides means to determine the predisposition of individuals to schizophrenia as well as means for the diagnosis of such diseases and for the prognosis/detection of an eventual treatment response to agents acting on schizophrenia.

Throughout this application, various references are referred to within parentheses. The disclosures of these publications in their entireties are hereby incorporated by reference into this application to more fully describe the state of the art to which this invention pertains.

### BACKGROUND OF THE INVENTION

Recently, there has been an increasing interest in a new class of genetic diseases caused by abnormal expansions of tracts of trinucleotide repeats. Specifically, an increasing number of human neurodegenerative diseases are recognized to be caused by expansion of a CAG repeat within the protein-coding region of the disease gene. The expanded repeat encodes an expanded tract of glutamines within the protein. Whereas a normal repeat length has no pathological consequence, expansion of the glutamine tract beyond a critical threshold leads to neuronal loss and a degenerative phenotype. To date, eight glutamine-repeat diseases have been identified, including Huntington's disease (HD), spinobulbar muscular atrophy (SBMA), dentatorubral-pallidolusyan atrophy (DRPLA), and five spinocerebellar ataxias (SCAs 1, 2, 6, 7, and SCA3/MJD). These diseases all affect the nervous system and share a number of common features that are detailed hereafter. First, although normal chromosomes are polymorphic with respect to repeat length, they show very low mutation rates. However, mutant chromosomes with long repeats are highly mutable and tend to increase their repeat number in successive generations. Second, as a general rule, increasing disease severity and/or decreasing age of onset of symptoms correlate with increasing size of triplet expansions. These molecular features can explain the phenomenon of anticipation, which is understood today as the tendency for the disease to manifest at an earlier age in successive generations. In particular, recent reports have suggested that anticipation may be a feature of both schizophrenia and bipolar affective disorder (Ross et al., 1993; Basset et al., 1994; McInnis et al., 1993).

These diseases likely share a common pathophysiology at the protein level: the expanded polyglutamine tract confers a dominant, toxic property upon these otherwise unrelated proteins. The longer the repeat, the earlier the onset and the more severe the disease. For Huntington's disease (HD) in particular, several authors have shown the existence of a correlation between the number of CAG repeats present upstream the huntingtin (Huntington Disease's protein) coding sequence and both the severity and the age of onset of this pathology. For example, Brinkman et al. (1997) have used a large cohort of patients and their study has shown that CAG repeat length is the major determinant of age at onset in HD. By assessing the CAG size alone, these authors were able to predict the likelihood that an individual would be affected by a particular age, for the vast majority of persons tested. This study showed that the lower limit of CAG repeat size in individuals who manifest with HD is 36. These authors showed that there is a trend to increasing penetrance with increasing repeat length in the 36-41 repeat range.

Increasing evidence indicates that expanded polyglutamine itself drives the degenerative process. Isolated, expanded glutamine tracts cause neurodegeneration in transgenic mice and cell death in transfected cells, and an expanded glutamine repeat inserted into a non-disease protein causes neurodegeneration in transgenic mice. Recent evidence indicates that neuronal nuclear inclusions (NI) formed by the disease protein are a unifying pathological feature of these diseases. Although it is unknown whether NI cause the disease or simply reflect the disease process, the fact that they are preferentially found in susceptible neurons indicates that they are intimately linked to disease progression. Analysis of NI in transgenic animals and in HD brain reveals occasional fibrils within the NI consistent with amyloid-like deposition.

An important, unexplained feature of glutamine-repeat diseases is the differing neuronal selectivity among the various diseases. Each disease is characterized by distinct, yet overlapping, patterns of neurodegeneration. Selective patterns of neurodegeneration occur despite the fact that the disease proteins tend to be widely expressed in the brain and elsewhere in the body. Several factors may contribute to cell specificity, including the particular protein context within which the glutamine resides, specific interactions with other proteins whose expression is spatially or temporally restricted, and posttranslational modifications. It has been shown that huntingtin and DRPLA (dentatorubral-pallidoluysian atrophy) proteins were able to interact selectively with the enzyme GADPH. Moreover, a huntingtin-associated protein (HAP-1), whose expression is enriched in brain, has also been shown to bind to huntingtin, this binding being enhanced by an expanded polyglutamine repeat, the length of which is known to correlate with the age of disease onset.

The conformational structure of the triplet nucleotide repeats may also be involved in the development of the associated pathology. Computer modeling of the secondary structure of the huntin gtin mRNA predicts the formation of a stable stem-loop sequence encoded by the CAG repeat, which becomes more stable as the trinucleotide repeat is lengthened. Structures predicted by such modeling are useful in suggesting mRNA sequences that may be involved in regulating the expression of the mRNAs. Mc Laughlin et al. (1996) have identified cytoplasmic RNA-binding proteins that interact with trinucleotide CAG repeats in a tissue-specific and CAG length-dependent manner, using RNA probes designed on the basis of the Huntington disease gene sequence. Three speculative models have been hypothesized by these authors, which are the followings: (1) RNA-binding protein interaction with CAG repeats of huntingtin mRNA may alter the amount of huntingtin protein produced; (2) the protein-RNA interaction may affect the subcellular distribution of the huntingtin mRNA; or (3) the RNA-protein interaction may facilitate the altered expression of other proteins.

On the other hand, a defective gene involved in brain disorder is not necessarily associated with the presence of trinucleotide repeats in its coding sequence. This is the case, for example, for a gene involved in the X-linked hypohydrotic ectodermal dysplasia (HED) that has been recently isolated and which does not contains any repeat in its coding sequence, and which has been named TED (Genebank Accession number AF087142). Hypohydrotic ectodermal dysplasia (HED) affected males show mental defects, such as moderately severe mental retardation, which may be associated with hypotrichosis, abnormal teeth, and absent sweat glands.

There is a strong need in the art to identify new genes and new proteins that are likely to be involved in the development of diseases affecting the central nervous system, both for diagnostic and therapeutic purposes. Some typical candidate genes are those harboring CAG nucleotide repeats in their coding sequences.

Among the central nervous system diseases, schizophrenia is one of the most severe and debilitating. It usually starts in late adolescence or early adult life and often becomes chronic and disabling. Men and women are at equal risk of developing this illness; however, most males become ill between 16 and 25 years old; females develop symptoms between 25 and 30.

People with schizophrenia often experience both “positive” symptoms (delusions, hallucinations, disorganized thinking, agitation) and “negative” symptoms (lack of drive or initiative, social withdrawal, apathy, emotional unresponsiveness).

Schizophrenia affects 1% of the world population. There is an estimated 45 million people with schizophrenia in the world, more than 33 million of them in the developing countries.

This disease places a heavy burden on the patient's family and relatives, both in terms of the direct and indirect costs involved and the social stigma associated with the illness, sometimes over generations. Such stigma often leads to isolation and neglect.

Moreover, schizophrenia accounts for a fourth of all mental health costs and takes up one in three psychiatric hospital beds. Most schizophrenia patients are never able to work. The cost of schizophrenia to society is enormous. In the United States, for example, the direct cost of treatment of schizophrenia has been estimated to be close to 0.5% of the gross national product.

Standardized mortality ratios (SMRs) for schizophrenic patients are estimated to be two to four times higher than the general population, and their life expectancy overall is 20% shorter than for general population. The most common cause of death (in 10% of patients), is suicide—the risk is 20 times higher than for the general population. Deaths from heart disease and from diseases of the respiratory and digestive system are also increased among schizophrenic patients.

There is no cure for schizophrenia. The objective of treatment is to reduce the severity of the symptoms, if possible to the point of remission. Antipsychotic medications are the most common and most valuable treatment for schizophrenia. They can be described through four drugs.

The initial drug, chlorpromazine (Thorazine), has revolutionized the treatment of schizophrenic patients by reducing positive (psychotic) symptoms and preventing their recurrence. Patients have been able to leave mental hospitals and live in community programs or their own homes. But these drugs are far from ideal. Some 20% to 30% of patients do not respond to them at all, and others eventually relapse. The drugs are known as neuroleptics because they produce serious neurological side effects, including rigidity and tremors in the arms and legs, muscle spasms, abnormal body movements, and akathisia (restless pacing and fidgeting). These side effects are so troublesome that many patients simply refuse to take the drugs. Besides, neuroleptics do not improve the so-called negative symptoms of schizophrenia and the side effects may even exacerbate these symptoms. Thus, despite the clear beneficial effects of the drugs, even some patients who have a good short-term response will ultimately deteriorate in overall functioning.

These deficiencies of the standard neuroleptics have stimulated a search for new treatments which leads to a new class of drugs named atypical neuroleptics. The first atypical neuroleptic, Clozapine, is effective for about one third of patients who do not respond to standard drugs. It seems to reduce negative as well as positive symptoms, or at least exacerbates negative symptoms less than standard drugs do. Moreover, it has beneficial effects on overall functioning and may reduce the chance of suicide in schizophrenic patients. It does not produce the troubling neurological symptoms of the standard neuroleptics and raise blood levels of the hormone prolactin, excess of which may cause menstrual irregularities and infertility in women, impotence or breast enlargement in men. Many patients who cannot tolerate standard neuroleptics are able to take clozapine. However, clozapine has serious limitations. It was originally withdrawn from the market because it can cause agranulocytosis, a potentially lethal failure of the capacity to produce white blood cells. Agranulocytosis remains a threat that requires careful monitoring and periodic blood tests. Clozapine can also cause seizures and other disturbing side effects—drowsiness, lowered blood pressure, drooling, bed-wetting, and weight gain. Thus it is usually taken only by patients who do not respond to other drugs.

Researchers have developed new antipsychotic drugs that have the virtues of clozapine without its defects. One of these drugs is risperidone (Risperdal). Early studies suggest that it is as effective as standard neuroleptic drugs for positive symptoms and may be somewhat more effective for negative symptoms. It produces more neurological side effects than clozapine but fewer than standard neuroleptics. However, it raises prolactin levels. Risperidone is now prescribed for a broad range of psychotic patients, and many clinicians seem to use it before clozapine for patients who do not respond to standard drugs, because they regard it as safer. Another one is Olanzapine (Zyprexa) which is at least as effective as standard drugs for positive symptoms and more effective for negative symptoms. It has few neurological side effects at ordinary clinical doses, and it does not significantly raise prolactin levels. Although it does not produce most of clozapine's most troubling side effects, including agranulocytosis, some patients taking olanzapine may become sedated or dizzy, develop dry mouth, or gain weight. In rare cases liver function tests become transiently abnormal.

Outcome studies in schizophrenia are usually based on hospital treatment samples and may not be representative of the population of schizophrenia patients. At the extremes of outcome, 20% of patients seem to recover completely after one episode of psychosis, whereas 14-19% of patients develop a chronic unremitting psychosis and never fully recover. In general, clinical outcome at five years seems to follow the rule of thirds: with about 35% of patients in the poor outcome category; 36% in the good outcome category, and the remainder with intermediate outcome. Prognosis in schizophrenia does not seem to worsen after five years.

Whatever the reasons, there is increasing evidence that leaving untreated for long periods early in course of the illness may negatively affect the outcome. However, their use is often delayed for patients experiencing a first episode of the illness. The patients may not realize that they are ill, or they may be afraid to seek help; family members sometimes hope the problem will simply disappear or cannot persuade the patient to seek treatment; clinicians may hesitate to prescribe antipsychotic medications when the diagnosis is uncertain because of potential side effects. Indeed, at the first manifestation of the disease, schizophrenia is difficult to distinguish from bipolar manic-depressive disorders, severe depression, drug-related disorders, and stress-related disorders. Since the optimum treatments differ among these diseases, the long term prognosis of the disorder also differs the beginning of the treatment.

All the known molecules used for the treatment of schizophrenia have side effects and act against the symptoms of schizophrenia. There is a strong need for new molecules devoid of side effects and directed against targets which are involved in causal events of schizophrenia. Therefore, tools allowing to find these targets are necessary and useful.

Schizophrenia is now considered to be a brain disease and emphasis is placed on biological determinants. Neuroimaging and neuropathological studies showed evidence of brain abnormalities in schizophrenic patients. The timing of these pathological changes is unclear but is likely to be a defect in early brain development. Profound changes have also occurred in hypotheses concerning neurotransmitter abnormalities in schizophrenia. The dopamine hypothesis has been extensively revised and is no longer considered as a primary causative model.

The aggregation of schizophrenia in families, the evidence from twin and adoption studies, and the lack of variation in incidence world wide, indicate that schizophrenia is primarily a genetic condition, although environmental risk factors are also involved at some level as necessary, sufficient, or interactive causes.

For example, schizophrenia occurs in 1% of the general population. But, if there is one grandparent with schizophrenia, the risk of getting the illness increases to about 3% ; one parent with Schizophrenia, to about 10%. When both parents have schizophrenia, the risk percentage rises to approximately 40%.

However, the persistence of schizophrenia in the population despite low fertility and high mortality, suggests that genetic transmission occurs principally through persons who do not have the illness.

Consequently, there is a strong need to identify genes involved in schizophrenia. The knowledge of these genes will permit to understand the schizophrenia etiology and could lead to drugs and medications which are directed against the cause of the disease and not only against their symptoms.

There is also a strong need for means for detecting a susceptibility to schizophrenia for preventing or following up the development of the disease. Diagnosis tools could be also useful. Indeed, early identification of subjects at risk of developing schizophrenia would enable early and/or prophylactic treatment o be given.

Moreover, a valuable assessment of the eventual efficacy of a medicament as well as the patent's eventual tolerance to it may permit to enhance the benefit/risk ratio of schizophrenia treatment.

### SUMMARY OF THE INVENTION

The present invention pertains to a nucleic acid molecule comprising the genomic sequence of a human gene harboring triplet nucleotide repeats, which is mainly expressed in brain, and which has been named G713 by the inventors. The G713 genomic sequence comprises regulatory sequences located both upstream (5′-end) and downstream (3′-end) of the transcribed portion of said gene, these regulatory sequences being also part of the invention.

The invention also deals with the complete cDNA sequence encoding the G713 protein, as well as with the corresponding translation product. Another object of the invention concerns the murine cDNA corresponding to the murine orthologue of the human G713 gene.

The invention is also directed to biallelic markers that are located within the G713 genomic sequence, these biallelic markers representing useful tools in order to identify a statistically significant association between specific alleles of G713 and one or several disorders, preferably brain disorders, and most preferably psychiatric disorders like schizophrenia and bipolar disorder.

Oligonucleotide probes or primers hybridizing specifically with a G713 genomic or cDNA sequence are also part of the present invention.

A further object of the invention consists of recombinant vectors comprising any of the nucleic acid sequences above described, and in particular of recombinant vectors comprising a G713 regulatory sequence or a sequence encoding a G713 protein, as well as of cell hosts comprising said nucleic acid sequences or recombinant vectors.

The invention is also directed to methods for the screening of substances or molecules modulating the expression of G713.

The present invention also comprises subject matter stemming from the identification of genetic associations between alleles of biallelic markers located on the human chromosome 13q31-q33 locus and a disease, as confirmed and characterized in a panel of human subjects. Based on the determination of this association, the invention provides a genetic association between alleles of biallelic markers located on the human chromosome 13q31-q33 locus and schizophrenia. The invention also provides appropriate tools for establishing further genetic associations between alleles of biallelic markers on the 13q31-13q33 locus and either side effects or benefits resulting from the administration of agents acting on schizophrenia or schizophrenia symptoms, like chlorpromazine, clozapine, risperidone, olanzapine, sertindole, quetiapine and ziprasidone. The invention also provides appropriate tools for establishing further genetic associations between alleles of biallelic markers on the 13q31-13q33 locus and a trait.

Methods and products are provided for the molecular detection of a genetic susceptibility in humans to schizophrenia. They can be used for diagnosis, staging, prognosis and monitoring of this disease, which processes can be further included within treatment approaches. The invention also provides for the efficient design and evaluation of suitable therapeutic solutions including individualized strategies for optimizing drug usage, and screening of potential new medicament candidates.

### BRIEF DESCRIPTION OF THE DRAWINGS

FIG. 1: Calculated physical properties of the human G713 protein.

FIG. 2: Prediction of the two-dimensional structure of the G713 protein, according to the method of Chou-Fasman.

FIG. 3: Prediction of the two-dimensional structure of the human G713 protein, according to the method of Garnier-Osguthorpe-Robson.

FIG. 4: Calculated physical properties of the mouse G713 protein.

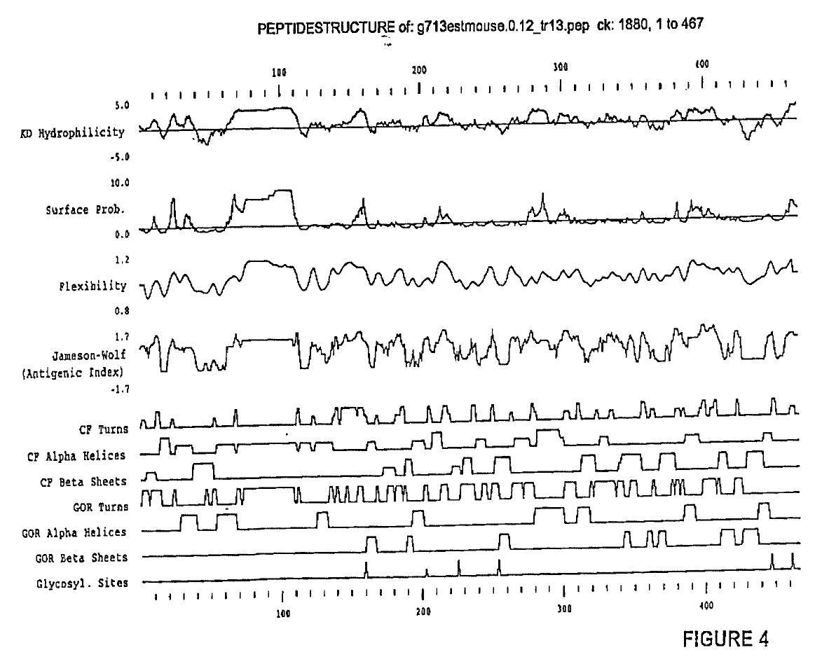

FIG. 5: Prediction of the two-dimensional structure of the mouse G713 protein, according to the method of Chou-Fasman.

FIG. 6: Prediction of the two-dimensional structure of the mouse G713 protein according to the method of Garnier-Osguthorpe-Robson.

FIG. 7: Block diagram of an exemplary computer system.

FIG. 8: Flow diagram illustrating one embodiment of a process 200 for comparing a new nucleotide or protein sequence with a database of sequences in order to determine the homology levels between the new sequence and the sequences in the database.

FIG. 9: Flow diagram illustrating one embodiment of a process 250 in a computer for determining whether two sequences are homologous.

FIG. 10: Flow diagram illustrating one embodiment of an identifier process 300 for detecting the presence of a feature in a sequence.

FIG. 11: Distribution of the different possible two markers-haplotypes ordered by decreasing ranges of p-values (increasing statistical significance).

FIG. 12: Distribution of the different possible three markers-haplotypes ordered by decreasing ranges of p-values (increasing statistical significance).

### DETAILED DESCRIPTION OF THE INVENTION

The present invention concerns polynucleotides and polypeptides related to the G713 human and mouse genes, which are potentially involved in brain disorders, particularly neuronal disorders like, for example schizophrenia or bipolar disorder.

The identification of genes involved in a particular trait such as a specific central nervous system disorder, like schizophrenia, can be carried out through two main strategies currently used for genetic mapping: linkage analysis and association studies. Linkage analysis requires the study of families with multiple affected individuals and is now useful in the detection of mono- or oligogenic inherited traits. Conversely, association studies examine the frequency of marker alleles in unrelated trait (T+) individuals compared with trait negative (T−) controls, and are generally employed in the detection of polygenic inheritance.

Candidate Region on the Chromosome 13 (Linkage Analysis)

The studies of genetic link or of “linkage” are based on the principle according to which two neighboring sequences on a chromosome do not present (or very rarely present) recombinations by crossing-over during meiosis. To do this, chromosomal markers, like microsatellite markers, have been localized with precision on the genome. Genetic link analysis calculates the probabilities of recombinations on the target gene with the chromosomal markers used, according to the genealogical tree, the transmission of the disease, and the transmission of the markers. Thus, if a particular allele of a given marker is transmitted with the disease more often than chance would have it (recombination level between 0 and 0.5), it is possible to deduce that the target gene in question is found in the neighborhood of the marker.

Using this technique, it has been possible to localize several genes of genetic predisposition to familial cancers. In order to be able to be included in a genetic link studies, the families affected by a hereditary form of the disease must satisfy the “informativeness” criteria: several affected subjects (and whose constitutional DNA is available) per generation, and at best having a large number of siblings.

By linkage analysis, a candidate region for schizophrenia has been identified on chromosome 13. Starting from the results of this linkage analysis, the inventors have identified a novel candidate gene for the predisposition to central nervous system disorders, like neuronal disorders such as schizophrenia or bipolar disorder, as it will be further described in details in the present specification. This gene has been named G713 by the inventors.

The G713 gene of the invention is located on chromosome 13, and more precisely on the 13q33 locus of this chromosome. Results of previous linkage studies have shown that chromosome 13 is likely to harbor a schizophrenia susceptibility locus on 13q32 (Blouin et al., 1998; Lin et al., 1997).

The G713 mRNA and the G713 protein share a significant homology with respectively the transcription and the translation products of a gene named TED which is involved in hypohydrotic ectodermal dysplasia, a disease associated with mental retardation. More precisely, the strongest homology found between the two mRNAs is of about 66% nucleotide identity in a stretch of 398 consecutive nucleotides of each of the G713 and the TED mRNAs, without any gap. The strongest protein homology between the G713 and the TED proteins is of 85% amino acid identity in a stretch of 39 consecutive amino acids of each protein, without any gap.

Consequently, one aim of the present invention is to provide for polynucleotides derived from the G713 gene, particularly those useful to design suitable means for detecting the presence of this gene in a test sample or alternatively the G713 mRNA molecules that are present in a test sample. Other polynucleotides of the invention are useful to design suitable means to express a desired polynucleotide of interest. The invention also relates to a G713 polypeptide.

Linkage analyses such as those noted above which led to the observation of a candidate region for schizophrenia on the chromosome 13q32 locus (Blouin et al., 1998) have generally been applied to map simple genetic traits that show clear Mendelian inheritance patterns and which have a high penetrance, but this method suffers from a variety of drawbacks. First, linkage analysis is limited by its reliance on the choice of a genetic model suitable for each studied trait. Furthermore, the resolution attainable using linkage analysis is limited, and complementary studies are required to refine the analysis of the typical 20 Mb regions initially identified through this method. In addition, linkage analysis have proven difficult when applied to complex genetic traits, such as those due to the combined action of multiple genes and/or environmental factors. In such cases, too large an effort and cost are needed to recruit the adequate number of affected families required for applying linkage analysis to these situations. Finally, linkage analysis cannot be applied to the study of traits for which no large informative families are available.

In addition to providing the G713 polynucleotides and polypeptides discussed above, the present inventors have also discovered alternative means in order to conduct association studies rather than linkage analysis between markers located on the chromosome 13q31-q33 locus and a trait, preferably schizophrenia. More particularly, the inventors have identified biallelic markers and sets of biallelic markers located on the human chromosome 13q31-q33, which biallelic markers or set of biallelic markers have one allele or haplotypes associated with schizophrenia, as it will be further described in details in the present specification. The identification of these biallelic markers in association with schizophrenia has allowed them to narrow the chromosomal region suspected to contain a genetic determinant involved in predisposition to schizophrenia from about 20 Mb to about 2 Mb. The determination of a narrow chromosomal region harboring a genetic determinant involved in predisposition to schizophrenia was the necessary step towards the identification of the causal or co-factor gene located therein. The borders of this region are defined by two AFM genetic markers: AFM248tf1-D13S174 and AFM102xd12-D13S1311, the nucleotide sequences of these markers being both publicly available in the Genbank database.

The association found between the biallelic markers described herein and predisposition to schizophrenia represent a strong presumption on the presence of at least one schizophrenia predisposition gene in this particular genomic region.

These identified polymorphisms are used in the design of assays for the reliable detection of genetic susceptibility to schizophrenia. They can also be used in the design of drug screening protocols to provide an accurate and efficient evaluation of the therapeutic and side-effect potential of new or already existing.

Definitions

Before describing the invention in greater detail, the following definitions are set forth to illustrate and define the meaning and scope of the terms used to describe the invention herein.

Unless otherwise indicated, G713 is used throughout the present description to designate a nucleic acid derived from the human G713 genomic or mRNA molecules.

The term “heterologous protein”, when used herein, is intended to designate any protein or polypeptide other than the G713 protein. More particularly, the heterologous protein is a compound which can be used as a marker in further experiments with a G713 regulatory region.

The term “isolated” requires that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “purified” does not require absolute purity; rather, it is intended as a relative definition. Purification of starting material or natural material to at least one order of magnitude, preferably two or three orders, and more preferably four or five orders of magnitude is expressly contemplated. As an example, purification from 0.1% concentration to 10% concentration is two orders of magnitude.

Throughout the present specification, the expression “nucleotide sequence” may be employed to designate indifferently a polynucleotide or a nucleic acid. More precisely, the expression “nucleotide sequence” encompasses the nucleic material itself and is thus not restricted to the sequence information (i.e. the succession of letters chosen among the four base letters) that biochemically characterizes a specific DNA or RNA molecule.

The term “polynucleotide” is understood to mean deoxyribonucleic acid or ribonucleic acid fragments or, more generally, polynucleotides or oligonucleotides where the bases, inter-nucleotide phosphate linkages, or alternatively the ribose rings of the bases, can be chemically modified in a known manner. This may be especially oligonucleotides with α or β anomers, oligonucleotides with inter-nucleotide linkage of the phosphorothioate or methyl phosphonate type, or alternatively oligothionucleotide.

As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)₂, and F(ab′)₂ fragments.

As used herein, an “antigenic determinant” is the portion of an antigen molecule, in this case a G713 polypeptide, that determines the specificity of the antigen-antibody reaction. An “epitope” refers to an antigenic determinant of a polypeptide. An epitope can comprise as few as 3 amino acids in a spatial conformation which is unique to the epitope. Generally an epitope comprises at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by Geysen et al. 1984; PCT Publication No. WO 84/03564; and PCT Publication No. WO 84/03506, the disclosures of which are herein incorporated by reference in their entireties.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is the replacement of one nucleotide by another nucleotide at the polymorphic site. Deletion of a single nucleotide or insertion of a single nucleotide also gives rise to single nucleotide polymorphisms. In the context of the present invention, “single nucleotide polymorphism” preferably refers to a single nucleotide substitution. Typically, between different individuals, the polymorphic site may be occupied by two different nucleotides.

The term “biallelic polymorphism” and “biallelic marker” are used interchangeably herein to refer to a single nucleotide polymorphism having two alleles at a fairly high frequency in the population. A “biallelic marker allele” refers to the nucleotide variants present at a biallelic marker site. Typically, the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker”.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

As used herein, the term “G713-related biallelic marker” relates to a set of biallelic markers in linkage disequilibrium with the G713 gene or a G713 nucleotide sequence. The term G713-related biallelic marker encompasses the biallelic markers A1 to A11 disclosed in Table 2 and any biallelic markers in linkage disequilibrium therewith. The preferred G713-related biallelic marker alleles of the present invention include each one the alleles described in Table 2 individually or in groups consisting of all the possible combinations of the alleles listed.

As used herein, the term “13q31-qc33-related biallelic marker” relates to a set of biallelic markers residing in the human chromosome 13q31-q33 region. The term 13q31-q33-related biallelic marker encompasses all of the biallelic markers A12 to A49 disclosed in Table 7 as well as biallelic markers in linkage disequilibrium therewith. The preferred chromosome 13q31-q33-related biallelic marker alleles of the present invention include each one the alleles described in Table 7 individually or in groups consisting of all the possible combinations of the alleles listed.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined hereinbelow) which can be used to identify a specific polynucleotide sequence present in samples, said nucleic acid segment comprising a nucleotide sequence complementary of the specific polynucleotide sequence to be identified.

The terms “trait” and “phenotype” are used interchangeably herein and refer to any visible, detectable or otherwise measurable property of an organism such as symptoms of, or susceptibility to a disease for example. Typically the terms “trait” or “phenotype” are used herein to refer to symptoms of, or susceptibility to a disease, a beneficial response to or side effects related to a treatment. Preferably, said trait can be, without to be limited to, cancers, developmental diseases, and neurological diseases.

The term “allele” is used herein to refer to variants of a nucleotide sequence. A biallelic polymorphism has two forms. Diploid organisms may be homozygous or heterozygous for an allelic form.

The term “heterozygosity rate” is used herein to refer to the incidence of individuals in a population, which are heterozygous at a particular allele. In a biallelic system the heterozygosity rate is on average equal to 2Pₐ(1-Pₐ), where Pₐ is the frequency of the least common allele. In order to be useful in genetic studies a genetic marker should have an adequate level of heterozygosity to allow a reasonable probability that a randomly selected person will be heterozygous.

The term “genotype” as used herein refers the identity of the alleles present in an individual or a sample. In the context of the present invention a genotype preferably refers to the description of the biallelic marker alleles present in an individual or a sample. The term “genotyping” a sample or an individual for a biallelic marker consists of determining the specific allele or the specific nucleotide(s) carried by an individual at a biallelic marker.

The term “mutation” as used herein refers to a difference in DNA sequence between or among different genomes or individuals which has a frequency below 1%.

The term “haplotype” refers to a combination of alleles present in an individual or a sample on a single chromosome. In the context of the present invention a haplotype preferably refers to a combination of biallelic marker alleles found in a given individual and which may be associated with a phenotype.

The term “upstream” is used herein to refer to a location which, is toward the 5′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4th edition, 1995).

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

As used herein, the term “operably linked” refers to a linkage of polynucleotide elements in a functional relationship. For instance, a promoter or enhancer is operably linked to a coding sequence if it affects the transcription of the coding sequence.

Variants and Fragments

1-Polynucleotides

The invention also relates to variants and fragments of the polynucleotides described herein, particularly of a G713 or a 13q31-q33 polynucleotide, and particularly of a G713 or a 13q31-q33 polynucleotide containing one or more biallelic markers according to the invention.

Variants of polynucleotides, as the term is used herein, are polynucleotides that differ from a reference polynucleotide. A variant of a polynucleotide may be a naturally occurring variant such as a naturally occurring allelic variant, or it may be a variant that is not known to occur naturally. Such non-naturally occurring variants of the polynucleotide may be made by mutagenesis techniques, including those applied to polynucleotides, cells or organisms. Generally, differences are limited so that the nucleotide sequences of the reference and the variant are closely similar overall and, in many regions, identical.

Changes in the nucleotide of a variant may be silent, which means that they do not alter the amino acids encoded by the polynucleotide.

However, nucleotide changes may also result in amino acid substitutions, additions, deletions, fusions and truncations in the polypeptide encoded by the reference sequence. The substitutions, deletions or additions may involve one or more nucleotides. The variants may be altered in coding or non-coding regions or both. Alterations in the coding regions may produce conservative or non-conservative amino acid substitutions, deletions or additions.

In the context of the present invention, particularly preferred embodiments of a G713 polynucleotide are those in which the polynucleotides encode polypeptides which retain substantially the same biological function or activity as the mature G713 protein.

A G713 polynucleotide fragment is a polynucleotide having a sequence that entirely is the same as part but not all of a given nucleotide sequence, preferably the nucleotide sequence of a G713 gene, and variants thereof. The fragment can be a portion of an exon or of an intron of a G713 gene. It can also be a portion of the regulatory sequences of the G713 gene, preferably of the promoter. Preferably, such fragments comprise at least one of the biallelic markers A1 to A11 or a biallelic marker in linkage disequilibrium with one or more of the biallelic markers A1 to A11.

Variants of G713 and 13q31-q33 polynucleotides according to the invention include, without being limited to, nucleotide sequences at least 95% identical to a nucleic acid selected from the group consisting of SEQ ID Nos 1-4, 6 and 31-69 or to any polynucleotide fragment of at least 8 consecutive nucleotides from a nucleic acid selected from the group consisting of SEQ ID Nos 1-4, 6 and 31-69 and preferably at least 99% identical, more particularly at least 99.5% identical, and most preferably at least 99.8% identical to a nucleic acid selected from the group consisting of SEQ ID Nos 1-4, 6 and 31-69 or to any polynucleotide fragment of at least 8 consecutive nucleotides of these nucleic acids.

Such fragments may be “free-standing”, i.e. not part of or fused to other polynucleotides, or they may be comprised within a single larger polynucleotide of which they form a part or region. However, several fragments may be comprised within a single larger polynucleotide.

As representative examples of polynucleotide fragments of the invention, there may be mentioned those which have from about 4, 6, 8, 15, 20, 25, 40, 10 to 30, 30 to 55, 50 to 100, 75 to 100 or 100 to 200 nucleotides in length. Preferred are those fragments having about 47 nucleotides in length and containing at least one of the G713 or 13q31-q33 biallelic markers which are described herein. It will of course be understood that the polynucleotides of SEQ ID 1-4, 6 and 31-69 can be shorter or longer, although it is preferred that they at least contain the biallelic marker of the primer which can be located at one end of the fragment.

2-Polypeptides

The invention also relates to variants, fragments, analogs and derivatives of the polypeptides described herein, including mutated human and mouse G713 proteins.

The variant may be 1) one in which one or more of the amino acid residues are substituted with a conserved or non-conserved amino acid residue and such substituted amino acid residue may or may not be one encoded by the genetic code, or 2) one in which one or more of the amino acid residues includes a substituent group, or 3) one in which the mutated G713 is fused with another compound, such as a compound to increase the half-life of the polypeptide (for example, polyethylene glycol), or 4) one in which the additional amino acids are fused to the mutated G713, such as a leader or secretory sequence or a sequence which is employed for purification of the mutated G713 or a preprotein sequence. Such variants are deemed to be within the scope of those skilled in the art.

A polypeptide fragment is a polypeptide having a sequence that entirely is the same as part but not all of a given polypeptide sequence, preferably a polypeptide encoded by a G713 gene and variants thereof.

Such fragments may be “free-standing”, i.e. not part of or fused to other polypeptides, or they may be comprised within a single larger polypeptide of which they form a part or region. However, several fragments may be comprised within a single larger polypeptide.

As representative examples of polypeptide fragments of the invention, there may be mentioned those which have from about 5, 6, 7, 8, 9 or 10 to 15, 10 to 20, 15 to 40, or 30 to 55 amino acids long. Preferred are those fragments containing at least one amino acid mutation in the G713 protein.

More particularly, a variant G713 polypeptide comprises amino acid changes ranging from 1, 2, 3, 4, 5, 10 to 20 substitutions, additions or deletions of one amino acid, preferably from 1 to 10, more preferably from 1 to 5 and most preferably from 1 to 3 substitutions, additions or deletions of one amino acid. The preferred amino acid changes are those which have little or no influence on the biological activity or the capacity of the variant G713 polypeptide to be recognized by antibodies raised against a native G713 protein.

By homologous peptide according to the present invention is meant a polypeptide containing one or several amino acid additions, deletions and/or substitutions in the amino acid sequence of a G713 polypeptide. In the case of an aminoacid substitution, one or several -consecutive or non-consecutive-amino acids are replaced by “equivalent” amino acids.

The expression “equivalent” amino acid is used herein to designate any amino acid that may be substituted for one of the amino acids having similar properties, such that one skilled in the art of peptide chemistry would expect the secondary structure and hydropathic nature of the polypeptide to be substantially unchanged. Generally, the following groups of amino acids represent equivalent changes: (1) Ala, Pro, Gly, Glu, Asp, Gln, Asn, Ser, Thr; (2) Cys, Ser, Tyr, Thr; (3) Val, lle, Leu, Met, Ala, Phe; (4) Lys, Arg, His; (5) Phe, Tyr, Trp, His.

By an equivalent aminoacid according to the present invention is also meant the replacement of a residue in the L-form by a residue in the D form or the replacement of a Glutamic acid (E) residue by a Pyro-glutamic acid compound. The synthesis of peptides containing at least one residue in the D-form is, for example, described by Koch (1977).

A specific, but not restrictive, embodiment of a modified peptide molecule of interest according to the present invention, which consists in a peptide molecule which is resistant to proteolysis, is a peptide in which the —CONH— peptide bond is modified and replaced by a (CH₂NH) reduced bond, a (NHCO) retro inverso bond, a (CH₂—O) methylene-oxy bond, a (CH₂—S) thiomethylene bond, a (CH₂CH₂) carba bond, a (CO—CH₂) cetomethylene bond, a (CHOH—CH₂) hydroxyethylene bond), a (N—N) bound, a E-alcene bond or also a —CH═CH— bond.

The polypeptide according to the invention could have post-translational modifications. For example, it can present the following modifications: acylation, disulfide bond formation, prenylation, carboxymethylation and phosphorylation.

Complementary Polynucleotides

For the purpose of the present invention, a first polynucleotide is deemed to be complementary to a second polynucleotide when each base in the first polynucleotide is paired with its complementary base. Complementary bases are, generally, A and T (or A and U), or C and G.

Identity Between Nucleic Acids or Polypeptides

The terms “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology is evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, 1988; Altschul et al., 1990; Thompson et al., 1994; Higgins et al., 1996; Altschul et al., 1993). In a particularly preferred embodiment, protein and nucleic acid sequence homologies are evaluated using the Basic Local Alignment Search Tool (“BLAST”) which is well known in the art (see, e.g., Karlin and Altschul, 1990; Altschul et al., 1990, 1993, 1997). In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., 1992; Henikoff and Henikoff, 1993). Less preferably, the PAM or PAM250 matrices may also be used (see, e.g., Schwartz and Dayhoff, eds., 1978). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul, 1990).

Stringent Hybridization Conditions

By way of example and not limitation, procedures using conditions of high stringency are as follows: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in prehybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. Other conditions of high stringency which may be used are well known in the art and as cited in Sambrook et al., 1989; and Ausubel et al., 1989, are incorporated herein in their entirety. These hybridization conditions are suitable for a nucleic acid molecule of about 20 nucleotides in length. There is no need to say that the hybridization conditions described above are to be adapted according to the length of the desired nucleic acid, following techniques well known to the one skilled in the art. The suitable hybridization conditions may for example be adapted according to the teachings disclosed in the book of Hames and Higgins (1985) or in Sambrook et al.(1989).

BRIEF DESCRIPTION OF THE SEQUENCES PROVIDED IN THE SEQUENCE
LISTING

| SEQ ID | DESCRIPTION |
|---|---|
| 1 | 5′-regulatory region + Exon 1 + 5′-end of Intron 1 of hG713 |
| 2 | 3′-end of Intron 1 + Exon 2 of human G713 + 5′-end of Intron 2 of hG713 |
| 3 | 3′-end of Intron 2 + Exon 3 + 3′-regulatory region of hG713 |
| 4 | cDNA of hG713 |
| 5 | Protein encoded by the cDNA of SEQ ID No 4 |
| 6 | cDNA of the mouse G713 |
| 7 | Protein encoded by the cDNA of SEQ ID No 6 |
| 8-25 | Primers used for isolating the G713 cDNA |
| 26-30 | Primers used for isolating the mG713 cDNA |
| 31 | Candidate genomic nucleotide sequence located in the region of the |
| | biallelic markers associated with schizophrenia and containing a |
| | sequence specifically expressed in individuals affected by schizophrenia. |
| 32-69 | Amplification fragments containing the nucleotide sequence of the |
| | amplicons which comprise the biallelic markers A12 to A49 located on the |
| | human chromosome 13q31-q33 locus. |
| 70 | SEQ ID No PU contains a primer containing the additional PU 5′ |
| | sequence described further in Examples 1(c) and 2(b) |
| 71 | SEQ ID No RP contains a primer containing the additional RP 5′ |
| | sequence described further in Examples 1(c) and 2(b) |

In accordance with the regulations relating to Sequence Listings, the following codes have been used in the Sequence Listing to indicate the locations of biallelic markers within the sequences and to identify each of the alleles present at the polymorphic base. The code “r” in the sequences indicates that one allele of the polymorphic base is a guanine, while the other allele is an adenine. The code “y” in the sequences indicates that one allele of the polymorphic base is a thymine, while the her allele is a cytosine. The code “m” in the sequences indicates that one allele of the polymorphic base is an adenine, while the other allele is an cytosine. The code “k” in e sequences indicates that one allele of the polymorphic base is a guanine, while the her allele is a thymine. The code “s” in the sequences indicates that one allele of the polymorphic base is a guanine, while the other allele is a cytosine. The code “w” in the sequences indicates that one allele of the polymorphic base is an adenine, while the her allele is an thymine. The nucleotide code of the original allele at each biallelic marker position has been designated “allele 1” in Tables 2 and 7, and the alternative allele has been designated “allele 2” in Tables 2 and 7.

In some instances, the polymorphic bases of the biallelic markers alter the identity of an amino acids in the encoded polypeptide. This is indicated in the accompanying Sequence Listing by use of the feature VARIANT, placement of an Xaa at the position of the polymorphic amino acid, and definition of Xaa as the two alternative amino acids. For example if one allele of a biallelic marker is the codon CAC, which encodes histidine, while the other allele of the biallelic marker is CAA, which encodes glutamine, the Sequence Listing for the encoded polypeptide will contain an Xaa at the location of the polymorphic amino acid. In this instance, Xaa would be defined as being histidine or glutamine.

In other instances, Xaa may indicate an amino acid whose identity is unknown. In this instance, the feature UNSURE is used, placement of an Xaa at the position of the unknown amino acid and definition of Xaa as being any of the 20 amino acids or being unknown.

STRATEGY USED FOR IDENTIFYING BOTH mRNA AND GENOMIC SEQUENCES OF THE G713 GENE

a) Isolation of Partial G713 cDNA Molecules

Isolation of a First Partial cDNA (1.3 kb)

Starting from the results of linkage analysis indicating that a valuable central nervous system disorder candidate gene might be located on the chromosome 13q33 locus, an analysis of integrated data of the CEPH-Genethon human genome map (http://www.genethon.fr/genethon_en.html) with Genemap of the human genome (http:/Hwww.ncbi.nlm.nih.gov/SCIENCE96/) allowed the identification of several clusters of cDNA expressed at least in human brain and assigned to chromosome 13. None of the selected transcripts coded for known human genes. One of the selected transcripts has been chosen for further study. A portion of this cDNA fragment is present in an anonymous EST cDNA clone—clone 46473—belonging to the Soares cDNA library of infant brain. Two end sequences of this clone are referenced in the Genbank database, respectively under the accession numbers H09867 (5′-end sequence of clone 46473) and H09780 (3′-end sequence of clone 46473). These end sequences were used to design the two following primers:

Forward primer g713LF1, designed from the sequence of Genbank Accession number H089867: 5′-CGCTTGCTTCTGTCTGTGTAACC-3′ (SEQ ID No 8), and

Reverse primer g713LR1, designed from the sequence of Genbank Accession number H09780: 5′-GTATTTGCGCAGACCATTTTAAGATT-3′ (SEQ ID No 9).

5-extension of the First Partial cDNA

A Long Range PCR amplification of the cDNA from the human fetal brain Marathon™ ready library (Clontech, Palo Alto, Calif., USA, Cat. No. 7402-1) was performed with the pair of primers g713LF1 and g713LR1. A cDNA fragment of a length of 1.3 kb was amplified, said amplified fragment being subsequently cloned in the proprietary pGenDel vector (U.S. patent application Ser. No. 09/058,746, filed Apr. 10, 1998). The insert has been sequenced by several cycles of primer walking. Sequencing confirmed the presence of structures identical to the sequences of Genbank accession numbers H09867 and H09780, respectively at the 5′-end and at the 3′-end of this 1.3 kb cDNA fragment. Analysis of the entire sequence of the 1.3 kb cDNA fragment showed the absence of any potential coding Open Reading Frame.

In order to isolate the complete 5′ portion of the cDNA containing the above 1.3 kb fragment, a RACE (Rapid Amplification of cDNA Ends) has been performed on the cDNA from the human fetal brain Marathon™ ready library using the following primers:

Primer 713.LF1.5.1: 5′-ACTGTCTGATTCCACCTATTATGGAG-3′ (SEQ ID No 10), and

Primer g713.LF1.5.1n: 5′-TGATTCCACCTATTATGGAGAGCAC-3′ (SEQ ID No 11).

Amplification led to the production of a heterogeneous product that has been sequenced with the following nested primer:

Primer g713RACE5R1: 5′-GGGTAGAAGGGAGACTTAGG-3′ (SEQ ID No 12). Sequencing gave a 68 bp sequence of very poor quality that contains AT rich repeats.

Another sequencing primer was designed from the 68 bp sequence, which is the following:

Primer g713RACE5R-49: 5′-GGGCATAGCAATCATTC-3′ (SEQ ID No 13).

This primer has been successfully used to determine the partial sequence of the amplified product resulting from the 5′-RACE reaction. This partial sequence has been compared with the nucleotide sequences referenced in Genbank and has been found to be highly homologous to a partial transcript named CTG-A4 (Genbank Accession Number L10374) containing CTG repeats.

Isolation of a 3.2 kb G713 cDNA Molecule

cDNA from the human fetal brain Marathon™ ready library was amplified with the following pair of primers:

Primer derived from the 5′-end of the CTG-A4 sequence (g713CTGLF132) 5′-GGCTGTGCGTTCCCAAAATA-3′ (SEQ ID No 14); and

Primer derived from the 3′ end of the previously sequenced 1.3 kb cDNA fragment (g713LR1): 5′-GTATTTGCGCAGACCATTTTAAGATT-3′ (SEQ ID No 9).

The amplification reaction yielded to a 3.2 kb cDNA fragment that has been sequenced by primer walking and sub-cloning. Physical linkage between the CTG-A4 fragment and the 1.3 kb fragment was confirmed and a new AT rich repeat between them was identified and sequenced.

3′-extension of the First Partial cDNA

In order to amplify cDNA extending towards the 3′-end of the first partial cDNA, the following primers derived from the 3′-end of the 3.2 kb cDNA described above have been designed:

Primer (g713RACE3N): 5′-AAAAATGTTTCGTTCCAGTCTGTTAAGA-3′ (SEQ ID No 15); and

Primer (g713RACE3Nn): 5′-ATTGCTAGAATTGTTTAGCAGTACATGCA-3′ (SEQ ID No 16).

The amplification reaction of the cDNA from the human fetal brain Marathon™ ready library yielded to a 2.5 kb cDNA fragment. A partial sequence of this 2.5 kb cDNA fragment presented a high homology with two ESTs referenced in Genbank under the Accession numbers AA424106 and AA424056. ESTs AA424106 and AA424056 are respectively the 5′-end sequence and the 3′-end sequence of the cDNA clone no. 759953 from the Soares total fetus Nb2HF8 9w human cDNA library. It was found that this publicly available clone terminates in a poly-A tract and contains a polyadenylation signal.

Isolation of a Longer G713 cDNA (First Attempt to Isolate the Full Length G713 cDNA)

A first strand cDNA synthesis specific primer has been designed from the 3′-end sequence of the cDNA clone n° 759953, this primer (SG1polyA) sequence being the following:

5′-TTTTTTTTTTTTTGACAGAG-3′ (SEQ ID No 17). A cDNA has been synthesized with the SG1polyA primer, using as template a human fetal brain mRNA library (Clontech, Palo Alto, Calif., USA, Cat. Ref. 64019-1). The resulting cDNA produced has then been used as a substrate for a Long Range PCR amplification with the following pair of primers:

Primer g713CTGLF132 described above, derived from the 5-end of the G713 transcript: 5′-GGCTGTGCGTTCCCAAAATA-3′ (SEQ ID No 14); and

Primer SG1 LR100 derived from the Genbank nucleic acid sequence referenced under the accession number AA424056: 5′-TTTGCCATTTAGCTTAGCAGTACCA-3′ (SEQ ID No 18).

The Long Range PCR amplification reaction yielded to a cDNA fragment of 4.5 kb in length that has been sequenced by primer walking with specially designed specific primers.

b) Isolation of the G713 Genomic Sequences

A BAC library covering the whole human genome has been screened with the two following STSs:

STS-g713, derived from the 3′-end of the above described 4.5 kb transcript, which is amplified by the following pairs of primers:

Primer 1: 5′-AATATTCTTAACAGACTGGAAC-3′ (SEQ ID No 19);

Primer 2: 5′-CTTTATAGCTATGAAATTTCCC-3′ (146 55) (SEQ ID No 20); and

STS g34301, derived from the 5′half of the above described 4.5 kb transcript and containing CAG repeats, this STS being amplified by the following pair of primers:

Primer 1: 5′-CTGATCACTTGTGGTTCTGCGCCG-340 (SEQ ID No 21);

Primer 2: AGGACTCCCCCATGCTCGCCAG-3′ (183 67) (SEQ ID No 22).

Three positive BACs were selected after performing the screening with these two above STSs.

STS-g713 positive BAC no. B0106A08 was subdoned in the vector pGen Del (described in the U.S. patent application Ser. No. 09/058,746, filed Apr. 10, 1998) and has been sequenced. The G713 Exons and the 5′- and 3′-adjacent intronic sequences from BAC no. B0106A08 were sequenced directly with the help of the cDNA sequencing primers. BAC no. B0106A08 has been found to contain a portion of the first intron and the two last exons of the G713 gene.

STS-g34301 positive BACs no. B1090E12 and no. B0852B05 have been partially sequenced with the help of the g713 cDNA primers. Both BACs contain the first exon and a portion of the first intron of the G713 gene but do not contain any of the two last exons. The end sequences of the inserts from the BACs no. B0106A08, B1090E12 and B0852B05 were determined and were used to generate STSs for further screening of the BAC library in order to clone the entire intron 1.

c) Isolating the Full Length cDNA of G713

Sequences immediately upstream of the above described G713 transcript have been determined by several rounds of primer walking using BAC DNA of either BAC no. B1090E12 or no. B0852B05. Complex repeats were found in these regions, which explain the previous failure of the inventors to sequence the 5′-end of the G713 cDNA by RACE PCR, as described hereinbefore.

A series of Long Range PCR primers was generated from this region and was used in combination with the following primers:

Primer SG1LR1102, derived from Exon 2 of G713: 5′-AAAATACTGGGAACAGAGCCAGG-3′ (SEQ ID No: 23); and

Primer specific of SG1polyA: 5′-TTTTTTTTTTTTTGACAGAG-3′ (SEQ ID No: 17), in order to amplify a cDNA fragment containing Exon 1 and Exon 2 of the G713 cDNA.

This reconstruction experiments indicate that mRNA from the G713 gene starts at least few hundred bases upstream of the previously determined cDNA sequence. The last primer giving detectable amplification from G713 specific cDNA is Primer SG1LF790 (5′-GCACTTAGAGCGCGGGGT-3′-SEQ ID No 24).

The nearly full length clone of G713 has been produced by amplification from the first strand SG1polyA (5′-TTTTTTTTTTTTTGACAGAG-3′-SEQ ID No 17) specific DNA with the following primers:

Primer SG1LF834: 5′-GCCGGAGGCAGCCCA-3′ (SEQ ID No 25); and

Primer SG1LR100: 5′-TTTGCCATTTAGCTTAGCAGTACCA-3′ (SEQ ID No 18).

This molecule has been cloned and sequenced in order to confirm the deduced full transcript structure, which is described in the nucleic acid sequence of SEQ ID No 4.

G713 Genomic Polynucleotide, cDNA and Associated Regulatory Regions

G713 Genomic Sequences

The invention concerns a purified, isolated or recombinant nucleic acid encoding the G713 polypeptide. The present invention concerns the genomic sequence of G713, and in a particular aspect deals with a purified or isolated nucleic acid encoding a G713 polypeptide, wherein said nucleic acid comprises a polynucleotide comprising the whole exons of the G713 gene. In a specific embodiment, such a purified or isolated nucleic acid may comprise, consist essentially of, or consist of, from 5′-end to 3′-end, the polynucleotide of SEQ ID No 1, the polynucleotide of SEQ ID No 2, the polynucleotide of SEQ ID No 3.

The invention also encompasses a purified, isolated, or recombinant polynucleotide comprising a nucleotide sequence having at least 70, 75, 80, 85, 90, or 95% nucleotide identity with a nucleotide sequence of SEQ ID Nos. 1,2 or 3 or a complementary sequence thereto or a fragment thereof. The nucleotide differences as regards to the nucleotide sequence of SEQ ID Nos. 1, 2 or 3 may be generally randomly distributed throughout the entire nucleic acid. Nevertheless, preferred nucleic acids are those wherein the nucleotide differences as regards to the nucleotide sequence of SEQ ID Nos. 1,2 or 3 are predominantly located outside the coding sequences contained in the exons.

Another object of the invention consists of a purified, isolated, or recombinant nucleic acid that hybridizes with the nucleotide sequence of SEQ ID Nos. 1, 2 or 3 or a complementary sequence thereto or a variant thereof, under the stringent hybridization conditions as defined below.

Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID Nos. 1, 2 or 3 or the complements thereof, wherein said contiguous span comprises at least 1, 2, 3, 5, or 10 of the following nucleotide positions of SEQ ID Nos 1,2 and 3:

1 to 1000, 1001 to 2000, 2001 to 3000, 3001 to 4000 and 4001 to 5222 of SEQ ID No. 1;

1 to 1000, 1001 to 2000, 2001 to 3000, 3001 to 4000 and 4001 to 5000, 5001 to 6000, 6001 to 7000, 7001 to 8000, 8001 to 9000, 9001 to 10000, 10001 to 11000, 11001 to 12000, 12001 to 13000, 13001 to 14000, 14001 ot 15000, 15001 to 16000, 16001 to 17000, 17001 to 18000, 18001 to 19000, 19001 to 20000 and 20001 to 21278 of SEQ ID No 2; and

1 to 1000, 1001 to 2000, 2001 to 3000, 3001 to 4000 and 4001 to 5000, 5001 to 6000, 6001 to 7000, 7001 to 8000, 8001 to 9000, 9001 to 10000, 10001 to 11000, 11001 to 12000, 12001 to 13000, 13001 to 14000, 14001 ot 15000, 15001 to 16000, 16001 to 17000, 17001 to 18000, 18001 to 19000, 19001 to 20000, 20001 to 21000 and 21001 to 21636 of SEQ ID No 3.

Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID Nos 1, 2 or 3 or the complements thereof, wherein said contiguous span comprises at least one of the following nucleotide positions of:

SEQ ID No 1: 1 to 3236, 3547 to 3585 and 4649 to 5222, or a variant thereof or a sequence complementary thereto;

SEQ ID No 2: 1 to 16155 and 16331 to 21278 or a variant thereof or a sequence complementary thereto; and

SEQ ID No 3: 1 to 5531, 6844 to 7237, 7798 to 8184, 8667 to 9074, and 9356 to 21636, or a variant thereof or a sequence complementary thereto.

Further preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID Nos. 1, 2 or 3 or the complements thereof, wherein said contiguous span comprises a biallelic marker selected from the group of consisting of the biallelic markers A1 to A11. It should be noted that nucleic acid fragments of any size and sequence may also be comprised by the polynucleotides described in this section.

Further preferred embodiments of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1 or the complements thereof, wherein said contiguous span comprises the nucleotides AGAG at positions 3606 to 3609 of SEQ ID No 1.

As noted, the G713 genomic nucleic acid comprises 3 exons. Exon 1 starts at the nucleotide in position 3076 and ends at the nucleotide in position 4643 of the nucleotide sequence of SEQ ID No 1; exon 2 starts at the nucleotide in position 16157 and ends at the nucleotide in position 16329 of the nucleotide sequence of SEQ ID No 2; exon 3 starts at the nucleotide in position 5537 and ends at the nucleotide in position 9359 of the nucleotide sequence of SEQ ID No 3. Thus, the invention embodies purified, isolated, or recombinant polynucleotides comprising a nucleotide sequence selected from the group consisting of the 3 exons of the G713 gene, or a sequence complementary thereto. The invention also deals with purified, isolated, or recombinant nucleic acids comprising a combination of at least two exons of the G713 gene, wherein the polynucleotides are arranged within the nucleic acid, from the 5′-end to the 3′-end of said nucleic acid, in the same order as in SEQ ID Nos 1, 2 and 3.

The G713 genomic nucleic acid also comprises intronic polynucleotides that are located respectively at the 3′-end of Exon 1, both at the 5′-end and at the 3′-end of exon 2, and at the 5′-end of Exon 3, these intronic polynucleotides being respectively contained in the nucleic acids of SEQ ID Nos 1 to 3. The nucleic acids defining the G713 intronic polynucleotides, as well as their fragments and variants, may be used as oligonucleotide primers or probes in order to detect the presence of a copy of the G713 gene in a test sample, or alternatively in order to amplify a target nucleotide sequence within the G713 intronic sequences.

These nucleic acids of the invention, as well as their fragments and variants, may be used as oligonucleotide primers or probes in order to detect the presence of a copy of the G713 gene in a test sample, or alternatively in order to amplify a target nucleotide sequence within the G713 intronic sequences.

While this section is entitled “Genomic Sequences,” it should be noted that nucleic acid fragments of any size and sequence may also be comprised by the polynucleotides described in this section, flanking the genomic sequences of G713 on either side or between two or more such genomic sequences.

Human G713 cDNA

The inventors have discovered that the expression of the human G713 gene leads to the production of at least one mRNA molecule, the cDNA sequence of which is set forth in SEQ ID No 4.

A portion of a cDNA whose sequence is closely related to the G713 cDNA has been previously isolated by Li et al. (1993) and termed CTG-A4; the corresponding nucleotide sequence is referenced in the Genbank database as the accession number L10374. The sequence disclosed under the Genbank Accession Number L10374 has 99% nucleic acid homology with a portion of 2047 consecutive nucleotides of the G713 cDNA.

More precisely, Li et al. have screened a human brain cDNA library with a (CTG)₁₀ probe in order to clone the cDNA inserts that hybridize thereto. 40 positive clones were selected, one of which was named CTG-A4. The CTG-A4 insert was assigned to human chromosome 13. Among the 8 novel partial cDNAs isolated by Li et al., several have repeat lengths that are highly polymorphic, making them valuable as PCR typeable linkage markers. This is not the case for the CTG-A4 polynucleotide, that showed only a slight heterozygosity (20%) with only 2 alleles detected.

An object of the invention is thus a purified, isolated, or recombinant nucleic acid comprising the nucleotide sequence of SEQ ID No 4, complementary sequences thereto, as well as allelic variants, and fragments thereof. Moreover, preferred polynucleotides of the invention include purified, isolated, or recombinant G713 cDNAs consisting of, consisting essentially of, or comprising the sequence of SEQ ID No 4. Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4 or the complements thereof, wherein said contiguous span comprises at least one of the following nucleotide positions of SEQ ID No 4: 1 to 519 and 2563 to 5566. Additional preferred embodiments of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4 or the complements thereof, wherein said contiguous span comprises at least one of the following nucleotide positions of SEQ ID No 4: 1 to 166, 473 to 519, 3020 to 3445, 3990 to 4394 and 4874 to 5281.

The Open Reading Frame encoding the G713 protein spans from the nucleotide in position 659 and the nucleotide in position 2032 of the nucleotide sequence of SEQ ID No 4. A purified or isolated nucleic acid comprising the G713 ORF is an object of the present invention.

The cDNA of SEQ ID No 4 includes a 5′-UTR region. This 5′-UTR region starts from the nucleotide at position 1 and ends at the nucleotide in position 658 of SEQ ID No 4. The cDNA of SEQ ID No 4 includes a 3′-UTR region starting from the nucleotide at position 2033 and ending at the nucleotide at position 5566 of SEQ ID No 4. Consequently, the invention concerns a purified, isolated, and recombinant nucleic acid comprising a nucleotide sequence of the 5′UTR of the G713 cDNA, a sequence complementary thereto, or an allelic variant thereof. The invention also concerns a purified, isolated, and recombinant nucleic acid comprising a nucleotide sequence of the 3′UTR of the G713 cDNA, a sequence complementary thereto, or an allelic variant thereof.

The cDNA of SEQ ID No 4 harbors several polyadenylation signals, located at the following nucleotide positions of SEQ ID No 4: 2531 to 2536, 2538 to 2543, 2873 to 2878, 3307 to 3312, 3843 to 3848, 3859 to 3864, to 4524 to 4529 and 5536 to 5541.

Another object of the invention consists of a purified or isolated nucleic acid comprising the nucleotide sequence of SEQ ID No 4 or fragments thereof. Preferred G713 cDNA fragments are those located outside the Open Reading Frame, such as the 5′-UTR and the 3′-UTR nucleic acid sequences. The most preferred fragments of the nucleotide sequence of SEQ ID No 4 are comprised in the fragment located between the nucleotide in position 1 and the nucleotide in position 519 of the nucleotide sequence of SEQ ID No 4 and in the fragment located between the nucleotide in position 2563 and the nucleotide in position 5566 of the nucleotide sequence of SEQ ID No 4.

The invention also pertains to a purified or isolated nucleic acid having at least having at least 85, 90, 95, 97, 98 or 99% of nucleotide identity with the nucleotide sequence of SEQ ID No 4, preferably 99.5% and most preferably 99.8% nucleotide identity with the nucleotide sequence of SEQ ID No 4, or a sequence complementary thereto or a biologically active fragment thereof.

The nucleotide differences as regards to the nucleotide sequence of SEQ ID No 4 are generally randomly distributed throughout the entire nucleic acid. Nevertheless, preferred nucleic acids are those wherein the nucleotide differences as regards to the nucleotide sequence of SEQ ID No 4 are predominantly located outside the coding sequences, and more precisely in the 5′-UTR and the 3′-UTR sequences contained in the nucleotide sequence of SEQ ID No 4.

While this section is entitled “G713 cDNA”, it should be noted that nucleic acid fragments of any size and sequence may also be comprised by the polynucleotides described in this section, flanking the genomic sequences of G713 on either side or between two or more such genomic sequences.

Murine Othologue of G713

The inventors have also found that the murine genome harbored a gene that is orthologue to G713, which will also be termed murine G713 or mG713. More precisely, the inventors have isolated a murine mRNA containing an Open Reading Frame that share a strong nucleic aid homology with G713 and which encodes for a protein having about 88% amino acid identity with the G713 protein.

Thus, an object of the present invention concerns a purified or isolated nucleic acid comprising the nucleotide sequence of SEQ ID No 6, complementary sequences thereto, as well as allelic variants or fragments or variants thereof. Moreover, preferred polynucleotides of the invention include purified, isolated, or recombinant G713 cDNAs consisting of, consisting essentially of, or comprising the sequence of SEQ ID No 6. Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 6 or the complements thereof

The Open Reading Frame encoding the mG713 protein spans from the nucleotide in position 51 and the nucleotide in position 1450 of the nucleotide sequence of SEQ ID No 6. A purified or isolated nucleic acid comprising the mG713 ORF is an object of the present invention.

The cDNA of SEQ ID No 6 includes a 5′-UTR region. This 5′-UTR region starts from the nucleotide at position 1 and ends at the nucleotide in position 50 of SEQ ID No 6. The cDNA of SEQ ID No 6 includes a 3′-UTR region starting from the nucleotide at position 1451 and ending at the nucleotide at position 1791 of SEQ ID No 6. Consequently, the invention concerns a purified, isolated, and recombinant nucleic acid comprising a nucleotide sequence of the 5′UTR of the mG713 cDNA, a sequence complementary thereto, or an allelic variant thereof. The invention also concerns a purified, isolated, and recombinant nucleic acid comprising a nucleotide sequence of the 3′UTR of the mG713 cDNA, a sequence complementary thereto, or an allelic variant thereof.

Another object of the invention consists of a purified or isolated nucleic acid comprising the nucleotide sequence of SEQ ID No 6 or fragments thereof.

The invention also pertains to a purified or isolated nucleic acid having at least 85, 90, 95, 97, 98 or 99% of nucleotide identity with the nucleotide sequence of SEQ ID No 6, preferably 99.5% and most preferably 99.8% nucleotide identity with the nucleotide sequence of SEQ ID No 6.

The nucleotide differences as regards to the nucleotide sequence of SEQ ID No 6 are generally randomly distributed throughout the entire nucleic acid. Nevertheless, preferred nucleic acids are those wherein the nucleotide differences as regards to the nucleotide sequence of SEQ ID No 6 are predominantly located outside the coding sequences, and more precisely in the 5′-UTR and the 3′-UTR sequences contained in the nucleotide sequence of SEQ ID No 6.

Regulatory Sequences

As already mentioned hereinbefore, the genomic sequence of the G713 gene contains regulatory sequences both in the non-coding 5′-flanking region and in the non-coding 3′-flanking region that border the G713 coding region containing the three exons of this gene.

The longest 5′-regulatory sequence of the G713 gene is localized between the nucleotide in position 1076 and the nucleotide in position 3075 of the nucleotide sequence of SEQ ID No 1.

The longest 3′-regulatory sequence of the G713 gene is localized between the nucleotide in position 16330 and the nucleotide in position 18329 of the nucleotide sequence of SEQ ID No 3.

Polynucleotides derived from the G713 regulatory regions described above are useful in order to detect the presence of at least a copy of a nucleotide sequence containing SEQ ID Nos 1 or 3 in a test sample.

Thus, a further object of the present invention consists of a purified or isolated nucleic acid that hybridizes under stringent hybridization conditions with a polynucleotide comprising the nucleotide positions 1076 to 3075 of SEQ ID No 1, or the nucleotide positions 16330 to 18329 of SEQ ID No 3, or a sequence complementary thereto.

The promoter activity of the regulatory regions contained in the G713 nucleotide sequence of SEQ ID No 1 can be assessed as described below.

In order to identify the relevant biologically active polynucleotide fragments or variants of SEQ ID Nos 1 or 3, the one skill in the art will refer to Sambrook et al. (Sambrook, J. Fritsch, E. F., and T. Maniatis. 1989. Molecular Cloning: a Laboratory Manual. 2^{nd} Ed., Cold Spring Harbor Laboratory Press, Cold Spring Harbor, N.Y.) which describes the use of a recombinant vector carrying a marker gene (i.e. beta galactosidase, chloramphenicol acetyl transferase, etc.) the expression of which will be detected when placed under the control of a biologically active polynucleotide fragments or variants of SEQ ID Nos 1 or 3. Genomic sequences located upstream of the first exon of the G713 gene are cloned into a suitable promoter reporter vector, such as the pSEAP-Basic, pSEAP-Enhancer, pβgal-Basic, pβgal-Enhancer, or pEGFP-1 Promoter Reporter vectors available from Clontech, or pGL2-basic or pGL3-basic promoterless luciferase reporter gene vector from Promega. Briefly, each of these promoter reporter vectors include multiple cloning sites positioned upstream of a reporter gene encoding a readily assayable protein such as secreted alkaline phosphatase, luciferase, beta galactosidase, or green fluorescent protein. The sequences upstream the G713 coding region are inserted into the cloning sites upstream of the reporter gene in both orientations and introduced into an appropriate host cell. The level of reporter protein is assayed and compared to the level obtained from a vector which lacks an insert in the cloning site. The presence of an elevated expression level in the vector containing the insert with respect to the control vector indicates the presence of a promoter in the insert. If necessary, the upstream sequences can be cloned into vectors which contain an enhancer for increasing transcription levels from weak promoter sequences. A significant level of expression above that observed with the vector lacking an insert indicates that a promoter sequence is present in the inserted upstream sequence.

Promoter sequences within the upstream genomic DNA may be further defined by constructing nested 5′ and/or 3′ deletions in the upstream DNA using conventional techniques such as Exonuclease III or appropriate restriction endonuclease digestion. The resulting deletion fragments can be inserted into the promoter reporter vector to determine whether the deletion has reduced or obliterated promoter activity, such as described, for example, by Coles et al. (1998). In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. This type of assay is well-known to those skilled in the art and is described in WO 97/17359, U.S. Pat. No. 5,374,544, EP 582 796, U.S. Pat. No. 5,698,389, U.S. Pat. 5,643,746, U.S. Pat. No. 5,502,176, and U.S. Pat. No. 5,266,488, the disclosures of which are incorporated herein by reference in their entireties.

The strength and the specificity of the promoter of the G713 gene can be assessed through the expression levels of a detectable polynucleotide operably linked to the G713 promoter in different types of cells and tissues. The detectable polynucleotide may be either a polynucleotide that specifically hybridizes with a predefined oligonucleotide probe, or a polynucleotide encoding a detectable protein, including a G713 polypeptide or a fragment or a variant thereof. This type of assay is well-known to those skilled in the art and is described in U.S. Pat. No. 5,502,176, and U.S. Pat. No. 5,266,488, incorporated herein by reference. In one embodiment, the efficacy of the promoter of the G713 gene is assessed in normal and cancer cells.

Polynucleotides carrying the regulatory elements located both at the 5′ end and at the 3′ end of the G713 coding region may be advantageously used to control the transcriptional and translational activity of an heterologous polynucleotide of interest.

Thus, the present invention also concerns a purified or isolated nucleic acid comprising a polynucleotide which is selected from the group consisting of the nucleotide sequences SEQ ID Nos 1 and 3, or a sequence complementary thereto or a biologically active fragment or variant thereof.

Preferred fragments of the nucleic acid of SEQ ID No 1 have a length of about 400 nucleotides, more particularly about 300 nucleotides, more preferably 200 nucleotides and most preferably about 100 nucleotides.

Preferred fragments of the nucleic acid of SEQ ID No 3 have a length of about 600 nucleotides, more particularly about 300 nucleotides, more preferably 200 nucleotides and most preferably about 100 nucleotides.

By a biologically active polynucleotide derivative of regulatory polynucleotides of SEQ ID Nos 1 or 3 is intended a polynucleotide comprising or alternatively consisting in a fragment of said polynucleotide which is functional as a regulatory region for expressing a recombinant polypeptide or a recombinant polynucleotide in a recombinant cell host. It could act either as an enhancer or as a repressor.

For the purpose of the invention, a nucleic acid or polynucleotide is “functional” as a regulatory region for expressing a recombinant polypeptide or a recombinant polynucleotide if said regulatory polynucleotide contains nucleotide sequences which contain transcriptional and translational regulatory information, and such sequences are “operably linked” to nucleotide sequences which encode the desired polypeptide or the desired polynucleotide. An operable linkage is a linkage in which the regulatory nucleic acid and the DNA sequence sought to be expressed are linked in such a way as to permit gene expression.

More precisely, two DNA molecules (such as a polynucleotide containing a promoter region and a polynucleotide encoding a desired polypeptide or polynucleotide) are said to be “operably linked” if the nature of the linkage between the two polynucleotides does not (1) result in the introduction of a frame-shift mutation or (2) interfere with the ability of the polynucleotide containing the promoter to direct the transcription of the coding polynucleotide. The promoter polynucleotide would be operably linked to a polynucleotide encoding a desired polypeptide or a desired polynucleotide if the promoter is capable of effecting transcription of the polynucleotide of interest.

The regulatory polynucleotides of the invention may be prepared from any of the nucleotide sequence of SEQ ID Nos 1 or 3 by cleavage using suitable restriction enzymes, as described for example in the book of Sambrook et al. (1989). Table 5 details the restriction map of the G713 5′-regulatory nucleic acid of SEQ ID No 1. The left column indicates the name of the restriction enzyme preceded by the number of recognition sites for this enzyme present in the nucleotide sequence of SEQ ID No 1, excepted when a “0” is indicated in the column “Position” which indicates the absence of any recognition site for the enzyme in the nucleotide sequence of SEQ ID No 1. The second column discloses the sequence recognized by each enzyme and a “′” denotes the site of enzymatic cleavage. Third column depicts the nucleotide position of the nucleotide sequence of SEQ ID No 1 wherein the cleavage occurs. The fourth and fifth columns present the lengths of the nucleic acid fragments generated after enzymatic cleavage.

The regulatory polynucleotides may also be prepared by digestion of any of SEQ ID Nos 1 or 3 by an exonuclease enzyme, such as for example Bal31 (Wabiko et al., 1986).

These regulatory polynucleotides can also be prepared by nucleic acid chemical synthesis, as described elsewhere in the specification, where oligonucleotide probes or primers synthesis is disclosed.

The regulatory polynucleotides according to the invention may be advantageously part of a recombinant expression vector that may be used to express a coding sequence in a desired host cell or host organism. The recombinant expression vectors according to the invention are described elsewhere in the specification.

A preferred 5′-regulatory polynucleotide of the invention includes the 5′-untranslated region (5′-UTR) located between the nucleotide at position 1076 and the nucleotide at position 3075 of SEQ ID No 1, or a biologically active fragment or variant thereof.

A preferred 3′-regulatory polynucleotide of the invention includes a 3′-non coding region consisting in the nucleotide sequence starting from the nucleotide in position 16330 and ending at the nucleotide in position 18329 of the nucleic acid of SEQ ID No 3.

A further object of the invention consists of a purified or isolated nucleic acid comprising:

a) a nucleic acid comprising a regulatory polynucleotide of nucleotide positions 1076 to 3075 of SEQ ID No 1 or a biologically active fragment or variant thereof;

b) a polynucleotide encoding a desired polypeptide or nucleic acid operably linked to the regulatory polynucleotide of nucleotide positions 1076 to 3075 of SEQ ID No 1 or its biologically active fragment or variant thereof;

c) optionally, a nucleic acid comprising a regulatory polynucleotide of nucleotide positions 16330 to 18329 of SEQ ID No 3 or a biologically active fragment or variant thereof.

In a specific embodiment of the nucleic acid defined above, said nucleic acid includes the 5′-untranslated region (5′-UTR) located between the nucleotide at position 1076 and the nucleotide at position 3075 of SEQ ID No 1, or a biologically active fragment or variant thereof.

In a second specific embodiment of the nucleic acid defined above, said nucleic acid includes the 3′-untranslated region (3′-UTR) consisting in the nucleotide sequence starting from the nucleotide in position 16330 and ending a the nucleotide in position 18329 of the nucleic acid of SEQ ID No 3.

The regulatory polynucleotide of nucleotide positions 1076 to 3075 of SEQ ID No 1, or its biologically active fragments or variants, is advantageously operably linked at the 5′-end of the polynucleotide encoding the desired polypeptide or polynucleotide.

The regulatory polynucleotide of nucleotide positions 16330 to 18329 of SEQ ID No 3, or its biologically active fragments and variants, is advantageously placed at the 3′-end of the polynucleotide encoding the desired polypeptide or polynucleotide.

The desired polypeptide encoded by the above described nucleic acid may be of various nature or origin, encompassing proteins of prokaryotic or eukaryotic origin. Among the polypeptides expressed under the control of a G713 regulatory region, there may be cited bacterial, fungal or viral antigens. Also encompassed are eukaryotic proteins such as intracellular proteins, like “house keeping” proteins, membrane-bound proteins, like receptors, and secreted proteins like the numerous endogenous mediators such as cytokines. Indeed, the desired polypeptide may be either the human or the mouse G713 protein, especially one of the proteins of the amino acid sequences of SEQ ID No 5 or SEQ ID No 7, or a fragment or variant thereof.

The desired nucleic acids encoded by the above described polynucleotide, a usually a RNA molecule, may be complementary to a desired coding polynucleotide, for example to the human or mouse G713 coding sequence, and thus useful as an antisense polynucleotide.

Such a polynucleotide may be included in a recombinant expression vector in order to express the desired polypeptide or the desired nucleic acid in host cell or in a host organism. Suitable recombinant vectors that contain a polynucleotide such as described hereinbefore are disclosed elsewhere in the specification.

Coding Regions

The G713 open reading frame is contained in the corresponding mRNA of SEQ ID No 4 and is a further object of the present invention.

More precisely, the effective human G713 coding sequence (CDS) is comprised between the nucleotide at position 659 (first nucleotide of the ATG codon) and the nucleotide at position 2032 (end nucleotide of the TAA codon) of SEQ ID No 4. A purified or isolated polynucleotide comprising the G713 coding region defined above is another object of the invention.

Further, the effective mouse G713 coding sequence (CDS) is comprised between the nucleotide at position 51 (first nucleotide of the ATG codon) and the nucleotide at position 1453 (end nucleotide of the TGA codon) of SEQ ID No 6. A purified or isolated polynucleotide comprising the mouse G713 coding region defined above is another object of the invention.

The above disclosed polynucleotide that contains the coding sequence of the G713 gene of the invention may be expressed in a desired host cell or a desired host organism, when this polynucleotide is placed under the control of suitable expression signals. The expression signals may be either the expression signals contained in the regulatory regions in the G713 gene of the invention or in contrast be exogenous regulatory nucleic sequences. Such a polynucleotide, when placed under the suitable expression signals, may also be inserted in a vector for its expression.

Genomic DNA of Human Chromosome 13q31-q33 Gene Expressed in Schizophrenia Cases

The present invention also concerns the genomic sequence of a schizophrenia candidate gene located on the 13q31-q33 locus and specifically expressed in humans affected by schizophrenia. The present invention encompasses said schizophrenia candidate gene, or genomic sequences consisting of, consisting essentially of, or comprising the sequence of SEQ ID No 31, a sequence complementary thereto, as well as fragments and variants thereof. These polynucleotides may be purified, isolated, or recombinant.

The invention also encompasses a purified, isolated, or recombinant polynucleotide comprising a nucleotide sequence having at least 70, 75, 80, 85, 90, or 95% nucleotide identity with a nucleotide sequence of SEQ ID No 31 or a complementary sequence thereto or a fragment thereof. The nucleotide differences as regards to the nucleotide sequence of SEQ ID No 31 may be generally randomly distributed throughout the entire nucleic acid. Nevertheless, preferred nucleic acids are those wherein the nucleotide differences as regards to the nucleotide sequence of SEQ ID No 31 are predominantly located outside the coding sequences contained in the exons. These nucleic acids, as well as their fragments and variants, may be used as oligonucleotide primers or probes in order to detect the presence of a copy of this schizophrenia candidate gene in a test sample, or alternatively in order to amplify a target nucleotide sequence within said sequences.

Another object of the invention consists of a purified, isolated, or recombinant nucleic acid that hybridizes with the nucleotide sequence of SEQ ID No 31 or a complementary sequence thereto or a variant thereof, under the stringent hybridization conditions as defined above.

Particularly preferred nucleic acids of the invention include isolated, purified, or recombinant polynucleotides comprising a contiguous span of at least 12,15, 18, 20, 25, 30, 35, 40, 50, 60, 70, 80, 90, 100, 150, 200 or 500 nucleotides of SEQ ID No 31, or the complements thereof, wherein said contiguous span comprises at least one of the following nucleotide positions of SEQ ID No 31: 1 to 480 and 717 to 983. It should be noted that nucleic acid fragments of any size and sequence may also be comprised by the polynucleotides described in this section.

Probes and Primers

Polynucleotides derived from the G713 gene are useful in order to detect the presence of at least a copy of a nucleotide sequence of SEQ ID Nos 1 to 3, or a fragment, complement, or variant thereof in a test sample.

Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1 to 3 or the complements thereof, wherein said contiguous span comprises at least one of the following nucleotide positions of SEQ ID No 1 to 3:

1 to 3585 and 4644 to 5222 of SEQ ID No 1, or a variant thereof or a sequence complementary thereto;

1 to 16155 and 16331 to 21278 of SEQ ID No 2, or a variant thereof or a sequence complementary thereto; and

1 to 5531 and 6355 to 21636 of SEQ ID No 3, or a variant thereof or a sequence complementary thereto.

Other preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1 and 3 or the complements thereof, wherein said contiguous span comprises at least one of the following nucleotide positions of SEQ ID No 1 and 3:

1 to 3236, 3547 to 3585 and 4649 to 5222 of SEQ ID No 1, or a variant thereof or a sequence complementary thereto;

1 to 5531, 6844 to 7237, 7798 to 8184, 8667 to 9074, and 9356 to 21636 of SEQ ID No 3, or a variant thereof or a sequence complementary thereto.

Other probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID Nos. 1, 2 or 3 or the complements thereof, wherein said contiguous span comprises at least 1, 2, 3, 5, or 10 of the following nucleotide positions of SEQ ID Nos 1, 2 and 3:

1 to 1000, 1001 to 2000, 2001 to 3000, 3001 to 4000 and 4001 to 5222 of SEQ ID No. 1;

1 to 1000, 1001 to 2000, 2001 to 3000, 3001 to 4000 and 4001 to 5000, 5001 to 6000, 6001 to 7000, 7001 to 8000, 8001 to 9000, 9001 to 10000, 10001 to 11000, 11001 to 12000, 12001 to 13000, 13001 to 14000, 14001 ot 15000, 15001 to 16000, 16001 to 17000, 17001 to 18000, 18001 to 19000, 19001 to 20000 and 20001 to 21278 of SEQ ID No 2; and

1 to 1000, 1001 to 2000, 2001 to 3000, 3001 to 4000 and 4001 to 5000, 5001 to 6000, 6001 to 7000, 7001 to 8000, 8001 to 9000, 9001 to 10000, 10001 to 11000, 11001 to 12000, 12001 to 13000, 13001 to 14000, 14001 ot 15000, 15001 to 16000, 16001 to 17000, 17001 to 18000, 18001 to 19000, 19001 to 20000, 20001 to 21000 and 21001 to 21636 of SEQ ID No 3.

Further preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID Nos 1 to 3 or the complements thereof, wherein said contiguous span comprises allele 1 of a biallelic marker selected from the group consisting of A1 to A11; optionally said contiguous span comprises allele 2 of a biallelic marker selected from the group consisting of A1 to A11.

The invention also concerns a polymorphic marker comprising an insertion in the G713 gene. Embodiments of the invention thus include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1 or the complements thereof, wherein said contiguous span comprises the nucleotides AGAG at positions 3606 to 3609 of SEQ ID No 1.

Another object of the invention is a purified, isolated, or recombinant nucleic acid comprising the nucleotide sequence of SEQ ID No 4 or 6 complementary sequences thereto, as well as allelic variants, and fragments thereof. Moreover, preferred probes and primers of the invention include purified, isolated, or recombinant G713 cDNAs consisting of, consisting essentially of, or comprising the sequence of SEQ ID Nos 4 or 6. Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4, or the complements thereof, wherein said contiguous span comprises at least one of the following nucleotide positions of SEQ ID Nos 4: 1 to 519 and 2563 to 5566. Additional preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID Nos 4, or the complements thereof, wherein said contiguous span comprises 1 to 166, 473 to 519, 3020 to 3445, 3990 to 4394 and 4874 to 5281.

Thus, the invention also relates to nucleic acid probes characterized in that they hybridize specifically, under the stringent hybridization conditions defined above, with a nucleic acid selected from the group consisting of the nucleotide sequences 1 to 3236, 3547 to 3585 and 4649 to 5222 of SEQ ID No 1; 1 to 16155 and 16331 to 21278 of SEQ ID No 2; and 1 to 5531, 6844 to 7237, 7798 to 8184, 8667 to 9074, and 9356 to 21636 of SEQ ID No 3, or a variant thereof or a sequence complementary thereto.

In embodiments described in further detail herein in the section titled G713 and 13q31-q33-related biallelic markers, the invention encompasses isolated, purified, and recombinant polynucleotides consisting of, or consisting essentially of a contiguous span of 8 to 50 nucleotides of any one of SEQ ID Nos 1 to 4 or 6, and the complement thereof, wherein said span includes a G713-related biallelic marker in said sequence; optionally, wherein said G713-related biallelic marker is selected from the group consisting of A1 to A11, and the complements thereof, or optionally the biallelic markers in linkage disequilibrium therewith; optionally, wherein said contiguous span is 18 to 35 nucleotides in length and said biallelic marker is within 4 nucleotides of the center of said polynucleotide; optionally, wherein said polynucleotide consists of said contiguous span and said contiguous span is 25 nucleotides in length and said biallelic marker is at the center of said polynucleotide; optionally, wherein the 3′ end of said contiguous span is present at the 3′ end of said polynucleotide; and optionally, wherein the 3′ end of said contiguous span is located at the 3′ end of said polynucleotide and said biallelic marker is present at the 3′ end of said polynucleotide.

The formation of stable hybrids depends on the melting temperature (Tm) of the DNA. The Tm depends on the length of the primer or probe, the ionic strength of the solution and the G+C content. The higher the G+C content of the primer or probe, the higher is the melting temperature because G:C pairs are held by three H bonds whereas A:T pairs have only two. The GC content in the probes of the invention usually ranges between 10 and 75%, preferably between 35 and 60%, and more preferably between 40 and 55%.

A probe or a primer according to the invention may be between 8 and 2000 nucleotides in length, or is specified to be at least 12, 15, 18, 20, 25, 35, 40, 50, 60, 70, 80, 100, 250, 500, 1000 nucleotides in length. More particularly, the length of these probes can range from 8, 10, 15, 20, or 30 to 100 nucleotides, preferably from 10 to 50, more preferably from 15 to 30 nucleotides. Shorter probes tend to lack specificity for a target nucleic acid sequence and generally require cooler temperatures to form sufficiently stable hybrid complexes with the template. Longer probes are expensive to produce and can sometimes self-hybridize to form hairpin structures. The appropriate length for primers and probes under a particular set of assay conditions may be empirically determined by one of skill in the art.

The primers and probes can be prepared by any suitable method, including, for example, cloning and restriction of appropriate sequences and direct chemical synthesis by a method such as the phosphodiester method of Narang et al.(1979), the phosphodiester method of Brown et al.(1979), the diethylphosphoramidite method of Beaucage et al.(1981) and the solid support method described in EP 0 707 592. Detection probes are generally nucleic acid sequences or uncharged nucleic acid analogs such as, for example peptide nucleic acids which are disclosed in International Patent Application WO92/20702, the entire contents of which are hereby incorporated by reference, and morpholino analogs which are described in U.S. Pat. Nos. 5,185,444; 5,034,506 and 5,142,047, the entire contects of which are hereby incorporated by reference. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified, U.S. patent application Ser. No. 07/049,061 filed Apr. 19, 1993 describes modifications, which can be used to render a probe non-extendable.

Any of the polynucleotides of the present invention can be labeled, if desired, by incorporating any label known in the art to be detectable by spectroscopic, photochemical, biochemical, immunochemical, or chemical means. For example, useful labels include radioactive substances (including, ³²P, ³⁵S, ³H, ¹²⁵I), fluorescent dyes (including, 5-bromodesoxyuridin, fluorescein, acetylaminofluorene, digoxigenin) or biotin. Preferably, polynucleotides are labeled at their 3′ and 5′ ends. Examples of non-radioactive labeling of nucleic acid fragments are described in the French patent No. FR-7810975 or by Urdea et al (1988) or Sanchez-Pescador et al (1988). In addition, the probes according to the present invention may have structural characteristics such that they allow the signal amplification, such structural characteristics being, for example, branched DNA probes as those described by Urdea et al. (1991) or in European Patent No. EP 0 225 807 (Chiron).

A label can also be used to capture the primer, so as to facilitate the immobilization of either the primer or a primer extension product, such as amplified DNA, on a solid support. A capture label is attached to the primers or probes and can be a specific binding member which forms a binding pair with the solid phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

The probes of the present invention are useful for a number of purposes. They can be notably used in Southern hybridization to genomic DNA. The probes can also be used to detect PCR amplification products. They may also be used to detect mismatches in the G713 gene or mRNA using other techniques.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes® and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Consequently, the invention also comprises a method for detecting the presence of a nucleic acid comprising a nucleotide sequence selected from a group consisting of SEQ ID Nos 1 to 4 or 6, a fragment or a variant thereof and a complementary sequence thereto in a sample, said method comprising the following steps of:

a) bringing into contact a nucleic acid probe or a plurality of nucleic acid probes which can hybridize with a nucleotide sequence included in a nucleic acid selected form the group consisting of the nucleotide sequences of SEQ ID Nos 1 to 4 or 6, a fragment or a variant thereof and a complementary sequence thereto and the sample to be assayed; and

b) detecting the hybrid complex formed between the probe and a nucleic acid in the sample.

The invention further concerns a kit for detecting the presence of a nucleic acid comprising a nucleotide sequence selected from a group consisting of SEQ ID Nos 1 to 4 or 6, a fragment or a variant thereof and a complementary sequence thereto in a sample, said kit comprising:

a) a nucleic acid probe or a plurality of nucleic acid probes which can hybridize with a nucleotide sequence included in a nucleic acid selected form the group consisting of the nucleotide sequences of SEQ ID Nos 1 to 4 or 6, a fragment or a variant thereof and a complementary sequence thereto; and

b) optionally, the reagents necessary for performing the hybridization reaction.

In a first preferred embodiment of this detection method and kit, said nucleic acid probe or the plurality of nucleic acid probes are labeled with a detectable molecule. In a second preferred embodiment of said method and kit, said nucleic acid probe or the plurality of nucleic acid probes has been immobilized on a substrate. In a third preferred embodiment, the nucleic acid probe or the plurality of nucleic acid probes comprise either a sequence which is selected from the group consisting of the nucleotide sequences of P1 to P11 and the complementary sequence thereto, B1 to B11, C1 to C11, D1 to D11, E1 to E11 or a biallelic marker selected from the group consisting of A1 to A11 and the complements thereto.

Oligonucleotide Arrays

A substrate comprising a plurality of oligonucleotide primers or probes of the invention may be used either for detecting or amplifying targeted sequences in the G713 gene and may also be used for detecting mutations in the coding or in the non-coding sequences of the G713 gene.

Any polynucleotide provided herein may be attached in overlapping areas or at random locations on the solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips™, and has been generally described in U.S. Pat. No. 5,143,854 and PCT publications WO90/15070 and 92/10092. The entire contents of these documents is hereby incorporated by reference in their entireties.

These arrays may generally be produced using mechanical synthesis methods or light directed synthesis methods which incorporate a combination of photolithographic methods and solid phase oligonucleotide synthesis (Fodor et al., 1991). The immobilization of arrays of oligonucleotides on solid supports has been rendered possible by the development of a technology generally identified as “Very Large Scale Immobilized Polymer Synthesis” (VLSIPS™) in which, typically, probes are immobilized in a high density array on a solid surface of a chip. Examples of VLSIPS™ technologies are provided in U.S. Pat. Nos. 5,143,854; and 5,412,087 and in PCT Publications WO 90/15070, WO 92/10092 and WO 95/11995, the entire contents of which are hereby incorporated by reference, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256, the disclosures of which are incorporated herein by reference in their entireties.

In another embodiment of the oligonucleotide arrays of the invention, an oligonucleotide probe matrix may advantageously be used to detect mutations occurring in the G713 gene and preferably in its regulatory region. For this particular purpose, probes are specifically designed to have a nucleotide sequence allowing their hybridization to the genes that carry known mutations (either by deletion, insertion or substitution of one or several nucleotides). By known mutations, it is meant, mutations on the G713 gene that have been identified according, for example to the technique used by Huang et al.(1996) or Samson et al.(1996).

Another technique that is used to detect mutations in the G713 gene is the use of a high-density DNA array. Each oligonucleotide probe constituting a unit element of the high density DNA array is designed to match a specific subsequence of the G713 genomic DNA or cDNA. Thus, an array consisting of oligonucleotides complementary to subsequences of the target gene sequence is used to determine the identity of the target sequence with the wild gene sequence, measure its amount, and detect differences between the target sequence and the reference wild gene sequence of the G713 gene. In one such design, termed 4L tiled array, is implemented a set of four probes (A, C, G, T), preferably 15-nucleotide oligomers. In each set of four probes, the perfect complement will hybridize more strongly than mismatched probes.

Consequently, a nucleic acid target of length L is scanned for mutations with a tiled array containing 4L probes, the whole probe set containing all the possible mutations in the known wild reference sequence. The hybridization signals of the 15-mer probe set tiled array are perturbed by a single base change in the target sequence. As a consequence, there is a characteristic loss of signal or a “footprint” for the probes flanking a mutation position. This technique was described by Chee et al. in 1996.

Consequently, the invention concerns an array of nucleic acid molecules comprising at least one polynucleotide described above as probes and primers. Preferably, the invention concerns an array of nucleic acid comprising at least two polynucleotides described above as probes and primers.

A further object of the invention consists of an array of nucleic acid sequences comprising either at least one of the sequences selected from the group consisting of P1 to P49, B1 to B49, C1 to C49, D1 to D49, E1 to E49, the sequences complementary thereto, a fragment thereof of at least 8, 10, 12, 15, 18, 20, 25, 30, or 40 consecutive nucleotides thereof, and at least one sequence comprising a biallelic marker selected from the group consisting of A1 to A49 and the complements thereto.

The invention also pertains to an array of nucleic acid sequences comprising either at least two of the sequences selected from the group consisting of P1 to P49, B1 to B49, C1 to C49, D1 to D49, E1 to E49, the sequences complementary thereto, a fragment thereof of at least 8 consecutive nucleotides thereof, and at least two sequences comprising a biallelic marker selected from the group consisting of A1 to A49 and the complements thereof.

G713- and 13Q31-Q33-Related Biallelic Marker

The inventors have discovered nucleotide polymorphisms located within the genomic DNA containing the G713 gene, and among them “Single Nucleotide Polymorphisms” or SNPs that are also termed biallelic markers. The inventors have also discovered biallelic markers throughout the human chromosome 13q31-q33 locus.

The invention thus concerns G713-related biallelic markers. As used herein the term “G713-related biallelic marker” relates to a set of biallelic markers in linkage disequilibrium with the G713 gene. The term G713-related biallelic marker includes the biallelic markers designated A1 to A11 herein as well as an insertion of the nucleotides AGAG in the G713 gene, described above.

A portion of the G713 biallelic markers of the present invention are disclosed in Table 2. Their location on the G713 gene is indicated in Table 2 and also as a single base polymorphism in the features of in the related SEQ ID Nos 1 to 3. The pairs of primers allowing the amplification of a nucleic acid containing the polymorphic base of one G713 biallelic marker are listed in Table 1 of Example 1(c).

The invention also concerns 13q31-q33-related biallelic markers. As used herein the term “13q31-q33-related biallelic marker” relates to a set of biallelic markers in linkage disequilibrium with the chromosome 13q31-q33 locus. The term 13q31-q33-related biallelic marker includes the biallelic markers designated A12 to A49.

A portion of the 13q31-q33-related biallelic markers of the present invention are disclosed in Table 7. Their location as a single base polymorphism in the features of in the related SEQ ID Nos 32 to 65. The pairs of primers allowing the amplification of a nucleic acid containing the polymorphic base of each 13q31-q33-related biallelic marker are listed in Table 6 of Example 2(b).

The invention also relates to a purified and/or isolated nucleotide sequence comprising a polymorphic base of a G713- or 13q31-q33-related biallelic marker, preferably of a biallelic marker selected from the group consisting of A1 to A49, and the complements thereof. The sequence has between 8 and 1000 nucleotides in length, and preferably comprises a span of at least 8, 10, 12, 15, 18, 20, 25, 35, 40, 50, 60, 70, 80, 100, 250, 500 or 1000 contiguous nucleotides of a nucleotide sequence selected from the group consisting of SEQ ID Nos 1 to 3 and 32 to 69, or a variant thereof or a complementary sequence thereto. These nucleotide sequences comprise the polymorphic base of either allele 1 or allele 2 of the considered biallelic marker. Optionally, said biallelic marker may be within 6, 5, 4, 3, 2, or 1 nucleotides of the center of said polynucleotide or at the center of said polynucleotide. Optionally, the 3′ end of said contiguous span may be present at the 3′ end of said polynucleotide. Optionally, biallelic marker may be present at the 3′ end of said polynucleotide. Optionally, said contiguous span is 18 to 35 nucleotides in length and said biallelic marker is within 4 nucleotides of the center of said polynucleotide; optionally, said polynucleotide consists of said contiguous span and said contiguous span is 25 nucleotides in length and said biallelic marker is at the center of said polynucleotide; optionally, the 3′ end of said contiguous span is present at the 3′ end of said polynucleotide; and optionally, the 3′ end of said contiguous span is located at the 3′ end of said polynucleotide and said biallelic marker is present at the 3′ end of said polynucleotide. Optionally, said polynucleotide may further comprise a label. Optionally, said polynucleotide can be attached to solid support. In a further embodiment, the polynucleotides defined above can be used alone or in any combination.

The invention also relates to a purified and/or isolated nucleotide sequence comprising between 8 and 1000 nucleotides in length, and preferably at least 8, 10, 12, 15, 18, 20, 25, 35, 40, 50, 60, 70, 80, 100, 250, 500 or 1000 contiguous nucleotides of a nucleotide sequence selected from the group consisting of SEQ ID Nos 1 to 4, 6 and 32 to 69, or a variant thereof or a complementary sequence thereto. Optionally, the 3′ end of said polynucleotide may be located within or at least 2, 4, 6, 8, 10, 12, 15, 18, 20, 25, 50, 100, 250, 500, or 1000 nucleotides upstream of a G713- or 13q31-q33-related biallelic marker in said sequence. Optionally, said G713- or 13q31-q33-related biallelic marker is selected from the group consisting of A1 to A49; Optionally, the 3′ end of said polynucleotide may be located within or at least 2, 4, 6, 8, 10, 12, 15, 18, 20, 25, 50, 100, 250, 500, or 1000 nucleotides upstream of a G713- or 13q31-q33-related biallelic marker in said sequence. Optionally, the 3′ end of said polynucleotide may be located 1 nucleotide upstream of a G713- or 13q31-q33-related biallelic marker in said sequence. Optionally, said polynucleotide may further comprise a label. Optionally, said polynucleotide can be attached to solid support. In a further embodiment, the polynucleotides defined above can be used alone or in any combination.

In a preferred embodiment, the sequences comprising a polymorphic base of one of the biallelic markers listed in Tables 2 and 7 are selected from the group consisting of the nucleotide sequences that have a contiguous span of, that consist of, that are comprised in, or that comprises a polynucleotide selected from the group consisting of the nucleic acids of the sequences set forth as the amplicons listed in Tables 1 and 6 or a variant thereof or a complementary sequence thereto.

The invention further concerns a nucleic acid encoding the G713 protein, wherein said nucleic acid comprises a polymorphic base of a biallelic marker selected from the group consisting of A1 to A11 and the complements thereof.

The invention also encompasses the use of any polynucleotide for, or any polynucleotide for use in, determining the identity of one or more nucleotides at a G713- or 13q31-q33-related biallelic marker. In addition, the polynucleotides of the invention for use in determining the identity of one or more nucleotides at a G713- or 13q31-q33-related biallelic marker encompass polynucleotides with any further limitation described in this disclosure, or those following, specified alone or in any combination. Optionally, said G713-related biallelic marker is selected from the group consisting of A1 to A11, and the complements thereof, or optionally the biallelic markers in linkage disequilibrium therewith; optionally, said 13q31-q33-related biallelic marker is selected from the group consisting of A12 to A49, and the complements thereof, or optionally the biallelic markers in linkage disequilibrium therewith; optionally, said 13q31-q33-related biallelic marker is selected from the group consisting of A16 to A20 and the complements thereof, or optionally the biallelic markers in linkage disequilibrium therewith; optionally, said 13q31-q33-related biallelic marker is selected from the group consisting of A14, A15, A17, A18, A27, A28, A34, A35, A38 and the complements thereof, or optionally the biallelic markers in linkage disequilibrium therewith; Optionally, said polynucleotide may comprise a sequence disclosed in the present specification; Optionally, said polynucleotide may consist of, or consist essentially of any polynucleotide described in the present specification; Optionally, said determining may be performed in a hybridization assay, sequencing assay, microsequencing assay, or an enzyme-based mismatch detection assay; Optionally, said polynucleotide may be attached to a solid support, array, or addressable array; Optionally, said polynucleotide may be labeled. A preferred polynucleotide may be used in a hybridization assay for determining the identity of the nucleotide at a G713- or 13q31-q33-related biallelic marker. Another preferred polynucleotide may be used in a sequencing or microsequencing assay for determining the identity of the nucleotide at a G713- or 13q31-q33-related biallelic marker. A third preferred polynucleotide may be used in an enzyme-based mismatch detection assay for determining the identity of the nucleotide at a G713- or 13q31-q33-related biallelic marker. A fourth preferred polynucleotide may be used in amplifying a segment of polynucleotides comprising a G713- or 13q31-q33-related biallelic marker. Optionally, any of the polynucleotides described above may be attached to a solid support, array, or addressable array; optionally, said polynucleotide may be labeled.

Additionally, the invention encompasses the use of any polynucleotide for, or any polynucleotide for use in, amplifying a segment of nucleotides comprising a G713- or 13q31-q33-related biallelic marker. In addition, the polynucleotides of the invention for use in amplifying a segment of nucleotides comprising a G713- or 13q31-q33-related biallelic marker encompass polynucleotides with any further limitation described in this disclosure, or those following, specified alone or in any combination: optionally, said G713-related biallelic marker is selected from the group consisting of A1 to A11, and the complements thereof, or optionally the biallelic markers in linkage disequilibrium therewith; optionally, said 13q31-q33-related biallelic marker is selected from the group consisting of A12 to A49, and the complements thereof, or optionally the biallelic markers in linkage disequilibrium therewith; optionally, said 13q31-q33-related biallelic marker is selected from the group consisting of A16 to A20 and the complements thereof, or optionally the biallelic markers in linkage disequilibrium therewith; optionally, said 13q31-q33-related biallelic marker is selected from the group consisting of A14, A15, A17, A18, A27, A28, A34, A35, A38 and the complements thereof, or optionally the biallelic markers in linkage disequilibrium therewith; optionally, said polynucleotide may comprise a sequence disclosed in the present specification; optionally, said polynucleotide may consist of, or consist essentially of any polynucleotide described in the present specification; optionally, said amplifying may be performed by a PCR or LCR. Optionally, said polynucleotide may be attached to a solid support, array, or addressable array. Optionally, said polynucleotide may be labeled.

The primers for amplification or sequencing reaction of a polynucleotide comprising a biallelic marker of the invention may be designed from the disclosed sequences for any method known in the art. A preferred set of primers are fashioned such that the 3′ end of the contiguous span of identity with a sequence selected from the group consisting of SEQ ID Nos 1 to 4, 6 and 32 to 69 or a sequence complementary thereto or a variant thereof is present at the 3′ end of the primer. Such a configuration allows the 3′ end of the primer to hybridize to a selected nucleic acid sequence and dramatically increases the efficiency of the primer for amplification or sequencing reactions. Allele specific primers may be designed such that a polymorphic base of a biallelic marker is at the 3′ end of the contiguous span and the contiguous span is present at the 3′ end of the primer. Such allele specific primers tend to selectively prime an amplification or sequencing reaction so long as they are used with a nucleic acid sample that contains one of the two alleles present at a biallelic marker. The 3′ end of the primer of the invention may be located within or at least 2, 4, 6, 8, 10, 12, 15, 18, 20, 25, 50, 100, 250, 500, or 1000 nucleotides upstream of a G713- or 13q31-q33-related biallelic marker in said sequence or at any other location which is appropriate for their intended use in sequencing, amplification or the location of novel sequences or markers. Thus, another set of preferred amplification primers comprise an isolated polynucleotide consisting essentially of a contiguous span of 8 to 50 nucleotides in a sequence selected from the group consisting of SEQ ID Nos 1 to 4, 6 and 32 to 69 or a sequence complementary thereto or a variant thereof, wherein the 3′ end of said contiguous span is located at the 3′end of said polynucleotide, and wherein the 3′end of said polynucleotide is located upstream of a G713- or 13q31-q33-related biallelic marker in said sequence. Preferably, those amplification primers comprise a sequence selected from the group consisting of the sequences B1 to B49 and C1 to C49. Primers with their 3′ ends located 1 nucleotide upstream of a biallelic marker of G713 or 13q31-q33 have a special utility as microsequencing assays. Preferred microsequencing primers are described in Tables 4 and 8. Optionally, said G713-related biallelic marker is selected from the group consisting of A1 to A11, and the complements thereof, or optionally the biallelic markers in linkage disequilibrium therewith; optionally, said 13q31-q33-related biallelic marker is selected from the group consisting of A12 to A49, and the complements thereof, or optionally the biallelic markers in linkage disequilibrium therewith; optionally, said 13q31-q33-related biallelic marker is selected from the group consisting of A16 to A20 and the complements thereof, or optionally the biallelic markers in linkage disequilibrium therewith; optionally, said 13q31-q33-related biallelic marker is selected from the group consisting of A14, A15, A17, A18, A27, A28, A34, A35, A38 and the complements thereof, or optionally the biallelic markers in linkage disequilibrium therewith; optionally, microsequencing primers are selected from the group consisting of the nucleotide sequences D1 to D49 and E1 to E49.

The probes of the present invention may be designed from the disclosed sequences for any method known in the art, particularly methods which allow for testing if a marker disclosed herein is present. A preferred set of probes may be designed for use in the hybridization assays of the invention in any manner known in the art such that they selectively bind to one allele of a biallelic marker, but not the other under any particular set of assay conditions. Preferred hybridization probes comprise the polymorphic base of either allele 1 or allele 2 of the considered biallelic marker. Optionally, said biallelic marker may be within 6, 5, 4, 3, 2, or 1 nucleotides of the center of the hybridization probe or at the center of said probe. In a preferred embodiment, the probes are selected in the group consisting of the sequences P1 to P49 and the complementary sequence thereto.

It should be noted that the polynucleotides of the present invention are not limited to having the exact flanking sequences surrounding the polymorphic bases which are enumerated in Sequence Listing. Rather, it will be appreciated that the flanking sequences surrounding the biallelic markers may be lengthened or shortened to any extent compatible with their intended use and the present invention specifically contemplates such sequences. The flanking regions outside of the contiguous span need not be homologous to native flanking sequences which actually occur in human subjects. The addition of any nucleotide sequence which is compatible with the nucleotides intended use is specifically contemplated.

Primers and probes may be labeled or immobilized on a solid support as described in “Oligonucleotide probes and primers”.

The polynucleotides of the invention which are attached to a solid support encompass polynucleotides with any further limitation described in this disclosure, or those following, specified alone or in any combination: Optionally, said polynucleotides may be specified as attached individually or in groups of at least 2, 5, 8, 10, 12, 15, 20, or 25 distinct polynucleotides of the invention to a single solid support. Optionally, polynucleotides other than those of the invention may attached to the same solid support as polynucleotides of the invention. Optionally, when multiple polynucleotides are attached to a solid support they may be attached at random locations, or in an ordered array. Optionally, said ordered array may be addressable.

The present invention also encompasses diagnostic kits comprising one or more polynucleotides of the invention with a portion or all of the necessary reagents and instructions for genotyping a test subject by determining the identity of a nucleotide at a G713- or 13q31-q33-related biallelic marker. The polynucleotides of a kit may optionally be attached to a solid support, or be part of an array or addressable array of polynucleotides. The kit may provide for the determination of the identity of the nucleotide at a marker position by any method known in the art including, but not limited to, a sequencing assay method, a microsequencing assay method, a hybridization assay method, or an enzyme-based mismatch detection assay method.

Methods for De Novo Identification of Biallelic Markers

Any of a variety of methods can be used to screen a genomic fragment for single nucleotide polymorphisms such as differential hybridization with oligonucleotide probes, detection of changes in the mobility measured by gel electrophoresis or direct sequencing of the amplified nucleic acid. A preferred method for identifying biallelic markers involves comparative sequencing of genomic DNA fragments from an appropriate number of unrelated individuals.

In a first embodiment, DNA samples from unrelated individuals are pooled together, following which the genomic DNA of interest is amplified and sequenced. The nucleotide sequences thus obtained are then analyzed to identify significant polymorphisms. One of the major advantages of this method resides in the fact that the pooling of the DNA samples substantially reduces the number of DNA amplification reactions and sequencing reactions, which must be carried out. Moreover, this method is sufficiently sensitive so that a biallelic marker obtained thereby usually demonstrates a sufficient frequency of its less common allele to be useful in conducting association studies.

In a second embodiment, the DNA samples are not pooled and are therefore amplified and sequenced individually. This method is usually preferred when biallelic markers need to be identified in order to perform association studies within candidate genes. Preferably, highly relevant gene regions such as promoter regions or exon regions may be screened for biallelic markers. A biallelic marker obtained using this method may show a lower degree of informativeness for conducting association studies, e.g. if the frequency of its less frequent allele may be less than about 10%. Such a biallelic marker will, however, be sufficiently informative to conduct association studies and it will further be appreciated that including less informative biallelic markers in the genetic analysis studies of the present invention, may allow in some cases the direct identification of causal mutations, which may, depending on their penetrance, be rare mutations.

The following is a description of the various parameters of a preferred method used by the inventors for the identification of the biallelic markers of the present invention.

Genomic DNA Samples

The genomic DNA samples from which the biallelic markers of the present invention are generated are preferably obtained from unrelated individuals corresponding to a heterogeneous population of known ethnic background. The number of individuals from whom DNA samples are obtained can vary substantially, preferably from about 10 to about 1000, preferably from about 50 to about 200 individuals. It is usually preferred to collect DNA samples from at least about 100 individuals in order to have sufficient polymorphic diversity in a given population to identify as many markers as possible and to generate statistically significant results. As for the source of the genomic DNA to be subjected to analysis, any test sample can be foreseen without any particular limitation. These test samples include biological samples, which can be tested by the methods of the present invention described herein, and include human and animal body fluids such as whole blood, serum, plasma, cerebrospinal fluid, urine, lymph fluids, and various external secretions of the respiratory, intestinal and genitourinary tracts, tears, saliva, milk, white blood cells, myelomas and the like; biological fluids such as cell culture supernatants; fixed tissue specimens including tumor and non-tumor tissue and lymph node tissues; bone marrow aspirates and fixed cell specimens. The preferred source of genomic DNA used in the present invention is from peripheral venous blood of each donor. Techniques to prepare genomic DNA from biological samples are well known to the skilled technician. Details of a preferred embodiment are provided in Example 1(a). The person skilled in the art can choose to amplify pooled or unpooled DNA samples.

DNA Amplification

The identification of biallelic markers in a sample of genomic DNA may be facilitated through the use of DNA amplification methods. DNA samples can be pooled or unpooled for the amplification step. DNA amplification techniques are well known to those skilled in the art.

Amplification techniques that can be used in the context of the present invention include, but are not limited to, the ligase chain reaction (LCR) described in EP-A-320 308, WO 93/20227 and EP-A-439 182, the entire contents of which are hereby incorporated by reference, the polymerase chain reaction (PCR, RT-PCR) and techniques such as the nucleic acid sequence based amplification (NASBA) described in Guatelli J. C., et al.(1990) and in Compton J.(1991), Q-beta amplification as described in European Patent Application No 4544610, strand displacement amplification as described in Walker et al.(1996) and EP A 684 315 and, target mediated amplification as described in PCT Publication WO 93/22461, the entire contents of which are hereby incorporated by reference.

LCR and Gap LCR are exponential amplification techniques, both depend on DNA ligase to join adjacent primers annealed to a DNA molecule. In Ligase Chain Reaction (LCR), probe pairs are used which include two primary (first and second) and two secondary (third and fourth) probes, all of which are employed in molar excess to target. The first probe hybridizes to a first segment of the target strand and the second probe hybridizes to a second segment of the target strand, the first and second segments being contiguous so that the primary probes abut one another in 5′ phosphate-3′hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes, which can be ligated to form a complementary, secondary ligated product. It is important to realize that the ligated products are functionally equivalent to either the target or its complement. By repeated cycles of hybridization and ligation, amplification of the target sequence is achieved. A method for multiplex LCR has also been described (WO 93/20227). Gap LCR (GLCR) is a version of LCR where the probes are not adjacent but are separated by 2 to 3 bases.

For amplification of mRNAs, it is within the scope of the present invention to reverse transcribe mRNA into cDNA followed by polymerase chain reaction (RT-PCR); or, to use a single enzyme for both steps as described in U.S. Pat. No. 5,322,770, the entire contents of which are hereby incorporated by reference, or, to use Asymmetric Gap LCR (RT-AGLCR) as described by Marshall et al.(1994). AGLCR is a modification of GLCR that allows the amplification of RNA. The PCR technology is the preferred amplification technique used in the present invention. A variety of PCR techniques are familiar to those skilled in the art. For a review of PCR technology, see White (1997) and the publication entitled “PCR Methods and Applications” (1991, Cold Spring Harbor Laboratory Press). In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195; 4,683,202; and 4,965,188, the disclosures of which are incorporated herein by reference in their entireties.

The PCR technology is the preferred amplification technique used to identify new biallelic markers. A typical example of a PCR reaction suitable for the purposes of the present invention is provided in Example 1(c).

One of the aspects of the present invention is a method for the amplification of the human G713 gene, particularly of a fragment of the genomic sequence of SEQ ID Nos 1 to 3 or of the cDNA sequences of SEQ ID Nos 4 or 6, or a fragment or a variant thereof in a test sample, preferably using the PCR technology. Another aspect is a method for the amplification of a nucleotide sequence of the human chromosome 13q31-q33 locus, particularly of a fragment of the genomic sequence of SEQ ID Nos 32 to 69, or a fragment or a variant thereof in a test sample, preferably using the PCR technology. This method comprises the steps of:

a) contacting a test sample with amplification reaction reagents comprising a pair of amplification primers as described above and located on either side of the polynucleotide region to be amplified, and

b) optionally, detecting the amplification products.

The invention also concerns a kit for the amplification of a G713 or chromosome 13q31-q33 sequence, particularly of a portion of the G713 genomic sequence of SEQ ID Nos 1 to 3, of the G713 cDNA sequences of SEQ ID Nos 6 or 11 or of the chromosome 13q31-q33 locus, or a variant thereof in a test sample, wherein said kit comprises:

a) a pair of oligonucleotide primers located on either side of the G713 or chromosome 13q31-q33 region to be amplified;

b) optionally, the reagents necessary for performing the amplification reaction.

In one embodiment of the above amplification method and kit, the amplification product is detected by hybridization with a labeled probe having a sequence which is complementary to the amplified region. In another embodiment of the above amplification method and kit, primers comprise a sequence which is selected from the group consisting of the nucleotide sequences of B1 to B49, C1 to C49, D1 to D49, and E1 to E49.

In a first embodiment of the present invention, biallelic markers are identified using genomic sequence information generated by the inventors. Sequenced genomic DNA fragments are used to design primers for the amplification of 500 bp fragments. These 500 bp fragments are amplified from genomic DNA and are scanned for biallelic markers. Primers may be designed using the OSP software (Hillier L. and Green P., 1991). All primers may contain, upstream of the specific target bases, a common oligonucleotide tail that serves as a sequencing primer. Those skilled in the art are familiar with primer extensions, which can be used for these purposes.

Preferred primers, useful for the amplification of genomic sequences encoding the candidate genes, focus on promoters, exons and splice sites of the genes. A biallelic marker presents a higher probability to be an eventual causal mutation if it is located in these functional regions of the gene. Preferred amplification primers of the invention include the nucleotide sequences B1 to B49 and C1 to C49, detailed further in Example 1(c), Table 1 and Example 2(b), Table 6.

Sequencing of Amplified Genomic DNA and Identification of Single Nucleotide Polymorphisms

The amplification products generated as described above, are then sequenced using any method known and available to the skilled technician. Methods for sequencing DNA using either the dideoxy-mediated method (Sanger method) or the Maxam-Gilbert method are widely known to those of ordinary skill in the art. Such methods are for example disclosed in Sambrook et al.(1989). Alternative approaches include hybridization to high-density DNA probe arrays as described in Chee et al.(1996).

Preferably, the amplified DNA is subjected to automated dideoxy terminator sequencing reactions using a dye-primer cycle sequencing protocol. The products of the sequencing reactions are run on sequencing gels and the sequences are determined using gel image analysis. The polymorphism search is based on the presence of superimposed peaks in the electrophoresis pattern resulting from different bases occurring at the same position. Because each dideoxy terminator is labeled with a different fluorescent molecule, the two peaks corresponding to a biallelic site present distinct colors corresponding to two different nucleotides at the same position on the sequence. However, the presence of two peaks can be an artifact due to background noise. To exclude such an artifact, the two DNA strands are sequenced and a comparison between the peaks is carried out. In order to be registered as a polymorphic sequence, the polymorphism has to be detected on both strands. The above procedure permits those amplification products, which contain biallelic markers to be identified. The detection limit for the frequency of biallelic polymorphisms detected by sequencing pools of 100 individuals is approximately 0.1 for the minor allele, as verified by sequencing pools of known allelic frequencies. However, more than 90% of the biallelic polymorphisms detected by the pooling method have a frequency for the minor allele higher than 0.25. Therefore, the biallelic markers selected by this method have a frequency of at least 0.1 for the minor allele and less than 0.9 for the major allele. Preferably at least 0.2 for the minor allele and less than 0.8 for the major allele, more preferably at least 0.3 for the minor allele and less than 0.7 for the major allele, thus a heterozygosity rate higher than 0.18, preferably higher than 0.32, more preferably higher than 0.42.

In another embodiment, biallelic markers are detected by sequencing individual DNA samples, the frequency of the minor allele of such a biallelic marker may be less than 0.1.

Validation of the Biallelic Markers of the Present Invention

The polymorphisms are evaluated for their usefulness as genetic markers by validating that both alleles are present in a population. Validation of the biallelic markers is accomplished by genotyping a group of individuals by a method of the invention and demonstrating that both alleles are present. Microsequencing is a preferred method of genotyping alleles. The validation by genotyping step may be performed on individual samples derived from each individual in the group or by genotyping a pooled sample derived from more than one individual. The group can be as small as one individual if that individual is heterozygous for the allele in question. Preferably the group contains at least three individuals, more preferably the group contains five or six individuals, so that a single validation test will be more likely to result in the validation of more of the biallelic markers that are being tested. It should be noted, however, that when the validation test is performed on a small group it may result in a false negative result if as a result of sampling error none of the individuals tested carries one of the two alleles. Thus, the validation process is less useful in demonstrating that a particular initial result is an artifact, than it is at demonstrating that there is a bona fide biallelic marker at a particular position in a sequence. For an indication of whether a particular biallelic marker has been validated, a * is placed next to the microsequencing primer in Table 4. All of the genotyping, haplotyping, association, and interaction study methods of the invention may optionally be performed solely with validated biallelic markers.

Evaluation of the Frequency of the Biallelic Markers of the Present Invention

The validated biallelic markers are further evaluated for their usefulness as genetic markers by determining the frequency of the least common allele at the biallelic marker site. The higher the frequency of the less common allele the greater the usefulness of the biallelic marker is association and interaction studies. The determination of the least common allele is accomplished by genotyping a group of individuals by a method of the invention and demonstrating that both alleles are present. This determination of frequency by genotyping step may be performed on individual samples derived from each individual in the group or by genotyping a pooled sample derived from more than one individual. The group must be large enough to be representative of the population as a whole. Preferably the group contains at least 20 individuals, more preferably the group contains at least 50 individuals, most preferably the group contains at least 100 individuals. Of course the larger the group the greater the accuracy of the frequency determination because of reduced sampling error. A biallelic marker wherein the frequency of the less common allele is 30% or more is termed a “high quality biallelic marker.” All of the genotyping, haplotyping, association, and interaction study methods of the invention may optionally be performed solely with high quality biallelic markers.

Methods for Genotyping an Individual for Biallelic Markers

Methods are provided to genotype a biological sample for one or more biallelic markers of the present invention, all of which may be performed in vitro. Such methods of genotyping comprise determining the identity of a nucleotide at a G713 or 13q31-q33-related biallelic marker site by any method known in the art. These methods find use in genotyping case-control populations in association studies as well as individuals in the context of detection of alleles of biallelic markers which are known to be associated with a given trait, in which case both copies of the biallelic marker present in individual's genome are determined so that an individual may be classified as homozygous or heterozygous for a particular allele.

These genotyping methods can be performed on nucleic acid samples derived from a single individual or pooled DNA samples.

Genotyping can be performed using similar methods as those described above for the identification of the biallelic markers, or using other genotyping methods such as those further described below. In preferred embodiments, the comparison of sequences of amplified genomic fragments from different individuals is used to identify new biallelic markers whereas microsequencing is used for genotyping known biallelic markers in diagnostic and association study applications.

One embodiment the invention provides methods of genotyping comprising determining the identity of a nucleotide at a G713-related biallelic marker or the complement thereof in a biological sample; optionally, wherein said G713-related biallelic marker is selected from the group consisting of A1 to A11, and the complements thereof, or optionally the biallelic markers in linkage disequilibrium therewith. In another embodiment the invention encompasses methods of genotyping comprising determining the identity of a nucleotide at a 13q31-q33-related biallelic marker or the complement thereof in a biological sample; optionally, wherein said 13q31-q33-related biallelic marker is selected from the group consisting of A12 to A49, and the complements thereof, or optionally the biallelic markers in linkage disequilibrium therewith; optionally, wherein said 13q31-q33-related biallelic marker is selected from the group consisting of A16 to A20, and the complements thereof, or optionally the biallelic markers in linkage disequilibrium therewith; optionally, wherein said 13q31-q33-related biallelic marker is selected from the group consisting of A14, A15, A17, A18, A27, A28, A34, A35, A38, and the complements thereof, or optionally the biallelic markers in linkage disequilibrium therewith; optionally, wherein said biological sample is derived from a single subject; optionally, wherein the identity of the nucleotides at said biallelic marker is determined for both copies of said biallelic marker present in said individual's genome; optionally, wherein said biological sample is derived from multiple subjects; Optionally, the genotyping methods of the invention encompass methods with any further limitation described in this disclosure, or those following, specified alone or in any combination; Optionally, said method is performed in vitro; optionally, further comprising amplifying a portion of said sequence comprising the biallelic marker prior to said determining step; Optionally, wherein said amplifying is performed by PCR, LCR, or replication of a recombinant vector comprising an origin of replication and said fragment in a host cell; optionally, wherein said determining is performed by a hybridization assay, a sequencing assay, a microsequencing assay, or an enzyme-based mismatch detection assay.

Source of Nucleic Acids for Genotyping

Any source of nucleic acids, in purified or non-purified form, can be utilized as the starting nucleic acid, provided it contains or is suspected of containing the specific nucleic acid sequence desired. DNA or RNA may be extracted from cells, tissues, body fluids and the like as described above. While nucleic acids for use in the genotyping methods of the invention can be derived from any mammalian source, the test subjects and individuals from which nucleic acid samples are taken are generally understood to be human.

Amplification of DNA Fragments Comprising Biallelic Markers

Methods and polynucleotides are provided to amplify a segment of nucleotides comprising one or more biallelic marker of the present invention. It will be appreciated that amplification of DNA fragments comprising biallelic markers may be used in various methods and for various purposes and is not restricted to genotyping. Nevertheless, many genotyping methods, although not all, require the previous amplification of the DNA region carrying the biallelic marker of interest. Such methods specifically increase the concentration or total number of sequences that span the biallelic marker or include that site and sequences located either distal or proximal to it. Diagnostic assays may also rely on amplification of DNA segments carrying a biallelic marker of the present invention. Amplification of DNA may be achieved by any method known in the art. Amplification techniques are described above in the section entitled, “DNA amplification.”

Some of these amplification methods are particularly suited for the detection of single nucleotide polymorphisms and allow the simultaneous amplification of a target sequence and the identification of the polymorphic nucleotide as it is further described below.

The identification of biallelic markers as described above allows the design of appropriate oligonucleotides, which can be used as primers to amplify DNA fragments comprising the biallelic markers of the present invention. Amplification can be performed using the primers initially used to discover new biallelic markers which are described herein or any set of primers allowing the amplification of a DNA fragment comprising a biallelic marker of the present invention.

In some embodiments the present invention provides primers for amplifying a DNA fragment containing one or more biallelic markers of the present invention. Preferred amplification primers are listed in Examples 1(c) and 2(b). It will be appreciated that the primers listed are merely exemplary and that any other set of primers which produce amplification products containing one or more biallelic markers of the present invention are also of use.

The spacing of the primers determines the length of the segment to be amplified. In the context of the present invention, amplified segments carrying biallelic markers can range in size from at least about 25 bp to 35 kbp. Amplification fragments from 25-3000 bp are typical, fragments from 50-1000 bp are preferred and fragments from 100-600 bp are highly preferred. It will be appreciated that amplification primers for the biallelic markers may be any sequence which allow the specific amplification of any DNA fragment carrying the markers. Amplification primers may be labeled or immobilized on a solid support as described in “Oligonucleotide probes and primers”.

Methods of Genotyping DNA Samples for Biallelic Markers

Any method known in the art can be used to identify the nucleotide present at a biallelic marker site. Since the biallelic marker allele to be detected has been identified and specified in the present invention, detection will prove simple for one of ordinary skill in the art by employing any of a number of techniques. Many genotyping methods require the previous amplification of the DNA region carrying the biallelic marker of interest. While the amplification of target or signal is often preferred at present, ultrasensitive detection methods which do not require amplification are also encompassed by the present genotyping methods. Methods well known to those skilled in the art that can be used to detect biallelic polymorphisms include methods such as, conventional dot blot analyzes, single strand conformational polymorphism analysis (SSCP) described by Orita et al.(1989), denaturing gradient gel electrophoresis (DGGE), heteroduplex analysis, mismatch cleavage detection, and other conventional techniques as described in Sheffield et al.(1991), White et al.(1992), Grompe et al.(1989 and 1993). Another method for determining the identity of the nucleotide present at a particular polymorphic site employs a specialized exonuclease-resistant nucleotide derivative as described in U.S. Pat. No. 4,656,127, the entire contents of which are hereby incorporated by reference.

Preferred methods involve directly determining the identity of the nucleotide present at a biallelic marker site by sequencing assay, enzyme-based mismatch detection assay, or hybridization assay. The following is a description of some preferred methods. A highly preferred method is the microsequencing technique. The term “sequencing” is generally used herein to refer to polymerase extension of duplex primer/template complexes and includes both traditional sequencing and microsequencing.

1) Sequencing Assays

The nucleotide present at a polymorphic site can be determined by sequencing methods. In a preferred embodiment, DNA samples are subjected to PCR amplification before sequencing as described above. DNA sequencing methods are described in “Sequencing Of Amplified Genomic DNA And Identification Of Single Nucleotide Polymorphisms”.

Preferably, the amplified DNA is subjected to automated dideoxy terminator sequencing reactions using a dye-primer cycle sequencing protocol. Sequence analysis allows the identification of the base present at the biallelic marker site.

2) Microsequencing Assays

In microsequencing methods, the nucleotide at a polymorphic site in a target DNA is detected by a single nucleotide primer extension reaction. This method involves appropriate microsequencing primers which, hybridize just upstream of the polymorphic base of interest in the target nucleic acid. A polymerase is used to specifically extend the 3′ end of the primer with one single ddNTP (chain terminator) complementary to the nucleotide at the polymorphic site. Next the identity of the incorporated nucleotide is determined in any suitable way.

Typically, microsequencing reactions are carried out using fluorescent ddNTPs and the extended microsequencing primers are analyzed by electrophoresis on ABI 377 sequencing machines to determine the identity of the incorporated nucleotide as described in EP 412 883, the disclosure of which is incorporated herein by reference in its entirety. Alternatively capillary electrophoresis can be used in order to process a higher number of assays simultaneously. An example of a typical microsequencing procedure that can be used in the context of the present invention is provided in Example 1(e).

Different approaches can be used for the labeling and detection of ddNTPs. A homogeneous phase detection method based on fluorescence resonance energy transfer has been described by Chen and Kwok (1997) and Chen et al.(1997). In this method, amplified genomic DNA fragments containing polymorphic sites are incubated with a 5′-fluorescein-labeled primer in the presence of allelic dye-labeled dideoxyribonucleoside triphosphates and a modified Taq polymerase. The dye-labeled primer is extended one base by the dye-terminator specific for the allele present on the template. At the end of the genotyping reaction, the fluorescence intensities of the two dyes in the reaction mixture are analyzed directly without separation or purification. All these steps can be performed in the same tube and the fluorescence changes can be monitored in real time. Alternatively, the extended primer may be analyzed by MALDI-TOF Mass Spectrometry. The base at the polymorphic site is identified by the mass added onto the microsequencing primer (see Haff and Smirnov, 1997).

Microsequencing may be achieved by the established microsequencing method or by developments or derivatives thereof. Alternative methods include several solid-phase microsequencing techniques. The basic microsequencing protocol is the same as described previously, except that the method is conducted as a heterogeneous phase assay, in which the primer or the target molecule is immobilized or captured onto a solid support. To simplify the primer separation and the terminal nucleotide addition analysis, oligonucleotides are attached to solid supports or are modified in such ways that permit affinity separation as well as polymerase extension. The 5′ ends and internal nucleotides of synthetic oligonucleotides can be modified in a number of different ways to permit different affinity separation approaches, e.g., biotinylation. If a single affinity group is used on the oligonucleotides, the oligonucleotides can be separated from the incorporated terminator regent. This eliminates the need of physical or size separation. More than one oligonucleotide can be separated from the terminator reagent and analyzed simultaneously if more than one affinity group is used. This permits the analysis of several nucleic acid species or more nucleic acid sequence information per extension reaction. The affinity group need not be on the priming oligonucleotide but could alternatively be present on the template. For example, immobilization can be carried out via an interaction between biotinylated DNA and streptavidin-coated microtitration wells or avidin-coated polystyrene particles. In the same manner, oligonucleotides or templates may be attached to a solid support in a high-density format. In such solid phase microsequencing reactions, incorporated ddNTPs can be radiolabeled (Syvänen, 1994) or linked to fluorescein (Livak and Hainer, 1994). The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such as p-nitrophenyl phosphate). Other possible reporter-detection pairs include: ddNTP linked to dinitrophenyl (DNP) and anti-DNP alkaline phosphatase conjugate (Harju et al., 1993) or biotinylated ddNTP and horseradish peroxidase-conjugated streptavidin with o-phenylenediamine as a substrate (WO 92/15712, the disclosure of which is incorporated herein by reference in its entirety). As yet another alternative solid-phase microsequencing procedure, Nyren et al.(1993) described a method relying on the detection of DNA polymerase activity by an enzymatic luminometric inorganic pyrophosphate detection assay (ELIDA).

Pastinen et al.(1997) describe a method for multiplex detection of single nucleotide polymorphism in which the solid phase minisequencing principle is applied to an oligonucleotide array format. High-density arrays of DNA probes attached to a solid support (DNA chips) are further described below.

In one aspect the present invention provides polynucleotides and methods to genotype one or more biallelic markers of the present invention by performing a microsequencing assay. Preferred microsequencing primers include the nucleotide sequences D1 to D49 and E1 to E49. It will be appreciated that the microsequencing primers listed in Examples 1(e) and 2(d) are merely exemplary and that, any primer having a 3′ end immediately adjacent to the polymorphic nucleotide may be used. Similarly, it will be appreciated that microsequencing analysis may be performed for any biallelic marker or any combination of biallelic markers of the present invention. One aspect of the present invention is a solid support which includes one or more microsequencing primers listed in Examples 1(e) and 2(d), or fragments comprising at least 8, 12, 15, 20, 25, 30, 40, or 50 consecutive nucleotides thereof, to the extent that such lengths are consistent with the primer described, and having a 3′ terminus immediately upstream of the corresponding biallelic marker, for determining the identity of a nucleotide at a biallelic marker site.

3) Mismatch Detection Assays Based on Polymerases and Ligases

In one aspect the present invention, there are provided polynucleotides and methods to determine the allele of one or more bialielic markers of the present invention in a biological sample, by mismatch detection assays based on polymerases and/or ligases. These assays are based on the specificity of polymerases and ligases. Polymerization reactions places particularly stringent requirements on correct base pairing of the 3′ end of the amplification primer and the joining of two oligonucleotides hybridized to a target DNA sequence is quite sensitive to mismatches close to the ligation site, especially at the 3′ end. Methods, primers and various parameters to amplify DNA fragments comprising biallelic markers of the present invention are further described above in “Amplification Of DNA Fragments Comprising Biallelic Markers”.

Allele Specific Amplification Primers

Discrimination between the two alleles of a biallelic marker can also be achieved by allele specific amplification, a selective strategy, whereby one of the alleles is amplified without amplification of the other allele. For allele specific amplification, at least one member of the pair of primers is sufficiently complementary with a region of a G713 or 13q31-q33 nucleotide sequence comprising the polymorphic base of a biallelic marker of the present invention to hybridize therewith and to initiate the amplification. Such primers are able to discriminate between the two alleles of a biallelic marker.

This is accomplished by placing the polymorphic base at the 3′ end of one of the amplification primers. Because the extension forms from the 3′end of the primer, a mismatch at or near this position has an inhibitory effect on amplification. Therefore, under appropriate amplification conditions, these primers only direct amplification on their complementary allele. Determining the precise location of the mismatch and the corresponding assay conditions are well within the ordinary skill in the art.

Ligation/Amplification Based Methods

The “Oligonucleotide Ligation Assay” (OLA) uses two oligonucleotides which are designed to be capable of hybridizing to abutting sequences of a single strand of a target molecules. One of the oligonucleotides is biotinylated, and the other is detectably labeled. If the precise complementary sequence is found in a target molecule, the oligonucleotides will hybridize such that their termini abut, and create a ligation substrate that can be captured and detected. OLA is capable of detecting single nucleotide polymorphisms and may be advantageously combined with PCR as described by Nickerson et al.(1990). In this method, PCR is used to achieve the exponential amplification of target DNA, which is then detected using OLA.

Other amplification methods which are particularly suited for the detection of single nucleotide polymorphism include LCR (ligase chain reaction), Gap LCR (GLCR) which are described above in “DNA Amplification”. LCR uses two pairs of probes to exponentially amplify a specific target. The sequences of each pair of oligonucleotides, is selected to permit the pair to hybridize to abutting sequences of the same strand of the target. Such hybridization forms a substrate for a template-dependant ligase. In accordance with the present invention, LCR can be performed with oligonucleotides having the proximal and distal sequences of the same strand of a biallelic marker site. In one embodiment, either oligonucleotide will be designed to include the biallelic marker site. In such an embodiment, the reaction conditions are selected such that the oligonucleotides can be ligated together only if the target molecule either contains or lacks the specific nucleotide that is complementary to the biallelic marker on the oligonucleotide. In an alternative embodiment, the oligonucleotides will not include the biallelic marker, such that when they hybridize to the target molecule, a “gap” is created as described in WO 90/01069, the disclosure of which is incorporated herein by reference in its entirety. This gap is then “filled” with complementary dNTPs (as mediated by DNA polymerase), or by an additional pair of oligonucleotides. Thus at the end of each cycle, each single strand has a complement capable of serving as a target during the next cycle and exponential allele-specific amplification of the desired sequence is obtained.

Ligase/Polymerase-mediated Genetic Bit Analysis™ is another method for determining the identity of a nucleotide at a preselected site in a nucleic acid molecule (WO 95/21271, the entire contents of which are hereby incorporated by reference). This method involves the incorporation of a nucleoside triphosphate that is complementary to the nucleotide present at the preselected site onto the terminus of a primer molecule, and their subsequent ligation to a second oligonucleotide. The reaction is monitored by detecting a specific label attached to the reaction's solid phase or by detection in solution.

4) Hybridization Assay Methods

A preferred method of determining the identity of the nucleotide present at a biallelic marker site involves nucleic acid hybridization. The hybridization probes, which can be conveniently used in such reactions, preferably include the probes defined herein. Any hybridization assay may be used including Southern hybridization, Northern hybridization, dot blot hybridization and solid-phase hybridization (see Sambrook et al., 1989).

Hybridization refers to the formation of a duplex structure by two single stranded nucleic acids due to complementary base pairing. Hybridization can occur between exactly complementary nucleic acid strands or between nucleic acid strands that contain minor regions of mismatch. Specific probes can be designed that hybridize to one form of a biallelic marker and not to the other and therefore are able to discriminate between different allelic forms. Allele-specific probes are often used in pairs, one member of a pair showing perfect match to a target sequence containing the original allele and the other showing a perfect match to the target sequence containing the alternative allele. Hybridization conditions should be sufficiently stringent that there is a significant difference in hybridization intensity between alleles, and preferably an essentially binary response, whereby a probe hybridizes to only one of the alleles. Stringent, sequence specific hybridization conditions, under which a probe will hybridize only to the exactly complementary target sequence are well known in the art (Sambrook et al., 1989). Stringent conditions are sequence dependent and will be different in different circumstances. Generally, stringent conditions are selected to be about 5° C. lower than the thermal melting point (Tm) for the specific sequence at a defined ionic strength and pH. Although such hybridization can be performed in solution, it is preferred to employ a solid-phase hybridization assay. The target DNA comprising a biallelic marker of the present invention may be amplified prior to the hybridization reaction. The presence of a specific allele in the sample is determined by detecting the presence or the absence of stable hybrid duplexes formed between the probe and the target DNA. The detection of hybrid duplexes can be carried out by a number of methods. Various detection assay formats are well known which utilize detectable labels bound to either the target or the probe to enable detection of the hybrid duplexes. Typically, hybridization duplexes are separated from unhybridized nucleic acids and the labels bound to the duplexes are then detected. Those skilled in the art will recognize that wash steps may be employed to wash away excess target DNA or probe as well as unbound conjugate. Further, standard heterogeneous assay formats are suitable for detecting the hybrids using the labels present on the primers and probes.

Two recently developed assays allow hybridization-based allele discrimination with no need for separations or washes (see Landegren U. et al., 1998). The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. Cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., 1995). In an alternative homogeneous hybridization based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., 1998).

The polynucleotides provided herein can be used to produce probes which can be used in hybridization assays for the detection of biallelic marker alleles in biological samples. These probes are characterized in that they preferably comprise between 8 and 50 nucleotides, and in that they are sufficiently complementary to a sequence comprising a biallelic marker of the present invention to hybridize thereto and preferably sufficiently specific to be able to discriminate the targeted sequence for only one nucleotide variation. A particularly preferred probe is 25 nucleotides in length. Preferably the biallelic marker is within 4 nucleotides of the center of the polynucleotide probe. In particularly preferred probes, the biallelic marker is at the center of said polynucleotide. Preferred probes comprise a nucleotide sequence selected from the group consisting of amplicons listed in Tables 1 and 6 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. Preferred probes comprise a nucleotide sequence selected from the group consisting of P1 to P49 and the sequences complementary thereto. In preferred embodiments the polymorphic base(s) are within 5, 4, 3, 2, 1, nucleotides of the center of the said polynucleotide, more preferably at the center of said polynucleotide.

Preferably the probes of the present invention are labeled or immobilized on a solid support. Labels and solid supports are further described in “Oligonucleotide Probes and Primers”. The probes can be non-extendable as described in “Oligonucleotide Probes and Primers”.

By assaying the hybridization to an allele specific probe, one can detect the presence or absence of a biallelic marker allele in a given sample. High-Throughput parallel hybridization in array format is specifically encompassed within “hybridization assays” and are described below.

5) Hybridization to Addressable Arrays of Oligonucleotides

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (e.g., the chip) at selected positions. Each DNA chip can contain thousands to millions of individual synthetic DNA probes arranged in a grid-like pattern and miniaturized to the size of a dime.

The chip technology has already been applied with success in numerous cases. For example, the screening of mutations has been undertaken in the BRCA1 gene, in S. cerevisiae mutant strains, and in the protease gene of HIV-1 virus (Hacia et al., 1996; Shoemaker et al., 1996; Kozal et al., 1996). Chips of various formats for use in detecting biallelic polymorphisms can be produced on a customized basis by Affymetrix (GeneChip™), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. EP 785280, the disclosure of which is incorporated herein by reference in its entirety, describes a tiling strategy for the detection of single nucleotide polymorphisms. Briefly, arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of nucleotides. Tiling strategies are further described in WO 95/11995, the entire contents of which are hereby incorporated by reference. In a particular aspect, arrays are tiled for a number of specific, identified biallelic marker sequences. In particular, the array is tiled to include a number of detection blocks, each detection block being specific for a specific biallelic marker or a set of biallelic markers. For example, a detection block may be tiled to include a number of probes, which span the sequence segment that includes a specific polymorphism. To ensure probes that are complementary to each allele, the probes are synthesized in pairs differing at the biallelic marker. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the biallelic marker. The monosubstituted probes provide internal controls for the tiled array, to distinguish actual hybridization from artefactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the biallelic marker are present in the sample. Hybridization and scanning may be carried out as described in WO92/10092, WO 95/11995 and U.S. Pat. No. 5,424,186, the entire contents of which are hereby incorporated by reference.

Thus, in some embodiments, the chips may comprise an array of nucleic acid sequences of fragments of about 15 nucleotides in length. In further embodiments, the chip may comprise an array including at least one of the sequences selected from the group consisting of amplicons listed in Tables 1 and 6 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. In preferred embodiments the polymorphic base is within 5, 4, 3, 2, 1, nucleotides of the center of the said polynucleotide, more preferably at the center of said polynucleotide. In some embodiments, the chip may comprise an array of at least 2, 3, 4, 5, 6, 7, 8 or more of these polynucleotides of the invention. Solid supports and polynucleotides of the present invention attached to solid supports are further described in “Oligonucleotide Probes And Primers”.

6) Integrated Systems

Another technique, which may be used to analyze polymorphisms, includes multicomponent integrated systems, which miniaturize and compartmentalize processes such as PCR and capillary electrophoresis reactions in a single functional device. An example of such technique is disclosed in U.S. Pat. No. 5,589,136, the disclosure of which is incorporated herein by reference in its entirety, which describes the integration of PCR amplification and capillary electrophoresis in chips. Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip to create functional microscopic valves and pumps with no moving parts.

For genotyping biallelic markers, the microfluidic system may integrate nucleic acid amplification, microsequencing, capillary electrophoresis and a detection method such as laser-induced fluorescence detection.

Methods of Genetic Analysis Using the Biallelic Markers of the Present Invention

Different methods are available for the genetic analysis of complex traits (see Lander and Schork, 1994). The search for disease-susceptibility genes is conducted using two main methods: the linkage approach in which evidence is sought for cosegregation between a locus and a putative trait locus using family studies, and the association approach in which evidence is sought for a statistically significant association between an allele and a trait or a trait causing allele (Khoury et al., 1993). In general, the biallelic markers of the present invention find use in any method known in the art to demonstrate a statistically significant correlation between a genotype and a phenotype. The biallelic markers may be used in parametric and non-parametric linkage analysis methods. Preferably, the biallelic markers of the present invention are used to identify genes associated with detectable traits using association studies, an approach which does not require the use of affected families and which permits the identification of genes associated with complex and sporadic traits.

The genetic analysis using the biallelic markers of the present invention may be conducted on any scale. The whole set of biallelic markers of the present invention or any subset of biallelic markers of the present invention corresponding to the candidate gene may be used. Further, any set of genetic markers including a biallelic marker of the present invention may be used. A set of biallelic polymorphisms that could be used as genetic markers in combination with the biallelic markers of the present invention has been described in WO 98/20165, the entire contents of which are hereby incorporated by reference. As mentioned above, it should be noted that the biallelic markers of the present invention may be included in any complete or partial genetic map of the human genome. These different uses are specifically contemplated in the present invention and claims.

Linkage Analysis

Linkage analysis is based upon establishing a correlation between the transmission of genetic markers and that of a specific trait throughout generations within a family. Thus, the aim of linkage analysis is to detect marker loci that show cosegregation with a trait of interest in pedigrees.

Parametric Methods

When data are available from successive generations there is the opportunity to study the degree of linkage between pairs of loci. Estimates of the recombination fraction enable loci to be ordered and placed onto a genetic map. With loci that are genetic markers, a genetic map can be established, and then the strength of linkage between markers and traits can be calculated and used to indicate the relative positions of markers and genes affecting those traits (Weir, 1996). The classical method for linkage analysis is the logarithm of odds (lod) score method (see Morton, 1955; Ott, 1991). Calculation of lod scores requires specification of the mode of inheritance for the disease (parametric method). Generally, the length of the candidate region identified using linkage analysis is between 2 and 20 Mb. Once a candidate region is identified as described above, analysis of recombinant individuals using additional markers allows further delineation of the candidate region. Linkage analysis studies have generally relied on the use of a maximum of 5,000 microsatellite markers, thus limiting the maximum theoretical attainable resolution of linkage analysis to about 600 kb on average.

Linkage analysis has been successfully applied to map simple genetic traits that show clear Mendelian inheritance patterns and which have a high penetrance (i.e., the ratio between the number of trait positive carriers of allele a and the total number of a carriers in the population). However, parametric linkage analysis suffers from a variety of drawbacks. First, it is limited by its reliance on the choice of a genetic model suitable for each studied trait. Furthermore, as already mentioned, the resolution attainable using linkage analysis is limited, and complementary studies are required to refine the analysis of the typical 2 Mb to 20 Mb regions initially identified through linkage analysis. In addition, parametric linkage analysis approaches have proven difficult when applied to complex genetic traits, such as those due to the combined action of multiple genes and/or environmental factors. It is very difficult to model these factors adequately in a lod score analysis. In such cases, too large an effort and cost are needed to recruit the adequate number of affected families required for applying linkage analysis to these situations, as recently discussed by Risch, N. and Merikangas, K. (1996).

Non-Parametric Methods

The advantage of the so-called non-parametric methods for linkage analysis is that they do not require specification of the mode of inheritance for the disease, they tend to be more useful for the analysis of complex traits. In non-parametric methods, one tries to prove that the inheritance pattern of a chromosomal region is not consistent with random Mendelian segregation by showing that affected relatives inherit identical copies of the region more often than expected by chance. Affected relatives should show excess “allele sharing” even in the presence of incomplete penetrance and polygenic inheritance. In non-parametric linkage analysis the degree of agreement at a marker locus in two individuals can be measured either by the number of alleles identical by state (IBS) or by the number of alleles identical by descent (IBD). Affected sib pair analysis is a well-known special case and is the simplest form of these methods.

The biallelic markers of the present invention may be used in both parametric and non-parametric linkage analysis. Preferably biallelic markers may be used in non-parametric methods which allow the mapping of genes involved in complex traits. The biallelic markers of the present invention may be used in both IBD- and IBS-methods to map genes affecting a complex trait. In such studies, taking advantage of the high density of biallelic markers, several adjacent biallelic marker loci may be pooled to achieve the efficiency attained by multi-allelic markers (Zhao et al., 1998).

Population Association Studies

The present invention comprises methods for identifying if the G713 gene or a 13q31-q33 gene or nucleotide sequence is associated with a detectable trait using the biallelic markers of the present invention. In one embodiment the present invention comprises methods to detect an association between a biallelic marker allele or a biallelic marker haplotype and a trait. Further, the invention comprises methods to identify a trait causing allele in linkage disequilibrium with any biallelic marker allele of the present invention.

As described above, alternative approaches can be employed to perform association studies: genome-wide association studies, candidate region association studies and candidate gene association studies. In a preferred embodiment, the biallelic markers of the present invention are used to perform candidate gene association studies. The candidate gene analysis clearly provides a short-cut approach to the identification of genes and gene polymorphisms related to a particular trait when some information concerning the biology of the trait is available. Further, the biallelic markers of the present invention may be incorporated in any map of genetic markers of the human genome in order to perform genome-wide association studies. Methods to generate a high-density map of biallelic markers has been described in U.S. Provisional Patent application Ser. No. 60/082,614. The biallelic markers of the present invention may further be incorporated in any map of a specific candidate region of the genome (a specific chromosome or a specific chromosomal segment for example).

As mentioned above, association studies may be conducted within the general population and are not limited to studies performed on related individuals in affected families. Association studies are extremely valuable as they permit the analysis of sporadic or multifactor traits. Moreover, association studies represent a powerful method for fine-scale mapping enabling much finer mapping of trait causing alleles than linkage studies. Studies based on pedigrees often only narrow the location of the trait causing allele. Association studies using the biallelic markers of the present invention can therefore be used to refine the location of a trait causing allele in a candidate region identified by Linkage Analysis methods. Moreover, once a chromosome segment of interest has been identified, the presence of a candidate gene such as a candidate gene of the present invention, in the region of interest can provide a shortcut to the identification of the trait causing allele. Biallelic markers of the present invention can be used to demonstrate that a candidate gene is associated with a trait. Such uses are specifically contemplated in the present invention.

Determining the Frequency of a Biallelic Marker Allele or of a Biallelic Marker Haplotype in a Population

Association studies explore the relationships among frequencies for sets of alleles between loci.

Determining the Frequency of an Allele in a Population

Allelic frequencies of the biallelic markers in a populations can be determined using one of the methods described above under the heading “Methods for genotyping an individual for biallelic markers”, or any genotyping procedure suitable for this intended purpose. Genotyping pooled samples or individual samples can determine the frequency of a biallelic marker allele in a population. One way to reduce the number of genotypings required is to use pooled samples. A major obstacle in using pooled samples is in terms of accuracy and reproducibility for determining accurate DNA concentrations in setting up the pools. Genotyping individual samples provides higher sensitivity, reproducibility and accuracy and; is the preferred method used in the present invention. Preferably, each individual is genotyped separately and simple gene counting is applied to determine the frequency of an allele of a biallelic marker or of a genotype in a given population.

The invention also relates to methods of estimating the frequency of an allele in a population comprising: a) genotyping individuals from said population for said biallelic marker according to the method of the present invention; b) determining the proportional representation of said biallelic marker in said population. In addition, the methods of estimating the frequency of an allele in a population of the invention encompass methods with any further limitation described in this disclosure, or those following, specified alone or in any combination; optionally, wherein a G713-related biallelic marker is selected from the group consisting of A1 to A11, and the complements thereof, or optionally the biallelic markers in linkage disequilibrium therewith; optionally, wherein a 13q31-q33-related biallelic marker is selected from the group consisting of A12 to A49, and the complements thereof, or optionally the biallelic markers in linkage disequilibrium therewith; optionally, wherein said 13q31-q33-related biallelic marker is selected from the group consisting of A14, A15, A17, A18, A27, A28, A34, A35, A38 and the complements thereof, or optionally the bialielic markers in linkage disequilibrium therewith; optionally, wherein said 13q31-q33-related biallelic marker is selected from the group consisting of A16 to A20, and the complements thereof, or optionally the biallelic markers in linkage disequilibrium therewith; optionally, determining the frequency of a biallelic marker allele in a population may be accomplished by determining the identity of the nucleotides for both copies of said biallelic marker present in the genome of each individual in said population and calculating the proportional representation of said nucleotide at said G713- or 13q31-q33-related biallelic marker for the population; Optionally, determining the proportional representation may be accomplished by performing a genotyping method of the invention on a pooled biological sample derived from a representative number of individuals, or each individual, in said population, and calculating the proportional amount of said nucleotide compared with the total.

Determining the Frequency of a Haplotype in a Population

The gametic phase of haplotypes is unknown when diploid individuals are heterozygous at more than one locus. Using genealogical information in families gametic phase can sometimes be inferred (Perlin et al., 1994). When no genealogical information is available different strategies may be used. One possibility is that the multiple-site heterozygous diploids can be eliminated from the analysis, keeping only the homozygotes and the single-site heterozygote individuals, but this approach might lead to a possible bias in the sample composition and the underestimation of low-frequency haplotypes. Another possibility is that single chromosomes can be studied independently, for example, by asymmetric PCR amplification (see Newton et al, 1989; Wu et al., 1989) or by isolation of single chromosome by limit dilution followed by PCR amplification (see Ruano et al., 1990). Further, a sample may be haplotyped for sufficiently close biallelic markers by double PCR amplification of specific alleles (Sarkar, G. and Sommer S. S., 1991). These approaches are not entirely satisfying either because of their technical complexity, the additional cost they entail, their lack of generalization at a large scale, or the possible biases they introduce. To overcome these difficulties, an algorithm to infer the phase of PCR-amplified DNA genotypes introduced by Clark, A. G.(1990) may be used. Briefly, the principle is to start filling a preliminary list of haplotypes present in the sample by examining unambiguous individuals, that is, the complete homozygotes and the single-site heterozygotes. Then other individuals in the same sample are screened for the possible occurrence of previously recognized haplotypes. For each positive identification, the complementary haplotype is added to the list of recognized haplotypes, until the phase information for all individuals is either resolved or identified as unresolved. This method assigns a single haplotype to each multiheterozygous individual, whereas several haplotypes are possible when there are more than one heterozygous site. Alternatively, one can use methods estimating haplotype frequencies in a population without assigning haplotypes to each individual. Preferably, a method based on an expectation-maximization (EM) algorithm (Dempster et al., 1977) leading to maximum-likelihood estimates of haplotype frequencies under the assumption of Hardy-Weinberg proportions (random mating) is used (see Excoffier L. and Slatkin M., 1995). The EM algorithm is a generalized iterative maximum-likelihood approach to estimation that is useful when data are ambiguous and/or incomplete. The EM algorithm is used to resolve heterozygotes into haplotypes. Haplotype estimations are further described below under the heading “Statistical Methods.” Any other method known in the art to determine or to estimate the frequency of a haplotype in a population may be used. The invention also encompasses methods of estimating the frequency of a haplotype for a set of biallelic markers in a population, comprising the steps of: a) genotyping at least one G713- or 13q31-q33-related biallelic marker according to a method of the invention for each individual in said population; b) genotyping a second biallelic marker by determining the identity of the nucleotides at said second biallelic marker for both copies of said second biallelic marker present in the genome of each individual in said population; and c) applying a haplotype determination method to the identities of the nucleotides determined in steps a) and b) to obtain an estimate of said frequency. In addition, the methods of estimating the frequency of a haplotype of the invention encompass methods with any further limitation described in this disclosure, or those following, specified alone or in any combination: optionally, wherein said G713-related biallelic marker is selected from the group consisting of A1 to A11, and the complements thereof, or optionally the biallelic markers in linkage disequilibrium therewith; optionally, wherein said 13q31-q33-related biallelic marker is selected from the group consisting of A12 to A49, and the complements thereof, or optionally the biallelic markers in linkage disequilibrium therewith; optionally, wherein said 13q31-q33-related biallelic marker is selected from the group consisting of A16 to A20, and the complements thereof, or optionally the biallelic markers in linkage disequilibrium therewith; optionally, wherein said 13q31-q33-related biallelic marker is selected from the group consisting of A14, A15, A17, A18, A27, A28, A34, A35, A38, and the complements thereof, or optionally the biallelic markers in linkage disequilibrium therewith; optionally, said haplotype determination method is performed by asymmetric PCR amplification, double PCR amplification of specific alleles, the Clark algorithm, or an expectation-maximization algorithm.

Linkage Disequilibrium Analysis

Linkage disequilibrium is the non-random association of alleles at two or more loci and represents a powerful tool for mapping genes involved in disease traits (see Ajioka R. S. et al., 1997). Biallelic markers, because they are densely spaced in the human genome and can be genotyped in greater numbers than other types of genetic markers (such as RFLP or VNTR markers), are particularly useful in genetic analysis based on linkage disequilibrium.

When a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: one finds the disease mutation only in the presence of a specific set of marker alleles. Through subsequent generations recombination events occur between the disease mutation and these marker polymorphisms, and the disequilibrium gradually dissipates. The pace of this dissipation is a function of the recombination frequency, so the markers closest to the disease gene will manifest higher levels of disequilibrium than those that are further away. When not broken up by recombination, “ancestral” haplotypes and linkage disequilibrium between marker alleles at different loci can be tracked not only through pedigrees but also through populations. Linkage disequilibrium is usually seen as an association between one specific allele at one locus and another specific allele at a second locus.

The pattern or curve of disequilibrium between disease and marker loci is expected to exhibit a maximum that occurs at the disease locus. Consequently, the amount of linkage disequilibrium between a disease allele and closely linked genetic markers may yield valuable information regarding the location of the disease gene. For fine-scale mapping of a disease locus, it is useful to have some knowledge of the patterns of linkage disequilibrium that exist between markers in the studied region. As mentioned above the mapping resolution achieved through the analysis of linkage disequilibrium is much higher than that of linkage studies. The high density of biallelic markers combined with linkage disequilibrium analysis provides powerful tools for fine-scale mapping. Different methods to calculate linkage disequilibrium are described below under the heading “Statistical Methods”.

Population-Based Case-Control Studies of Trait-Marker Associations

As mentioned above, the occurrence of pairs of specific alleles at different loci on the same chromosome is not random and the deviation from random is called linkage disequilibrium. Association studies focus on population frequencies and rely on the phenomenon of linkage disequilibrium. If a specific allele in a given gene is directly involved in causing a particular trait, its frequency will be statistically increased in an affected (trait positive) population, when compared to the frequency in a trait negative population or in a random control population. As a consequence of the existence of linkage disequilibrium, the frequency of all other alleles present in the haplotype carrying the trait-causing allele will also be increased in trait positive individuals compared to trait negative individuals or random controls. Therefore, association between the trait and any allele (specifically a biallelic marker allele) in linkage disequilibrium with the trait-causing allele will suffice to suggest the presence of a trait-related gene in that particular region. Case-control populations can be genotyped for biallelic markers to identify associations that narrowly locate a trait causing allele. As any marker in linkage disequilibrium with one given marker associated with a trait will be associated with the trait. Linkage disequilibrium allows the relative frequencies in case-control populations of a limited number of genetic polymorphisms (specifically biallelic markers) to be analyzed as an alternative to screening all possible functional polymorphisms in order to find trait-causing alleles. Association studies compare the frequency of marker alleles in unrelated case-control populations, and represent powerful tools for the dissection of complex traits.

Case-Control Populations (Inclusion Criteria)

Population-based association studies do not concern familial inheritance but compare the prevalence of a particular genetic marker, or a set of markers, in case-control populations. They are case-control studies based on comparison of unrelated case (affected or trait positive) individuals and unrelated control (unaffected, trait negative or random) individuals. Preferably the control group is composed of unaffected or trait negative individuals. Further, the control group is ethnically matched to the case population. Moreover, the control group is preferably matched to the case-population for the main known confusion factor for the trait under study (for example age-matched for an age-dependent trait). Ideally, individuals in the two samples are paired in such a way that they are expected to differ only in their disease status. The terms “trait positive population”, “case population” and “affected population” are used interchangeably herein.

An important step in the dissection of complex traits using association studies is the choice of case-control populations (see Lander and Schork, 1994). A major step in the choice of case-control populations is the clinical definition of a given trait or phenotype. Any genetic trait may be analyzed by the association method proposed here by carefully selecting the individuals to be included in the trait positive and trait negative phenotypic groups. Four criteria are often useful: clinical phenotype, age at onset, family history and severity. The selection procedure for continuous or quantitative traits (such as blood pressure for example) involves selecting individuals at opposite ends of the phenotype distribution of the trait under study, so as to include in these trait positive and trait negative populations individuals with non-overlapping phenotypes. Preferably, case-control populations comprise phenotypically homogeneous populations. Trait positive and trait negative populations comprise phenotypically uniform populations of individuals representing each between 1 and 98%, preferably between 1 and 80%, more preferably between 1 and 50%, and more preferably between 1 and 30%, most preferably between 1 and 20% of the total population under study, and preferably selected among individuals exhibiting non-overlapping phenotypes. The clearer the difference between the two trait phenotypes, the greater the probability of detecting an association with biallelic markers. The selection of those drastically different but relatively uniform phenotypes enables efficient comparisons in association studies and the possible detection of marked differences at the genetic level, provided that the sample sizes of the populations under study are significant enough.

In preferred embodiments, a first group of between 50 and 300 trait positive individuals, preferably about 100 individuals, are recruited according to their phenotypes. A similar number of control individuals are included in such studies.

Association Analysis

The invention also comprises methods of detecting an association between a genotype and a phenotype, comprising the steps of: a) determining the frequency of at least one G713- or 13q31-q33-related biallelic marker in a trait positive population according to a genotyping method of the invention; b) determining the frequency of said G713- or 13q31-q33-related biallelic marker in a control population according to a genotyping method of the invention; and c) determining whether a statistically significant association exists between said genotype and said phenotype. In addition, the methods of detecting an association between a genotype and a phenotype of the invention encompass methods with any further limitation described in this disclosure, or those following, specified alone or in any combination: optionally, wherein said G713-related biallelic marker is selected from the group consisting of A1 to A11, and the complements thereof, or optionally the biallelic markers in linkage disequilibrium therewith; optionally, wherein said 13q31-q33-related biallelic marker is selected from the group consisting of A12 to A49, and the complements thereof, or optionally the biallelic markers in linkage disequilibrium therewith; optionally, wherein said 13q31-q33-related biallelic marker is selected from the group consisting of A16 to A20, and the complements thereof, or optionally the biallelic markers in linkage disequilibrium therewith; optionally, wherein said 13q31-q33-related biallelic marker is selected from the group consisting of A14, A15, A17, A18, A27, A28, A34, A35, A38, and the complements thereof, or optionally the biallelic markers in linkage disequilibrium therewith. Optionally, said control population may be a trait negative population, or a random population; Optionally, each of said genotyping steps a) and b) may be performed on a pooled biological sample derived from each of said populations; Optionally, each of said genotyping of steps a) and b) is performed separately on biological samples derived from each individual in said population or a subsample thereof.

The general strategy to perform association studies using biallelic markers derived from a region carrying a candidate gene is to scan two groups of individuals (case-control populations) in order to measure and statistically compare the allele frequencies of the biallelic markers of the present invention in both groups. If a statistically significant association with a trait is identified for at least one or more of the analyzed biallelic markers, one can assume that: either the associated aliele is directly responsible for causing the trait (i.e. the associated allele is the trait causing allele), or more likely the associated allele is in linkage disequilibrium with the trait causing allele. The specific characteristics of the associated allele with respect to the candidate gene function usually give further insight into the relationship between the associated allele and the trait (causal or in linkage disequilibrium). If the evidence indicates that the associated allele within the candidate gene is most probably not the trait causing allele but is in linkage disequilibrium with the real trait causing allele, then the trait causing allele can be found by sequencing the vicinity of the associated marker, and performing further association studies with the polymorphisms that are revealed in an iterative manner.

Association studies are usually run in two successive steps. In a first phase, the frequencies of a reduced number of biallelic markers from the candidate gene are determined in the trait positive and control populations. In a second phase of the analysis, the position of the genetic loci responsible for the given trait is further refined using a higher density of markers from the relevant region. However, if the candidate gene under study is relatively small in length, as is the case for G713, a single phase may be sufficient to establish significant associations.

Haplotype Analysis

As described above, when a chromosome carrying a disease allele first appears in a population as a result of either mutation or migration, the mutant allele necessarily resides on a chromosome having a set of linked markers: the ancestral haplotype. This haplotype can be tracked through populations and its statistical association with a given trait can be analyzed. Complementing single point (allelic) association studies with multi-point association studies also called haplotype studies increases the statistical power of association studies. Thus, a haplotype association study allows one to define the frequency and the type of the ancestral carrier haplotype. A haplotype analysis is important in that it increases the statistical power of an analysis involving individual markers.

In a first stage of a haplotype frequency analysis, the frequency of the possible haplotypes based on various combinations of the identified biallelic markers of the invention is determined. The haplotype frequency is then compared for distinct populations of trait positive and control individuals. The number of trait positive individuals, which should be, subjected to this analysis to obtain statistically significant results usually ranges between 30 and 300, with a preferred number of individuals ranging between 50 and 150. The same considerations apply to the number of unaffected individuals (or random control) used in the study. The results of this first analysis provide haplotype frequencies in case-control populations, for each evaluated haplotype frequency a p-value and an odd ratio are calculated. If a statistically significant association is found the relative risk for an individual carrying the given haplotype of being affected with the trait under study can be approximated.

An additional embodiment of the present invention encompasses methods of detecting an association between a haplotype and a phenotype, comprising the steps of: a) estimating the frequency of at least one haplotype in a trait positive population, according to a method of the invention for estimating the frequency of a haplotype; b) estimating the frequency of said haplotype in a control population, according to a method of the invention for estimating the frequency of a haplotype; and c) determining whether a statistically significant association exists between said haplotype and said phenotype. In addition, the methods of detecting an association between a haplotype and a phenotype of the invention encompass methods with any further limitation described in this disclosure, or those following: optionally, wherein said G713-related biallelic marker is selected from the group consisting of A1 to A11, and the complements thereof, or optionally the biallelic markers in linkage disequilibrium therewith; optionally, wherein said 13q31-q33-related biallelic marker is selected from the group consisting of A12 to A49, and the complements thereof, or optionally the biallelic markers in linkage disequilibrium therewith; optionally, wherein said 13q31-q33-related biallelic marker is selected from the group consisting of A16 to A20, and the complements thereof, or optionally the biallelic markers in linkage disequilibrium therewith; optionally, wherein said 13q31-q33-related biallelic marker is selected from the group consisting of A14, A15, A17, A18, A27, A28, A34, A35, A38, and the complements thereof, or optionally the biallelic markers in linkage disequilibrium therewith. Optionally, said control population is a trait negative population, or a random population. Optionally, said method comprises the additional steps of determining the phenotype in said trait positive and said control populations prior to step c).

Interaction Analysis

The biallelic markers of the present invention may also be used to identify patterns of biallelic markers associated with detectable traits resulting from polygenic interactions. The analysis of genetic interaction between alleles at unlinked loci requires individual genotyping using the techniques described herein. The analysis of allelic interaction among a selected set of biallelic markers with appropriate level of statistical significance can be considered as a haplotype analysis. Interaction analysis comprises stratifying the case-control populations with respect to a given haplotype for the first loci and performing a haplotype analysis with the second loci with each subpopulation.

Statistical methods used in association studies are further described below.

Testing for Linkage in the Presence of Association

The biallelic markers of the present invention may further be used in TDT (transmission/disequilibrium test). TDT tests for both linkage and association and is not affected by population stratification. TDT requires data for affected individuals and their parents or data from unaffected sibs instead of from parents (see Spielmann S. et al., 1993; Schaid D. J. et al., 1996, Spielmann S. and Ewens W. J., 1998). Such combined tests generally reduce the false—positive errors produced by separate analyses.

Statistical Methods

In general, any method known in the art to test whether a trait and a genotype show a statistically significant correlation may be used.

1) Methods in Linkage Analysis

Statistical methods and computer programs useful for linkage analysis are well-known to those skilled in the art (see Terwilliger J. D. and Ott J., 1994; Ott J., 1991).

2) Methods to Estimate Haplotype Frequencies in a Population

As described above, when genotypes are scored, it is often not possible to distinguish heterozygotes so that haplotype frequencies cannot be easily inferred. When the gametic phase is not known, haplotype frequencies can be estimated from the multilocus genotypic data. Any method known to person skilled in the art can be used to estimate haplotype frequencies (see Lange K., 1997; Weir, B. S., 1996) Preferably, maximum-likelihood haplotype frequencies are computed using an Expectation-Maximization (EM) algorithm (see Dempster et al., 1977; Excoffier L. and Slatkin M., 1995). This procedure is an iterative process aiming at obtaining maximum-likelihood estimates of haplotype frequencies from multi-locus genotype data when the gametic phase is unknown. Haplotype estimations are usually performed by applying the EM algorithm using for example the EM-HAPLO program (Hawley M. E. et al., 1994) or the Arlequin program (Schneider et al., 1997). The EM algorithm is a generalized iterative maximum likelihood approach to estimation and is briefly described below.

Please note that in the present section, “Methods To Estimate Haplotype Frequencies In A Population,” of this text, phenotypes will refer to multi-locus genotypes with unknown phase. Genotypes will refer to known-phase multi-locus genotypes.

A sample of N unrelated individuals is typed for K markers. The data observed are the unknown-phase K-locus phenotypes that can categorized in F different phenotypes. Suppose that we have H underlying possible haplotypes (in case of K biallelic markers, H=2^{K}).

For phenotype j, suppose that cⱼ genotypes are possible. We thus have the following equation

$$P j = {\sum }_{i = 1}^{{c}_{1}} p r ( {genotype}_{i} ) = {\sum }_{i = 1}^{{c}_{j}} p r ( {h}_{k} , {h}_{l} ) \qquad \text{Equation 1}$$

where Pj is the probability of the phenotype j, hₖ and hₗ are the two haplotypes constituent the genotype i. Under the Hardy-Weinberg equilibrium, pr(hₖ,h_{d}) becomes:

pr(hₖ, hₗ)=pr(hₖ)² if hₖ=hₗ, pr(hₖ,hₗ)=2pr(hₖ)·pr(hₗ) if hₖ‥hₗ. Equation 2

The successive steps of the E-M algorithm can be described as follows:

Starting with initial values of the of haplotypes frequencies, noted p₁⁽⁰⁾, p₂⁽⁰⁾, . . . p_{H}⁽⁰⁾, these initial values serve to estimate the genotype frequencies (Expectation step) and then estimate another set of haplotype frequencies (Maximization step), noted p₁⁽¹⁾, p₂⁽¹⁾, . . . p_{H}⁽¹⁾, these two steps are iterated until changes in the sets of haplotypes frequency are very small.

A stop criterion can be that the maximum difference between haplotype frequencies between two iterations is less than 10⁻⁷. These values can be adjusted according to the desired precision of estimations.

At a given iteration s, the Expectation step comprises calculating the genotypes frequencies by the following equation:

$$\begin{matrix}p {r ( {genotype}_{i} )}^{( s )} = p r ( {phenotype}_{j} ) \cdot p {r ( {genotype}_{i} \vert {phenotype}_{j} )}^{( s )} \\ = \frac{{n}_{j}}{N} \cdot \frac{p {r ( {h}_{k} , {h}_{l} )}^{( s )}}{{P}_{j}^{( s )}}\end{matrix} \qquad \text{Equation 3}$$

where genotype i occurs in phenotype j, and where hₖ and hₗ constitute genotype i. Each probability is derived according to eq. 1, and eq. 2 described above.

Then the Maximization step simply estimates another set of haplotype frequencies given the genotypes frequencies. This approach is also known as the gene-counting method (Smith, 1957).

$${P}_{t}^{( s + 1 )} = \frac{1}{2} {\sum }_{j = 1}^{F} {\sum }_{i = 1}^{{c}_{j}} {δ}_{i t} \cdot p {r ( {genotype}_{i} )}^{( s )} \qquad \text{Equation 4}$$

Where δᵢₜ it is an indicator variable which count the number of time haplotype t in genotype i. It takes the values of 0, 1 or 2.

To ensure that the estimation finally obtained is the maximum-likelihood estimation several values of departures are required. The estimations obtained are compared and if they are different the estimations leading to the best likelihood are kept.

3) Methods to Calculate Linkage Disequilibrium Between Markers

A number of methods can be used to calculate linkage disequilibrium between any two genetic positions, in practice linkage disequilibrium is measured by applying a statistical association test to haplotype data taken from a population.

Linkage disequilibrium between any pair of biallelic markers comprising at least one of the biallelic markers of the present invention (Mᵢ, Mⱼ) having alleles (aᵢ/bᵢ) at marker Mᵢ and alleles (aⱼ/bⱼ) at marker Mⱼ can be calculated for every allele combination (aᵢ,aⱼ, aᵢ,bⱼ, bᵢ,aⱼ and bᵢ,bⱼ), according to the Piazza formula:

Δₐᵢₐⱼ=√θ4−√(θ4+θ3) (θ4+θ2), where:

θ4=−−=frequency of genotypes not having allele aᵢ at Mᵢ and not having allele aⱼ at Mⱼ

θ3=−+=frequency of genotypes not having allele aᵢ at Mᵢ and having allele aⱼ at Mⱼ

θ2=+−=frequency of genotypes having allele aᵢ at Mᵢ and not having allele aⱼ at Mⱼ

Linkage disequilibrium (LD) between pairs of biallelic markers (Mᵢ, Mᵢ) can also be calculated for every allele combination (ai, aj ai, bj, bᵢ, aⱼ and bᵢ, bⱼ), according to the maximum-likelihood estimate (MLE) for delta (the composite genotypic disequilibrium coefficient), as described by Weir (Weir B. S., 1996). The MLE for the composite linkage disequilibrium is:

Dₐᵢₐⱼ=(2n₁+n₂+n₃+n₄/2)/N−2(pr(aᵢ). pr(aⱼ))

Where n₁=Σ phenotype (aᵢ/aᵢ, aⱼ/aⱼ), n₂=Σ phenotype (aᵢ/aᵢ, aⱼ/bⱼ), n₃=Σ phenotype (aᵢ/bᵢ, aⱼ/aⱼ), n4=Σ phenotype (aᵢ/bᵢ, aⱼ/bⱼ) and N is the number of individuals in the sample.

This formula allows linkage disequilibrium between alleles to be estimated when only genotype, and not haplotype, data are available.

Another means of calculating the linkage disequilibrium between markers is as follows. For a couple of biallelic markers, Mᵢ (aᵢ/bᵢ) and Mⱼ(aⱼ/bⱼ), fitting the Hardy-Weinberg equilibrium, one can estimate the four possible haplotype frequencies in a given population according to the approach described above.

The estimation of gametic disequilibrium between ai and aj is simply:

Dₐᵢₐⱼ=pr(haplotype(aᵢ, aⱼ))−pr(aᵢ).pr(aⱼ).

Where pr(aᵢ) is the probability of allele aᵢ and pr(aⱼ) is the probability of allele aⱼ and where pr(haplotype (aᵢ, aⱼ)) is estimated as in Equation 3 above.

For a couple of biallelic marker only one measure of disequilibrium is necessary to describe the association between Mᵢ and Mⱼ.

Then a normalized value of the above is calculated as follows:

D′ₐᵢₐⱼ=Dₐᵢₐⱼ/max(−pr(aᵢ). pr(aⱼ), −pr(bᵢ). pr(bⱼ)) with Dₐᵢₐⱼ<0

D′ₐᵢₐⱼ=Dₐᵢₐⱼ/max(pr(bᵢ). pr(aⱼ), pr(aᵢ). pr(bⱼ)) with Dₐᵢₐⱼ>0

The skilled person will readily appreciate that other linkage disequilibrium calculation methods can be used.

Linkage disequilibrium among a set of biallelic markers having an adequate heterozygosity rate can be determined by genotyping between 50 and 1000 unrelated individuals, preferably between 75 and 200, more preferably around 100.

4) Testing for Association

Methods for determining the statistical significance of a correlation between a phenotype and a genotype, in this case an allele at a biallelic marker or a haplotype made up of such alleles, may be determined by any statistical test known in the art and with any accepted threshold of statistical significance being required. The application of particular methods and thresholds of significance are well with in the skill of the ordinary practitioner of the art.

Testing for association is performed by determining the frequency of a biallelic marker allele in case and control populations and comparing these frequencies with a statistical test to determine if their is a statistically significant difference in frequency which would indicate a correlation between the trait and the biallelic marker allele under study. Similarly, a haplotype analysis is performed by estimating the frequencies of all possible haplotypes for a given set of biallelic markers in case and control populations, and comparing these frequencies with a statistical test to determine if their is a statistically significant correlation between the haplotype and the phenotype (trait) under study. Any statistical tool useful to test for a statistically significant association between a genotype and a phenotype may be used. Preferably the statistical test employed is a chi-square test with one degree of freedom. A P-value is calculated (the P-value is the probability that a statistic as large or larger than the observed one would occur by chance).

Statistical Significance

In preferred embodiments, significance for diagnosis purposes, either as a positive basis for further diagnostic tests or as a preliminary starting point for early preventive therapy, the p value related to a biallelic marker association is preferably about 1×10⁻² or less, more preferably about 1×10⁻⁴ or less, for a single biallelic marker analysis and about 1×10⁻³ or less, still more preferably 1×10⁻⁶ or less and most preferably of about 1×10⁻⁸ or less, for a haplotype analysis involving two or more markers. These values are believed to be applicable to any association studies involving single or multiple marker combinations. The skilled person can use the range of values set forth above as a starting point in order to carry out association studies with biallelic markers of the present invention. In doing so, significant associations between the biallelic markers of the present invention and a trait can be revealed and used for diagnosis and drug screening purposes.

Phenotypic Permutation

In order to confirm the statistical significance of the first stage haplotype analysis described above, it might be suitable to perform further analyses in which genotyping data from case-control individuals are pooled and randomized with respect to the trait phenotype. Each individual genotyping data is randomly allocated to two groups, which contain the same number of individuals as the case-control populations used to compile the data obtained in the first stage. A second stage haplotype analysis is preferably run on these artificial groups, preferably for the markers included in the haplotype of the first stage analysis showing the highest relative risk coefficient. This experiment is reiterated preferably at least between 100 and 10000 times. The repeated iterations allow the determination of the probability to obtain the tested haplotype by chance.

Assessment of Statistical Association

To address the problem of false positives similar analysis may be performed with the same case-control populations in random genomic regions. Results in random regions and the candidate region are compared as described in a co-pending U.S. Provisional Patent Application entitled “Methods, Software And Apparati For Identifying Genomic Regions Harboring A Gene Associated With A Detectable Trait,” U.S. Ser. No. 60/107,986, filed Nov. 10, 1998, the contents of which are incorporated herein by reference.

5) Evaluation of Risk Factors

The association between a risk factor (in genetic epidemiology the risk factor is the presence or the absence of a certain allele or haplotype at marker loci) and a disease is measured by the odds ratio (OR) and by the relative risk (RR). If P(R⁺) is the probability of developing the disease for individuals with R and P(R⁻) is the probability for individuals without the risk factor, then the relative risk is simply the ratio of the two probabilities, that is:

RR=P(R⁺)/P(R⁻)

In case-control studies, direct measures of the relative risk cannot be obtained because of the sampling design. However, the odds ratio allows a good approximation of the relative risk for low-incidence diseases and can be calculated:

OR=(F⁺/(1−F⁺))/(F/(1−F⁻))

F⁺ is the frequency of the exposure to the risk factor in cases and F⁻ is the frequency of the exposure to the risk factor in controls. F⁺ and F⁻ are calculated using the allelic or haplotype frequencies of the study and further depend on the underlying genetic model (dominant, recessive, additive . . . ).

One can further estimate the attributable risk (AR) which describes the proportion of individuals in a population exhibiting a trait due to a given risk factor. This measure is important in quantifying the role of a specific factor in disease etiology and in terms of the public health impact of a risk factor. The public health relevance of this measure lies in estimating the proportion of cases of disease in the population that could be prevented if the exposure of interest were absent. AR is determined as follows:

AR=P_{E}(RR−1)/(P_{E}(RR−1)+1)

AR is the risk attributable to a biallelic marker allele or a biallelic marker haplotype. P_{E} is the frequency of exposure to an allele or a haplotype within the population at large; and RR is the relative risk which, is approximated with the odds ratio when the trait under study has a relatively low incidence in the general population.

Association of 13q31-q33 Biallelic Markers of the Invention with “a trait”

In one preferred embodiment of the invention, a correlation was found between the biallelic markers comprised in BAC 5 and BAC 9, the DNA inserts of which are contained in the human chromosome 13q31-q33 region and schizophrenia, results of the association study are further described in details in Example 2(f). BAC B1 to BAC B9 are referred to throughout the present specification simply to illustrate the experimental procedures used by the inventors to identify the biallelic markers described herein, more particularly the biallelic markers in association with schizophrenia. Once the biallelic markers of the invention have been discovered and the association of a number of them with schizophrenia established, the one skilled in the art is enabled to reproduce the teachings of the present specification with the knowledge of the methods described herein as well as with the knowledge of the nucleic acid sequences disclosed in the appended Sequence Listing, without any need to use again any of the BACs B1 to B9 that only represent the starting material of the inventors.

More precisely, the biallelic markers 99-15663-298, 99-15665-398, 99-15672-166 and 99-15664-185 which are located in BAC 5 show a slight association with schizophrenia, and more particularly with familial cases of schizophrenia. Comparably, the biallelic markers 99-5919-215, 99-5862-167, 99-16032-292 and 99-16038-118 which are located in BAC 9 also show a slight association with schizophrenia.

The inventors also considered the LD values between every set of two biallelic markers of the human chromosome 13q31-q33 region for cases and controls. Indeed, a difference of LD between two markers in the cases compared to the controls can reveal an association of these biallelic markers with the studied trait. The inventors noticed that the highest relative difference in LD value between cases and controls was observed for BAC 5 and BAC 9.

Similar association studies can also be carried out with other biallelic markers within the scope of the invention, preferably with biallelic markers in LD with the markers associated with schizophrenia as described above, including the biallelic markers of SEQ ID Nos 32-69.

Similar association studies can be carried out by the skilled technician using the biallelic markers of the invention defined above, with different trait + and trait − populations. Suitable further examples of association studies using biallelic markers of the human chromosome 13q31-q33 region, including the biallelic markers of SEQ ID Nos 32-69, involve studies on the following populations:

a trait+population suffering from schizophrenia treated with agents acting against schizophrenia or against schizophrenia symptoms and suffering from side-effects resulting from this treatment and an trait − population suffering from schizophrenia treated with the same agents without any substantial side-effects, or

a trait + population suffering from schizophrenia treated with agents acting against schizophrenia or schizophrenia symptoms showing a beneficial response and a trait − population suffering from schizophrenia treated with same agents without any beneficial response.

Haplotype Frequency Analysis

From the data resulting from the association analysis between alleles of the biallelic markers located on BAC 5 of the human chromosome 13q31-q33 region and schizophrenia, several haplotypes were shown to be statistically associated (see Table 15). For example, a preferred haplotype comprises the two biallelic markers 99-15672-166 (allele T) and 99-15664-185 (allele T). This haplotype is significantly associated with schizophrenia with a p-value of 2.5×10⁻⁵. Among 1000 random permutation iterations between cases and controls, only 1‰ of the resulting p-values are equal or below to the one experimentally obtained in Table 15 for the haplotype 1. These results clearly validate the statistical significance of the haplotype 1 of the present invention. Furthermore, three markers-haplotypes and the four markers haplotype comprising the two biallelic markers 99-15672-166 (allele T) and 99-15664-185 (allele T) are also considered to be significant of an association with schizophrenia (haplotypes 7, 8 and 11 of Table 15).

The haplotype analysis described above shows that a gene linked to schizophrenia susceptibility lies at proximity of the markers defining haplotype 1 on the human genome.

From the results from Tables 16 and 17 with the biallelic markers located on BAC 9, the inserts of which are comprised in the human chromosome 13q31-q33 region, several haplotypes were shown to be significantly associated with schizophrenia. For example, a preferred haplotype (haplotype 5 in Table 16 and 17) comprises the two biallelic markers 99-5862-167 (allele C) and 99-16032-292 (allele C). This haplotype is considered to be significant of an association with schizophrenia with a p-value less than 10⁻⁶. Among 1000 permutation iterations, none of the resulting p-values are equal or below to the p-value experimentally obtained for the considered haplotype in Table 16 and in Table 17. These results clearly validate the statistical significance of the haplotype of the present invention. Three markers-haplotypes (haplotypes 18, 19 and 17 of Tables 16 and 17) and one four-markers haplotype (haplotype 25 of Tables 16 and 17) comprising the biallelic marker 99-5862-167 (allele C), and more frequently the two biallelic markers 99-5862-167 (allele C) and 99-16032-292 (C), are also considered to be significant of an association with schizophrenia. Indeed they present a p-value inferior to 10⁻⁶.

The haplotypes 5, 17, 18, 19 and 25 of Tables 16 and 17 are associated with familial schizophrenia and are thus located in a region harboring a gene involved in the predisposition or in the development of schizophrenia.

The highest significant association with schizophrenia has been obtained for haplotypes combining the biallelic markers 99-15672-166 (allele T) and 99-15664-185 (allele T) located on BAC 5 with the biallelic markers 99-5862-167 (allele T) and 99-16032-292 (allele C) located on BAC 9. Several haplotypes, more particularly three markers-haplotypes 7, 8 and 9 and the four markers-haplotype 11 of Table 18 are highly significant of an association with schizophrenia with a p-value less than 10⁻⁶. Moreover, haplotypes 7 and 11 present a p-value less than 10⁻¹⁰. Among 50,000 permutation iterations, less than 2‰ of the resulting p-values are equal or below to the experimentally obtained p-values for haplotypes 7, 8, and 11 of Table 18.

Additionally, the data from Example 2(h)(iv) demonstrate that when all the possible combinations (haplotypes) of two or three markers among the markers listed in Table 7 are studied for their association with schizophrenia, the haplotypes that are the most strongly associated with schizophrenia only contain biallelic markers located in BAC B5 and/or BAC B9.

Moreover, a selection (1%) of the two markers- and the three markers-haplotypes giving the more significant p-value has been performed, and then the number of selected haplotypes has been restricted to those for which the estimated haplotype frequency in the cases population was not less than 0.2 (20%). All these selected haplotypes contained biallelic markers located in BAC B5 and/or BAC B9 (data not shown).

Without wishing to be bound by any particular theory, the inventors believe that in order to be sufficiently significant to be reliable for diagnosis purposes, either as a positive basis for further diagnostic tests or as a preliminary starting point for early preventive therapy, the p value related to a biallelic marker association is preferably about 1×10⁻² or less, more preferably about 1×₁₀₋₄ or less, for a single biallelic marker analysis and about 1×10³ or less, still more preferably 1×10⁻⁶ or less and most preferably of about 1×10⁻⁸ or less, for a haplotype analysis involving several markers. These values are believed to be applicable to any association studies involving single or multiple marker combinations.

The skilled person can use the range of values set forth above as a starting point in order to carry out association studies with other biallelic markers of the human chromosome 13q31-q33 region, or with markers from other genomic DNA sequences. In doing so, further significant associations between biallelic markers of the human chromosome 13q31-q33 region and schizophrenia can be revealed and used for diagnosis and drug screening purposes.

Using the method described above and evaluating the associations for single marker alleles or for haplotypes permits an estimation of the risk a corresponding carrier has to develop a given trait, and particularly in the context of the present invention, a disease, preferably schizophrenia. Significance thresholds of relative risks are to be adapted to the reference sample population used.

It is difficult to evaluate accurately quantified boundaries for the so-called “significant risk”. Indeed, and as it has been demonstrated previously, several traits observed in a given population are multifactorial in that they are not only the result of a single genetic predisposition but also of other factors such as environmental factors or the presence of further, apparently unrelated, haplotype associations. Thus, the evaluation of a significant risk must take these parameters into consideration in order to, in a certain manner, weigh the potential importance of external parameters in the development of a given trait.

Without wishing to be bound to any invariable model or theory based on the above statistical analyses, the inventors believe that a “significant risk” to develop a given trait is evaluated differently depending on the trait under consideration.

It will of course be understood by practitioners skilled in the treatment of schizophrenia that the present invention does not intend to provide an absolute identification of individuals who could be at risk of developing schizophrenia but rather to indicate a certain degree or likelihood of developing the disease.

However, this information is extremely valuable as it can, in certain circumstances, be used to initiate preventive treatments or to allow an individual carrying a significant haplotype to foresee warning signs such as minor symptoms. In diseases such as schizophrenia, the knowledge of a potential predisposition, even if this predisposition is not absolute, might contribute in a very significant manner to treatment efficacy. Similarly, a diagnosed predisposition to a potential side-effect could immediately direct the physician toward a treatment for which such side-effects have not been observed during clinical trials.

Identification of Biallelic Markers in Linkage Disequilibrium With the Biallelic Markers of the Invention

Once a first biallelic marker has been identified in a genomic region of interest, the practitioner of ordinary skill in the art, using the teachings of the present invention, can easily identify additional biallelic markers in linkage disequilibrium with this first marker. As mentioned before any marker in linkage disequilibrium with a first marker associated with a trait will be associated with the trait. Therefore, once an association has been demonstrated between a given biallelic marker and a trait, the discovery of additional biallelic markers associated with this trait is of great interest in order to increase the density of biallelic markers in this particular region. The causal gene or mutation will be found in the vicinity of the marker or set of markers showing the highest correlation with the trait.

Identification of additional markers in linkage disequilibrium with a given marker involves: (a) amplifying a genomic fragment comprising a first biallelic marker from a plurality of individuals; (b) identifying of second biallelic markers in the genomic region harboring said first biallelic marker; (c) conducting a linkage disequilibrium analysis between said first biallelic marker and second biallelic markers; and (d) selecting said second biallelic markers as being in linkage disequilibrium with said first marker. Subcombinations comprising steps (b) and (c) are also contemplated.

Methods to identify biallelic markers and to conduct linkage disequilibrium analysis are described herein and can be carried out by the skilled person without undue experimentation. The present invention then also concerns biallelic markers which are in linkage disequilibrium with the biallelic markers A1 to A11 and A12 to A49, and which are expected to present similar characteristics in terms of their respective association with a given trait. Preferably, the invention concerns biallelic markers which are in linkage disequilibrium with the 13q31-q33-related biallelic markers A16 to A20.

Identification of Functional Mutations

Mutations in a candidate gene such as a 13q31-q33 gene or G713, for example, which are responsible for a detectable phenotype or trait may be identified by comparing the sequences of the candidate gene from trait positive and control individuals. Once a positive association is confirmed with a biallelic marker of the present invention, the identified locus can be scanned for mutations. In a preferred embodiment, functional regions such as exons and splice sites, promoters and other regulatory regions of the candidate gene are scanned for mutations. In a preferred embodiment the sequence of the candidate gene is compared in trait positive and control individuals. Preferably, trait positive individuals carry the haplotype shown to be associated with the trait and trait negative individuals do not carry the haplotype or allele associated with the trait. The detectable trait or phenotype may comprise a variety of manifestations of altered G713 or the 13q31-q33 candidate gene function.

The mutation detection procedure is essentially similar to that used for biallelic marker identification. The method used to detect such mutations generally comprises the following steps:

amplification of a region of the G713 or 13q31-q33 candidate gene gene comprising a biallelic marker or a group of biallelic markers associated with the trait from DNA samples of trait positive patients and trait-negative controls;

sequencing of the amplified region;

comparison of DNA sequences from trait positive and control individuals;

determination of mutations specific to trait-positive patients.

In one embodiment, said biallelic marker is a G713-related biallelic marker selected from the group consisting of A1 to A11, and the complements thereof. In another embodiment, said biallelic marker is a 13q31-q33-related biallelic marker selected from the group consisting of A12 to A49, and the complements thereof. In preferred embodiment, said 13q31-q33-related biallelic marker is selected from the group consisting of A16 to A20, and the complements thereof. It is preferred that candidate polymorphisms be then verified by screening a larger population of cases and controls by means of any genotyping procedure such as those described herein, preferably using a microsequencing technique in an individual test format. Polymorphisms are considered as candidate mutations when present in cases and controls at frequencies compatible with the expected association results. Polymorphisms are considered as candidate “trait-causing” mutations when they exhibit a statistically significant correlation with the detectable phenotype.

Biallelic Markers of the Invention in Methods of Genetic Diagnostics

The biallelic markers of the present invention can also be used to develop diagnostics tests capable of identifying individuals who express a detectable trait as the result of a specific genotype or individuals whose genotype places them at risk of developing a detectable trait at a subsequent time. The trait analyzed using the present diagnostics may be any detectable trait, including central nervous system diseases such as schizophrenia. Such a diagnosis can be useful in the staging, monitoring, prognosis and/or prophylactic or curative therapy of such diseases.

The diagnostic techniques of the present invention may employ a variety of methodologies to determine whether a test subject has a biallelic marker pattern associated with an increased risk of developing a detectable trait or whether the individual suffers from a detectable trait as a result of a particular mutation, including methods which enable the analysis of individual chromosomes for haplotyping, such as family studies, single sperm DNA analysis or somatic hybrids.

The present invention provides diagnostic methods to determine whether an individual is at risk of developing a disease or suffers from a disease resulting from a mutation or a polymorphism in a G713 or 13q31-q33 gene. The present invention also provides methods to determine whether an individual has a susceptibility to a particular disease such as schizophrenia.

These methods involve obtaining a nucleic acid sample from the individual and, determining, whether the nucleic acid sample contains at least one allele or at least one biallelic marker haplotype, indicative of a risk of developing the trait or indicative that the individual expresses the trait as a result of possessing a particular G713 or 13q31-q33 polymorphism or mutation (trait-causing allele).

Preferably, in such diagnostic methods, a nucleic acid sample is obtained from the individual and this sample is genotyped using methods described above in “Methods Of Genotyping DNA Samples For Biallelic Markers.” The diagnostics may be based on a single biallelic marker or a on group of biallelic markers. In each of these methods, a nucleic acid sample is obtained from the test subject and the biallelic marker pattern of one or more of the biallelic markers A1 to A49 is determined.

In one embodiment, a PCR amplification is conducted on the nucleic acid sample to amplify regions in which polymorphisms associated with a detectable phenotype have been identified. The amplification products are sequenced to determine whether the individual possesses one or more G713 or 13q31-q33 polymorphisms associated with a detectable phenotype. The primers used to generate amplification products may comprise the primers listed in Tables 1 and 6. Alternatively, the nucleic acid sample is subjected to microsequencing reactions as described above to determine whether the individual possesses one or more G713 or 13q31-q33 polymorphisms associated with a detectable phenotype resulting from a mutation or a polymorphism in a G713 or 13q31-q33 gene. The primers used in the microsequencing reactions may include the primers listed in Tables 4 and 8, respectively. In another embodiment, the nucleic acid sample is contacted with one or more allele specific oligonucleotide probes which, specifically hybridize to one or more G713 or 13q31-q33 alleles associated with a detectable phenotype. The probes used in the hybridization assay may include the probes listed in Tables 3 and 7, respectively. In another embodiment, the nucleic acid sample is contacted with a second G713 or 13q31-q33 oligonucleotide capable of producing an amplification product when used with the allele specific oligonucleotide in an amplification reaction. The presence of an amplification product in the amplification reaction indicates that the individual possesses one or more G713 or 13q31-q33 alleles associated with a detectable phenotype.

In a preferred embodiment the identity of the nucleotide present at, at least one, 13q31-q33-related biallelic marker selected from the group consisting of A12 to A49 and the complements thereof, preferably at least one biallelic marker selected from the group consisting of A16 to A20, and the complements thereof, is determined and the detectable trait is schizophrenia. Diagnostic kits comprise any of the polynucleotides of the present invention.

These diagnostic methods based on G713 and 13q31-q33 related biallelic markers are extremely valuable as they can, in certain circumstances, be used to initiate preventive treatments or to allow an individual carrying a significant haplotype to foresee warning signs such as minor symptoms. G713 and 13q31-q33 diagnostics, which analyze and predict response to a drug or side effects to a drug, may be used to determine whether an individual should be treated with a particular drug. For example, if the diagnostic indicates a likelihood that an individual will respond positively to treatment with a particular drug, the drug may be administered to the individual. Conversely, if the diagnostic indicates that an individual is likely to respond negatively to treatment with a particular drug, an alternative course of treatment may be prescribed. A negative response may be defined as either the absence of an efficacious response or the presence of toxic side effects.

Clinical drug trials represent another application for the markers of the present invention. One or more markers indicative of response to an agent acting against schizophrenia or to side effects to an agent acting against schizophrenia may be identified using the methods described above. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

Prevention and Treatment of Schizophrenia

Notably because the risk of suicide, it is important to detect schizophrenia susceptibility of individuals. Consequently, the invention also concerns a method for the treatment of schizophrenia comprising the following steps:

selecting an individual whose DNA comprises alleles of a biallelic marker or of a group of biallelic markers of the human chromosome 13q31-q33 region, associated with schizophrenia;

following up said individual for the appearance (and optionally the development) of the symptoms related to schizophrenia; and

administering a treatment acting against schizophrenia or against schizophrenia symptoms to said individual at an appropriate stage of the disease.

In one embodiment, the biallelic marker is one of those defined in SEQ ID Nos 32 to 69. In a preferred embodiment, the biallelic marker is selected from the group consisting of A16 to A20.

Another embodiment of the present invention consists of a method for the treatment of schizophrenia comprising the following steps:

selecting an individual whose DNA comprises alleles of a bialielic marker or of a group of biallelic markers of the human chromosome 13q31-q33 region gene, associated with schizophrenia;

administering a preventive treatment of schizophrenia to said individual.

In one embodiment, the biallelic marker is one of those defined in SEQ ID Nos 32 to 69. In a preferred embodiment, the biallelic marker is selected from the group consisting of A16 to A20.

In a further embodiment, the present invention concerns a method for the treatment of schizophrenia comprising the following steps:

selecting an individual whose DNA comprises alleles of a biallelic marker or of a group of biallelic markers of the human chromosome 13q31-q33 region associated with schizophrenia;

administering a preventive treatment of schizophrenia to said individual;

following up said individual for the appearance and the development of schizophrenia symptoms; and optionally

administering a treatment acting against schizophrenia or against schizophrenia symptoms to said individual at the appropriate stage of the disease.

In one embodiment, the biallelic marker is one of those defined in SEQ ID Nos 32 to 69. In a preferred embodiment, the biallelic marker is selected from the group consisting of A16 to A20.

To enlighten the choice of the appropriate beginning of the treatment of schizophrenia, the present invention also concerns a method for the treatment of schizophrenia comprising the following steps:

selecting an individual suffering from schizophrenia whose DNA comprises alleles of a biallelic marker or of a group of biallelic markers of the human chromosome 13q31-q33 region associated with the gravity of schizophrenia or of the schizophrenia symptoms; and

administering a treatment acting against schizophrenia or schizophrenia symptoms to said individual.

In one embodiment, the biallelic marker is one of those defined in SEQ ID Nos 32 to 69. In a preferred embodiment, the biallelic marker is selected from the group consisting of A16 to A20.

The invention also concerns a method for the treatment of schizophrenia in a selected population of individuals. The method comprises:

selecting an individual suffering from schizophrenia and whose DNA comprises alleles of a biallelic marker or of a group of biallelic markers of the human chromosome 13q31-q33 region associated with a positive response to treatment with an effective amount of a medicament acting against schizophrenia or schizophrenia symptoms,

and/or whose DNA does not comprise alleles of a biallelic marker or of a group of biallelic markers of the human chromosome 13q31-q33 region associated with a negative response to treatment with said medicament; and

administering at suitable intervals an effective amount of said medicament to said selected individual.

In some embodiments, the biallelic marker is one of those defined in SEQ ID Nos 32 to 69.

In the context of the present invention, a “positive response” to a medicament can be defined as comprising a reduction of the symptoms related to the disease.

In the context of the present invention, a “negative response” to a medicament can be defined as comprising either a lack of positive response to the medicament which does not lead to a symptom reduction or which leads to a side-effect observed following administration of the medicament.

The invention also relates to a method of determining whether a subject is likely to respond positively to treatment with a medicament.

The method comprises identifying a first population of individuals who respond positively to said medicament and a second population of individuals who respond negatively to said medicament. One or more biallelic markers is identified in the first population which is associated with a positive response to said medicament or one or more biallelic markers is identified in the second population which is associated with a negative response to said medicament. The biallelic markers may be identified using the techniques described herein.

A DNA sample is then obtained from the subject to be tested. The DNA sample is analyzed to determine whether it comprises alleles of one or more biallelic markers associated with a positive response to treatment with the medicament and/or alleles of one or more biallelic markers associated with a negative response to treatment with the medicament.

In some embodiments, the medicament may be administered to the subject in a clinical trial if the DNA sample contains alleles of one or more biallelic markers associated with a positive response to treatment with the medicament and/or if the DNA sample lacks alleles of one or more biallelic markers associated with a negative response to treatment with the medicament. In preferred embodiments, the medicament is a drug acting against schizophrenia. In one embodiment, the biallelic marker is one of those defined in SEQ ID Nos 32 to 69.

Using the method of the present invention, the evaluation of drug efficacy may be conducted in a population of individuals likely to respond favorably to the medicament.

Another aspect of the invention is a method of using a medicament comprising obtaining a DNA sample from a subject, determining whether the DNA sample contains alleles of one or more biallelic markers associated with a positive response to the medicament and/or whether the DNA sample contains alleles of one or more biallelic markers associated with a negative response to the medicament, and administering the medicament to the subject if the DNA sample contains alleles of one or more biallelic markers associated with a positive response to the medicament and/or if the DNA sample lacks alleles of one or more biallelic markers associated with a negative response to the medicament.

The invention also concerns a method for the clinical testing of a medicament, preferably a medicament acting against schizophrenia or schizophrenia symptoms. The method comprises the following steps:

administering a medicament, preferably a medicament susceptible of acting against schizophrenia or schizophrenia symptoms to a heterogeneous population of individuals,

identifying a first population of individuals who respond positively to said medicament and a second population of individuals who respond negatively to said medicament,

identifying biallelic markers in said first population which are associated with a positive response to said medicament,

selecting individuals whose DNA comprises biallelic markers associated with a positive response to said medicament, and

administering said medicament to said individuals.

Such methods are deemed to be extremely useful to increase the benefit/risk ratio resulting from the administration of medicaments which may cause undesirable side effects and/or be inefficacious to a portion of the patient population to which it is normally administered.

Once an individual has been diagnosed as suffering from schizophrenia, selection tests are carried out to determine whether the DNA of this individual comprises alleles of a biallelic marker or of a group of biallelic markers associated with a positive response to treatment or with a negative response to treatment which may include either side effects or unresponsiveness.

The selection of the patient to be treated using the method of the present invention can be carried out through the detection methods described above. The individuals which are to be selected are preferably those whose DNA does not comprise alleles of a biallelic marker or of a group of biallelic markers associated with a negative response to treatment. The knowledge of an individual's genetic predisposition to unresponsiveness or side effects to particular medicaments allows the clinician to direct treatment toward appropriate drugs against schizophrenia or schizophrenia symptoms.

Once the patient's genetic predispositions have been determined, the clinician can select appropriate treatment for which negative response, particularly side effects, has not been reported or has been reported only marginally for the patient.

Expression of a G713 Regulatory or Coding Polynucleotide of the Invention

Any of the regulatory polynucleotides or the coding polynucleotides of the invention may be inserted into recombinant vectors for expression in a recombinant host cell or a recombinant host organism.

Thus, the present invention also encompasses a family of recombinant vectors that contains either a regulatory polynucleotide selected from the group consisting of any one of the regulatory polynucleotides derived from the G713 genomic sequence, a coding polynucleotide or from the G713 genomic sequence or the G713 cDNA, or also a coding polynucleotide from the mouse G713 cDNA.

Consequently, the present invention further deals with a recombinant vector comprising either a regulatory polynucleotide contained in one of the nucleic acids of SEQ ID Nos 1 and 3, or a polynucleotide comprising the G713 coding sequence, or both.

In a first preferred embodiment, a recombinant vector of the invention is used as an expression vector: (a) the G713 regulatory sequence comprised therein drives the expression of a coding polynucleotide operably linked thereto; (b) the G713 coding sequence is operably linked to regulation sequences allowing its expression in a suitable cell host and/or host organism.

In a second preferred embodiment, a recombinant vector of the invention is used to amplify the inserted polynucleotide derived from a G713 genomic sequence selected from the group consisting of the nucleic acids of SEQ ID Nos 1 to 3 or a G713 cDNA of SEQ ID Nos 4 or 6 in a suitable cell host, this polynucleotide being amplified with the replication of the recombinant vector.

More particularly, the present invention relates to expression vectors which include nucleic acids encoding a G713 protein, preferably the human or murine G713 protein selected from the group consisting of the amino acid sequences of SEQ ID Nos 5, and 7 described therein, under the control of a regulatory sequence selected among the G713 regulatory polynucleotides, or alternatively under the control of an exogenous regulatory sequence.

A recombinant expression vector comprising a nucleic acid selected from the group consisting of nucleotide positions 1076 to 3075 of SEQ ID No 1, or biologically active fragments or variants thereof, is also part of the present invention.

The invention also encompasses a recombinant expression vector comprising

a) a nucleic acid comprising a regulatory polynucleotide of nucleotide positions 1076 to 3075 of SEQ ID No 1, or a biologically active fragment or variant thereof;

b) a polynucleotide encoding a polypeptide or a polynucleotide of interest operably linked with said nucleic acid.

c) optionally, a nucleic acid comprising a 3′-regulatory polynucleotide, preferably a 3′-regulatory polynucleotide of the invention, or a biologically active fragment or variant thereof.

The nucleic acid comprising the nucleotide sequence of SEQ ID No 1 or a biologically active fragment or variant thereof may also comprises the 5′-UTR sequence located between the nucleotide at position 1 and the nucleotide at position 658 of SEQ ID No 4, or a biologically active fragment or variant thereof.

The invention also pertains to a recombinant vector useful for the expression of the G713 coding sequence, wherein said vector comprises a nucleic acid of SEQ ID No 4 or a nucleic acid having at least 99.5% nucleotide identity with a polynucleotide of SEQ ID No 4.

The invention also deals with a recombinant vector useful for the expression of the murine G713 coding sequence, wherein said vector comprises a nucleic acid of SEQ ID No 6 or a nucleic acid having at least 95% nucleotide identity with a polynucleotide of SEQ ID No 6.

Some of the elements which can be found in the vectors of the present invention are described in further detail in the following sections.

a) Vectors

A recombinant vector according to the invention comprises, but is not limited to, a YAC (Yeast Artificial Chromosome), a BAC (Bacterial Artificial Chromosome), a phage, a phagemid, a cosmid, a plasmid or even a linear DNA molecule which may consist of a chromosomal, non-chromosomal and synthetic DNA. Such a recombinant vector can comprise a transcriptional unit comprising an assembly of:

(1) a genetic element or elements having a regulatory role in gene expression, for example promoters or enhancers. Enhancers are cis-acting elements of DNA, usually from about 10 to 300 bp in length that act on the promoter to increase the transcription.

(2) a structural or coding sequence which is transcribed into mRNA and eventually translated into a polypeptide, and

(3) appropriate transcription initiation and termination sequences. Structural units intended for use in yeast or eukaryotic expression systems preferably include a leader sequence enabling extracellular secretion of translated protein by a host cell. Alternatively, where a recombinant protein is expressed without a leader or transport sequence, it may include an N-terminal residue. This residue may or may not be subsequently cleaved from the expressed recombinant protein to provide a final product.

Generally, recombinant expression vectors will include origins of replication, selectable markers permitting transformation of the host cell, and a promoter derived from a highly expressed gene to direct transcription of a downstream structural sequence. The heterologous structural sequence is assembled in appropriate phase with translation initiation and termination sequences, and preferably a leader sequence capable of directing secretion of the translated protein into the periplasmic space or the extracellular medium.

The selectable marker genes for selection of transformed host cells are preferably dihydrofolate reductase or neomycin resistance for eukaryotic cell culture, TRP1 for S. cerevisiae or tetracycline, rifampicin or ampicillin resistance in E. coli, or levan saccharase for mycobacteria.

As a representative but non-limiting example, useful expression vectors for bacterial use can comprise a selectable marker and a bacterial origin of replication derived from commercially available plasmids comprising genetic elements of pBR322 (ATCC 37017). Such commercial vectors include, for example, pKK223-3 (Pharmacia, Uppsala, Sweden), and GEM1 (Promega Biotec, Madison, Wis., USA).

Large numbers of suitable vectors and promoters are known to those of skill in the art, and commercially available, such as bacterial vectors: pQE70, pQE60, pQE-9 (Qiagen), pbs, pD10, phagescript, psiX174, pbluescript SK, pbsks, pNH8A, pNH16A, pNH18A, pNH46A (Stratagene); ptrc99a, pKK223-3, pKK233-3, pDR540, pRIT5 (Pharmacia); or eukaryotic vectors: pWLNEO, pSV2CAT, pOG44, pXT1, pSG (Stratagene); pSVK3, pBPV, pMSG, pSVL (Pharmacia); baculovirus transfer vector pVL1392/1393 (Pharmingen); pQE-30 (QlAexpress).

A suitable vector for the expression of a G713 polypeptide of SEQ ID No 5 or 7 is a baculovirus vector that can be propagated in insect cells and in insect cell lines. A specific suitable host vector system is the pVL1392/1393 baculovirus transfer vector (Pharmingen) that is used to transfect the SF9 cell line (ATCC N^{o} CRL 1711) which is derived from Spodoptera frugiperda.

Other suitable vectors for the expression of a G713 polypeptide of SEQ ID Nos 5 or 7 in a baculovirus expression system include those described by Chai et al. (1993), Vlasak et al. (1983) and Lenhard et al. (1996).

Mammalian expression vectors will comprise an origin of replication, a suitable promoter and enhancer, and also any necessary ribosome binding sites, polyadenylation site, splice donor and acceptor sites, transcriptional termination sequences, and 5′-flanking non-transcribed sequences. DNA sequences derived from the SV40 viral genome, for example SV40 origin, early promoter, enhancer, splice and polyadenylation sites may be used to provide the required non-transcribed genetic elements.

b) Promoters

The suitable promoter regions used in the expression vectors according to the present invention are chosen taking into account the cell host in which the heterologous gene has to be expressed.

A suitable promoter may be heterologous with respect to the nucleic acid for which it controls the expression or alternatively can be endogenous to the native polynucleotide containing the coding sequence to be expressed. Additionally, the promoter is generally heterologous with respect to the recombinant vector sequences within which the construct promoter/coding sequence has been inserted.

Preferred bacterial promoters are the LacI, LacZ, the T3 or T7 bacteriophage RNA polymerase promoters, the polyhedrin promoter, or the p10 protein promoter from baculovirus (Kit Novagen) (Smith et al., 1983; O'Reilly et al., 1992), the lambda PR promoter or also the trc promoter.

Promoter regions can be selected from any desired gene using, for example, CAT (chloramphenicol transferase) vectors and more preferably pKK232-8 and pCM7 vectors. Particularly preferred bacterial promoters include lacI, lacZ, T3, T7, gpt, lambda PR, PL and trp. Eukaryotic promoters include CMV immediate early, HSV thymidine kinase, early and late SV40, LTRs from retrovirus, and mouse metallothionein-L. Selection of a convenient vector and promoter is well within the level of ordinary skill in the art.

The choice of a promoter is well within the ability of a person skilled in the field of genetic egineering. For example, one may refer to the book of Sambrook et al. (1989) or also to the procedures described by Fuller et al. (1996).

The vector containing the appropriate DNA sequence as described above, more preferably G713 gene regulatory polynucleotide, a polynucleotide encoding a G713 polypeptide of SEQ ID Nos 5 or 7 or both of these polynucleotides, can be utilized to transform an appropriate host to allow the expression of the desired polypeptide or polynucleotide.

c) Other Types of Vectors

The in vivo expression of a G713 polypeptide of SEQ ID Nos 5 or 7 may be useful in order to correct a genetic defect related to the expression of the native gene in a host organism or to the production of a biologically inactive G713 protein.

Consequently, the present invention also deals with recombinant expression vectors mainly designed for the in vivo production of a G713 polypeptide of SEQ ID Nos 5 or 7 by the introduction of the appropriate genetic material in the organism of the patient to be treated. This genetic material may be introduced in vitro in a cell that has been previously extracted from the organism, the modified cell being subsequently reintroduced in the said organism, directly in vivo into the appropriate tissue.

By “vector” according to this specific embodiment of the invention is intended either a circular or a linear DNA molecule.

One specific embodiment for a method for delivering a protein or peptide to the interior of a cell of a vertebrate in vivo comprises the step of introducing a preparation comprising a physiologically acceptable carrier and a naked polynucleotide operatively coding for the polypeptide of interest into the interstitial space of a tissue comprising the cell, whereby the naked polynucleotide is taken up into the interior of the cell and has a physiological effect.

In a specific embodiment, the invention provides a composition for the in vivo production of the G713 protein or polypeptide described herein. It comprises a naked polynucleotide operatively coding for this polypeptide, in solution in a physiologically acceptable carrier, and suitable for introduction into a tissue to cause cells of the tissue to express the said protein or polypeptide.

Compositions comprising a polynucleotide are described in PCT application N^{o} WO 90/11092 (Vical Inc.) and also in PCT application N^{o} WO 95/11307 (Institut Pasteur, INSERM, Université d'Ottawa) as well as in the articles of Tacson et al. (1996) and of Huygen et al. (1996).

The amount of vector to be injected to the desired host organism varies according to the site of injection. As an indicative dose, it will be injected between 0, 1 and 100 μg of the vector in an animal body, preferably a mammal body, for example a mouse body.

In another embodiment of the vector according to the invention, it may be introduced in vitro in a host cell, preferably in a host cell previously harvested from the animal to be treated and more preferably a somatic cell such as a muscle cell. In a subsequent step, the cell that has been transformed with the vector coding for the desired G713 polypeptide or the desired fragment thereof is reintroduced into the animal body in order to deliver the recombinant protein within the body either locally or systemically.

In one specific embodiment, the vector is derived from an adenovirus. Preferred adenovirus vectors according to the invention are those described by Feldman and Steg (1996) or Ohno et al. (1994). Another preferred recombinant adenovirus according to this specific embodiment of the present invention is the human adenovirus type 2 or 5 (Ad 2 or Ad 5) or an adenovirus of animal origin ( French patent application N^{o} FR-93.05954).

Retrovirus vectors and adeno-associated virus vectors are generally understood to be the recombinant gene delivery systems of choice for the transfer of exogenous polynucleotides in vivo, particularly to mammals, including humans. These vectors provide efficient delivery of genes into cells, and the transferred nucleic acids are stably integrated into the chromosomal DNA of the host

Particularly preferred retroviruses for the preparation or construction of retroviral in vitro or in vitro gene delivery vehicles of the present invention include retroviruses selected from the group consisting of Mink-Cell Focus Inducing Virus, Murine Sarcoma Virus, Reticuloendotheliosis virus and Rous Sarcoma virus. Particularly preferred Murine Leukemia Viruses include the 4070A and the 1504A viruses, Abelson (ATCC No VR-999), Friend (ATCC No VR-245), Gross (ATCC No VR-590), Rauscher (ATCC No VR-998) and Moloney Murine Leukemia Virus (ATCC No VR-1 90; PCT Application No WO 94/24298). Particularly preferred Rous Sarcoma Viruses include Bryan high titer (ATCC Nos VR-334, VR-657, VR-726, VR-659 and VR-728). Other preferred retroviral vectors are those described in Roth et al. (Roth J. A. et al., 1996), PCT Application No WO 93/25234, PCT Application No WO 94/ 06920, Roux et al., 1989, Julan et al., 1992 and Neda et al., 1991, the entire contents of which are hereby incorporated by reference.

Yet another viral vector system that is contemplated by the invention consists in the adeno-associated virus (AAV). The adeno-associated virus is a naturally occurring defective virus that requires another virus, such as an adenovirus or a herpes virus, as a helper virus for efficient replication and a productive life cycle (Muzyczka et al., 1992). It is also one of the few viruses that may integrate its DNA into non-dividing cells, and exhibits a high frequency of stable integration (Flotte et al., 1992; Samulski et al., 1989; McLaughlin et al., 1989). One advantageous feature of AAV derives from its reduced efficacy for transducing primary cells relative to transformed cells.

Other compositions containing a vector of the invention advantageously comprise an oligonucleotide fragment of a nucleic sequence selected from the group consisting of nucleotides 1076 to 3075 of SEQ ID No 1 and nucleotides 16330 to 18329 of SEQ ID No 3 as an antisense tool that inhibits the expression of the corresponding G713 gene. Preferred methods using antisense polynucleotide according to the present invention are the procedures described by Sczakiel et al. (1995) or those described in WO 95/24223, the entire contents of which are hereby incorporated by reference.

Preferably, the antisense tools are chosen among the polynucleotides (15-200 bp long) that are complementary to the 5′end of the G713 mRNA. In another embodiment, a combination of different antisense polynucleotides complementary to different parts of the desired targeted gene are used.

Preferred antisense polynucleotides according to the present invention are complementary to a sequence of the mRNAs of G713 that contains the translation initiation codon ATG.

Host Cells

Another object of the invention consists in host cell that have been transformed or transfected with one of the polynucleotides described therein, and more precisely a polynucleotide either comprising a G713 regulatory polynucleotide or the coding sequence of a G713 polypeptide, preferably a G713 polypeptide having the amino acid sequence of SEQ ID No 5 or 7. Are included host cells that are transformed (prokaryotic cells) or that are transfected (eukaryotic cells) with a recombinant vector such as one of those described above.

A recombinant host cell of the invention comprises any one of the polynucleotides or the recombinant vectors described therein.

A preferred recombinant host cell according to the invention comprises a polynucleotide selected from the following group of polynucleotides:

a) a purified or isolated nucleic acid encoding a G713 polypeptide, or a polypeptide fragment or variant thereof.

b) a purified or isolated nucleic acid comprising at least 20 consecutive nucleotides of a polynucleotide selected from the group consisting of the nucleotide sequences of SEQ ID Nos 4 and 6.

c) a purified or isolated nucleic acid comprising the nucleotide positions 1076 to 3075 of SEQ ID No 1 or a biologically active fragment or variant of the nucleotide positions 1076 to 3075 of SEQ ID No 1.

d) a purified or isolated nucleic acid comprising a 3′-regulatory sequence of the G713 gene, or a biologically active fragment or variant thereof.

e) a polynucleotide consisting of:

(1) a nucleic acid comprising a regulatory polynucleotide of nucleotide positions 1076 to 3075 of SEQ ID No 1 or a biologically active fragment or variant thereof;

(2) a polynucleotide encoding a desired polypeptide or nucleic acid.

(3) Optionally, a nucleic acid comprising a 3′-regulatory sequence, preferably a 3′-regulatory sequence of the G713 gene, or a biologically active fragment or variant thereof, wherein sequences (1), (2) and (3) are operably linked to one other.

Another preferred recombinant cell host according to the present invention is characterized in that its genome or genetic background (including chromosome, plasmids) is modified by the nucleic acid coding for a G713 polypeptide of SEQ ID No 5 or 7.

Preferred host cells used as recipients for the expression vectors of the invention are the following:

a) Prokaryotic host cells: Escherichia coli strains (I.E. DH5-α strain) or Bacillus subtilis.

b) Eukaryotic host cells: HeLa cells (ATCC No. CCL2; No. CCL2.1; No. CCL2.2), Cv 1 cells (ATCC No. CCL70), COS cells (ATCC No. CRL1650; No. CRL1651), Sf-9 cells (ATCC No. CRL1711).

The constructs in the host cells can be used in a conventional manner to produce the gene product encoded by the recombinant sequence.

Following transformation of a suitable host and growth of the host to an appropriate cell density, the selected promoter is induced by appropriate means, such as temperature shift or chemical induction, and cells are cultivated for an additional period.

Cells are typically harvested by centrifugation, disrupted by physical or chemical means, and the resulting crude extract retained for further purification.

Microbial cells employed in the expression of proteins can be disrupted by any convenient method, including freeze-thaw cycling, sonication, mechanical disruption, or use of cell lysing agents. Such methods are well known by the skill artisan.

Transgenic Animals

The terms “transgenic animals” or “host animals” are used herein to designate animals that have their genome genetically and artificially manipulated so as to include one of the nucleic acids according to the invention. Preferred animals are non-human mammals and include those belonging to a genus selected from Mus (e.g. mice), Rattus (e.g. rats) and Oryctogalus (e.g. rabbits) which have their genome artificially and genetically altered by the insertion of a nucleic acid according to the invention.

The transgenic animals of the invention all include within a plurality of their cells a cloned recombinant or synthetic DNA sequence, more specifically one of the purified or isolated nucleic acids comprising a G713 coding sequence, a G713 regulatory polynucleotide or a DNA sequence encoding an antisense polynucleotide such as described in the present specification.

First preferred transgenic animals according to the invention contain in their somatic cells and/or in their germ line cells a polynucleotide selected from the following group of polynucleotides:

a) a purified or isolated nucleic acid encoding a G713 polypeptide, or a polypeptide fragment or variant thereof.

b) a purified or isolated nucleic comprising at least 20 consecutive nucleotides of a polynucleotide selected from the group consisting of the nucleotide sequences of SEQ ID Nos 4 and 6.

c) a purified or isolated nucleic acid comprising the nucleotide positions 1076 to 3075 of SEQ ID No 1 or a biologically active fragment or variant of the nucleotide positions 1076 to 3075 of SEQ ID No 1.

d) a purified or isolated nucleic acid comprising a 3′-regulatory sequence of the G713 gene, or a biologically active fragment or variant thereof.

e) a polynucleotide consisting of:

(1) a nucleic acid comprising a regulatory polynucleotide of nucleotide positions 1076 to 3075 of SEQ ID No 1 or a biologically active fragment or variant thereof;

(2) a polynucleotide encoding a desired polypeptide or nucleic acid.

(3) Optionally, a nucleic acid comprising a 3′-regulatory sequence, preferably a 3′ regulatory sequence of the G713 gene, or a biologically active fragment or variant thereof, wherein sequences (1), (2) and (3) are operably linked to one other.

The replacement of the native genomic G713 sequence by a defective copy of said sequence may be preformed by techniques of gene targeting. Such techniques are notably described by Burright et al. (1997), Bates et al. (1997), Mangiarini et al. (1996, 1997), Davies et al. (1997a, 1997b), which are herein incorporated by reference.

Second preferred transgenic animals of the invention have the murine G713 gene replaced either by a defective copy of the murine G713 gene or by an interrupted copy of the human G713 gene. A “defective copy” of a murine or a human G713 gene, is intended to designate a modified copy of these genes that is not or poorly transcribed in the resulting recombinant host animal or a modified copy of these genes leading to the absence of synthesis of the corresponding translation product or alternatively leading to a modified and/or truncated translation product lacking the biological activity of the wild type G713 protein. The altered translation product thus contains amino acid modifications, deletions and substitutions. Modifications and deletions may render the naturally occurring gene nonfunctional, thus leading to a “knockout animal”. These transgenic animals are critical for the creation of animal models of human diseases, and for eventual treatment of disorders or diseases of the central nervous system, like schizophrenia or bipolar disorder. Examples of such knockout mice are described in the PCT Applications Nos WO 97/34641, WO 96/12792 and WO 98/02354, which are herein incorporated by reference.

The endogenous murine G713 gene can be interrupted by the insertion, between two contiguous nucleotides of said gene, of a part of all of a marker gene placed under the control of the appropriate promoter, for example the endogenous promoter of the endogenous murine G713 gene. The marker gene may be the neomycin resistance gene (neo) that may e operably linked to the phosphoglycerate kinase-1 (PGK-1) promoter, as described in the PCT Application No WO 98/02534.

Thus, the invention is also directed to a transgenic animal contain in their somatic cells and/or in their germ line cells a polynucleotide selected from the following group of polynucleotides:

a) a defective copy of the human G713 gene;

b) a defective copy of the murine G713 gene;

c) a defective copy of the endogenous G713 gene, wherein the expression “endogenous G713 gene” designates a G713 gene that is naturally present within the genome of the animal host to be genetically modified.

The invention also concerns a method for obtaining transgenic animals, wherein said methods comprise the steps of:

a) replacing the endogenous copy of the animal G713 gene by a nucleic acid selected from the group consisting of a defective copy of the human G713 gene, a defective copy of the murine G713 gene and a defective copy of the endogenous G713 gene in animal cells, preferably embryonic stem cells (ES);

b) introducing the recombinant animal cells obtained at step a) in embryos, notably blastocysts of the animal;

c) selecting the resulting transgenic animals, for example by detecting the defective copy of a G713 gene with one or several primers or probes according to the invention;

Optionally, the transgenic animals may be bred together in order to obtain homozygous transgenic animals for the defective copy of the G713 gene introduced.

The transgenic animals of the invention thus contain specific sequences of exogenous genetic material such as the nucleotide sequences described above in detail.

In a first preferred embodiment, these transgenic animals may be good experimental models in order to study the diverse pathologies related to central nervous system disorders like schizophrenia or bipolar disorder, in particular concerning the transgenic animals within the genome of which has been inserted one or several copies of a polynucleotide encoding a native G713 protein, or alternatively a mutant G713 protein.

In a second preferred embodiment, these transgenic animals may express a desired polypeptide of interest under the control of the regulatory polynucleotides of the G713 gene, leading to good yields in the synthesis of this protein of interest, and eventually a tissue specific expression of this protein of interest.

Since it is possible to produce transgenic animals of the invention using a variety of different sequences, a general description will be given of the production of transgenic animals by referring generally to exogenous genetic material. This general description can be adapted by those skilled in the art in order to incorporate the DNA sequences into animals. For more details regarding the production of transgenic animals, and specifically transgenic mice, it may be referred to Sandou et al. (1994) and also to U.S. Pat. Nos. 4,873,191, issued Oct. 10, 1989, 5,968,766, issued Dec. 16, 1997 and 5,387,742, issued Feb. 28, 1995, these documents being herein incorporated by reference to disclose methods for producing transgenic mice.

Transgenic animals of the present invention are produced by the application of procedures which result in an animal with a genome that incorporates exogenous genetic material which is integrated into the genome. The procedure involves obtaining the genetic material, or a portion thereof, which encodes either a G713 coding sequence, a G713 regulatory polynucleotide or a DNA sequence encoding an antisense polynucleotide such as described in the present specification.

A recombinant polynucleotide of the invention is inserted into an embryonic or ES stem cell line. The insertion is made using electroporation. The cells subjected to electroporation are screened (e.g. Southern blot analysis) to find positive cells which have integrated the exogenous recombinant polynucleotide into their genome. An illustrative positive-negative selection procedure that may be used according to the invention is described by Mansour et al. (1988). Then, the positive cells are isolated, cloned and injected into 3.5 days old blastocysts from mice. The blastocysts are then inserted into a female host animal and allowed to grow to term. The offsprings of the female host are tested to determine which animals are transgenic e.g. include the inserted exogenous DNA sequence and which are wild-type.

Thus, the present invention also concerns a transgenic animal containing a nucleic acid, a recombinant expression vector or a recombinant host cell according to the invention.

G713 and Murine G713 Polypeptide and Peptide Fragments

The present invention also concerns a method for producing one of the polypeptides described herein, and especially a polypeptide selected from the group consisting of the amino acid sequences of SEQ ID Nos 5 and 7 or a fragment or a variant thereof, wherein said method comprises the steps of:

a) culturing, in an appropriate culture medium, a cell host previously transformed or transfected with the recombinant vector comprising a nucleic acid encoding a G713 polypeptide, or a fragment or a variant thereof;

b) harvesting the culture medium thus conditioned or lyse the cell host, for example by sonication or by an osmotic shock;

c) separating or purifying, from the said culture medium, or from the pellet of the resultant host cell lysate the thus produced polypeptide of interest.

d) Optionally characterizing the produced polypeptide of interest.

In a specific embodiment of the above method, step a) is preceded by a step wherein the nucleic acid coding for a G713 polypeptide, or a fragment or a variant thereof, is inserted in an appropriate vector, optionally after an appropriate cleavage of this amplified nucleic acid with one or several restriction endonucleases. The nucleic acid coding for a G713 polypeptide or a fragment or a variant thereof may be the resulting product of an amplification reaction using a pair of primers according to the invention (by SDA, TAS, 3SR NASBA, TMA etc.).

The polypeptides according to the invention may be characterized by binding an immunoaffinity chromatography column on which polyclonal or monoclonal antibodies directed to a polypeptide selected from the group consisting of the amino acid sequences of seq id nos 5 and 7, or a fragment or a variant thereof, have previously been immobilized. Purification of the recombinant proteins or peptides according to the present invention may be carried out by passage onto a nickel or cupper affinity chromatography column. The nickel chromatography column may contain the ni-nta resin (porath et al., 1975). The polypeptides or peptides thus obtained may be purified, for example by high performance liquid chromatography, such as reverse phase and/or cationic exchange hplc, (rougeot et al.,1994). The reason to prefer this kind of peptide or protein purification is the lack of byproducts found in the elution samples which renders the resultant purified protein or peptide more suitable for a therapeutic use.

G713 Polypeptide (Human)

The term “G713 polypeptides” is used herein to embrace all of the proteins and polypeptides of the present invention. Also forming part of the invention are polypeptides encoded by the polynucleotides of the invention, as well as fusion polypeptides comprising such polypeptides. The invention embodies G713 proteins from humans, including isolated or purified G713 proteins consisting of, consisting essentially of, or comprising the sequence of SEQ ID No 5.

The present invention embodies isolated, purified, and recombinant polypeptides comprising a contiguous span of at least 6 amino acids, preferably at least 8 to 10 amino acids, more preferably at least 12, 15, 20, 25, 30, 40, 50, or 100 amino acids of SEQ ID No 5.

The invention also encompasses a purified, isolated, or recombinant polypeptides comprising an amino acid sequence having at least 70, 75, 80, 85, 90, 95, 98 or 99% amino acid identity with the amino acid sequence of SEQ ID No. 5 or a fragment thereof. In a preferred embodiment, a variant polypeptide comprises amino acid changes ranging from 1, 2, 3, 4, 5, 10 to 20 substitutions, additions or deletions of one amino acid, preferably from 1 to 10, more preferably from 1 to 5 and most preferably from 1 to 3 substitutions, additions or deletions of one amino acid. The preferred amino acid changes are those which have little or no influence on the biological activity or the capacity of the variant G713 polypeptide to be recognized by antibodies raised against a native G713 protein. In a second preferred embodiment, a mutated G713 polypeptide comprises amino acid changes ranging from 1 to about 200 deletions of one amino acid and of at least one aminoacid substitution or addition, preferably from 1 to 10, 20 or 30 amino acid substitutions or additions. The amino acid substitutions are generally non conservative in terms of polarity, charge, hydrophilicity properties of the substitute amino acid when compared with the native amino acid. The amino acid changes occurring in such a mutated G713 polypeptide may be determinant for the biological activity or for the capacity of the mutated G713 polypeptide to be recognized by antibodies raised against a native G713.

The G713 polypeptide of the amino acid sequence of SEQ ID No 5 has 458 amino acids in length. This polypeptide has a strong amino acid sequence identity with the mouse G713 polypeptide of SEQ ID No 7, specifically 87.9% nucleic acid identity.

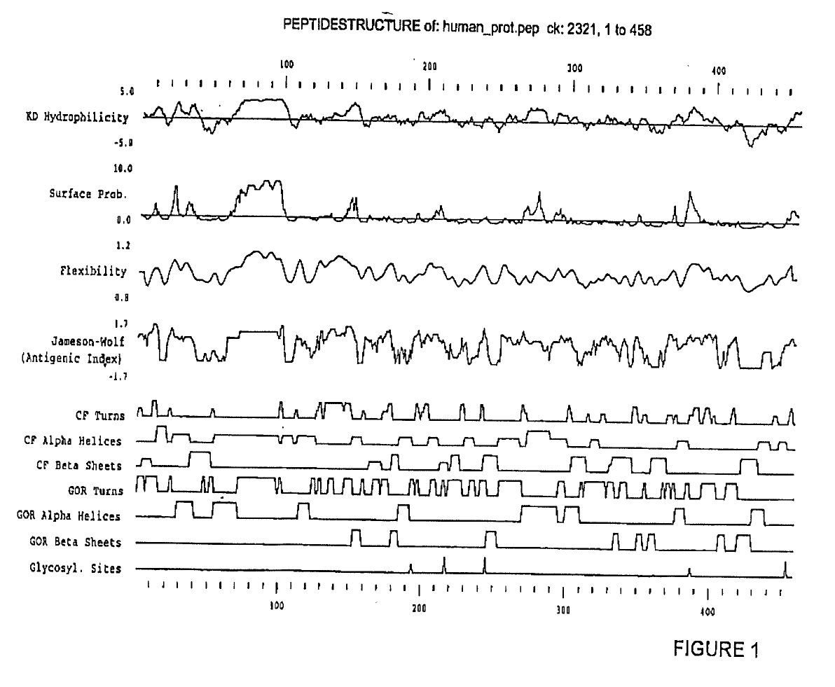

As shown in FIG. 1, a particular region of the G713 polypeptide located in its N-terminal portion has interesting features. A large hydrophilic region begins at the amino acid in position 68 (R) and ends at the amino acid in position 101 (P) of the amino acid sequence of G713. A large region having a good probability to be exposed to the outer environment begins at the amino acid in position 62 (A) and ends at the amino acid in position 101 (P) of the amino acid sequence of G713. A large region having good antigenicity properties begins at the amino acid in position 63 (K) and ends at the amino acid in position 102 (S) of the amino acid sequence of G713.

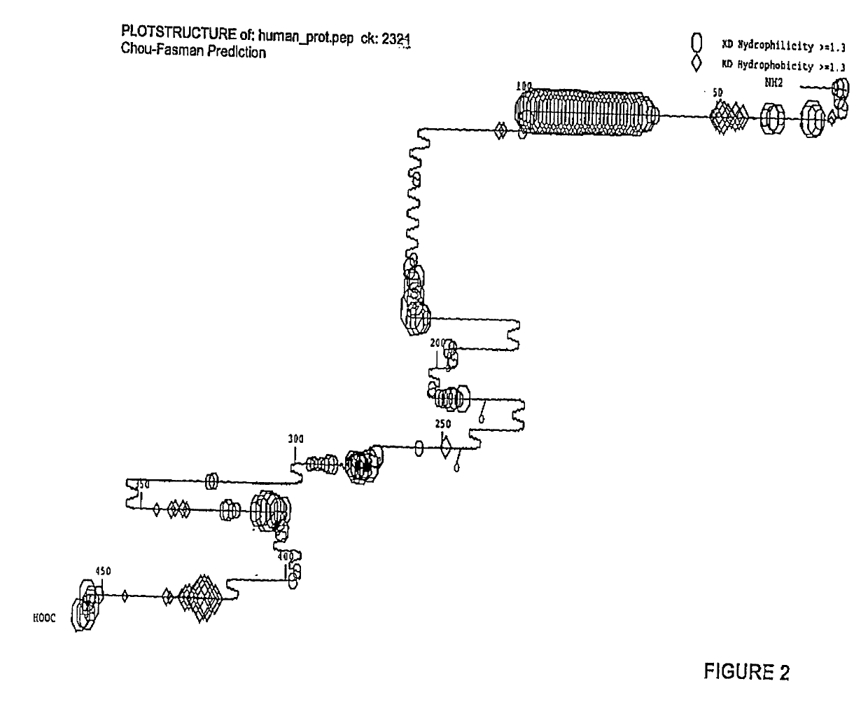

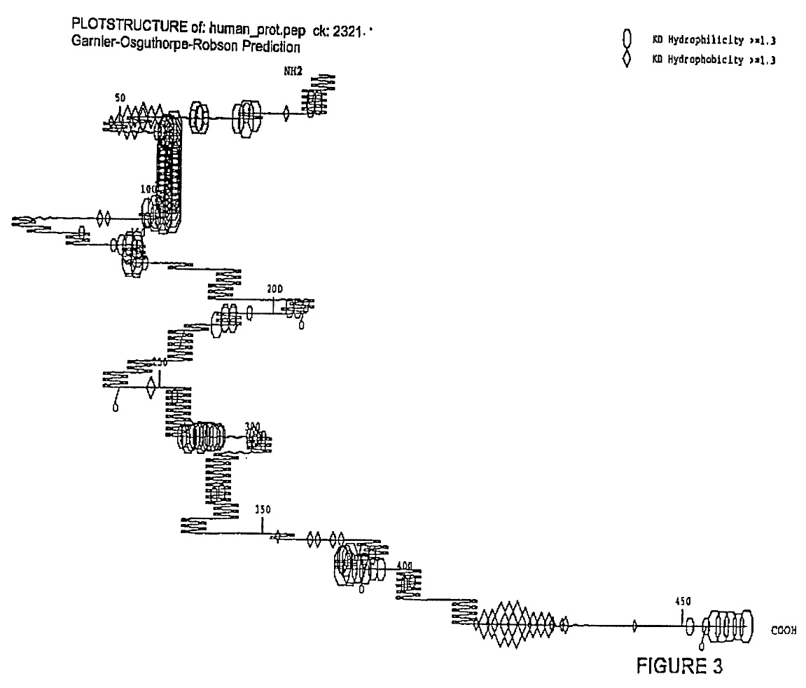

FIGS. 2 and 3 depict the two-dimensional structure of the G713 protein according, respectively, to the Chou and Fasman method and to the Gamier-Ogsuthorpe-Robson method. These two models confirm that region spanning between the amino acid around the position 60 and the amino acid around the position 115 of the G713 protein has particular hydrophilicity properties that make this peptide stretch valuable, notably for the production of antibodies specific to this protein.

Thus, a polypeptide comprising a peptide sequence corresponding to the amino acid sequence beginning at the amino acid in position 62 and ending at the amino acid in position 102 of the G713 protein may be used for raising specific antibodies to a G713 protein, and specifically the G713 protein of the amino acid sequence of SEQ ID No 5. Peptide fragments of this polypeptide of interest are also part of the invention. Such peptide fragments have advantageously an amino acid sequence length of at least 8 consecutive amino acids of the polypeptide of interest, and preferably between 10 and 40 amino acids in length, more preferably between 15 and 30 amino acids in length. Another polypeptide of interest according to the present invention consists of a polypeptide comprising a peptide sequence beginning at the amino acid in position 203 and ending at the amino acid in position 458 of the amino acid sequence of SEQ ID No 5 or a peptide fragment thereof.

Both the human and the murine G713 polypeptides are cysteine rich, both having a total of 21 cysteins. Of interest also in view of G713's structure which contains one transmembrane domain, 9 of these cysteins are organized in a domain resembling the frizzled domain (Fz). In particular, said Fz-like domain is located at amino acid positions 304 to 379 of SEQ ID No 5 in the human G713 polypeptide and amino acid positions 313 to 388 of SEQ ID No 7 in the murine polypeptide.

A candidate structure for the G713 polypeptide comprises, consists essentially of or consists of, from the N-terminal to the C-terminal, a protein binding or membrane associated domain, an external domain, a transmembrane domain, and a cytoplasmic domain. The transmembrane domain is located at amino acid positions 417 to 437 in the human G713 polypeptide of SEQ ID No 5, corresponding to amino acid positions 426 to 446 in the murine G713 polypeptide of SEQ ID No 7.

The G713 polypeptide contains, as noted above, a hydrophobic segment located at amino acid positions 40 to 60 in SEQ ID Nos 5 and 7. This domain is indicative of a membrane association and may further comprise a signal peptide domain. Thus, embodiments of the invention include, but are not limited to, peptide fragments of said domain, a G713 polypeptide comprising said domain, fragments of said domain, or specifically lacking said domain. A preferred G713 polypeptide fragment comprises, consists essentially of, or consists of a G713 signal sequence. Signal sequences can have particular use in the targeting of a desired compound for secretion or insertion into the cell membrane. In an exemplary but not limiting example, signal sequences may be fused to a desired polypeptide of interest to direct secretion of said polypeptide, or insertion of said polypeptide into the cell membrane.

The invention further concerns a protein binding domain comprising a hydrophobic domain located at amino acid positions 40 to 60 of SEQ ID Nos 5 and 7. Said protein binding domain is conserved at an exceptionally high rate in the human and murine G713, especially in relation to conservation expected among membrane-associated domains, indicative of a domain essential for binding a target protein. Thus, while not limited to such, embodiments of the invention can include polynucleotides encoding a G713 signal or protein binding sequence, vectors and host cells comprising said polynucleotide, and fusion proteins comprising a G713 signal peptide.

Such polypeptides of interest or its peptide fragments may be obtained either by proteolytic cleavage of the G713 protein or by chemical synthesis.

In a specific embodiment of this polypeptide of interest or its peptide fragments in which they are used to prepare polyclonal or monoclonal antibodies against the G713 protein, this polypeptide or peptide fragments are preferably covalently or non-covalently bound to a carrier molecule, such as human or bovine serum albumin (HSA or BSA).

A further object of the present invention concerns a purified or isolated polypeptide which is encoded by a nucleic acid comprising nucleotide positions 1076 to 3075 of SEQ ID No 1or fragments or variants thereof.

Such a mutated G713 protein may be the target of diagnostic tools, such as specific monoclonal or polyclonal antibodies, useful for detecting the mutated G713 protein in a sample.

Murine G713 Polypeptide

The term “G713 polypeptides” is used herein to embrace all of the proteins and polypeptides of the present invention. Also forming part of the invention are polypeptides encoded by the polynucleotides of the invention, as well as fusion polypeptides comprising such polypeptides. The invention embodies G713 proteins from humans, including isolated or purified G713 proteins consisting of, consisting essentially of, or comprising the sequence of SEQ ID No 7.

The present invention embodies isolated, purified, and recombinant polypeptides comprising a contiguous span of at least 6 amino acids, preferably at least 8 to 10 amino acids, more preferably at least 12, 15, 20, 25, 30, 40, 50, or 100 amino acids of SEQ ID No 7. In other preferred embodiments the contiguous stretch of amino acids comprises the site of a mutation or functional mutation, including a deletion, addition, swap or truncation of the amino acids in the G713 protein sequence.

The invention also encompasses a purified, isolated, or recombinant polypeptides comprising an amino acid sequence having at least 70, 75, 80, 85, 90, 95, 98 or 99% amino acid identity with the amino acid sequence of SEQ ID No. 7 or a fragment thereof. The G713 polypeptide of the amino acid sequence of SEQ ID No 7 has 467 amino acids in length. As already mentioned, this polypeptide has a strong amino acid sequence identity with the human G713 polypeptide of SEQ ID No 5, specifically 87.9% nucleic acid identity.

As shown in FIG. 1, a particular region of the murine G713 polypeptide located in its N-terminal portion have interesting features. A large hydrophilic region begins at the amino acid in position 66 (R) and ends at the amino acid in position 112 (P) of the amino acid sequence of murine G713. A large region having a good probability to be exposed to the outer environment begins at the amino acid in position 63 (K) and ends at the amino acid in position 112 (P) of the amino acid sequence of murine G713. A large region having good antigenicity properties begins at the amino acid in position 63 (K) and ends at the amino acid in position 113 (S) of the amino acid sequence of murine G713.

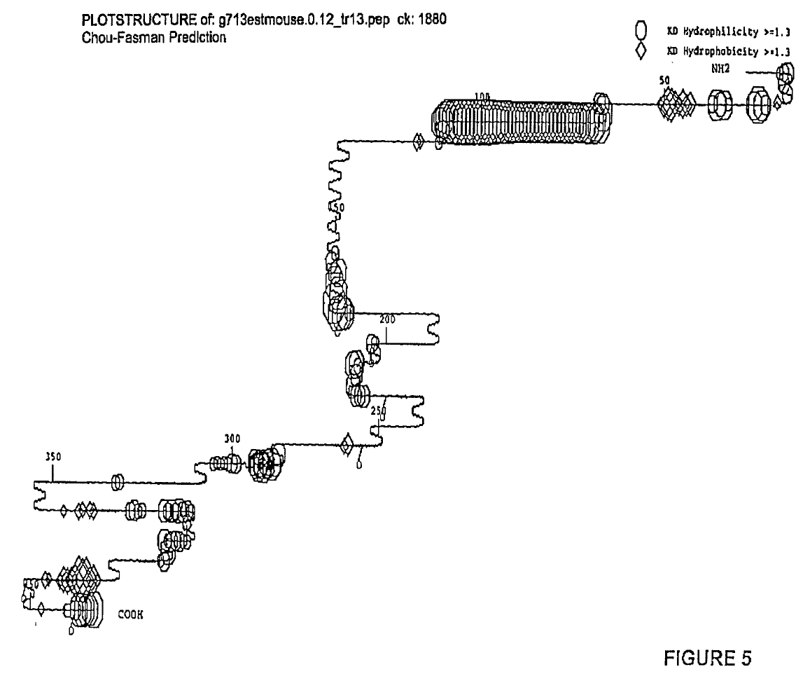

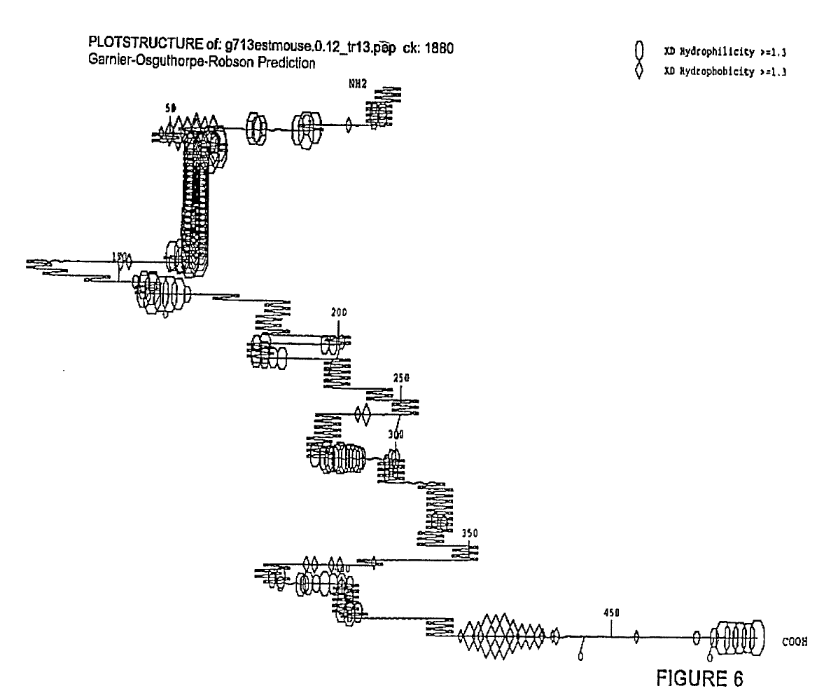

FIGS. 5 and 6 depict the two-dimensional structure of the G713 protein according, respectively, to the Chou and Fasman method and to the Gamier-Ogsuthorpe-Robson method. These two models confirm that region spanning between the amino acid around the position 65 and the amino acid around the position 120 of the G713 protein has particular hydrophilicity properties that make this peptide strectch valuable, notably for the production of antibodies specific to this protein.

Thus, a polypeptide comprising a peptide sequence corresponding to the amino acid sequence beginning at the amino acid in position 63 and ending at the amino acid in position 113 of the murine G713 protein may be used for raising specific antibodies to a G713 protein, and specifically the murine G713 protein of the amino acid sequence of SEQ ID No 7. Peptide fragments of this polypeptide of interest are also part of the invention. Such peptide fragments have advantageously an amino acid sequence length of at least 8 consecutive amino acids of the polypeptide of interest, and preferably between 10 and 40 amino acids in length, more preferably between 15 and 30 amino acids in length.

Such a polypeptide of interest or its peptide fragments may be obtained either by proteolytic cleavage of the murine G713 protein or by chemical synthesis.

In a specific embodiment of this polypeptide of interest or its peptide fragments in which they are used to prepare polyclonal or monoclonal antibodies against the murine G713 protein, this polypeptide or peptide fragments are preferably covalently or non-covalently bound to a carrier molecule, such as human or bovine serum albumin (HSA or BSA).

A further object of the present invention concerns a purified or isolated polypeptide which is encoded by a nucleic acid comprising a nucleotide sequence of SEQ ID No 6 or fragments or variants thereof.

In the case of an amino acid substitution in the amino acid sequence of a polypeptide according to the invention, one or several—consecutive or non-consecutive—amino acids are replaced by “equivalent” amino acids. The expression “equivalent” amino acid is used herein to designate any amino acid that may be substituted for one of the amino acids belonging to the native protein structure without decreasing the binding properties of the corresponding peptides to the antibodies raised against the human or murine G713 protein of the amino acid sequence of SEQ ID No 5 or 7. In other words, the “equivalent” amino acids are those which allow the generation or the synthesis of a polypeptide with a modified sequence when compared to the amino acid sequence of the native human or murine G713 protein, said modified polypeptide being able to bind to the antibodies raised against the human or murine G713 protein of the amino acid sequence of SEQ ID No 5 or 7 and/or to induce antibodies recognizing the parent polypeptide consisting in the human or murine G713 polypeptide of the amino acid sequence of SEQ ID No 5 or 7.

These equivalent amino acids may be determined either by their structural homology with the initial amino acids to be replaced, by the similarity of their net charge, and optionally by the results of the cross-immunogenicity between the parent peptides and their modified counterparts. The peptides containing one or several “equivalent” amino acids must retain their specificity and affinity properties to the biological targets of the parent protein, as it can be assessed by a ligand binding assay or an ELISA assay. By an equivalent amino acid is also meant the replacement of a residue in the L-form by a residue in the D form or the replacement of a Glutamic acid (E) residue by a Pyro-glutamic acid compound. The synthesis of peptides containing at least one residue in the D-form is, for example, described by Koch (1977).

A specific embodiment of a modified G713 peptide molecule of interest according to the present invention, includes, but is not limited to, a peptide molecule which is resistant to proteolysis, is a peptide in which the —CONH— peptide bond is modified and replaced by a (CH2NH) reduced bond, a (NHCO) retro inverso bond, a (CH2—O) methylene-oxy bond, a (CH2—S) thiomethylene bond, a (CH2CH2) carba bond, a (CO—CH2) cetomethylene bond, a (CHOH—CH2) hydroxyethylene bond), a (N—N) bound, a E-alcene bond or also a —CH═CH— bond.

The invention also encompasses a human or murine G713 polypeptide or a fragment or a variant thereof in which at least one peptide bound has been modified as described above.

The polypeptides according to the invention may also be prepared by the conventional methods of chemical synthesis, either in a homogenous solution or in solid phase. As an illustrative embodiment of such chemical polypeptide synthesis techniques, it may be cited the homogenous solution technique described by Houbenweyl (1974). The human or murine G713 polypeptide, or a fragment or a variant thereof may thus be prepared by chemical synthesis in liquid or solid phase by successive couplings of the different amino acid residues to be incorporated (from the N-terminal end to the C-terminal end in liquid phase, or from the C-terminal end to the N-terminal end in solid phase) wherein the N-terminal ends and the reactive side chains are previously blocked by conventional groups. For solid phase synthesis the technique described by Merrifield (1965) may be used in particular.

Antibodies

Any G713 polypeptide or whole protein may be used to generate antibodies capable of specifically binding to an expressed G713 protein or fragments thereof as described. Any of the human or murine G713 polypeptides of SEQ ID Nos 5 or 7 or one of their peptide fragments of interest can be used for the preparation of polyclonal or monoclonal antibodies.

Antibody compositions of the invention may also be capable of specifically binding or specifically bind to a variant of the G713 protein of SEQ ID Nos 5 or 7. For an antibody composition to specifically bind to a first variant of G713, it must demonstrate at least a 5%, 10%, 15%, 20%, 25%, 50%, or 100% greater binding affinity for a full length first variant of the G713 protein than for a full length second variant of the G713 protein in an ELISA, RIA, or other antibody-based binding assay. The invention concerns antibody compositions, either polyclonal or monoclonal, capable of selectively binding, or selectively bind to an epitope-containing a polypeptide comprising a contiguous span of at least 6 amino acids, preferably at least 8 to 10 amino acids, more preferably at least 12, 15, 20, 25, 30, 40, 50, or 100 amino acids of SEQ ID No 5 or 7. In a particularly preferred embodiment said contiguous span comprises at least 6, preferably at least 8 to 10, more preferably at least 12, 15, 20, 25, 30, 40, 50, or 100 contiguous amino acids of SEQ ID No 5 or 7, including:

i) at least one of the amino acid positions 62 to 102 or 203 to 458 of SEQ ID No 5; and/or

ii) at least one of the amino acid positions 1 to 467 of SEQ ID No 7.

The invention also concerns a purified or isolated antibody capable of specifically binding to a mutated G713 protein or to a fragment or variant thereof comprising an epitope of the mutated G713 protein. In another preferred embodiment, the present invention concerns an antibody capable of binding to a polypeptide comprising at least 10 consecutive amino acids of a G713 protein and including at least one of the amino acids which can be encoded by the trait causing mutations.

The invention also concerns the use in the manufacture of antibodies of a polypeptide comprising a contiguous span of at least 6 amino acids, preferably at least 8 to 10 amino acids, more preferably at least 12, 15, 20, 25, 30, 40, 50, or 100 amino acids of SEQ ID No 5 or 7. In a preferred embodiment, said contiguous span of SEQ ID No 5 or 7 comprises at of least 6, preferably at least 8 to 10, more preferably at least 12, 15, 20, 25, 30, 40, 50, or 100 contiguous amino acids of SEQ ID No 5 or 7, including:

ii) at least 1, 2, 3, 5 or 10 of the amino acid positions 62 to 102 or 203 to 458 of SEQ ID No 5; and/or

iii) at least 1, 2, 3, 5 or 10 of the amino acid positions 1 to 467 of SEQ ID No 7.

Non-human animals or mammals, whether wild-type or transgenic, which express a different species of G713 than the one to which antibody binding is desired, and animals which do not express G713 (i.e. a G713 knock out animal as described herein) are particularly useful for preparing antibodies. G713 knock out animals will recognize all or most of the exposed regions of a G713 protein as foreign antigens, and therefore produce antibodies with a wider array of G713 epitopes. Moreover, smaller polypeptides with only 10 to 30 amino acids may be useful in obtaining specific binding to any one of the G713 proteins. In addition, the humoral immune system of animals which produce a species of G713 that resembles the antigenic sequence will preferentially recognize the differences between the animal's native G713 species and the antigen sequence, and produce antibodies to these unique sites in the antigen sequence. Such a technique will be particularly useful in obtaining antibodies that specifically bind to any one of the G713 proteins.

Antibody preparations prepared according to either protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

Antibodies of the invention include chimeric single chain Fv antibody fragments (Martineau et al., 1998), antibody fragments obtained through phage display libraries (Ridder et al., 1995; Vaughan et al., 1995) and humanized antibodies (Reinmann et al., 1997; Leger et al., 1997).

The antibodies of the invention may be labeled by any one of the radioactive, fluorescent or enzymatic labels known in the art.

Consequently, the invention is also directed to a method for detecting specifically the presence of a G713 polypeptide according to the invention in a biological sample, said method comprising the following steps:

a) bringing into contact the biological sample with a polyclonal or monoclonal antibody that specifically binds a G713 polypeptide comprising an amino acid sequence of SEQ ID No 5 or 7, or to a peptide fragment or variant thereof; and

b) detecting the antigen-antibody complex formed.

The invention also concerns a diagnostic kit for detecting in vitro the presence of a G713 polypeptide according to the present invention in a biological sample, wherein said kit comprises:

a) a polyclonal or monoclonal antibody that specifically binds a G713 polypeptide comprising an amino acid sequence of SEQ ID No 5 or 7, or to a peptide fragment or variant thereof, optionally labeled;

b) a reagent allowing the detection of the antigen-antibody complexes formed, said reagent carrying optionally a label, or being able to be recognized itself by a labeled reagent, more particularly in the case when the above-mentioned monoclonal or polyclonal antibody is not labeled by itself.

Example of methods of preparing antibodies are provided in Example 1(f).

Methods for Screening Substances Interacting with a G713 Polypeptide

For the purpose of the present invention, a ligand means a molecule, such as a protein, a peptide, an antibody or any synthetic chemical compound capable of binding to the human or murine G713 protein or one of its fragments or variants or to modulate the expression of the polynucleotide coding for G713 or a fragment or variant thereof.

In the ligand screening method according to the present invention, a biological sample or a defined molecule to be tested as a putative ligand of the human or murine G713 protein is brought into contact with the corresponding purified human or murine G713 protein, for example the corresponding purified recombinant human or murine G713 protein produced by a recombinant cell host as described hereinbefore, in order to form a complex between this protein and the putative ligand molecule to be tested.

Another object of the present invention consists of methods and kits for the screening of candidate substances that interact with a human or murine G713 polypeptide.

The present invention pertains to methods for screening substances of interest that interact with a human or murine G713 protein or one fragment or variant thereof. By their capacity to bind covalently or non-covalently to a human or murine G713 protein or to a fragment or variant thereof, these substances or molecules may be advantageously used both in vitro and in vivo.

In vitro, said interacting molecules may be used as detection means in order to identify the presence of a human or murine G713 protein in a sample, preferably a biological sample.

A method for the screening of a candidate substance comprises the following steps:

a) providing a polypeptide consisting of a human or murine G713 protein or a fragment or a variant thereof;

b) obtaining a candidate substance;

c) bringing into contact said polypeptide with said candidate substance;

d) detecting the complexes formed between said polypeptide and said candidate substance.

In one embodiment of the screening method defined above, the complexes formed between the polypeptide and the candidate substance are further incubated in the presence of a polyclonal or a monoclonal antibody that specifically binds to the human or murine G713 protein or to said fragment or variant thereof.

The invention further concerns a kit for the screening of a candidate substance interacting with the G713 polypeptide, wherein said kit comprises:

a) a G713 protein having an amino acid sequence selected from the group consisting of the amino acid sequences of SEQ ID Nos 5 and 7 or a peptide fragment or a variant thereof;

b) optionally means useful to detect the complex formed between the G713 protein or its peptide fragment or variant and the candidate substance.

In a preferred embodiment of the kit described above, the detection means consist in monoclonal or polyclonal antibodies directed against the G713 protein or a peptide fragment or a variant thereof.

Various candidate substances or molecules can be assayed for interaction with a human or murine G713 polypeptide. These substances or molecules include, without being limited to, natural or synthetic organic compounds or molecules of biological origin such as polypeptides. When the candidate substance or molecule consists of a polypeptide, this polypeptide may be the resulting expression product of a phage clone belonging to a phage-based random peptide library, or alternatively the polypeptide may be the resulting expression product of a cDNA library cloned in a vector suitable for performing a two-hybrid screening assay.

In another embodiment of the present screening method, increasing concentrations of a monoclonal or polyclonal antibody directed against a human or murine G713 protein or a fragment or a variant thereof is reacted with the considered G713 protein or with a fragment or variant thereof, simultaneously or prior to the addition of the candidate substance or molecule, when performing step c) of said method. By this technique, the detection and optionally the quantification of the complexes formed between the human or murine G713 protein or the fragment or variant thereof and the substance or molecule to be screened allows the one skilled in the art to determine the affinity value of said substance or molecule for said human or murine G713 protein or the fragment or variant thereof.

The invention also pertains to kits useful for performing the hereinbefore described screening method. Preferably, such kits comprise a human or a murine G713 polypeptide or a fragment or a variant thereof, and optionally means useful to detect the complex formed between the human or the murine G713 polypeptide or its fragment or variant and the candidate substance. In a preferred embodiment the detection means consist in monoclonal or polyclonal antibodies directed against the corresponding G713 polypeptide or a fragment or a variant thereof.

A. Candidate Ligands Obtained From Random Peptide Libraries

In a particular embodiment of the screening method, the putative ligand is the expression product of a DNA insert contained in a phage vector (Parmley and Smith, 1988). Specifically, random peptide phages libraries are used. The random DNA inserts encode for peptides of 8 to 20 amino acids in length (Oldenburg K. R. et al., 1992; Valadon P., et al., 1996; Lucas A. H., 1994; Westerink M. A. J., 1995; Castagnoli L. et al. (Felici F, 1991). According to this particular embodiment, the recombinant phages expressing a protein that binds to the immobilized G713 protein is retained and the complex formed between the G713 protein and the recombinant phage may be subsequently immunoprecipitated by a polyclonal or a monoclonal antibody directed against the G713 protein.

Once the ligand library in recombinant phages has been constructed, the phage population is brought into contact with the immobilized human or murine G713 protein. Then the preparation of complexes is washed in order to remove the non-specifically bound recombinant phages. The phages that bind specifically to the human or murine G713 protein are then eluted by a buffer (acid pH) or immunoprecipitated by the monoclonal antibody produced by the hybridoma anti-G713, and this phage population is subsequently amplified by an over-infection of bacteria (for example E. coli). The selection step may be repeated several times, preferably 2-4 times, in order to select the more specific recombinant phage clones. The last step consists in characterizing the peptide produced by the selected recombinant phage clones either by expression in infected bacteria and isolation, expressing the phage insert in another host-vector system, or sequencing the insert contained in the selected recombinant phages.

B. Candidate Ligands Obtained Through a Two-hybrid Screening Assay

The yeast two-hybrid system is designed to study protein-protein interactions in vivo (Fields and Song, 1989), and relies upon the fusion of a bait protein to the DNA binding domain of the yeast Gal4 protein. This technique is also described in the U.S. Pat. No. 5,667,973 and the U.S. Pat. No. 5,283,173 (Fields et al.) the technical teachings of both patents being herein incorporated by reference.

The general procedure of library screening by the two-hybrid assay may be performed as described by Harper et al. (1993) or as described by Cho et al. (1998) or also Fromont-Racine et al. (1997).

The bait protein or polypeptide consists of a human or murine G713 polypeptide or a fragment or variant thereof.

More precisely, the nucleotide sequence encoding the human or murine G713 polypeptide or a fragment or variant thereof is fused to a polynucleotide encoding the DNA binding domain of the GAL4 protein, the fused nucleotide sequence being inserted in a suitable expression vector, for example pAS2 or pM3.

Then, a human cDNA library is constructed in a specially designed vector, such that the human cDNA insert is fused to a nucleotide sequence in the vector that encodes the transcriptional domain of the GAL4 protein. Preferably, the vector used is the pACT vector. The polypeptides encoded by the nucleotide inserts of the human cDNA library are termed “pray” polypeptides.

A third vector contains a detectable marker gene, such as beta galactosidase gene or CAT gene that is placed under the control of a regulation sequence that is responsive to the binding of a complete Gal4 protein containing both the transcriptional activation domain and the DNA binding domain. For example, the vector pG5EC may be used.

Two different yeast strains are also used. As an illustrative but non limiting example the two different yeast strains may be the followings:

Y190, the phenotype of which is (MATa, Leu2-3, 112 ura3-12, trp1-901, his3-D200, ade2-101, gal4Dgal180D URA3 GAL-LacZ, LYS GAL-HIS3, cyh^{r});

Y187, the phenotype of which is (MATa gal4gal80his3 trp1-901 ade2-101 ura3-52 leu2-3, -112 URA3 GAL-lacZmet⁻), which is the opposite mating type of Y190.

Briefly, 20 μg of pAS2/G713 and 20 μg of pACT-cDNA library are co-transformed into yeast strain Y190. The transformants are selected for growth on minimal media lacking histidine, leucine and tryptophan, but containing the histidine synthesis inhibitor 3-AT (50 mM). Positive colonies are screened for beta galactosidase by filter lift assay. The double positive colonies (His⁺, beta-gal⁺) are then grown on plates lacking histidine, leucine, but containing tryptophan and cycloheximide (10 mg/ml) to select for loss of pAS2/G713 plasmids bu retention of pACT-cDNA library plasmids. The resulting Y190 strains are mated with Y187 strains expressing G713 or non-related control proteins; such as cyclophilin B, lamin, or SNF1, as Gal4 fusions as described by Harper et al. (1993) and by Bram et al. (Bram R J et al., 1993), and screened for beta galactosidase by filter lift assay. Yeast clones that are beta gal—after mating with the control Gal4 fusions are considered false positives.

In another embodiment of the two-hybrid method according to the invention, interaction between the human or murine G713 or a fragment or variant thereof with cellular proteins may be assessed using the Matchmaker Two Hybrid System 2 (Catalog No. K1604-1, Clontech). ). As described in the manual accompanying the Matchmaker Two Hybrid System 2 (Catalog No. K1604-1, Clontech), the disclosure of which is incorporated herein by reference, nucleic acids encoding the human or murine G713 protein or a portion thereof, are inserted into an expression vector such that they are in frame with DNA encoding the DNA binding domain of the yeast transcriptional activator GAL4. A desired cDNA, preferably human cDNA, is inserted into a second expression vector such that they are in frame with DNA encoding the activation domain of GAL4. The two expression plasmids are transformed into yeast and the yeast are plated on selection medium which selects for expression of selectable markers on each of the expression vectors as well as GAL4 dependent expression of the HIS3 gene. Transformants capable of growing on medium lacking histidine are screened for GAL4 dependent lacZ expression. Those cells which are positive in both the histidine selection and the lacZ assay contain interaction between G713 and the protein or peptide encoded by the initially selected cDNA insert.

Method for Screening Ligands that Modulate the Expression of the G713 Gene

Another subject of the present invention is a method for screening molecules that modulate the expression of the G713 protein. Such a screening method comprises the steps of:

a) cultivating a prokaryotic or an eukaryotic cell that has been transfected with a nucleotide sequence encoding the G713 protein, placed under the control of its own promoter;

b) bringing into contact the cultivated cell with a molecule to be tested;

c) quantifying the expression of the G713 protein.

Using DNA recombination techniques well known by the one skill in the art, the G713 protein encoding DNA sequence is inserted into an expression vector, downstream from its promoter sequence. As an illustrative example, the promoter sequence of the G713 gene is contained in the nucleic acid of nucleotide positions 1076 to 3075 of SEQ ID No 1.

The quantification of the expression of the G713 protein may be realized either at the mRNA level or at the protein level. In the latter case, polyclonal or monoclonal antibodies may be used to quantify the amounts of the G713 protein that have been produced, for example in an ELISA or a RIA assay.

In a preferred embodiment, the quantification of the G713 mRNA is realized by a quantitative PCR amplification of the cDNA obtained by a reverse transcription of the total mRNA of the cultivated G713-transfected host cell, using a pair of primers specific for G713.

The present invention also concerns a method for screening substances or molecules that are able to increase, or in contrast to decrease, the level of expression of the G713 gene. Such a method may allow the one skilled in the art to select substances exerting a regulating effect on the expression level of the G713 gene and which may be useful as active ingredients included in pharmaceutical compositions for treating patients suffering from deficiencies in the regulation of expression of the G713 gene.

Thus, is also part of the present invention a method for the screening of a candidate substance or molecule that modulates the expression of the G713 gene, wherein said method comprises the following steps:

a) providing a recombinant host cell containing a nucleic acid, wherein said nucleic acid comprises a 5′UTR sequence of the G713 cDNA of SEQ ID No 4, or one of its biologically active fragments or variants, the 5′UTR sequence or its biologically active fragment or variant being operably linked to a polynucleotide encoding a detectable protein;

b) obtaining a candidate substance, and;

c) determining the ability of the candidate substance to modulate the expression levels of the polynucleotide encoding the detectable protein.

In another specific embodiment of the above screening method, the nucleic acid that comprises a nucleotide sequence selected from the group consisting of the 5′UTR sequence of the G713 cDNA of SEQ ID No 6 or one of its biologically active fragments or variants, includes a promoter sequence which is exogenous with respect to the G713 5′UTR sequences defined therein.

The invention further deals with a kit for the screening of a candidate substance modulating the expression of the G713 gene, wherein said kit comprises: a recombinant vector that comprises a nucleic acid including a 5′UTR sequence of the G713 cDNA of SEQ ID No 6, or one of their biologically active fragments or variants, the 5′UTR sequence or its biologically active fragment or variant being operably linked to a polynucleotide encoding a detectable protein.

The invention also pertains to a method for screening of a candidate substance or molecule that modulates the expression of the G713 gene, this method comprises the following steps:

a) providing a recombinant cell host containing a nucleic acid, wherein said nucleic acid comprises a nucleotide sequence of SEQ ID No 4 or a biologically active fragment or variant thereof located upstream a polynucleotide encoding a detectable protein;

b) obtaining a candidate substance, and

c) determining the ability of the candidate substance to modulate the expression levels of the polynucleotide encoding the detectable protein.

Among the preferred polynucleotides encoding a detectable protein, there may be cited polynucleotides encoding beta galactosidase, green fluorescent protein (GFP) and chloramphenicol acetyl transferase (CAT).

The invention also pertains to kits useful for performing the hereinbefore described screening method. Preferably, such kits comprise a recombinant vector that allows the expression of a nucleotide sequence of SEQ ID No 4 or a biologically active fragment or variant thereof located upstream a polynucleotide encoding a detectable protein.

For the design of suitable recombinant vectors useful for performing the screening methods described above, it will be referred to the section of the present specification wherein the preferred recombinant vectors of the invention are detailed.

Expression levels and patterns of G713 may be analyzed by solution hybridization with long probes as described in International Patent Application No. WO 97/05277, the entire contents of which are incorporated herein by reference. Briefly, the G713 cDNA or the G713 genomic DNA described above, or fragments thereof, is inserted at a cloning site immediately downstream of a bacteriophage (T3, T7 or SP6) RNA polymerase promoter to produce antisense RNA. Preferably, the G713 insert comprises at least 100 or more consecutive nucleotides of the genomic DNA sequence or the cDNA sequences, particularly those comprising at least one of SEQ ID Nos 1 T 04 OR 6 or those encoding a mutated G713. The plasmid is linearized and transcribed in the presence of ribonucleotides comprising modified ribonucleotides (i.e. biotin-UTP and DIG-UTP). An excess of this doubly labeled RNA is hybridized in solution with mRNA isolated from cells or tissues of interest. The hybridizations are performed under standard stringent conditions (40-50° C. for 16 hours in an 80% formamide, 0.4 M NaCl buffer, pH 7-8). The unhybridized probe is removed by digestion with ribonucleases specific for single-stranded RNA (i.e. RNases CL3, T1, Phy M, U2 or A). The presence of the biotin-UTP modification enables capture of the hybrid on a microtitration plate coated with streptavidin. The presence of the DIG modification enables the hybrid to be detected and quantified by ELISA using an anti-DIG antibody coupled to alkaline phosphatase.

Methods for Inhibiting the Expression of a G713 Gene

Other therapeutic compositions according to the present invention comprise advantageously an oligonucleotide fragment of the nucleic sequence of the human or murine G713 as an antisense tool that inhibits the expression of the corresponding G713 gene. Preferred methods using antisense polynucleotide according to the present invention are the procedures described by Sczakiel et al. (1995).

Preferably, the antisense tools are choosen among the polynucleotides (15-200 bp long) that are complementary to the 5′end of the human or murine G713 mRNA. In another embodiment, a combination of different antisense polynucleotides complementary to different parts of the desired targetted gene are used.

Preferred antisense polynucleotides according to the present invention are complementary to a sequence of the human or murine mRNAs of G713 that contains the translation initiation codon ATG

The antisense nucleic acids should have a length and melting temperature sufficient to permit formation of an intracellular duplex having sufficient stability to inhibit the expression of the human or murine G713 mRNA in the duplex. Strategies for designing antisense nucleic acids suitable for use in gene therapy are disclosed in Green et al., (1986) and Izant and Weintraub, (1984), the disclosures of which are incorporated herein by reference.

In some strategies, antisense molecules are obtained by reversing the orientation of the human or murine G713 coding region with respect to a promoter so as to transcribe the opposite strand from that which is normally transcribed in the cell. The antisense molecules may be transcribed using in vitro transcription systems such as those which employ T7 or SP6 polymerase to generate the transcript. Another approach involves transcription of human or murine G713 antisense nucleic acids in vivo by operably linking DNA containing the antisense sequence to a promoter in a suitable expression vector.

Alternatively, suitable antisense strategies are those described by Rossi et al. (1991), in the International Applications Nos. WO 94/23026, WO 95/04141, WO 92/18522 and in the European Patent Application No. EP 0 572 287 A2

An alternative to the antisense technology that is used according to the present invention consists in using ribozymes that will bind to a target sequence via their complementary polynucleotide tail and that will cleave the corresponding RNA by hydrolyzing its target site (namely “hammerhead ribozymes”). Briefly, the simplified cycle of a hammerhead ribozyme consists of (1) sequence specific binding to the target RNA via complementary antisense sequences; (2) site-specific hydrolysis of the cleavable motif of the target strand; and (3) release of cleavage products, which gives rise to another catalytic cycle. Indeed, the use of long-chain antisense polynucleotide (at least 30 bases long) or ribozymes with long antisense arms are advantageous. A preferred delivery system for antisense ribozyme is achieved by covalently linking these antisense ribozymes to lipophilic groups or to use liposomes as a convenient vector. Preferred antisense ribozymes according to the present invention are prepared as described by Sczakiel et al. (1995), the specific preparation procedures being referred to in said article being herein incorporated by reference.

Computer Related Embodiments

As used herein the term “nucleic acid codes of the invention” encompass the nucleotide sequences comprising, consisting essentially of, or consisting of any one of the following:

a) a contiguous span of at least 12, 15, 18, 20, 25, 30, 35, 40, 50, 60, 70, 80, 90, 100, 150, 200, 500, or 1000 nucleotides of SEQ ID Nos 1, 2 or 3, wherein said contiguous span comprises at least 1, 2, 3, 5, or 10 of the following nucleotide positions:

1 to 1000, 1001 to 2000, 2001 to 3000, 3001 to 4000 and 4001 to 5222 of SEQ ID No. 1;

1 to 1000, 1001 to 2000, 2001 to 3000, 3001 to 4000 and 4001 to 5000, 5001 to 6000, 6001 to 7000, 7001 to 8000, 8001 to 9000, 9001 to 10000, 10001 to 11000, 11001 to 12000, 12001 to 13000, 13001 to 14000, 14001 ot 15000, 15001 to 16000, 16001 to 17000, 17001 to 18000, 18001 to 19000, 19001 to 20000 and 20001 to 21278 of SEQ ID No 2; and

1 to 1000, 1001 to 2000, 2001 to 3000, 3001 to 4000 and 4001 to 5000, 5001 to 6000, 6001 to 7000, 7001 to 8000, 8001 to 9000, 9001 to 10000, 10001 to 11000, 11001 to 12000, 12001 to 13000, 13001 to 14000, 14001 ot 15000, 15001 to 16000, 16001 to 17000, 17001 to 18000, 18001 to 19000, 19001 to 20000, 20001 to 21000 and 21001 to 21636 of SEQ ID No 3;

b) a contiguous span of at least 12, 15, 18, 20, 25, 30, 35, 40, 50, 60, 70, 80, 90, 100, 150, 200, 500, or 1000 nucleotides of SEQ ID Nos 1, 2 or 3, or the complements thereof, wherein said contiguous span comprises at least one of the following nucleotide positions:

SEQ ID No 1: 1 to 3236, 3547 to 3585 and 4649 to 5222, or a variant thereof or a sequence complementary thereto;

SEQ ID No 2: 1 to 16155 and 16331 to 21278 or a variant thereof or a sequence complementary thereto; and

SEQ ID No 3: 1 to 5531, 6844 to 7237, 7798 to 8184, 8667 to 9074, and 9356 to 21636, or a variant thereof or a sequence complementary thereto;

c) a contiguous span of at least 12, 15, 18, 20, 25, 30, 35, 40, 50, 60, 70, 80, 90, 100, 150, 200, 500, or 1000 nucleotides of SEQ ID No 31 or the complements thereof, wherein said contiguous span comprises nucleotide positions 1 to 480 and 717 to 983 of SEQ ID No 31;

d) a contiguous span of at least 12, 15, 18, 20, 25, 30, 35, 40, 50, 60, 70, 80, 90, 100, 150, 200, 500, or 1000 nucleotides of SEQ ID No 4, or the complements thereof, wherein said contiguous span of SEQ ID No 4 comprises at least one of the following nucleotide positions of SEQ ID No 4: 1 to 519 and 2563 to 5566;

e) a contiguous span of at least 12, 15, 18, 20, 25, 30, 35, 40, 50, 60, 70, 80, 90, 100, 150, 200, 500, or 1000 nucleotides of SEQ ID No 6, or the complements thereof;

f) a contiguous span of at least 12, 15, 18, 20, 25, 30, 35, 40, 50, 60, 70, 80, 90, 100, 150, 200, 500, or 1000 nucleotides of SEQ ID Nos 32 to 69, or the complements thereof; and,

g) a nucleotide sequence complementary to any one of the preceding nucleotide sequences.

The “nucleic acid codes of the invention” further encompass nucleotide sequences homologous to:

a) a contiguous span of at least 12, 15, 18, 20, 25, 30, 35, 40, 50, 60, 70, 80, 90, 100, 150, 200, 500, or 1000 nucleotides of SEQ ID Nos 1 to 3, wherein said contiguous span comprises at least one of the following nucleotide positions:

SEQ ID No 1: 1 to 3236, 3547 to 3585 and 4649 to 5222, or a variant thereof or a sequence complementary thereto;

SEQ ID No 2: 1 to 16155 and 16331 to 21278 or a variant thereof or a sequence complementary thereto; and

SEQ ID No 3: 1 to 5531, 6844 to 7237, 7798 to 8184, 8667 to 9074, and 9356 to 21636 or a variant thereof or a sequence complementary thereto;

b) a contiguous span of at least 12, 15, 18, 20, 25, 30, 35, 40, 50, 60, 70, 80, 90, 100, 150, 200, 500, or 1000 nucleotides of SEQ ID No 4 or the complements thereof, wherein said contiguous span of SEQ ID No 4 comprises at least one of the following nucleotide positions of SEQ ID No 4: 1 to 519 and 2563 to 5566; and,

c) a contiguous span of at least 12, 15, 18, 20, 25, 30, 35, 40, 50, 60, 70, 80, 90, 100, 150, 200, 500, or 1000 nucleotides of SEQ ID No 6, or the complements thereof;

d) sequences complementary to all of the preceding sequences.

Homologous sequences refer to a sequence having at least 99%, 98%, 97%, 96%, 95%, 90%, 85%, 80%, or 75% homology to these contiguous spans. Homology may be determined using any method described herein, including BLAST2N with the default parameters or with any modified parameters. Homologous sequences also may include RNA sequences in which uridines replace the thymines in the nucleic acid codes of the invention. It will be appreciated that the nucleic acid codes of the invention can be represented in the traditional single character format (See the inside back cover of Stryer, Lubert. Biochemistry, 3^{rd} edition. W. H Freeman & Co., New York.) or in any other format or code which records the identity of the nucleotides in a sequence.

As used herein the term “polypeptide codes of the invention” encompass the polypeptide sequences comprising a contiguous span of at least 6, 8, 10, 12, 15, 20, 25, 30, 40, 50, or 100 amino acids of SEQ ID Nos 5 or 7. It will be appreciated that the polypeptide codes of the invention can be represented in the traditional single character format or three letter format (See the inside back cover of Stryer, Lubert. Biochemistry, 3^{rd} edition. W. H Freeman & Co., New York.) or in any other format or code which records the identity of the polypeptides in a sequence.

It will be appreciated by those skilled in the art that the nucleic acid codes of the invention and polypeptide codes of the invention can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the nucleic acid codes of the invention, or one or more of the polypeptide codes of the invention. Another aspect of the present invention is a computer readable medium having recorded thereon at least 2, 5, 10, 15, 20, 25, 30, or 50 nucleic acid codes of the invention. Another aspect of the present invention is a computer readable medium having recorded thereon at least 2, 5, 10, 15, 20, 25, 30, or 50 polypeptide codes of the invention.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

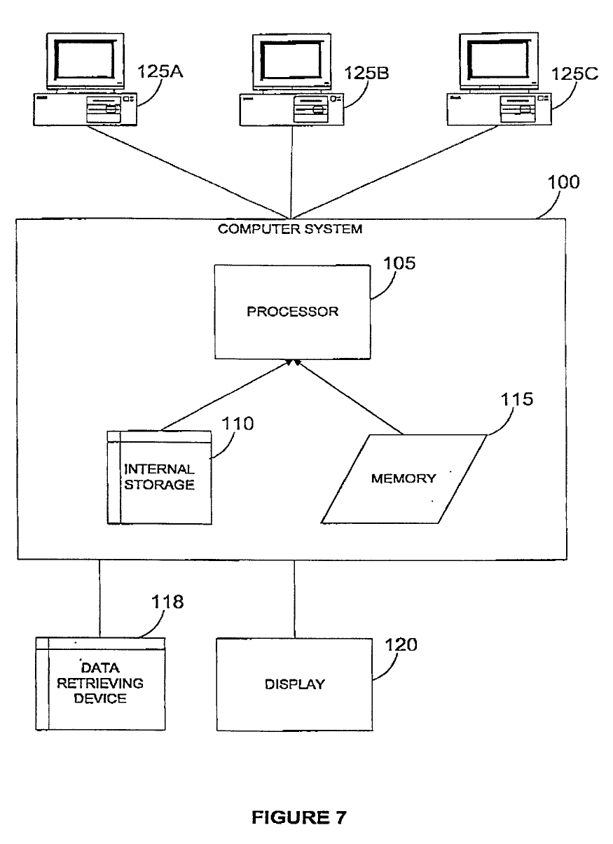

Embodiments of the present invention include systems, particularly computer systems which store and manipulate the sequence information described herein. One example of a computer system 100 is illustrated in block diagram form in FIG. 7. As used herein, “a computer system” refers to the hardware components, software components, and data storage components used to analyze the nucleotide sequences of the nucleic acid codes of the invention or the amino acid sequences of the polypeptide codes of the invention. In one embodiment, the computer system 100 is a Sun Enterprise 1000 server (Sun Microsystems, Palo Alto, Calif.). The computer system 100 preferably includes a processor for processing, accessing and manipulating the sequence data. The processor 105 can be any well-known type of central processing unit, such as the Pentium III from Intel Corporation, or similar processor from Sun, Motorola, Compaq or International Business Machines.

Preferably, the computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one particular embodiment, the computer system 100 includes a processor 105 connected to a bus which is connected to a main memory 115 (preferably implemented as RAM) and one or more internal data storage devices 110, such as a hard drive and/or other computer readable media having data recorded thereon. In some embodiments, the computer system 100 further includes one or more data retrieving device 118 for reading the data stored on the internal data storage devices 110.

The data retrieving device 118 may represent, for example, a floppy disk drive, a compact disk drive, a magnetic tape drive, etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device.

The computer system 100 includes a display 120 which is used to display output to a computer user. It should also be noted that the computer system 100 can be linked to other computer systems 125a-c in a network or wide area network to provide centralized access to the computer system 100.

Software for accessing and processing the nucleotide sequences of the nucleic acid codes of the invention or the amino acid sequences of the polypeptide codes of the invention (such as search tools, compare tools, and modeling tools etc.) may reside in main memory 115 during execution.

In some embodiments, the computer system 100 may further comprise a sequence comparer for comparing the above-described nucleic acid codes of the invention or the polypeptide codes of the invention stored on a computer readable medium to reference nucleotide or polypeptide sequences stored on a computer readable medium. A “sequence comparer” refers to one or more programs which are implemented on the computer system 100 to compare a nucleotide or polypeptide sequence with other nucleotide or polypeptide sequences and/or compounds including but not limited to peptides, peptidomimetics, and chemicals stored within the data storage means. For example, the sequence comparer may compare the nucleotide sequences of nucleic acid codes of the invention or the amino acid sequences of the polypeptide codes of the invention stored on a computer readable medium to reference sequences stored on a computer readable medium to identify homologies, motifs implicated in biological function, or structural motifs. The various sequence comparer programs identified elsewhere in this patent specification are particularly contemplated for use in this aspect of the invention.

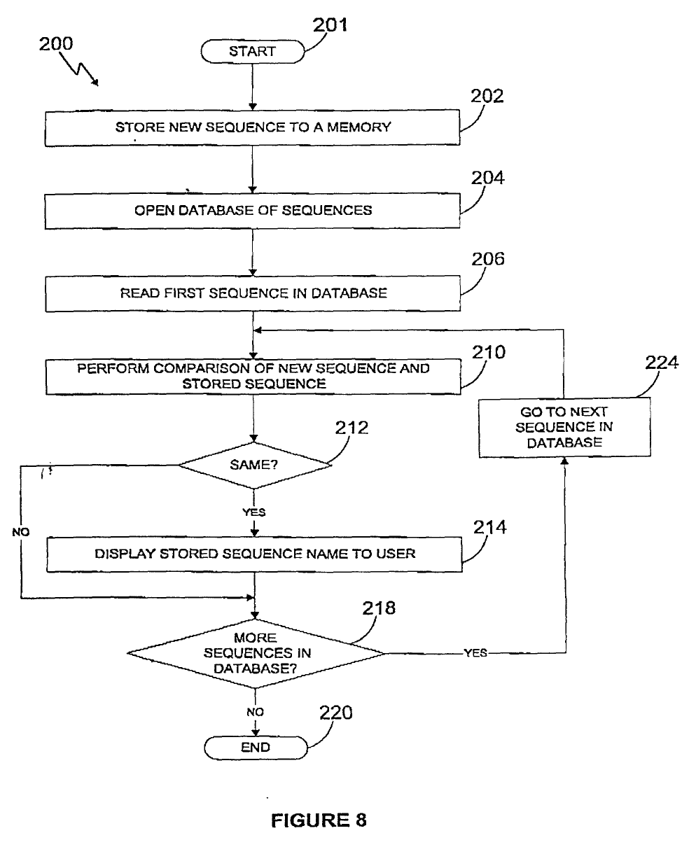

FIG. 8 is a flow diagram illustrating one embodiment of a process 200 for comparing a new nucleotide or protein sequence with a database of sequences in order to determine the homology levels between the new sequence and the sequences in the database. The database of sequences can be a private database stored within the computer system 100, or a public database such as GENBANK, PIR OR SWISSPROT that is available through the Internet.

The process 200 begins at a start state 201 and then moves to a state 202 wherein the new sequence to be compared is stored to a memory in a computer system 100. As discussed above, the memory could be any type of memory, including RAM or an internal storage device.

The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the homology level between the two tested sequences. The parameters that control whether gaps or other features are introduced into a sequence during comparison are normally entered by the user of the computer system.

Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200.

If a determination is made that the two sequences are the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database.

It should be noted that if a determination had been made at the decision state 212 that the sequences were not homologous, then the process 200 would move immediately to the decision state 218 in order to determine if any other sequences were available in the database for comparison.

Accordingly, one aspect of the present invention is a computer system comprising a processor, a data storage device having stored thereon a nucleic acid code of the invention or a polypeptide code of the invention, a data storage device having retrievably stored thereon reference nucleotide sequences or polypeptide sequences to be compared to the nucleic acid code of the invention or polypeptide code of the invention and a sequence comparer for conducting the comparison. The sequence comparer may indicate a homology level between the sequences compared or identify structural motifs in the nucleic acid code of the invention and polypeptide codes of the invention or it may identify structural motifs in sequences which are compared to these nucleic acid codes and polypeptide codes. In some embodiments, the data storage device may have stored thereon the sequences of at least 2, 5, 10, 15, 20, 25, 30, or 50 of the nucleic acid codes of the invention or polypeptide codes of the invention.

Another aspect of the present invention is a method for determining the level of homology between a nucleic acid code of the invention and a reference nucleotide sequence, comprising the steps of reading the nucleic acid code and the reference nucleotide sequence through the use of a computer program which determines homology levels and determining homology between the nucleic acid code and the reference nucleotide sequence with the computer program. The computer program may be any of a number of computer programs for determining homology levels, including those specifically enumerated herein, including BLAST2N with the default parameters or with any modified parameters. The method may be implemented using the computer systems described above. The method may also be performed by reading 2, 5, 10, 15, 20, 25, 30, or 50 of the above described nucleic acid codes of the invention through the use of the computer program and determining homology between the nucleic acid codes and reference nucleotide sequences.

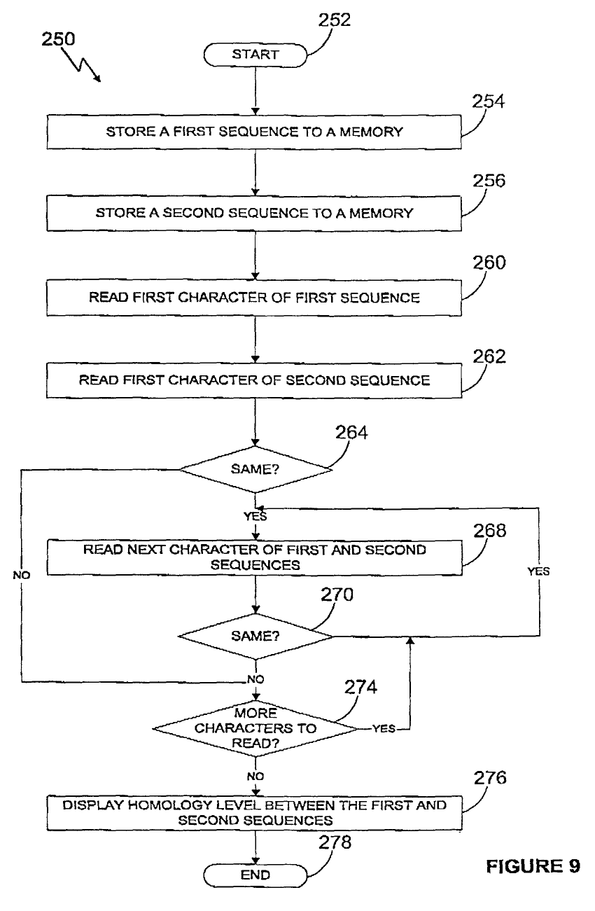

FIG. 9 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it should be in the single letter amino acid code so that the first and sequence sequences can be easily compared.

A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read.

If there aren't any more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with a every character in a second sequence, the homology level would be 100%.

Alternatively, the computer program may be a computer program which compares the nucleotide sequences of the nucleic acid codes of the present invention, to reference nucleotide sequences in order to determine whether the nucleic acid code of the invention differs from a reference nucleic acid sequence at one or more positions. Optionally such a program records the length and identity of inserted, deleted or substituted nucleotides with respect to the sequence of either the reference polynucleotide or the nucleic acid code of the invention. In one embodiment, the computer program may be a program which determines whether the nucleotide sequences of the nucleic acid codes of the invention contain one or more single nucleotide polymorphisms (SNP) with respect to a reference nucleotide sequence. These single nucleotide polymorphisms may each comprise a single base substitution, insertion, or deletion.

Another aspect of the present invention is a method for determining the level of homology between a polypeptide code of the invention and a reference polypeptide sequence, comprising the steps of reading the polypeptide code of the invention and the reference polypeptide sequence through use of a computer program which determines homology levels and determining homology between the polypeptide code and the reference polypeptide sequence using the computer program.

Accordingly, another aspect of the present invention is a method for determining whether a nucleic acid code of the invention differs at one or more nucleotides from a reference nucleotide sequence comprising the steps of reading the nucleic acid code and the reference nucleotide sequence through use of a computer program which identifies differences between nucleic acid sequences and identifying differences between the nucleic acid code and the reference nucleotide sequence with the computer program. In some embodiments, the computer program is a program which identifies single nucleotide polymorphisms the method may be implemented by the computer systems described above and the method illustrated in FIG. 9. The method may also be performed by reading at least 2, 5, 10, 15, 20, 25, 30, or 50 of the nucleic acid codes of the invention and the reference nucleotide sequences through the use of the computer program and identifying differences between the nucleic acid codes and the reference nucleotide sequences with the computer program. In other embodiments the computer based system may further comprise an identifier for identifying features within the nucleotide sequences of the nucleic acid codes of the invention or the amino acid sequences of the polypeptide codes of the invention.

An “identifier” refers to one or more programs which identifies certain features within the above-described nucleotide sequences of the nucleic acid codes of the invention or the amino acid sequences of the polypeptide codes of the invention. In one embodiment, the identifier may comprise a program which identifies an open reading frame in the cDNAs codes of the invention.

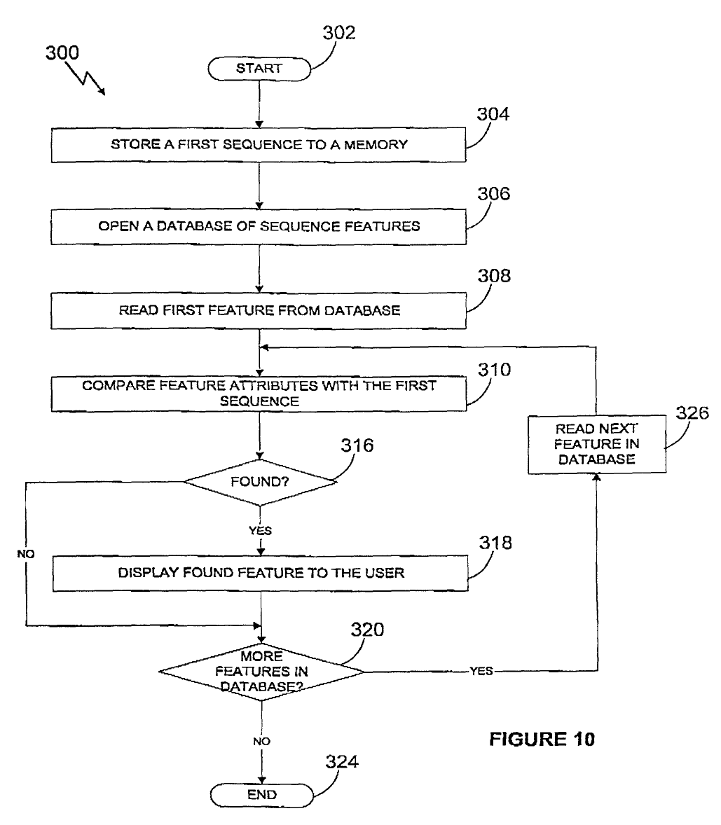

FIG. 10 is a flow diagram illustrating one embodiment of an identifier process 300 for detecting the presence of a feature in a sequence. The process 300 begins at a start state 302 and then moves to a state 304 wherein a first sequence that is to be checked for features is stored to a memory 115 in the computer system 100. The process 300 then moves to a state 306 wherein a database of sequence features is opened. Such a database would include a list of each feature's attributes along with the name of the feature. For example, a feature name could be “Initiation Codon” and the attribute would be “ATG”. Another example would be the feature name “TAATAA Box” and the feature attribute would be “TAATAA”. An example of such a database is produced by the University of Wisconsin Genetics Computer Group (www.gcg.com).

Once the database of features is opened at the state 306, the process 300 moves to a state 308 wherein the first feature is read from the database. A comparison of the attribute of the first feature with the first sequence is then made at a state 310. A determination is then made at a decision state 316 whether the attribute of the feature was found in the first sequence. If the attribute was found, then the process 300 moves to a state 318 wherein the name of the found feature is displayed to the user.

The process 300 then moves to a decision state 320 wherein a determination is made whether move features exist in the database. If no more features do exist, then the process 300 terminates at an end state 324. However, if more features do exist in the database, then the process 300 reads the next sequence feature at a state 326 and loops back to the state 310 wherein the attribute of the next feature is compared against the first sequence.

It should be noted, that if the feature attribute is not found in the first sequence at the decision state 316, the process 300 moves directly to the decision state 320 in order to determine if any more features exist in the database. In another embodiment, the identifier may comprise a molecular modeling program which determines the 3-dimensional structure of the polypeptides codes of the invention. In some embodiments, the molecular modeling program identifies target sequences that are most compatible with profiles representing the structural environments of the residues in known three-dimensional protein structures. (See, e.g., Eisenberg et al., U.S. Pat. No. 5,436,850 issued Jul. 25, 1995). In another technique, the known three-dimensional structures of proteins in a given family are superimposed to define the structurally conserved regions in that family. This protein modeling technique also uses the known three-dimensional structure of a homologous protein to approximate the structure of the polypeptide codes of the invention. (See e.g., Srinivasan, et al., U.S. Pat. No. 5,557,535 issued Sep. 17, 1996). Conventional homology modeling techniques have been used routinely to build models of proteases and antibodies. (Sowdhamini et al., (1997)). Comparative approaches can also be used to develop three-dimensional protein models when the protein of interest has poor sequence identity to template proteins. In some cases, proteins fold into similar three-dimensional structures despite having very weak sequence identities. For example, the three-dimensional structures of a number of helical cytokines fold in similar three-dimensional topology in spite of weak sequence homology.

The recent development of threading methods now enables the identification of likely folding patterns in a number of situations where the structural relatedness between target and template(s) is not detectable at the sequence level. Hybrid methods, in which fold recognition is performed using Multiple Sequence Threading (MST), structural equivalencies are deduced from the threading output using a distance geometry program DRAGON to construct a low resolution model, and a full-atom representation is constructed using a molecular modeling package such as QUANTA.

According to this 3-step approach, candidate templates are first identified by using the novel fold recognition algorithm MST, which is capable of performing simultaneous threading of multiple aligned sequences onto one or more 3-D structures. In a second step, the structural equivalencies obtained from the MST output are converted into interresidue distance restraints and fed into the distance geometry program DRAGON, together with auxiliary information obtained from secondary structure predictions. The program combines the restraints in an unbiased manner and rapidly generates a large number of low resolution model confirmations. In a third step, these low resolution model confirmations are converted into full-atom models and subjected to energy minimization using the molecular modeling package QUANTA. (See e.g., Aszódi et al., (1997)).

The results of the molecular modeling analysis may then be used in rational drug design techniques to identify agents which modulate the activity of the polypeptide codes of the invention.

Accordingly, another aspect of the present invention is a method of identifying a feature within the nucleic acid codes of the invention or the polypeptide codes of the invention comprising reading the nucleic acid code(s) or the polypeptide code(s) through the use of a computer program which identifies features therein and identifying features within the nucleic acid code(s) or polypeptide code(s) with the computer program. In one embodiment, computer program comprises a computer program which identifies open reading frames. In a further embodiment, the computer program identifies structural motifs in a polypeptide sequence. In another embodiment, the computer program comprises a molecular modeling program. The method may be performed by reading a single sequence or at least 2, 5, 10, 15, 20, 25, 30, or 50 of the nucleic acid codes of the invention or the polypeptide codes of the invention through the use of the computer program and identifying features within the nucleic acid codes or polypeptide codes with the computer program.

The nucleic acid codes of the invention or the polypeptide codes of the invention may be stored and manipulated in a variety of data processor programs in a variety of formats. For example, they may be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparers, identifiers, or sources of reference nucleotide or polypeptide sequences to be compared to the nucleic acid codes of the invention or the polypeptide codes of the invention. The following list is intended not to limit the invention but to provide guidance to programs and databases which are useful with the nucleic acid codes of the invention or the polypeptide codes of the invention. The programs and databases which may be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, 1990), FASTA (Pearson and Lipman, 1988), FASTDB (Brutlag et al., 1990), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the EMBL/Swissprotein database, the MDL Available Chemicals Directory database, the MDL Drug Data Report data base, the Comprehensive Medicinal Chemistry database, Derwents's World Drug Index database, the BioByteMasterFile database, the Genbank database, and the Genseqn database. Many other programs and data bases would be apparent to one of skill in the art given the present disclosure. Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

### EXAMPLE 1: G713

### Example 1(a)

Isolation of a mRNA Encoding the Murine G713 Polypeptide

A homology search in Genbank with the coding sequences from the human G713 transcription product revealed the presence of one mouse EST sequence (Accession number W89905) referenced in the database. This Genbank EST sequence has a 80% homology to the 5′-end of the human G713 transcript and to another mouse EST having the Accession number AA027647, with approximately the same degree of homology to the 3′-end of the human G713 coding sequence.

In order to amplify the murine G713 mRNA, the following pair of primers has been designed:

Primer 1 (g713CTGLF132): 5′-GGCTGTGCGTTCCCAAAATA-3′ (SEQ ID No 14); and

Primer 2: (moCTGR1511): 5′-TGTCCTCGAGCGTGGGG-3′ (SEQ ID No 26).

A Long Range PCR amplification has been performed using the Marathon Ready cDNA library from mouse brain (Clonetech, Ref. 7450-1, batch No 8010338) and a fragment of 1405 bp was amplified and sequenced by primer walking.

For excluding the presence of artefactual products, another couple or primers were designed, which are the following:

Primer 1 (moCTGLR20): 5′-CGGAGGAGGGGATACGGAAATTAAACC-3′ (SEQ ID No 27); and

Primer 2 (moCTG1440): 5′-TGGGTCACTGCTGCTCTGTGCCAAG-3′ (SEQ ID No 28).

A Long Range PCR amplification was performed using the mouse brain Marathon Ready cDNA library and a fragment of about 1.5 kb has been amplified. End sequencing of this amplification product confirmed its identity as the mouse G713 mRNA, as it is determined below.

A set of nested primers has been designed form the 3′-end of the above 1.5 kb fragment, which is the following:

Primer 1 (moCTG5RACE1): 5′-TCACAGTGTCCTCGGCCACT-3′ (SEQ ID No 29); and

Primer 2 (moCTG5RACEn): 5′-TCCTCCACACAGTGCTCACG-3′ (SEQ ID No 30).

These nested primers were used with the marathon primers AP1 and AP2 for performing a nested RACE reaction form the same mouse brain cDNA library. One fragment of approximately 700 bp was obtained and sequenced by primer walking. Contigation of the whole above mouse brain cDNA sequences resulted in a fragment covering the coding part of the mouse G713 cDNA.

### Example 1(b)

Detection of G713 Biallelic Markers: DNA Extraction

Donors were unrelated and healthy. They presented a sufficient diversity for being representative of a French heterogeneous population. The DNA from 100 individuals was extracted and tested for the detection of the biallelic markers.

30 ml of peripheral venous blood were taken from each donor in the presence of EDTA. Cells (pellet) were collected after centrifugation for 10 minutes at 2000 rpm. Red cells were lysed by a lysis solution (50 ml final volume: 10 mM Tris pH7.6; 5 mM MgCl₂; 10 mM NaCl). The solution was centrifuged (10 minutes, 2000 rpm) as many times as necessary to eliminate the residual red cells present in the supernatant, after resuspension of the pellet in the lysis solution.

The pellet of white cells was lysed overnight at 42° C. with 3.7 ml of lysis solution composed of:

3 ml TE 10-2 (Tris-HCl 10 mM, EDTA 2 mM)/NaCl 0.4 M

200 μl SDS 10%

500 82 l K-proteinase (2 mg K-proteinase in TE 10-2/NaCl 0.4 M).

For the extraction of proteins, 1 ml saturated NaCl (6M) (1/3.5 v/v) was added. After vigorous agitation, the solution was centrifuged for 20 minutes at 10000 rpm.

For the precipitation of DNA, 2 to 3 volumes of 100% ethanol were added to the previous supernatant, and the solution was centrifuged for 30 minutes at 2000 rpm. The DNA solution was rinsed three times with 70% ethanol to eliminate salts, and centrifuged for 20 minutes at 2000 rpm. The pellet was dried at 37° C., and resuspended in 1 ml TE 10-1 or 1 ml water. The DNA concentration was evaluated by measuring the OD at 260 nm (1 unit OD=50 μg/ml DNA).

To determine the presence of proteins in the DNA solution, the OD 260/OD 280 ratio was determined. Only DNA preparations having a OD 260/OD 280 ratio between 1.8 and 2 were used in the subsequent examples described below.

The pool was constituted by mixing equivalent quantities of DNA from each individual.

### Example 1(c)

Detection of the Biallelic Markers: Amplification of Genomic DNA by PCR

The amplification of specific genomic sequences of the DNA samples of example 1 (b) was carried out on the pool of DNA obtained previously. In addition, 50 individual samples were similarly amplified.

PCR assays were performed using the following protocol:

| | |
|---|---|
| Final volume | 25 μl |
| DNA | 2 ng/μl |
| MgCl₂ | 2 mM |
| dNTP (each) | 200 μM |
| primer (each) | 2.9 ng/μl |
| Ampli Taq Gold DNA polymerase | 0.05 unit/μl |
| PCR buffer (10x = 0.1 M TrisHCl pH 8.3 0.5 M KCl | 1x |

Each pair of first primers was designed using the sequence information of the G713 gene disclosed herein and the OSP software (Hillier & Green, 1991). This first pair of primers was about 20 nucleotides in length and had the sequences disclosed in Table 1 in the columns labeled PU and RP.

TABLE 1

| | | | Position range | | | Complementary | |
|---|---|---|---|---|---|---|---|
| | | | of amplification | | | position range of | |
| | SEQ | Primer | primer in SEQ | | Primer | amplification | |
| Amplicon | ID No | name | ID | | name | primer in SEQ ID | |
| 8-58 | 1 | B1 | 4572 | 4587 | C1 | 4990 | 5005 |
| 99-16063 | 2 | B2 | 3045 | 3062 | C2 | 3547 | 3565 |
| 99-16073 | 2 | B3 | 6058 | 6076 | C3 | 6493 | 6512 |
| 99-16074 | 2 | B4 | 9661 | 9678 | C4 | 10119 | 10136 |
| 99-13817 | 2 | B5 | 14754 | 14774 | C5 | 15183 | 15203 |
| 99-16066 | 2 | B6 | 20137 | 20155 | C6 | 20569 | 20588 |
| 99-13821 | 3 | B7 | 7946 | 7965 | C7 | 8454 | 8472 |
| 99-13525 | 3 | B8 | 15943 | 15960 | C8 | 16430 | 16447 |
| 99-13526 | 3 | B9 | 16950 | 16970 | C9 | 17381 | 17401 |
| 99-15215 | 3 | B10 | 15475 | 15495 | C10 | 15954 | 15974 |
| 99-15208 | 3 | B11 | 19315 | 19333 | C11 | 19797 | 19817 |

Preferably, the primers contained a common oligonucleotide tail upstream of the specific bases targeted for amplification which was useful for sequencing. Primers PU contain the following additional PU 5′ sequence:

TGTAAAACGACGGCCAGT; primers RP contain the following RP 5′ sequence:

CAGGAAACAGCTATGACC. The primer containing the additional PU 5′ sequence is listed in SEQ ID No 70. The primer containing the additional RP 5′ sequence is listed in SEQ ID No 71.

The synthesis of these primers was performed following the phosphoramidite method, on a GENSET UFPS 24.1 synthesizer.

DNA amplification was performed on a Genius II thermocycler. After heating at 95° C. for 10 min, 40 cycles were performed. Each cycle comprised: 30 sec at 95° C., 54° C. for 1 min, and 30 sec at 72° C. For final elongation, 10 min at 72° C. ended the amplification. The quantities of the amplification products obtained were determined on 96-well microtiter plates, using a fluorometer and Picogreen as intercalant agent (Molecular Probes).

### Example 1(d)

Detection of the Biallelic Markers: Sequencing of Amplified Genomic DNA and Identification of Polymorphisms

The sequencing of the amplified DNA obtained in example 1(c) was carried out on ABI 377 sequencers. The sequences of the amplification products were determined using automated dideoxy terminator sequencing reactions with a dye terminator cycle sequencing protocol. The products of the sequencing reactions were run on sequencing gels and the sequences were determined using gel image analysis (ABI Prism DNA Sequencing Analysis software (2.1.2 version) and the above mentioned proprietary “Trace” basecaller).

The sequence data were further evaluated using the above mentioned polymorphism analysis software designed to detect the presence of biallelic markers among the pooled amplified fragments. The polymorphism search was based on the presence of superimposed peaks in the electrophoresis pattern resulting from different bases occurring at the same position as described previously.

The localization of the biallelic markers are as shown in Table 2.

TABLE 2
Biallelic Markers

| | | | | Polymor- | | |
|---|---|---|---|---|---|---|
| | SEQ ID | | Localization in | phism | | BM position |
| BM | No | Marker Name | GENE gene | all1 | all2 | in SEQ ID |
| A1 | 1 | 8-58-301 | Intron 1 | C | T | 4872 |
| A2 | 2 | 99-16063-218 | Intron 1 | A | G | 3262 |
| A3 | 2 | 99-16073-282 | Intron 1 | C | T | 6231 |
| A4 | 2 | 99-16074-266 | Intron 1 | A | G | 9871 |
| A5 | 2 | 99-13817-215 | Intron 1 | C | T | 14968 |
| A6 | 2 | 99-16066-123 | Intron 2 | C | T | 20259 |
| A7 | 3 | 99-13821-332 | Exon 3 | C | T | 8277 |
| A8 | 3 | 99-13525-395 | 3′ portion of | A | G | 16053 |
| | | | genomic sequence | | | |
| A9 | 3 | 99-13526-368 | 3′ portion of | A | G | 17032 |
| | | | genomic sequence | | | |
| A10 | 3 | 99-15215-60 | 3′ portion of | C | T | 15915 |
| | | | genomic sequence | | | |
| A11 | 3 | 99-15208-87 | 3′ portion of | A | G | 19401 |
| | | | genomic sequence | | | |

BM refers to “biallelic marker”. AII1 and aII2 refer respectively to allele 1 and allele 2 of the biallelic marker.

TABLE 3

| | | | Position range | | |
|---|---|---|---|---|---|
| | SEQ ID | | of probes in | | |
| BM | NO | Marker Name | SEQ ID No | | Probes |
| A1 | 1 | 8-58-301 | 4849 | 4895 | P1 |
| A2 | 2 | 99-16063-218 | 3239 | 3285 | P2 |
| A3 | 2 | 99-16073-282 | 6208 | 6254 | P3 |
| A4 | 2 | 99-16074-266 | 9848 | 9894 | P4 |
| A5 | 2 | 99-13817-215 | 14945 | 14991 | P5 |
| A6 | 2 | 99-16066-123 | 20236 | 20282 | P6 |
| A7 | 3 | 99-13821-332 | 8254 | 8300 | P7 |
| A8 | 3 | 99-13525-395 | 16030 | 16076 | P8 |
| A9 | 3 | 99-13526-368 | 17009 | 17055 | P9 |
| A10 | 3 | 99-15215-60 | 15892 | 15938 | P10 |
| A11 | 3 | 99-15208-87 | 19378 | 19424 | P11 |

### Example 1(e)

Validation of the Polymorphisms Through Microsequencing

The biallelic markers identified in Example (d) were further confirmed and their respective frequencies were determined through microsequencing. Microsequencing was carried out for each individual DNA sample described in Example (b).

Amplification from genomic DNA of individuals was performed by PCR as described above for the detection of the biallelic markers with the same set of PCR primers (Table 1).

The preferred primers used in microsequencing were about 20 nucleotides in length and hybridized just upstream of the considered polymorphic base. According to the invention, the primers used in microsequencing are detailed in Table 4.

TABLE 4

| | | | | Position range | | | Complementary | |
|---|---|---|---|---|---|---|---|---|
| | | | | of | | | position range of | |
| | SEQ | | | microsequencin | | | microsequencing | |
| | ID | Biallelic | | g primer mis 1 in | | | primer mis. 2 in | |
| Marker Name | No. | Marker | Mis. 1 | SEQ ID No | | Mis. 2 | SEQ ID No | |
| 8-58-301 | 1 | A1 | D1 | 4853 | 4871 | E1 | 4873 | 4891 |
| 99-16063-218 | 2 | A2 | D2 | 3243 | 3261 | E2 | 3263 | 3281 |
| 99-16073-282 | 2 | A3 | D3 | 6212 | 6230 | E3 | 6232 | 6250 |
| 99-16074-266 | 2 | A4 | D4 | 9852 | 9870 | E4 | 9872 | 9890 |
| 99-13817-215 | 2 | A5 | D5 | 14949 | 14967 | E5 | 14969 | 14987 |
| 99-16066-123 | 2 | A6 | D6 | 20240 | 20258 | E6 | 20260 | 20278 |
| 99-13821-332 | 3 | A7 | D7 | 8258 | 8276 | E7 | 8278 | 8296 |
| 99-13525-395 | 3 | A8 | D8 | 16034 | 16052 | E8 | 16054 | 16072 |
| 99-13526-368 | 3 | A9 | D9 | 17013 | 17031 | E9 | 17033 | 17051 |
| 99-15215-60 | 3 | A10 | D10 | 15896 | 15914 | E10 | 15916 | 15634 |
| 99-15208-87 | 3 | A11 | D11 | 19382 | 19400 | E11 | 19402 | 19420 |

Mis 1 and Mis 2 respectively refer to microsequencing primers which hybridized with the non-coding strand of the G713 gene or with the coding strand of the G713 gene.

The microsequencing reaction was performed as follows:

After purification of the amplification products, the microsequencing reaction mixture was prepared by adding, in a 20 μl final volume: 10 pmol microsequencing oligonucleotide, 1 U Thermosequenase (Amersham E79000G), 1.25 μl Thermosequenase buffer (260 mM Tris HCl pH 9.5, 65 mM MgCl₂), and the two appropriate fluorescent ddNTPs (Perkin Elmer, Dye Terminator Set 401095) complementary to the nucleotides at the polymorphic site of each biallelic marker tested, following the manufacturer's recommendations. After 4 minutes at 94° C., 20 PCR cycles of 15 sec at 55° C., 5 sec at 72° C., and 10 sec at 94° C. were carried out in Tetrad PTC-225 thermocycler (MJ Research). The unincorporated dye terminators were then removed by ethanol precipitation. Samples were finally resuspended in formamide-EDTA loading buffer and heated for 2 min at 95° C. before being loaded on a polyacrylamide sequencing gel. The data were collected by an ABI PRISM 377 DNA sequencer and processed using the GENESCAN software (Perkin Elmer).

Following gel analysis, data were automatically processed with software that allows the determination of the allelese of biallelic marker present in each amplified fragment.

The software evaluates such factors as whether the intensities of the signals resulting from the above microsequencing procedures are weak, normal, or saturated, or whether the signals are ambiguous. In addition, the software identifies significant peaks (according to shape and height criteria). Among the significant peaks, peaks corresponding to the targeted site are identified based on their position. When two significant peaks are detected for the same position, each sample is categorized classification as homozygous or herozygous type based on the height ratio.

TABLE 5
Restriction map of the G713 5′ regulatory polynucleotide of SEQ ID No 4

| Name | Sequence | Position | Fragment | lengths |
|---|---|---|---|---|
| 1 AATII | | 0 | −1 | −1 |
| | | | 2001 | 2001 |
| 1 ACCI | GT′ATAC | 164 | 163 | 163 |
| | | | 1837 | 1837 |
| 1 AFLII | | 0 | −1 | −1 |
| | | | 2001 | 2001 |
| 1 AFLIII | | 0 | −1 | −1 |
| | | | 2001 | 2001 |
| 1 APAI | GGGCC′C | 1469 | 1468 | 8 |
| 2 APAI | GGGCC′C | 1477 | 8 | 524 |
| | | | 524 | 1468 |
| 1 APALI | G′TGCAC | 378 | 377 | 377 |
| 2 APALI | G′TGCAC | 1041 | 663 | 663 |
| | | | 960 | 960 |
| 1 ASUII | | 0 | −1 | −1 |
| | | | 2001 | 2001 |
| 1 AVRII | | 0 | −1 | −1 |
| | | | 2001 | 2001 |
| 1 BALI | | 0 | −1 | −1 |
| | | | 2001 | 2001 |
| 1 BAMHI | G′GATCC | 1127 | 1126 | 874 |
| | | | 874 | 1126 |
| 1 BCLI | T′GATCA | 109 | 108 | 108 |
| | | | 1892 | 1892 |
| 1 BGLII | | 0 | −1 | −1 |
| | | | 2001 | 2001 |
| 1 BSMI | CG′CATTC | 1138 | 1137 | 863 |
| | | | 863 | 1137 |
| 1 BSPMI | ACCTGCTGCT′ | 528 | 527 | 500 |
| 2 BSPMI | CGGTCGATGCAGGT | 1028 | 500 | 527 |
| | | | 973 | 973 |
| 1 BSPMII | TCCGG′A | 1857 | 1856 | 144 |
| | | | 144 | 1856 |
| 1 BSTEII | G′GTCACC | 349 | 348 | 280 |
| 2 BSTEII | G′GTGACC | 629 | 280 | 348 |
| | | | 1372 | 1372 |
| 1 BSTXI | CCATCCCT′TTGG | 317 | 316 | 316 |
| | | | 1684 | 1684 |
| 1 CLAI | | 0 | −1 | −1 |
| | | | 2001 | 2001 |
| 1 DRAI | TTT′AAA | 52 | 51 | 51 |
| 2 DRAI | TTT′AAA | 239 | 187 | 187 |
| | | | 1762 | 1762 |
| 1 DRAIII | CACTCG′GTG | 487 | 486 | 486 |
| | | | 1514 | 1514 |
| 1 EAEI | C′GGCCG | 1330 | 1329 | 19 |
| 2 EAEI | C′GGCCA | 1349 | 19 | 652 |
| | | | 652 | 1329 |
| 1 ECOB | | 0 | −1 | −1 |
| | | | 2001 | 2001 |
| 1 ECOK | | 0 | −1 | −1 |
| | | | 2001 | 2001 |
| 1 ECORI | | 0 | −1 | −1 |
| | | | 2001 | 2001 |
| 1 ECORV | | 0 | −1 | −1 |
| | | | 2001 | 2001 |
| 1 ESPI | | 0 | −1 | −1 |
| | | | 2001 | 2001 |
| 1 FSPI | TGC′GCA | 491 | 490 | 490 |
| | | | 1510 | 1510 |
| 1 HINCII | | 0 | −1 | −1 |
| | | | 2001 | 2001 |
| 1 HINDIII | | 0 | −1 | −1 |
| | | | 2001 | 2001 |
| 1 HPAI | | 0 | −1 | −1 |
| | | | 2001 | 2001 |
| 1 KPNI | | 0 | −1 | −1 |
| | | | 2001 | 2001 |
| 1 MLUI | | 0 | −1 | −1 |
| | | | 2001 | 2001 |
| 1 MSTII | | 0 | −1 | −1 |
| | | | 2001 | 2001 |
| 1 NAEI | GCC′GGC | 1534 | 1533 | 467 |
| | | | 467 | 1533 |
| 1 NCOI | | 0 | −1 | −1 |
| | | | 2001 | 2001 |
| 1 NDEI | | 0 | −1 | −1 |
| | | | 2001 | 2001 |
| 1 NHEI | | 0 | −1 | −1 |
| | | | 2001 | 2001 |
| 1 NOTI | GC′GGCCGC | 1330 | 1329 | 671 |
| | | | 671 | 1329 |
| 1 NRUI | | 0 | −1 | −1 |
| | | | 2001 | 2001 |
| 1 NSII | ATGCA′T | 333 | 332 | 332 |
| | | | 1668 | 1668 |
| 1 PFIMI | | 0 | −1 | −1 |
| | | | 2001 | 2001 |
| 1 PPUMI | GG′GTCCT | 400 | 399 | 361 |
| 2 PPUMI | AG′GTCCT | 761 | 361 | 399 |
| | | | 1240 | 1240 |
| 1 PSTI | | 0 | −1 | −1 |
| | | | 2001 | 2001 |
| 1 PVUI | | 0 | −1 | −1 |
| | | | 2001 | 2001 |
| 1 PVUII | | 0 | −1 | −1 |
| | | | 2001 | 2001 |
| 1 RSRII | CG′GTCCG | 1121 | 1120 | 880 |
| | | | 880 | 1120 |
| 1 SACI | GAGCT′C | 1563 | 1562 | 143 |
| 2 SACI | GAGCT′C | 1706 | 143 | 295 |
| | | | 295 | 1562 |
| 1 SALI | | 0 | −1 | −1 |
| | | | 2001 | 2001 |
| 1 SCAI | AGT′ACT | 19 | 18 | 18 |
| | | | 1982 | 1982 |
| 1 SNABI | | 0 | −1 | −1 |
| | | | 2001 | 2001 |
| 1 SPEI | | 0 | −1 | −1 |
| | | | 2001 | 2001 |
| 1 SPHI | | 0 | −1 | −1 |
| | | | 2001 | 2001 |
| 1 SSPI | | 0 | −1 | −1 |
| | | | 2001 | 2001 |
| 1 STUI | | 0 | −1 | −1 |
| | | | 2001 | 2001 |
| 1 STYI | C′CTTGG | 403 | 402 | 402 |
| | | | 1598 | 1598 |
| 1 TTHIIII | | 0 | −1 | −1 |
| | | | 2001 | 2001 |
| 1 XBAI | | 0 | −1 | −1 |
| | | | 2001 | 2001 |
| 1 XHOI | | 0 | −1 | −1 |
| | | | 2001 | 2001 |
| 1 XMAIII | C′GGCCG | 1330 | 1329 | 671 |
| | | | 671 | 1329 |
| 1 XMNI | | 0 | −1 | −1 |
| | | | 2001 | 2001 |

### Example 1(f)

Preparation of Antibody Compositions to the G713 Protein

Substantially pure protein or polypeptide is isolated from transfected or transformed cells containing an expression vector encoding the G713 protein or a portion thereof. The concentration of protein in the final preparation is adjusted, for example, by concentration on an Amicon filter device, to the level of a few micrograms/ml. Monoclonal or polyclonal antibody to the protein can then be prepared as follows:

A. Monoclonal Antibody Production by Hybridoma Fusion

Monoclonal antibody to epitopes in the G713 protein or a portion thereof can be prepared from murine hybridomas according to the classical method of Kohler, G. and Milstein, C., (1975) or derivative methods thereof. Also see Harlow, E., and D. Lane. 1988.

Briefly, a mouse is repetitively inoculated with a few micrograms of the G713 protein or a portion thereof over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, (1980), and derivative methods thereof. Selected positive clones can be expanded and their monoclonal antibody product harvested for use. Detailed procedures for monoclonal antibody production are described in Davis, L. et al. Basic Methods in Molecular Biology Elsevier, New York. Section 21-2.

B. Polyclonal Antibody Production by Immunization

Polyclonal antiserum containing antibodies to heterogeneous epitopes in the G713 protein or a portion thereof can be prepared by immunizing suitable non-human animal with the G713 protein or a portion thereof, which can be unmodified or modified to enhance immunogenicity. A suitable non-human animal is preferably a non-human mammal is selected, usually a mouse, rat, rabbit, goat, or horse. Alternatively, a crude preparation which has been enriched for G713 concentration can be used to generate antibodies. Such proteins, fragments or preparations are introduced into the non-human mammal in the presence of an appropriate adjuvant (e.g. aluminum hydroxide, RIBI, etc.) which is known in the art. In addition the protein, fragment or preparation can be pretreated with an agent which will increase antigenicity, such agents are known in the art and include, for example, methylated bovine serum albumin (mBSA), bovine serum albumin (BSA), Hepatitis B surface antigen, and keyhole limpet hemocyanin (KLH). Serum from the immunized animal is collected, treated and tested according to known procedures. If the serum contains polyclonal antibodies to undesired epitopes, the polyclonal antibodies can be purified by immunoaffinity chromatography.

Effective polyclonal antibody production is affected by many factors related both to the antigen and the host species. Also, host animals vary in response to site of inoculations and dose, with both inadequate or excessive doses of antigen resulting in low titer antisera. Small doses (ng level) of antigen administered at multiple intradermal sites appears to be most reliable. Techniques for producing and processing polyclonal antisera are known in the art, see for example, Mayer and Walker (1987). An effective immunization protocol for rabbits can be found in Vaitukaitis, J. et al. (1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., (1973). Plateau concentration of antibody is usually in the range of 0.1 to 0.2 mg/ml of serum (about 12 μM). Affinity of the antisera for the antigen is determined by preparing competitive binding curves, as described, for example, by Fisher, D., (1980).

Antibody preparations prepared according to either the monoclonal or the polyclonal protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

### Example 2

Schizophrenia-related Biallelic Markers

### Example 2(a)

Detection of SG2 Biallelic Markers: DNA Extraction

Donors were unrelated and healthy. They presented a sufficient diversity for being representative of a French heterogeneous population. The DNA from 100 individuals was extracted and tested for the detection of the biallelic markers.

Briefly, 30 ml of peripheral venous blood were taken from each donor in the presence of EDTA. Cells (pellet) were collected after centrifugation for 10 minutes at 2000 rpm. Red cells were lysed by a lysis solution (50 ml final volume: 10 mM Tris pH7.6; 5 mM MgCl₂; 10 mM NaCl). The solution was centrifuged (10 minutes, 2000 rpm) as many times as necessary to eliminate the residual red cells present in the supernatant, after resuspension of the pellet in the lysis solution.

The pellet of white cells was lysed overnight at 42° C. with 3.7 ml of lysis solution composed of:

3 ml TE 10-2 (Tris-HCl 10 mM, EDTA 2 mM)/NaCl 0.4 M

200 μl SDS 10%

500 μl K-proteinase (2 mg K-proteinase in TE 10-2/NaCl 0.4 M).

For the extraction of proteins, 1 ml saturated NaCl (6M) (1/3.5 v/v) was added. After vigorous agitation, the solution was centrifuged for 20 minutes at 10000 rpm.

For the precipitation of DNA, 2 to 3 volumes of 100% ethanol were added to the previous supernatant, and the solution was centrifuged for 30 minutes at 2000 rpm. The DNA solution was rinsed three times with 70% ethanol to eliminate salts, and centrifuged for 20 minutes at 2000 rpm. The pellet was dried at 37° C., and resuspended in 1 ml TE 10-1 or 1 ml water. The DNA concentration was evaluated by measuring the OD at 260 nm (1 unit OD=50 μg/ml DNA).

To determine the presence of proteins in the DNA solution, the OD 260/OD 280 ratio was determined. Only DNA preparations having a OD 260/OD 280 ratio between 1.8 and 2 were used in the subsequent examples described below.

The pool was constituted by mixing equivalent quantities of DNA from each individual.

### Example 2(b)

Detection of Biallelic Markers: Amplification of Genomic DNA by PCR

The amplification of specific genomic sequences of the DNA samples of example 2(a) was carried out on the pool of DNA obtained previously. In addition, 50 individual samples were similarly amplified.

PCR assays were performed using the following protocol:

| | |
|---|---|
| Final volume | 25 μl |
| DNA | 2 ng/μl |
| MgCl₂ | 2 mM |
| dNTP (each) | 200 μM |
| primer (each) | 2.9 ng/μl |
| Ampli Taq Gold DNA polymerase | 0.05 unit/μl |
| PCR buffer (10x = 0.1 M TrisHCl pH 8.3 0.5 M KCl | 1x |

Each pair of first primers was designed using the sequence information of the human chromosome 13q31-q33 region of interest disclosed herein and the OSP software (Hillier & Green, 1991). This first pair of primers was about 20 nucleotides in length and had the sequences disclosed in Table 6.

TABLE 6

| | | | | | | Complementary | |
|---|---|---|---|---|---|---|---|
| | | | Position range of | | | position range of | |
| | SEQ | Primer | amplification | | Primer | amplification | |
| Amplicon | ID No | name | primer in SEQ ID | | name | primer in SEQ ID | |
| 99-15663 | 32 | B12 | 1 | 18 | C12 | 430 | 450 |
| 99-15665 | 33 | B13 | 1 | 20 | C13 | 458 | 476 |
| 99-15672 | 34 | B14 | 1 | 18 | C14 | 533 | 551 |
| 99-15664 | 35 | B15 | 1 | 19 | C15 | 483 | 502 |
| 99-5919 | 36 | B16 | 1 | 19 | C16 | 435 | 455 |
| 99-5862 | 37 | B17 | 1 | 20 | C17 | 430 | 450 |
| 99-16032 | 38 | B18 | 1 | 19 | C18 | 384 | 403 |
| 99-16038 | 39 | B19 | 1 | 19 | C19 | 456 | 476 |
| 99-5897 | 40 | B20 | 1 | 18 | C20 | 475 | 492 |
| 99-13601 | 41 | B21 | 1 | 19 | C21 | 500 | 517 |
| 99-13925 | 42 | B22 | 1 | 20 | C22 | 513 | 533 |
| 99-13929 | 43 | B23 | 1 | 19 | C23 | 460 | 480 |
| 99-14021 | 44 | B24 | 1 | 18 | C24 | 460 | 477 |
| 99-14359 | 45 | B25 | 1 | 18 | C25 | 457 | 475 |
| 99-14364 | 46 | B26 | 1 | 19 | C26 | 453 | 473 |
| 99-15056 | 47 | B27 | 1 | 18 | C27 | 482 | 502 |
| 99-15229 | 48 | B28 | 1 | 20 | C28 | 476 | 494 |
| 99-15232 | 49 | B29 | 1 | 18 | C29 | 467 | 485 |
| 99-15241 | 50 | B30 | 1 | 19 | C30 | 444 | 464 |
| 99-15244 | 51 | B31 | 1 | 20 | C31 | 532 | 550 |
| 99-15252 | 52 | B32 | 1 | 18 | C32 | 433 | 452 |
| 99-15253 | 53 | B33 | 1 | 19 | C33 | 459 | 477 |
| 99-15256 | 54 | B34 | 1 | 18 | C34 | 439 | 456 |
| 99-15261 | 55 | B35 | 1 | 19 | C35 | 481 | 501 |
| 99-15280 | 56 | B36 | 1 | 18 | C36 | 521 | 541 |
| 99-15353 | 57 | B37 | 1 | 18 | C37 | 495 | 514 |
| 99-15355 | 58 | B38 | 1 | 18 | C38 | 471 | 489 |
| 99-15685 | 59 | B39 | 1 | 18 | C39 | 449 | 468 |
| 99-15695 | 60 | B40 | 1 | 18 | C40 | 481 | 500 |
| 99-15703 | 61 | B41 | 1 | 18 | C41 | 452 | 472 |
| 99-15870 | 62 | B42 | 1 | 21 | C42 | 452 | 470 |
| 99-16321 | 63 | B43 | 1 | 20 | C43 | 451 | 469 |
| 99-16333 | 64 | B44 | 1 | 19 | C44 | 524 | 544 |
| 99-5873 | 65 | B45 | 1 | 18 | C45 | 457 | 475 |
| 99-5912 | 66 | B46 | 11 | 31 | C46 | 494 | 511 |
| 99-6012 | 67 | B47 | 1 | 19 | C47 | 467 | 485 |
| 99-6080 | 68 | B48 | 1 | 18 | C48 | 509 | 529 |
| 99-7308 | 69 | B49 | 1 | 18 | C49 | 469 | 489 |

Preferably, the primers contained a common oligonucleotide tail upstream of the specific bases targeted for amplification which was useful for sequencing. Primers PU contain the following additional PU 5′ sequence: TGTAAAACGACGGCCAGT; primers RP contain the following RP 5′ sequence: CAGGAAACAGCTATGACC. The primer containing the additional PU 5′ sequence is listed in SEQ ID No 70. The primer containing the additional RP 5′ sequence is listed in SEQ ID No 71.

The synthesis of these primers was performed following the phosphoramidite method, on a GENSET UFPS 24.1 synthesizer.

DNA amplification was performed on a Genius II thermocycler. After heating at 95° C. for 10 min, 40 cycles were performed. Each cycle comprised: 30 sec at 95° C., 54° C. for 1 min, and 30 sec at 72° C. For final elongation, 10 min at 72° C. ended the amplification. The quantities of the amplification products obtained were determined on 96-well microtiter plates, using a fluorometer and Picogreen as intercalant agent (Molecular Probes).

### Example 2(c)

Detection of Biallelic Markers: Sequencing of Amplified Genomic DNA and Identification of Polymorphisms

The sequencing of the amplified DNA obtained in example 2(b) was carried out on ABI 377 sequencers. The sequences of the amplification products were determined using automated dideoxy terminator sequencing reactions with a dye terminator cycle sequencing protocol. The products of the sequencing reactions were run on sequencing gels and the sequences were determined using gel image analysis ABI Prism DNA Sequencing Analysis software (2.1.2 version) and the above mentioned proprietary “Trace” basecaller.

The sequence data were further evaluated using the above mentioned polymorphism analysis software designed to detect the presence of biallelic markers among the pooled amplified fragments. The polymorphism search was based on the presence of superimposed peaks in the electrophoresis pattern resulting from different bases occurring at the same position as described previously.

34 fragments of amplification were analyzed. In these segments, 34 biallelic markers were detected. The localization of these biallelic markers is as shown in Table 7.

TABLE 7

| | | | Polymor- | | | BM | Position of | | |
|---|---|---|---|---|---|---|---|---|---|
| | | | phism | | SEQ | position in | probes in | | |
| Amplicon | BM | Marker Name | all1 | all2 | ID No | SEQ ID No | SEQ ID No | | Probes |
| 99-15663 | A12 | 99-15663-298 | C | T | 32 | 298 | 275 | 321 | P12 |
| 99-15665 | A13 | 99-15665-398 | A | G | 33 | 398 | 375 | 421 | P13 |
| 99-15672 | A14 | 99-15672-166 | C | T | 34 | 166 | 143 | 189 | P14 |
| 99-15664 | A15 | 99-15664-185 | G | T | 35 | 185 | 162 | 208 | P15 |
| 99-5919 | A16 | 99-5919-215 | A | G | 36 | 205 | 182 | 228 | P16 |
| 99-5862 | A17 | 99-5862-167 | C | T | 37 | 157 | 134 | 180 | P17 |
| 99-16032 | A18 | 99-16032-292 | A | C | 38 | 292 | 269 | 315 | P18 |
| 99-16038 | A19 | 99-16038-118 | A | G | 39 | 118 | 95 | 141 | P19 |
| 99-5897 | A20 | 99-5897-143 | A | C | 40 | 133 | 110 | 156 | P20 |
| 99-13601 | A21 | 99-13601-360 | A | G | 41 | 360 | 337 | 383 | P21 |
| 99-13925 | A22 | 99-13925-97 | A | G | 42 | 97 | 74 | 120 | P22 |
| 99-13929 | A23 | 99-13929-201 | A | C | 43 | 201 | 178 | 224 | P23 |
| 99-14021 | A24 | 99-14021-108 | A | G | 44 | 108 | 85 | 131 | P24 |
| 99-14359 | A25 | 99-14359-314 | G | C | 45 | 314 | 291 | 337 | P25 |
| 99-14364 | A26 | 99-14364-415 | C | T | 46 | 316 | 293 | 339 | P26 |
| 99-15056 | A27 | 99-15056-99 | C | T | 47 | 99 | 76 | 122 | P27 |
| 99-15229 | A28 | 99-15229-412 | A | G | 48 | 412 | 389 | 435 | P28 |
| 99-15232 | A29 | 99-15232-291 | G | T | 49 | 291 | 268 | 314 | P29 |
| 99-15241 | A30 | 99-15241-347 | A | G | 50 | 347 | 324 | 370 | P30 |
| 99-15244 | A31 | 99-15244-196 | A | G | 51 | 196 | 173 | 219 | P31 |
| 99-15252 | A32 | 99-15252-404 | C | T | 52 | 404 | 381 | 427 | P32 |
| 99-15253 | A33 | 99-15253-382 | C | T | 53 | 382 | 359 | 405 | P33 |
| 99-15256 | A34 | 99-15256-392 | C | T | 54 | 392 | 369 | 415 | P34 |
| 99-15261 | A35 | 99-15261-202 | A | G | 55 | 200 | 177 | 223 | P35 |
| 99-15280 | A36 | 99-15280-432 | C | T | 56 | 432 | 409 | 455 | P36 |
| 99-15353 | A37 | 99-15353-428 | C | T | 57 | 428 | 405 | 451 | P37 |
| 99-15355 | A38 | 99-15355-150 | C | T | 58 | 150 | 127 | 173 | P38 |
| 99-15685 | A39 | 99-15685-227 | A | G | 59 | 227 | 204 | 250 | P39 |
| 99-15695 | A40 | 99-15695-428 | C | T | 60 | 428 | 405 | 451 | P40 |
| 99-15703 | A41 | 99-15703-310 | C | T | 61 | 310 | 287 | 333 | P41 |
| 99-15870 | A42 | 99-15870-400 | A | G | 62 | 400 | 377 | 423 | P42 |
| 99-16321 | A43 | 99-16321-287 | A | C | 63 | 287 | 264 | 310 | P43 |
| 99-16333 | A44 | 99-16333-194 | A | G | 64 | 194 | 171 | 217 | P44 |
| 99-5873 | A45 | 99-5873-159 | C | T | 65 | 149 | 126 | 172 | P45 |
| 99-5912 | A46 | 99-5912-49 | A | G | 66 | 49 | 26 | 72 | P46 |
| 99-6012 | A47 | 99-6012-220 | G | T | 67 | 210 | 187 | 233 | P47 |
| 99-6080 | A48 | 99-6080-99 | C | T | 68 | 89 | 66 | 112 | P48 |
| 99-7308 | A49 | 99-7308-157 | C | T | 69 | 156 | 133 | 179 | P49 |

BM refers to “biallelic marker”. All1 and all2 refer respectively to allele 1 and allele 2 of the biallelic marker.

### Example 2(d)

Validation of the Polymorphisms Through Microsequencing

The biallelic markers identified in example 2(c) were further confirmed and their respective frequencies were determined through microsequencing. Microsequencing was carried out for each individual DNA sample described in Example 2(a).

Amplification from genomic DNA of individuals was performed by PCR as described above for the detection of the biallelic markers with the same set of PCR primers (Table 6).

The preferred primers used in microsequencing were about 19 nucleotides in length and hybridized just upstream of the considered polymorphic base. According to the invention, the primers used in microsequencing are detailed in Table 8.

TABLE 8

| | | | | | | | Complementary | |
|---|---|---|---|---|---|---|---|---|
| | | | | Position range of | | | position range of | |
| | | | | microsequencing | | | microsequencing | |
| | Biallelic | SEQ | | primer mis. 1 in | | | primer mis. 2 in | |
| Marker Name | Marker | ID No. | Mis. 1 | SEQ ID No. | | Mis. 2 | SEQ ID No | |
| 99-15663-298 | A12 | 32 | D12 | 279 | 297 | E12 | 299 | 317 |
| 99-15665-398 | A13 | 33 | D13 | 379 | 397 | E13 | 399 | 417 |
| 99-15672-166 | A14 | 34 | D14 | 147 | 165 | E14 | 167 | 185 |
| 99-15664-185 | A15 | 35 | D15 | 166 | 184 | E15 | 186 | 204 |
| 99-5919-215 | A16 | 36 | D16 | 186 | 204 | E16 | 206 | 224 |
| 99-5862-167 | A17 | 37 | D17 | 138 | 156 | E17 | 158 | 176 |
| 99-16032-292 | A18 | 38 | D18 | 273 | 291 | E18 | 293 | 311 |
| 99-16038-118 | A19 | 39 | D19 | 99 | 117 | E19 | 119 | 137 |
| 99-5897-143 | A20 | 40 | D20 | 114 | 132 | E20 | 134 | 152 |
| 99-13601-360 | A21 | 41 | D21 | 341 | 359 | E21 | 361 | 379 |
| 99-13925-97 | A22 | 42 | D22 | 78 | 96 | E22 | 98 | 116 |
| 99-13929-201 | A23 | 43 | D23 | 182 | 200 | E23 | 202 | 220 |
| 99-14021-108 | A24 | 44 | D24 | 89 | 107 | E24 | 109 | 127 |
| 99-14359-314 | A25 | 45 | D25 | 295 | 313 | E25 | 315 | 333 |
| 99-14364-415 | A26 | 46 | D26 | 297 | 315 | E26 | 317 | 335 |
| 99-15056-99 | A27 | 47 | D27 | 80 | 98 | E27 | 100 | 118 |
| 99-15229-412 | A28 | 48 | D28 | 393 | 411 | E28 | 413 | 431 |
| 99-15232-291 | A29 | 49 | D29 | 272 | 290 | E29 | 292 | 310 |
| 99-15241-347 | A30 | 50 | D30 | 328 | 346 | E30 | 348 | 366 |
| 99-15244-196 | A31 | 51 | D31 | 177 | 195 | E31 | 197 | 215 |
| 99-15252-404 | A32 | 52 | D32 | 385 | 403 | E32 | 405 | 423 |
| 99-15253-382 | A33 | 53 | D33 | 363 | 381 | E33 | 383 | 401 |
| 99-15256-392 | A34 | 54 | D34 | 373 | 391 | E34 | 393 | 411 |
| 99-15261-202 | A35 | 55 | D35 | 181 | 199 | E35 | 201 | 219 |
| 99-15280-432 | A36 | 56 | D36 | 413 | 431 | E36 | 433 | 451 |
| 99-15353-428 | A37 | 57 | D37 | 409 | 427 | E37 | 429 | 447 |
| 99-15355-150 | A38 | 58 | D38 | 131 | 149 | E38 | 151 | 169 |
| 99-15685-227 | A39 | 59 | D39 | 208 | 226 | E39 | 228 | 246 |
| 99-15695-428 | A40 | 60 | D40 | 409 | 427 | E40 | 429 | 447 |
| 99-15703-310 | A41 | 61 | D41 | 291 | 309 | E41 | 311 | 329 |
| 99-15870-400 | A42 | 62 | D42 | 381 | 399 | E42 | 401 | 419 |
| 99-16321-287 | A43 | 63 | D43 | 268 | 286 | E43 | 288 | 306 |
| 99-16333-194 | A44 | 64 | D44 | 175 | 193 | E44 | 195 | 213 |
| 99-5873-159 | A45 | 65 | D45 | 130 | 148 | E45 | 150 | 168 |
| 99-5912-49 | A46 | 66 | D46 | 30 | 48 | E46 | 50 | 68 |
| 99-6012-220 | A47 | 67 | D47 | 191 | 209 | E47 | 211 | 229 |
| 99-6080-99 | A48 | 68 | D48 | 70 | 88 | E48 | 90 | 108 |
| 99-7308-157 | A49 | 69 | D49 | 137 | 155 | E49 | 157 | 175 |

The microsequencing reaction was performed as follows:

After purification of the amplification products, the microsequencing reaction mixture was prepared by adding, in a 20 μl final volume: 10 pmol microsequencing oligonucleotide, 1 U Thermosequenase (Amersham E79000G), 1.25 μl Thermosequenase buffer (260 mM Tris HCl pH 9.5, 65 mM MgCl₂), and the two appropriate fluorescent ddNTPs (Perkin Elmer, Dye Terminator Set 401095) complementary to the nucleotides at the polymorphic site of each biallelic marker tested, following the manufacturer's recommendations. After 4 minutes at 94° C., 20 PCR cycles of 15 sec at 55° C., 5 sec at 72° C., and 10 sec at 94° C. were carried out in a Tetrad PTC-225 thermocycler (MJ Research). The unincorporated dye terminators were then removed by ethanol precipitation. Samples were finally resuspended in formamide-EDTA loading buffer and heated for 2 min at 95° C. before being loaded on a polyacrylamide sequencing gel. The data were collected by an ABI PRISM 377 DNA sequencer and processed using the GENESCAN software (Perkin Elmer).

Following gel analysis, data were automatically processed with software that allows the determination of the alleles of biallelic markers present in each amplified fragment.

The software evaluates such factors as whether the intensities of the signals resulting from the above microsequencing procedures are weak, normal, or saturated, or whether the signals are ambiguous. In addition, the software identifies significant peaks (according to shape and height criteria). Among the significant peaks, peaks corresponding to the targeted site are identified based on their position. When two significant peaks are detected for the same position, each sample is categorized classification as homozygous or heterozygous type based on the height ratio.

### Example 2(e)

Association Study Between Schizophrenia and the Biallelic Markers of the Invention: Collection of DNA Samples from Affected and Non-affected Individuals

a) Affected Population

All the samples were collected from a large epidemiological study of schizophrenia undertaken in hospital centers of Quebec from October 1995 to April 1997. The population was composed of French Caucasian individuals. The study design consisted in the ascertainment of cases and two of their first degree relatives (parents or siblings).

As a whole, 956 schizophrenic cases were ascertained according to the following inclusion criteria:

the diagnosis had been done by a psychiatrist;

the diagnosis had been done at least 3 years before recruitment time, in order to exclude individuals suffering from transient manic-depressive psychosis or depressive disorders;

the patient ancestors had been living in Quebec for at least 6 generations;

it was possible to get a blood sample from 2 close relatives.

Among the 956 schizophrenic ascertained cases, 834 individuals were included in the study for the following reasons:

for the included individual cases, the diagnosis of schizophrenia was established according to the DSM-IV (Diagnostic and Statistical Manual, Fourth edition, Revised 1994, American Psychiatric Press);

samples from individuals suffering from schizoaffective disorder were discarded;

individuals suffering from catatonic schizophrenia were also excluded from the population of schizophrenic cases;

were also excluded the individuals having a first degree relative or 2 or more second degree relatives suffering from depression or mood disorder;

individuals having had severe head trauma, severe obstretical complications, encephalitis, or meningitis before onset of symptoms were also excluded;

has also been excluded from the population of schizophrenic cases a patient suffering from epilepsy and treated with anticonvulsants. The age at onset was not added as an inclusion criteria.

b) Unaffected Population

Control cases were respectively ascertained based on the following cumulative criteria:

the individual must not be affected by schizophrenia or any other psychiatric disorder;

the individual must have 35 years old or more;

the individual must belong to the French-Canadian population;

the individual must have one or two first degree relative available for blood sampling.

Controls were matched with cases sex when possible. The unaffected population retained for the study was composed of 214 individuals, and more particularly of 141 individuals randomly selected among them.

The different populations included in the association study of this patent are characterized in more detail in Table 9.

TABLE 9

| | |
|---|---|
| Cases | Controls |

Probands

| | | |
|---|---|---|
| Sample size | 216 | 214 |
| Gender | | |
| Male | 152 | 115 |
| Female | 64 | 98 |
| Familial history of psychosis¹ | | |
| positive | 83 | — |
| none | 133 | 214 |

Relatives of

| | | |
|---|---|---|
| Sample size | 417 | 424 |
| Nber of mothers | 169 | 120 |
| Nber of fathers | 94 | 78 |
| Nber of sibs | 154 | 226 |
| Nber of trios father-mother- | 73 | 60 |
| proband | | |

¹close relatives (first or second degree)

As seen in Table 9 above, 216 Proband cases were finally selected among the initial 834 available individuals (upper part of the Table), wherein 417 relatives of these Proband cases were also included in this study.

c) Cases and Control Populations Selected for the Association Study

For the control populations, the Proband cases under study were 214, wherein 424 relatives of these Proband cases were also taken into account for this study.

The association data that are presented in the Examples 2(f) to 2(h) were obtained on a population size detailed in Table 10 below, wherein the individuals have been randomly selected from the populations detailed before in Table 9.

TABLE 10

Probands

| | Cases | Controls |
|---|---|---|
| Sample size | 141 | 141 |
| Gender | | |
| Male | 96 | 96 |
| Female | 45 | 45 |
| Familial history of psychosis | | |
| Positive | 78 | — |
| None | 63 | 141 |

Both case and control populations form two groups, each group consisting of unrelated individuals that do not share a known common ancestor. Additionally, the individuals of the control population were selected among those having no family history of schizophrenia or schizophrenic disorder.

### Example 2(f)

Association Study Between Schizophrenia and the Biallelic Markers of the Invention: Genotyping of Affected and Control Individuals

a) BACs Covering the Genomic Region of Interest (13q31-c/33)

Nine BACs were selected that cover the region of interest and several biallelic markers were generated in each of these BACs, as described in Examples 2(a) to 2(c), for performing the association study detailed hereafter. The nine BACs used as well as the biallelic markers contained therein are depicted in Table 11. The BACs used can eventually be ordered on the basis of the mapping information of ESTs or STSs sequences respectively contained in these BACs and referenced in nucleic acid sequences databases.

TABLE 11

| | Size | # | # polymorphic | # of SNPs genotyped | SNPs |
|---|---|---|---|---|---|
| BAC | (kb) | amplicons | amplicons | (mean distance (kb)) | genotyped |
| B1 | 125 | 29 | 5 | 3 (1/41) | 99-14359/314 |
| | | | | | 99-16321/287 |
| | | | | | 99-16333/194 |
| | 100 | 16 | 0 | 0 | 0 |
| B2 | 120 | 2 | 4 | 3 (1/40) | 99-7308/157 |
| | | | | | 99-14364/415 |
| | | | | | 99-14021/108 |
| B3 | 125 | 12 | 10 | 4 (1/31) | 99-15232/291 |
| | | | | | 99-6080/99 |
| | | | | | 99-6012/220 |
| | | | | | 99-15229/412 |
| B4 | 100 | 11 | 2 | 2 (1/50) | 99-15241/347 |
| | | | | | 99-15244/196 |
| B5 | 115 | 22 | 5 | 4 (1/28) | 99-15663/298 |
| | | | | | 99-15665/398 |
| | | | | | 99-15672/166 |
| | | | | | 99-15664/185 |
| B6 | 300 | 53 | 13 | 9 (1/33) | 99-15056/99 |
| | | | | | 99-5873/159 |
| | | | | | 99-15252/404 |
| | | | | | 99-15256/392 |
| | | | | | 99-15261/202 |
| | | | | | 99-15280/432 |
| | | | | | 99-15355/150 |
| | | | | | 99-15253/382 |
| | | | | | 99-15353/428 |
| B7 | 85 | 22 | 10 | 3 (1/28) | 99-15685/227 |
| | | | | | 99-15695/428 |
| | | | | | 99-15703/310 |
| B8 | 130 | 52 | 15 | 4 (1/32.5) | 99-15870/400 |
| | | | | | 99-5897/143 |
| | | | | | 99-5862/167 |
| | | | | | 99-5919/215 |
| B9 | 225 | 31 | 11 | 5⁽¹⁾ (1/45) | 99-16032/292 |
| | | | | | 99-16038/118 |
| | | 249 | 67 | 34 | |

⁽¹⁾: 99-5897/143, 99-5862/167 and 99-5919/215 are also in bac B9

b) Results from the Genotyping

The general strategy to perform the association studies was to individually scan the DNA samples from all individuals in each of the populations described above in order to establish the allele frequencies of biallelic markers, and among them the biallelic markers of the invention, in the diploid genome of the tested individuals belonging to each of these populations.

Allelic frequencies of every biallelic marker in each population (cases and controls) were determined by performing microsequencing reactions on amplified fragments obtained by genomic PCR performed on the DNA samples from each individual. Genomic PCR and microsequencing were performed as detailed above in examples 2(a) to 2(c) using the described PCR and microsequencing primers.

Then, for each allele of the biallelic markers included in this study, the difference between the allelic frequency in the unaffected population and in the population affected by schizophrenia was calculated and the absolute value of the difference was determined. The more the difference in allelic frequency for a particular biallelic marker or a particular set of biallelic markers, the more probable an association between the genomic region harboring this particular biallelic marker or set of biallelic markers and schizophrenia.

The absolute value of the difference of allelic frequency between the affected and the unaffected population is observed for each of the biallelic markers used for this study, every biallelic marker being assigned to its respective BAC from BAC B1 to BAC B9. Biallelic markers located respectively on BAC B5 and on BAC B9 show a slight association with schizophrenia. These results are a first indication according which the presence of a genetic determinant involved in the predisposition or the development of schizophrenia, most probably a gene or at least one gene, may be located in the genomic inserts carried by these two BACs or in the surrounding genomic sequences of these BACs on chromosome 13q31-q33 region.

### Example 2(g)

Association Study Between Schizophrenia and the Biallelic Markers of the Invention: Comparison of Linkage Disequilibrium Between Cases and Controls

The values of Linkage Disequilibrium between every set of two markers located in the same BAC was determined, respectively for cases and controls. For BAC B1, wherein three biallelic markers were tested, three LD values were determined (99-16321 v. 99-14359; 99-16321 v. 99-16333; 99-14359 v. 99-16333), which LD values were respectively 1.00, 1.00 and 1.00 (complete Linkage Disequilibrium). From these LD values, a Mean Normalized LD value was calculated, which is equal to 1.00 in the case of the biallelic markers of BAC B1. The results are presented in Table 12 appended at the end of the specification.

For each BAC B1 to B9, the Mean normalized LD has been determined, respectively for the population of cases (the whole cases and the cases with an available familial history of schizophrenia) and for the population of controls. The right column discloses the values of the difference of LD between populations. The highest relative difference in LD value was observed for BACs B5, B8 and B9 respectively, indicating a non-random distribution of the alleles of the biallelic markers under consideration in these BACs between the cases and the controls.

More precisely, it appears that the relative difference in Mean normalized LD for BAC B5 is significantly higher when the comparison was made between familial cases and controls than when the comparison was made between the whole cases and the controls.

On another hand, a high relative difference in Mean normalized LD for BAC B9 is observed both for the comparison between familial cases and controls and for the comparison between the whole cases and the controls.

### Example 2(h)

Association Study Between Schizophrenia and the Biallelic Markers of the Invention: Haplotype Frequency Analysis

a) Hailotype Frequency Analysis on BAC B5.

One way of increasing the statistical power of individual markers is by performing haplotype association analysis.

Haplotype association analysis was performed for all possible combination of markers 99-15663/298, 99-15665/398, 99-15672/166 and 99-15664/185 in each population described in example 2(e).

For a given set of markers, peculiar attention is paid to the haplotype (Max-hap) giving the maximum difference of frequency between cases and controls. If a gene involved in the aetiology of the disease lies close to the markers then a specific haplotype is likely to harbor a morbid mutation.

The strength of association of the Max-hap between cases and controls is compared between set of markers using two approaches:

a test comparing frequency in cases and controls is constructed and, the p-value is derived assuming it follows a chi2 distribution with 1 degree of freedom,

another p-value is assessed using the permutation routine described above.

The stronger the difference in the frequency of this haplotype between cases and controls, the lower the p-value and the most likely a morbid mutation is harbored by the haplotype considered.

Haplotype Association Analysis in Whole Cases Population and in all Controls

The results of the statistical analysis of the whole cases versus the control population are presented in Table 13 appended at the end of this specification.

The analysis of all possible sets of two, three and four markers (99-15663/298, 99-15665/398, 99-15672/166 and 99-15664/185) available in the BAC B5 was performed.

The column frequency depicts the respective frequencies of the Max-hap in cases and in controls. The haplotype statistics column summarizes the p-value obtained with this haplotype as described above. The last two columns presents the LR test as described before.

From the data presented here all p-values are high and superior to 0.01; Moreover, the p-values obtained after random permutations were close to the p-values experimentally obtained; thus none of the set of markers considered give statistical significant differences of frequency between schizophrenic cases and healthy controls.

Haplotype Association Analysis in Cases With no Familial History of Psychosis and Controls

The results of the statistical analysis of the cases with no familial history of psychosis versus the control population are presented in Table 14 appended at the end of this specification.

From the data presented in Table 14, it can be observed a high p-value (>0.1) of the chi2 test in each sets of markers considered, thus none of them give statistical significant differences of frequency between schizophrenic cases and healthy controls.

Haplotype Association Analysis in Cases with Familial History of Psychosis and Controls

The results of the analysis of familial cases versus all controls are presented in the Table 15.

From the Table 15, it can be observed that the p-values are significant for several sets of markers (haplotype 1, 7, 8 and 11). Hence a noticeably high haplotype chi2 (17.79) is observed for haplotype 1 (allele T from marker 99-15672/166 and allele T from marker 99-15664/185).

The analysis of this BAC shows an indication that a gene involved in the predisposition or the development of schizophrenia may lie near BAC B5. The difference of results between cases with and without family history of psychosis is not contradictory with this conclusions but may suggest heterogeneity in the aetiology of the disease.

b) Haplotyoe Frequency Analysis on BAC B9

For every two, three, four and five marker sets involving markers 99-5897-143, 99-5862-167, 99-16032-292, 99-16038-118, and 99-5919-215 available in this BAC, haplotype association analysis was performed in this BAC with the strategy described above for every population described in Example 2(e).

Haplotype Association Analysis in all Schizophrenic Cases Versus Controls

The results of the statistical analysis of the whole cases versus the control population are presented in Table 16 appended at the end of this specification.

All the sets of markers exhibiting a low p-value are presented in Table 16. For different sets of markers, several Max-hap lead to chi2-associated p-value inferior to 10⁻⁵, particularly for one two markers-haplotype (haplotype 5), three three-markers haplotype (haplotype 18, 19 and 17) and one four markers-haplotype (haplotype 25), which is highly significant. This strength of association is corroborated by the permutation-associated p-value which is inferior to 10⁻³. From these results it can be concluded that a gene involved in the susceptibility to schizophrenia is likely to lie near this BAC.

Haplotype Association Analysis in Familial Schizophrenic Cases Versus Controls

The results of the statistical analysis of the familial schizophrenic cases versus the control population are presented in Table 17 appended at the end of the specification.

The same pattern of association is observed in the analysis of the sub-sample of familial cases versus healthy controls. Again several Max-hap leads to chi2-associated p-value inferior to 10⁻⁵. It can be observed a high Chi2 value and a significant low p-value (less than 10⁻⁶) for the majority of the haplotypes tested, and particularly for one two markers-haplotype (haplotype 5), for four three markers-haplotypes (haplotypes 19, 18, 17 and 11) and for three four markers-haplotypes (haplotypes 25, 21, 23 and 22) and for one five markers-haplotype (haplotype 26). For haplotypes 5, 11, 17, 18, 19 and 25, the p-value is less than 10⁻⁶, which is highly significant. Moreover, for each of these haplotypes, the corresponding p-value after permutation is much lower than the p-value calculated assuming that the test has a chi2 distribution which clearly indicates that the low chi2 p-value observed is not a random value.

From the results detailed above, it can be concluded that the haplotypes described in Table 17, and particularly haplotypes 5, 17, 18, 19 and 25, are in association with familial schizophrenia and are thus located in a region harboring a genetic determinant involved in the predisposition or in the development of schizophrenia.

It can be noticed, notably for haplotype 5, that the haplotype giving the highest difference of frequency is less represented in the cases than in the controls. Assuming that a sensitivity gene to schizophrenia maps near BAC B9, it can be expected that the Max-hap for a given set of associated markers leads to a positive difference. In the relation with the results obtained on BAC B5, the results can be explained by the fact that, in this particular case, there are two haplotype alleles (and not a single one) among the four possible haplotype alleles, that are associated with schizophrenia, as a result of a genetic event having occurred between the two markers 99-15672/166 and 99-15664/185 of BAC B5 and the two markers 99-58621167 and 99-16032/292 of BAC B9, for example a crossing-over event.

c) Haplotype Association Analysis Combining Markers of BAC B5 and BAC B9

To confirm this hypothesis, two markers from BAC B5 (99-15672/166 and 99-15664/185) and two markers from BAC B9 (99-5862/167 and 99-16032/192) were combined and the haplotype association analysis on familial cases against controls was performed.

The results of every sets of two, three and four markers combinations of these markers are presented in Table 18. Haplotype giving the maximum positive (MaxP) and negative (MaxN) difference of frequency between cases and controls are presented.

Every combination of markers involving 99-15672/166 and 99-15664/185 gives a highly significant p-value in the chi2 test. Notably the combination of the four markers gives a p-value inferior to 10⁻¹¹ which is the best value obtained. Hence, for haplotype 7, 8, 9 and 11, the Max-hap with combined markers of B9 and B5 always leads to a positive difference of frequency between cases and controls. These results solves the apparent contradiction of the results obtained on BAC B9, i.e the maximum difference of frequency observed is negative, and reinforces the conclusion of the existence of a gene involved in the predisposition or in the development of schizophrenia in this region.

d) Association Analysis With Haplotypes Containing Two or Three Markers Contained in Either Bacs B1 to B9

Starting from the results presented above, the inventors have studied extensively the statistical significance of association of all the possible two markers- and three markers-haplotypes (combinations of the markers listed in Table 7) with schizophrenia. The data are presented below.

Association Analysis with the Two Markers-Haplotypes

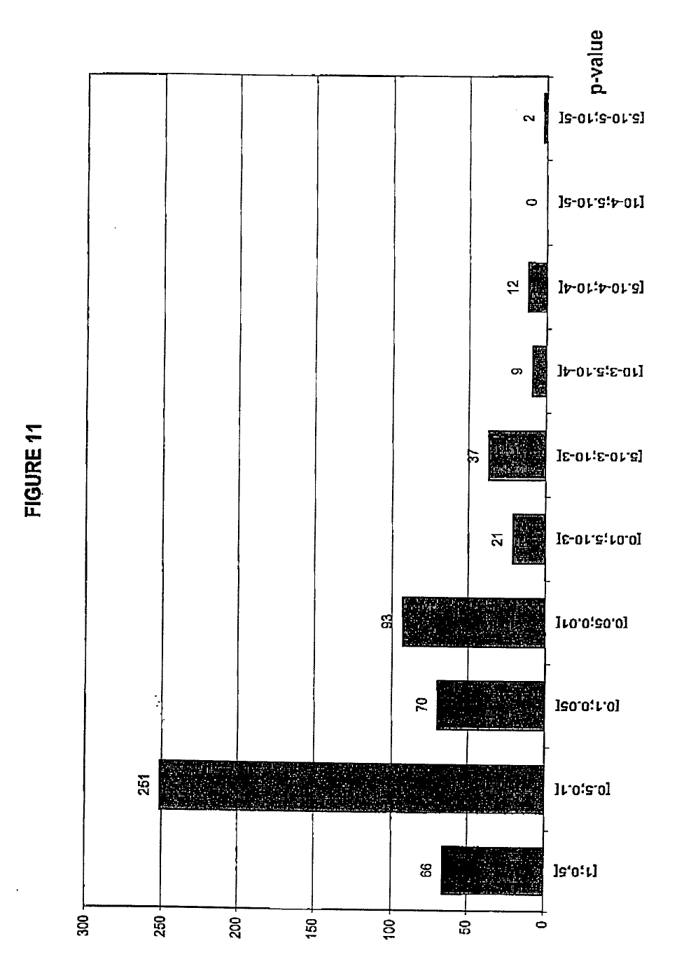

The statistical analysis of the association between haplotypes including all the combinations of two markers among the 34 biallelic markers of the invention listed in Table 7 was performed. The analysis was carried out by comparing the haplotype frequencies between controls (141 individuals) and schizophrenia familial cases (78 individuals). Then, the Chi2 value of the difference in haplotype frequency between the selected controls and cases was determined, and the corresponding p value with one degree of freedom was calculated. The results are presented in FIG. 11, wherein each bar of the histogram denotes the number of haplotypes (ordinate) having a p-value falling in a specified range (abscissa).

Among the 561 possible haplotypes studied, only two haplotypes (0.4%) were strongly associated with schizophrenia, with a p-value in the range between 5×10⁻⁵ and 1×10⁻⁵, which are the following:

Haplotype A: markers 99-15672/166 (allele T) and 99-15664/185 (allele T) (p-value=2.5×10⁻⁵), these markers being located on BAC B5 (see Table 11); Haplotype A is the same as haplotype 1 depicted in Table 15;

Haplotype B: markers 99-15664/185 (allele T) and 99-5862/167 (allele T) (p-value=3.9×10⁻⁵), these markers being located respectively on BAC B5 and BAC B9 (see Table 11); Haplotype B is the same as haplotype 2 depicted in Table 18;

These results confirm that genomic sequences within BAC B5 and BAC B9 may lie at the proximity of at least one gene involved in the susceptibility, the occurrence or the development of schizophrenia in human.

Association Analysis with Three Markers-Haplotypes

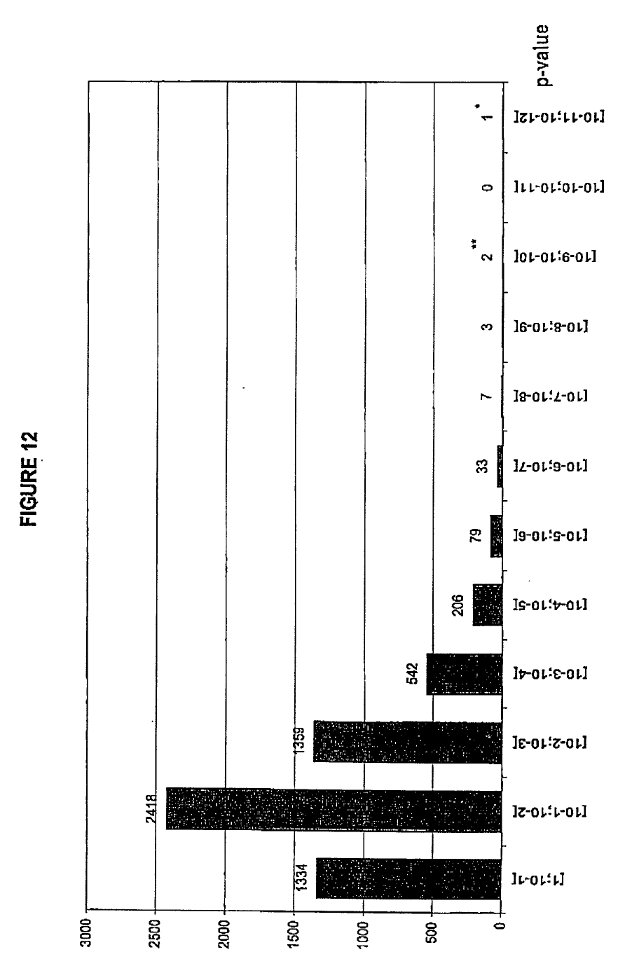

The statistical analysis of the association between haplotypes including all the combinations of three markers among the 34 biallelic markers of the invention listed in Table 7 was performed using the Chi2 test and calculating the resulting p value. The analysis was carried out by comparing the haplotype frequencies between controls (141 individuals) and schizophrenia familial cases (78 individuals). Then, the Chi2 value of the difference in haplotype frequency between the selected controls and cases was determined, and the corresponding p value with one degree of freedom was calculated. The results are presented in FIG. 12, wherein each bar of the histogram denotes the number of haplotypes (ordinate) having a p-value falling in a specified range (abscissa).

Among the 5984 haplotypes studied, only three haplotypes (0.05%) were strongly associated with schizophrenia, with a p-value in the range between 5×10⁻⁵ and 1×10⁻⁵, which are the following:

Haplotype A: markers 99-15672/166 (allele T) and 99-15664/185 (allele T) and 99-5862/167 (allele T) (p-value of 1.5×10⁻¹²), these markers being located respectively on BAC B5, BAC B5 and BAC B9 (see Table 11); Haplotype A is the same than haplotype 7 depicted in Table 18;

Haplotype B: markers 99-15672/166 (allele T), 99-5862/167 (allele T) and 99-16032/292 (allele C) (p-value=1.5×10⁻¹⁰), these markers being located respectively on BAC B5, BAC B9 and BAC B9 (see Table 11); Haplotype B is the same as haplotype 8 depicted in Table 18;

Haplotype C: markers 99-15672/166 (allele T), 99-15664/185 (allele T) and 99-5897/143 (allele A) (p-value in the range 10⁻⁹-10⁻¹⁰), these markers being located respectively on BAC B5, BAC B5 and BAC B9 (see Table 11)

These results further confirm the data analysis of the two markers-haplotypes described above, following which the genomic sequences within BAC B5 and BAC B9 may lie at the proximity of at least one gene involved in the susceptibility, the occurrence or the development of schizophrenia in human.

All documents and GenBank accession numbers cited herein are incorporated herein by reference in their entirety.

While the preferred embodiment of the invention has been illustrated and described, it will be appreciated that various changes can be made therein by the one skilled in the art without departing from the spirit and scope of the invention.

TABLE 12
Comparison of Linkage disequilibrium between cases and controls

| | Mean Normalized LD⁽¹⁾ | | | | |
|---|---|---|---|---|---|
| | all cases | famlial cases | controls | Relative difference (%)⁽²⁾ | |
| BAC | (N = 141) | (N = 78) | (N = 141) | all cases/all controls | Familial cases/controls |
| B1 | 1,00 | 1,00 | 1,00 | 0,00 | 0,00 |
| B2 | 0,48 | 0,38 | 0,48 | 0,69 | −21,53 |
| B3 | 0,72 | 0,78 | 0,75 | −4,01 | 4,68 |
| B4 | 1,00 | 1,00 | 1,00 | 0,00 | 0,00 |
| B5 | 0,60 | 0,68 | 0,57 | 6,78 | 19,76 |
| B6 | 0,57 | 0,57 | 0,58 | −2,62 | −1,52 |
| B7 | 0,23 | 0,23 | 0,21 | 6,25 | 9,37 |
| B8 | 0,22 | 0,27 | 0,21 | 2,80 | 25,70 |
| B9 | 0,44 | 0,52 | 0,28 | 57,52 | 85,92 |

⁽¹⁾: Mean of all nomalized LD between markers belonging to the same bac.
⁽²⁾: 100* (LD(cases)-LD(controls)/LD(controls))

TABLE 13
ANALYSIS ON BAC B5: ALL CASES/ALL CONTROLS

| | |
|---|---|
| DESCRIPTION | STATISTICS |

| | | sample size | HAPLOTYPE | | | | | | Statistics on a haplotype | | |
|---|---|---|---|---|---|---|---|---|---|---|---|
| | # | (Cases/ | 99-15663/ | | 99-15672/ | | Frequency | | | | p-value |
| haplotype # | mks | Controls) | 298 | 99-15665/398 | 166 | 99-15664/185 | Cases | Controls | X² | p-value (X²₁)⁽⁴⁾ | (permut)⁽⁶⁾ |
| haplotype 1 | 2 | 103/135 | | | T | T | 0,207 | 0,122 | 6,31 | 1,10E-02 | 2,60E-02 |
| haplotype 2 | | 130/137 | T | | T | | 0,192 | 0,116 | 5,92 | 1,40E-02 | 1,20E-02 |
| haplotype 3 | | 128/133 | | G | T | | 0,196 | 0,128 | 4,45 | 3,40E-02 | 4,00E-02 |
| haplotype 4 | | 138/133 | T | G | | | 0,301 | 0,229 | 3,54 | 6,80E-02 | 2,40E-02 |
| haplotype 5 | | 112/136 | T | | | G | 0,014 | 0,004 | 1,69 | 2,10E-01 | 2,90E-01 |
| haplotype 6 | | 110/131 | | G | | G | 0,015 | 0,004 | 1,54 | 2,10E-01 | 2,20E-01 |
| haplotype 7 | 3 | 102/135 | C | | T | T | 0,045 | 0,01 | 6,12 | 1,30E-02 | 9,00E-02 |
| haplotype 8 | | 100/130 | | A | T | T | 0,047 | 0,01 | 5,83 | 1,50E-02 | 9,00E-02 |
| haplotype 9 | | 128/132 | T | G | T | | 0,196 | 0,119 | 5,8 | 1,60E-02 | 2,00E-02 |
| haplotype 10 | | 110/131 | T | G | | G | 0,016 | 0,004 | 1,54 | 2,10E-01 | 3,40E-01 |
| haplotype 11 | 4 | 100/130 | C | A | T | T | 0,047 | 0,01 | 5,83 | 1,50E-02 | 8,00E-02 |

⁽¹⁾Frequency of the haplotype leading to the maximum chi square test
⁽²⁾Test on the frequency of this haplotype in cases vs in controls
⁽⁴⁾p-value assuming a chi-square distribution with 1 degree of freedom
⁽⁵⁾p-value obtained by permutating affected status in the sample
100 permutations for haplotypes 3, 5, 6, 7, 8, 10 and 11
500 permutations for haplotypes 1, 2, 4 and 9

TABLE 14
ANALYSIS ON BAC B5: CASES with no family history/ALL CONTROLS

| DESCRIPTION | | | | | | | STATISTICS | | | | |
|---|---|---|---|---|---|---|---|---|---|---|---|
| | | sample size | HAPLOTYPE | | | | | | Statistics on a haplotype⁽¹⁾ | | |
| | | (Cases/ | 99-15663/ | | 99-15672/ | | Frequency⁽¹⁾ | | | p-value | p-value |
| haplotype # | # mks | Controls) | 298 | 99-15664/398 | 166 | 99-15664/185 | Cases | Control | X² | (X²₁)⁽⁴⁾ | (permut)⁽⁵⁾ |
| haplotype 1 | | 45/135 | | | T | T | 0,11 | 0,12 | 0,11 | 7,40E-01 | 7,60E-01 |
| haplotype 2 | | 62/137 | T | | T | | 0,146 | 0,116 | 0,7 | 4,03E-01 | 4,10E-01 |
| haplotype 3 | | 60/133 | | G | T | | 0,153 | 0,128 | 0,43 | 5,12E-01 | 5,30E-01 |
| haplotype 4 | | 61/133 | T | G | | | 0,27 | 0,229 | 0,77 | 3,80E-01 | 2,10E-01 |
| haplotype 5 | | 46/136 | T | | | T | 0,21 | 0,22 | 0,09 | 7,64E-01 | 7,50E-01 |
| haplotype 6 | | 44/131 | | G | | T | 0,22 | 0,22 | 0,04 | 8,41E-01 | 7,40E-01 |
| haplotype 9 | | 45/135 | C | | T | T | 0,042 | 0,039 | 0,002 | 9,64E-01 | 9,00E-01 |
| haplotype 10 | | 48/180 | | A | T | T | 0,015 | 0,01 | 0,12 | 7,29E-01 | 9,50E-01 |
| haplotype 7 | | 60/132 | T | G | T | | 0,153 | 0,119 | 0,82 | 3,65E-01 | 3,90E-01 |
| haplotype 8 | | 44/131 | T | G | | T | 0,22 | 0,22 | 0,04 | 8,41E-01 | 7,70E-01 |
| haplotype 11 | | 43/130 | C | A | T | T | 0,015 | 0,01 | 0,12 | 7,29E-01 | 1,70E-01 |

⁽¹⁾Frequency of the haplotype leading to the maximum chi square test with familial cases
⁽²⁾Test on the frequency of this haplotype in cases vs in controls
⁽³⁾Likelihod ratio test
⁽⁴⁾p-value assuming a chi-square distribution with 1 degree of freedom
⁽⁵⁾p-value obtained by permutating affected status in the sample (*100 permutations)

TABLE 15
ANALYSIS ON BAC B5: CASES WITH FAMILY HISTORY/ALL CONTROLS

| DESCRIPTION | | | | | | | STATISTICS | | | | |
|---|---|---|---|---|---|---|---|---|---|---|---|
| | | sample size | HAPLOTYPE | | | | | | Statistics on a haplotype⁽²⁾ | | |
| | | (Cases/ | 99-15663/ | | 99-15672/ | | Frequency⁽¹⁾ | | | p-value | p-value |
| haplotype # | # mks | Controls) | 298 | 99-15665/398 | 166 | 99-15664/185 | Cases | Controls | X² | (X²₁)⁽⁴⁾ | (permut)⁽⁵⁾ |
| haplotype 1 | 2 | 58/135 | | | T | T | 0,3 | 0,122 | 17,79 | 2,50E-05 | 1,00E-03 |
| haplotype 2 | | 68/137 | T | | T | | 0,236 | 0,116 | 9,97 | 1,60E-03 | 2,00E-03 |
| haplotype 3 | | 68/133 | | G | T | | 0,236 | 0,128 | 7,64 | 5,50E-03 | 6,00E-03 |
| haplotype 4 | | 77/133 | T | G | | | 0,325 | 0,229 | 4,56 | 3,20E-02 | 1,60E-02 |
| haplotype 5 | | 66/136 | T | | | T | 0,287 | 0,220 | 2,13 | 1,40E-01 | 2,80E-01 |
| haplotype 6 | | 66/131 | | G | | T | 0,287 | 0,225 | 1,8 | 1,70E-01 | 4,00E-01 |
| haplotype 7 | 3 | 57/136 | C | | T | T | 0,081 | 0,010 | 13,65 | 2,10E-04 | 2,00E-03 |
| haplotype 8 | | 57/130 | | A | T | T | 0,081 | 0,010 | 12,69 | 3,70E-04 | 1,40E-02 |
| haplotype 9 | | 68/132 | T | G | T | | 0,236 | 0,119 | 9,18 | 2,40E-03 | 8,00E-03 |
| haplotype 10 | | 66/131 | T | G | | T | 0,287 | 0,225 | 1,8 | 1,70E-01 | 3,90E-01 |
| haplotype 11 | 4 | 57/130 | C | A | T | T | 0,081 | 0,010 | 12,69 | 3,70E-04 | 1,20E-02 |

⁽¹⁾Frequency of the haplotype leading to the maximum chi square test
⁽²⁾Test on the frequency of this haplotype in cases vs in controls
⁽⁴⁾p-value assuming a chi-square distribution with 1 degree of freedom
⁽⁵⁾p-value obtained by permutating affected status in the sample
100 permutations for haplotypes 5, 6 and 10
500 permutations for haplotypes 2, 3, 4, 7, 8, 9 and 11
1000 permutations for haplotype 1

TABLE 16
ANALYSIS ON BAC B9: ALL CASES/ALL CONTROLS

| DESCRIPTION | | | | | | | | STATISTICS | | | | |
|---|---|---|---|---|---|---|---|---|---|---|---|---|
| | | sample size | HAPLOTYPE | | | | | | | Statistics on a haplotype⁽²⁾ | | |
| | # | (Cases/ | 99-5897/ | | 99-16032/ | 99-16035/ | 99-5919/ | Frequency⁽¹⁾ | | | p-value | p-value |
| haplotype # | mks | Controls) | 143 | 99-5862/167 | 292 | 118 | 215 | Cases | Controls | X² | (X²₁)⁽⁴⁾ | (permut)⁽⁵⁾ |
| haplotype 5 | 2 | 131/134 | | C | C | | | 0,068 | 0,222 | 25,32 | 4,70E-07 | <1,00E-03 |
| haplotype 10 | | 135/139 | | | | A | G | 0,034 | 0,156 | 23,72 | 1,10E-06 | <1,00E-03 |
| haplotype 9 | | 132/138 | | | C | | G | 0,042 | 0,163 | 21,08 | 4,40E-06 | <1,00E-03 |
| haplotype 6 | | 133/135 | | C | | A | | 0,079 | 0,219 | 20,67 | 5,40E-06 | <1,00E-03 |
| haplotype 18 | 3 | 131/134 | | C | C | | G | 0,029 | 0,166 | 27,74 | 1,30E-07 | <1,00E-03 |
| haplotype 19 | | 133/135 | | C | | A | G | 0,034 | 0,162 | 24,79 | 6,40E-07 | <1,00E-03 |
| haplotype 17 | | 130/134 | | C | C | A | | 0,067 | 0,218 | 24,55 | 7,10E-07 | <1,00E-03 |
| haplotype 11 | | 125/133 | A | C | C | | | 0,066 | 0,216 | 23,62 | 1,10E-06 | <1,00E-03 |
| haplotype 20 | | 131/138 | | | C | A | G | 0,035 | 0,157 | 22,58 | 2,00E-06 | <1,00E-03 |
| haplotype 15 | | 126/137 | A | | C | | C | 0,036 | 0,166 | 21,53 | 3,40E-06 | <1,00E-03 |
| haplotype 16 | | 129/138 | A | | | A | G | 0,037 | 0,155 | 21,07 | 4,40E-06 | <1,00E-03 |
| haplotype 12 | | 127/134 | A | C | | A | | 0,078 | 0,217 | 19,93 | 7,70E-06 | <1,00E-03 |
| haplotype 25 | 4 | 130/134 | | C | C | A | G | 0,03 | 0,162 | 26,28 | 2,90E-07 | <1,00E-03 |
| haplotype 21 | | 124/133 | A | C | C | A | | 0,066 | 0,218 | 23,77 | 1,10E-06 | <1,00E-03 |
| haplotype 22 | | 125/133 | A | C | C | | G | 0,033 | 0,158 | 22,74 | 1,80E-06 | <1,00E-03 |
| haplotype 23 | | 127/134 | A | C | | A | G | 0,035 | 0,159 | 22,33 | 2,20E-06 | <1,00E-03 |
| haplotype 24 | | 125/137 | A | | C | A | G | 0,038 | 0,156 | 20,19 | 7,00E-06 | <1,00E-03 |
| haplotype 26 | 5 | 124/133 | A | C | C | A | G | 0,035 | 0,16 | 22,46 | 2,10E-06 | <1,00E-03 |

⁽¹⁾Frequency of the haplotype leading to the maximum chi square test
⁽²⁾Test on the frequency of this haplotype in cases vs in controls
⁽³⁾Test on the distribution of frequency possible with the set of markers in cases vs in controls (Likelihod ratio test)
⁽⁴⁾p-value assuming that the test has a chi-square distribution with 1 degree of freedom
⁽⁵⁾p-value obtained by permutating affected status in the sample (1000 permutations for all haplotypes)

TABLE 17
ANALYSIS ON BAC B9: CASES WITH FAMILY HISTORY/ALL CONTROLS

| DESCRIPTION | | | | | | | | STATISTICS | | | | |
|---|---|---|---|---|---|---|---|---|---|---|---|---|
| | | sample size | HAPLOTYPE | | | | | | | Statistics on a haplotype⁽²⁾ | | |
| | # | (Cases/ | 99-5897/ | | 99-16032/ | 99-16038/ | 99-5919/ | Frequency⁽¹⁾ | | | p-value | p-value |
| haplotype # | mks | Controls) | 143 | 99-5862/167 | 292 | 118 | 215 | Cases | Controls | X² | (X²₁)⁽⁴⁾ | (permut)⁽⁵⁾ |
| haplotype 5 | 2 | 74/133 | | C | C | | | 0,031 | 0,224 | 26,96 | 2,00E-07 | <1,00E-03 |
| haplotype 10 | | 77/138 | | | | A | G | 0,012 | 0,157 | 22,03 | 2,60E-06 | <1,00E-03 |
| haplotype 9 | | 75/137 | | | C | | G | 0,026 | 0,164 | 17,94 | 2,20E-05 | <1,00E-02 |
| haplotype 1 | | 70/136 | A | C | | | | 0,148 | 0,29 | 10,15 | 1,40E-03 | <1,00E-02 |
| haplotype 7 | | 76/137 | | C | | G | | 0,257 | 0,385 | 7,12 | 7,30E-03 | <1,00E-02 |
| haplotype 19 | 3 | 76/134 | | C | | A | G | 0 | 0,163 | 27,72 | 1,30E-07 | <1,00E-03 |
| haplotype 18 | | 74/133 | | C | C | | G | 0 | 0,167 | 27,71 | 1,30E-07 | <1,00E-03 |
| haplotype 17 | | 74/133 | | C | C | A | | 0,031 | 0,22 | 26,35 | 2,80E-07 | <1,00E-02 |
| haplotype 11 | | 68/132 | A | C | C | | | 0,024 | 0,218 | 26,3 | 2,90E-07 | <1,00E-02 |
| haplotype 12 | | 70/133 | A | C | | A | | 0,035 | 0,219 | 23,68 | 1,10E-06 | <1,00E-03 |
| haplotype 20 | | 75/137 | | | C | A | G | 0,013 | 0,158 | 21,27 | 3,90E-06 | <1,00E-02 |
| haplotype 16 | | 71/137 | A | | | A | G | 0,015 | 0,156 | 19,37 | 1,10E-05 | <1,00E-02 |
| haplotype 15 | | 69/136 | A | | C | | G | 0,015 | 0,167 | 18,95 | 1,30E-05 | <1,00E-02 |
| haplotype 25 | 4 | 74/133 | | C | C | A | G | 0 | 0,164 | 27,07 | 1,90E-07 | <1,00E-03 |
| haplotype 21 | | 68/132 | A | C | C | A | | 0,024 | 0,219 | 26,58 | 2,50E-07 | <1,00E-02 |
| haplotype 23 | | 70/133 | A | C | | A | G | 0 | 0,16 | 25 | 5,40E-07 | <1,00E-02 |
| haplotype 22 | | 68/132 | A | C | C | | G | 0 | 0,159 | 24,2 | 8,70E-07 | <1,00E-02 |
| haplotype 24 | | 69/136 | A | | C | A | G | 0,015 | 0,157 | 18,78 | 1,50E-05 | <1,00E-03 |
| haplotype 26 | 5 | 68/132 | A | C | C | A | G | 0 | 0,161 | 24,53 | 7,10E-07 | <1,00E-03 |

⁽¹⁾Frequency of the haplotype leading to the maximum chi square test
⁽²⁾Test on the frequency of this haplotype in cases vs in controls
⁽⁴⁾p-value assuming a chi-square distribution with 1 degree of freedom
⁽⁵⁾p-value obtained by permutating affected status in the sample (*100 permutations; **1000 permutations)
100 permulations for haplotypes 9, 1, 7, 17, 11, 20, 16, 15, 21, 22 and 23
1000 permutations for haplotypes 5, 10, 17, 18, 12, 25, 24 and 26

TABLE 18
HAPLOTYPE ANALYSIS ON BAC B5 (2 markers) and B9 (2 markers)

| | | | | | | | STATISTICS | | | | |
|---|---|---|---|---|---|---|---|---|---|---|---|
| DESCRIPTION | | | HAPLOTYPE | | | | | | Statistics on a haplotype⁽¹⁾ | | |
| | | sample size | 99- | 99- | 99- | 99- | Frequency⁽¹⁾ | | | pvalue | pvalue |
| haplotype # | #mks | (Cases/Controls) | 15672/166 | 15664/186 | 5862/167 | 16032/292 | Cases | Controls | X² | (X¹₁)⁽⁴⁾ | (permut)⁽⁵⁾ |

CASES WITH FAMILY HISTORY/ALL CONTROLS (maxM*)

| | | | | | | | | | | | |
|---|---|---|---|---|---|---|---|---|---|---|---|
| haplotype 1 | PT2 | 58/135 | T | T | | | 0,3 | 0,122 | 17,79 | 2,50E−06 | 1,00E−04 |
| haplotype 2 | | 66/134 | | T | T | | 0,286 | 0,12 | 16,86 | 3,90E−05 | 1,70E−03 |
| haplotype 3 | | 67/136 | T | | | C | 0,272 | 0,141 | 10,27 | 1,30E−03 | 4,30E−03 |
| haplotype 4 | | 67/134 | T | | T | | 0,342 | 0,206 | 8,79 | 3,00E−03 | 9,80E−03 |
| haplotype 5 | | 74/133 | | | T | C | 0,347 | 0,216 | 8,47 | 3,60E−03 | 1,10E−02 |
| haplotype 6 | | 65/134 | | T | | A | 0,218 | 0,135 | 4,5 | 3,40E−02 | 1,00E−01 |
| haplotype 7 | PT3 | 57/131 | T | T | T | | 0,296 | 0,042 | 49,02 | 1,50E−12 | 2,60E−04 |
| haplotype 8 | | 65/132 | T | | T | C | 0,289 | 0,056 | 40,72 | 1,60E−10 | 4,00E−05 |
| haplotype 9 | | 56/133 | T | T | | C | 0,17 | 0,023 | 27,34 | 1,70E−07 | 1,90E−02 |
| haplotype 10 | | 64/130 | | T | T | C | 0,192 | 0,083 | 9,79 | 1,70E−03 | 5,40E−02 |
| haplotype 11 | PT4 | 55/129 | T | T | T | C | 0,199 | 0,013 | 41,63 | 9,10E−11 | 2,00E−04 |

CASES WITH FAMILY HISTORY/ALL CONTROLS (maxS**)

| | | | | | | | | | | | |
|---|---|---|---|---|---|---|---|---|---|---|---|
| haplotype 1 | PT2 | 74/133 | | | C | C | 0,031 | 0,224 | 28,96 | 2,00E−07 | 1,00E−04 |
| haplotype 2 | | 67/136 | C | | | C | 0,094 | 0,311 | 23,33 | 1,30E−06 | 1,00E−04 |
| haplotype 3 | | 66/134 | | T | C | | 0,078 | 0,162 | 4,4 | 3,60E−02 | 1,20E−01 |
| haplotype 4 | | 65/134 | | G | | C | 0,218 | 0,317 | 4,24 | 3,80E−02 | 5,80E−02 |
| haplotype 5 | | 67/134 | C | | C | | 0,180 | 0,277 | 4,03 | 4,30E−02 | 4,80E−02 |
| haplotype 6 | | 58/135 | C | T | | | 0,079 | 0,145 | 3,17 | 7,40E−02 | 9,00E−02 |
| haplotype 7 | PT3 | 65/132 | C | | C | C | 0,01 | 0,146 | 17,68 | 2,60E−05 | 3,00E−04 |
| haplotype 8 | | 64/130 | | G | C | C | 0,035 | 0,177 | 15,32 | 3,70E−05 | 2,50E−03 |
| haplotype 9 | | 56/133 | C | T | | C | 0 | 0,098 | 11,83 | 5.60E−04 | 5.20E−03 |
| haplotype 10 | | 57/131 | T | T | C | | 0 | 0,079 | 9,48 | 2,10E−03 | 1,20E−01 |
| haplotype 11 | PT4 | 55/129 | C | G | C | C | 0,013 | 0,162 | 16,16 | 9,70E−05 | 1,60E−02 |

¹10000 permutations for haplotypes 1-6, 9 and 10; 50000 permutations for haplotypes 7,8 and 11¹
*maxM: Table of haplotypes giving the Maximum positive difference between cases/controls.
10000 permutations for haplotypes 1-6, 8 and 9; 50000 permutations for halotypes 7, 10 and 11.
**maxS: Table of haplotypes giving the Maximum negative dilference between cases/controls.
⁽¹⁾Frequency of the haplotype leading to the maximum chi square test
⁽²⁾Test on the frequency of this haplotype in cases vs in controls
⁽⁴⁾p-value assuming a chi-square distribution with 1 degree of Cryedom
⁽⁵⁾p-value obtained by permutating affected vs in the sample (*10000 permutations)

REFERENCES

Abbondanzo S J et al., 1993, Methods in Enzymology, Academic Press, New York, pp 803-823/Ajioka R. S. et al., Am. J. Hum. Genet., 60:1439-1447, 1997/Altschul et al., 1990, J. Mol. Biol. 215(3):403-410/Altschul et al., 1993, Nature Genetics 3:266-272/Altschul et al., 1997, Nuc. Acids Res. 25:3389-3402/Anton M. et al., 1995, J. Virol., 69: 4600-4606/Araki K et al. (1995) Proc. Natl. Acad. Sci. USA. 92(1):160-4./Aszódi et al., Proteins:Structure, Function, and Genetics, Supplement 1:38-42 (1997)/Ausubel et al. (1989)Current Protocols in Molecular Biology, Green Publishing Associates and Wiley lnterscience, N.Y./Barany F., 1991, Proc. Natl. Acad. Sci. USA, 88: 189-193/Basset A S et al., 1994, Am. J. Hum. Genet., 54: 864-870/Bates GP et al., 1997a, Hum. Mol. Genet., 6(10): 1633-1637/Bates G P et al., 1997b, Molecular Medicine today, November 1997, 508: 515/Baubonis W. (1993) Nucleic Acids Res. 21 (9):2025-9./Beaucage et al., Tetrahedron Lett 1981, 22: 1859-1862/Blouin J L et al., 1998, Nature Genetics, 20: 70-73//Bradley A., 1987, Production and analysis of chimaeric mice. In: E. J. Robertson (Ed.), Teratocarcinomas and embryonic stem cells: A practical approach. IRL Press, Oxford, pp.113./Bram R J et al., 1993, Mol. Cell Biol., 13: 4760-4769/Brinkman R R et al., Am. J. Hum. Genet., 60: 1202-1210//Brown E L, Belagaje R, Ryan M J, Khorana H G, Methods Enzymol 1979;68:109-151//Bruisten s. et al., 1993, AIDS Res. Hum. Retroviruses, 9: 259-265/Brutlag et al. Comp. App. Biosci. 6:237-245, 1990/Burg J L et al., 1996, Mol. and Cell. Probes, 10: 257-271/Burright et al., 1997, Brain Pathology, 7: 965-977/Bush et al., 1997, J. Chromatogr., 777: 311-328/Castagnoli L. et al. (Felici F.), 1991, J. Mol. Biol., 222:301-310/Chai H. et al. (1993) Biotechnol. Appl. Biochem.18:259-273./Chee et al. (1996) Science. 274:610-614./Chen and Kwok Nucleic Acids Research 25:347-353 1997/Chen et al. (1987) Mol. Cell. Biol. 7:2745-2752./Chen et al. Proc. Natl. Acad. Sci. USA 94/20 10756-10761,1997/Cho R J et al., 1998, Proc. Natl. Acad. Sci. USA, 95(7): 3752-3757/Chou J. Y., 1989, Mol. Endocrinol., 3:1511-1514./Clark A. G. (1990) Mol. Biol. Evol. 7:111-122./Coles R, Caswell R, Rubinsztein D C, Hum Mol Genet 1998;7:791-800/Compton J. (1991) Nature. 350(6313):91-92./Davies S W et al., Cell, 90: 537-548/Davis L. G., M. D. Dibner, and J. F. Baitey, Basic Methods in Molecular Biology, ed., Elsevier Press, NY, 1986/Dempster et al., (1977) J. R. Stat. Soc., 39B:1-38./Dent D S & Latchman D S (1993) The DNA mobility shift assay. In: Transcription Factors: A Practical Approach (Latchman D S, ed.) pp1-26. Oxford: IRL Press/Duck P. et al., 1990, Biotechniques, 9:142-147/Eckner R. et al. (1991) EMBO J. 10:3513-3522./Edwards et Leatherbarrow, Analytical Biochemistry, 246, 1-6 (1997)/Engvall, E., Meth. Enzymol. 70:419 (1980)/Excoffier L. and Slatkin M. (1995) Mol. Biol. Evol., 12(5): 921-927./Feldman and Steg, 1996, Medecine/Sciences, synthese, 12:47-55/Felici F., 1991, J. Mol. Biol., Vol. 222:301-310/Fields and Song, 1989, Nature, 340: 245-246/Fisher, D., Chap. 42 in: Manual of Clinical Immunology, 2d Ed. (Rose and Friedman, Eds.) Amer. Soc. For Microbiol., Washington, D.C. (1980)/Flotte et al. (1992) Am. J. Respir. Cell Mol. Biol. 7:349-356./Fodor et al. (1991) Science 251:767-777./Fraley et al. (1979) Proc. Natl. Acad. Sci. USA. 76:3348-3352./Fried M, Crothers D M, Nucleic Acids Res 1981;9:6505-6525/Fromont-Racine M. et al., 1997, Nature Genetics, 16(3): 277-282/Fuller S. A. et al. (1996) Immunology in Current Protocols in Molecular Biology, Ausubel et al.Eds, John Wiley & Sons, Inc., USA./Furth P. A. et al. (1994) Proc. Natl. Acad. Sci USA. 91:9302-9306./Garner M M, Revzin A, Nucleic Acids Res 1981;9:3047-3060/Geysen H. Mario et al. 1984. Proc. Natl. Acad. Sci. U.S.A. 81:3998-4002/Ghosh and Bacchawat, 1991, Targeting of liposomes to hepatocytes, IN: Liver Diseases, Targeted diagnosis and therapy using specific rceptors and ligands. Wu et al. Eds., Marcel Dekeker, New York, pp. 87-104./Gonnet et al., 1992, Science 256:1443-1445/Gopal (1985) Mol. Cell. Biol., 5:1188-1190./Gossen M. et al. (1992) Proc. Natl. Acad. Sci. USA. 89:5547-5551./Gossen M. et al. (1995) Science. 268:1766-1769./Graham et al. (1973) Virology 52:456-457./Green et al., Ann. Rev. Biochem. 55:569-597 (1986) Griffin et al. Science 245:967-971 (1989)/Grompe, M. (1993) Nature Genetics. 5:111-117./Grompe, M. et al. (1989) Proc. Natl. Acad. Sci. U.S.A. 86:5855-5892./Gu H. et al. (1993) Cell 73:1155-1164./Gu H. et al. (1994) Science 265:103-106./Guatelli J C et al. Proc. Natl. Acad. Sci. USA. 35:273-286./Hacia J G, Brody L C, Chee M S, Fodor S P, Collins F S, Nat Genet 1996;14(4):441-447/HaffL. A. and Smirnov I. P. (1997) Genome Research, 7:378-388./Hames B. D. and Higgins S. J. (1985) Nucleic Acid Hybridization: A Practical Approach. Hames and Higgins Ed., IRL Press, Oxford./Harju L, Weber T, Alexandrova L, Lukin M, Ranki M, Jalanko A, Clin Chem 1993;39(11Pt 1):2282-2287/Harland et al. (1985) J. Cell. Biol. 101:1094-1095./Harlow, E., and D. Lane. 1988. Antibodies A Laboratory Manual. Cold Spring Harbor Laboratory. pp. 53-242/Harper J W et al., 1993, Cell, 75: 805-816/Hawley M. E. et al. (1994) Am. J. Phys. Anthropol. 18:104./Henikoff and Henikoff, 1993, Proteins 17:49-61/Higgins et al., 1996, Methods Enzymol. 266:383-402/Hillier L. and Green P. Methods Appl., 1991,1:124-8./Hoess et al. (1986) Nucleic Acids Res. 14:2287-2300./Houbenweyl, 1974, in Meuthode der Organischen Chemie, E. Wunsch Ed., Volume 15-I et 15-II, Thieme, Stuttgart/Huang L. et al. (1996) Cancer Res 56(5):1137-1141./Huygen et al. (1996) Nature Medicine. 2(8):893-898./Izant J G, Weintraub H, Cell 1984 April;36(4):1007-15/Julan et al. (1992) J. Gen. Virol. 73:3251-3255./Kanegae Y. et al., Nucl. Acids Res. 23:3816-3821 (1995)./Karlin and Altschul, 1990, Proc. Natl. Acad. Sci. USA 87:2267-2268/Khoury J. et al., Fundamentals of Genetic Epidemiology, Oxford University Press, NY, 1993/Kievitis T. et al., 1991, J. Virol. Methods, 35: 91-92/Kim U-J. et al. (1996) Genomics 34:213-218./Klein et al. (1987) Nature. 327:70-73./Koch Y., 1977, Biochem. Biophys. Res. Commun., 74:488-491/Kohler, G. and Milstein, C., Nature 256:495(1975)/Koller et al. (1992) Annu. Rev. Immunol. 10:705-730./Kozal M J, Shah N, Shen N, Yang R, Fucini R, Merigan T C, Richman D D, Morris D, Hubbell E, Chee M,/Kwoh D Y et al., 1989, Proc. Natl. Acad. Sci. USA, 86: 1173-1177/Gingeras T R, Nat Med 1996;2(7):753-759/Lander and Schork, Science, 265, 2037-2048,1994/Landegren U. et al. (1998) Genome Research, 8:769-776./Lange K. (1997) Mathematical and Statistical Methods for Genetic Analysis. Springer, New York./Leger O J, et al., 1997, Hum Antibodies, 8(1): 3-16/Lenhard T. et al. (1996) Gene. 169:187-190./Li S H et al., 1993, Genomics, 16: 572-579/Lin M W et al., 1997, Hum. Genet., 99(3): 417-420/Lin Z, Floros J, 1998, Biotechniques, 24(6):937-940/Linton M. F. et al. (1993) J. Clin. Invest. 92:3029-3037./Liu Z. et al. (1994) Proc. Natl. Acad. Sci. USA. 91: 4528-4262./Livak et al., Nature Genetics, 9:341-342,1995/Livak K J, Hainer J W, Hum Mutat 1994;3(4):379-385/Lizardi P M et al., 1988, Bio/Technology, 6: 1197-1202/Lockhart et al. Nature Biotechnology 14: 1675-1680, 1996/Lucas A. H., 1994, In: Development and Clinical Uses of Haempophilus b Conjugate;/Mackey K, Steinkamp A, Chomczynski P, 1998, Mol Biotechnol, 9(1):1-5/Mangiarini L. et al., 1996, Cell, 87: 493-506/Mangiarini L. et al., 1997, Nature Genetics, 15: 197-200/Martineau P, Jones P, Winter G, 1998, J Mol Biol, 280(1):117-127/Mansour S. L. et al. (1988) Nature. 336:348-352./Marshall R. L. et al. (1994) PCR Methods and Applications. 4:80-84./McCormick et al. (1994) Genet. Anal. Tech. Appl. 11:158-164./McInnis et al., 1993, Am. J. Hum. Genet., 53: 385-390/McLaughlin B. A. et al. (1996) Am. J. Hum. Genet. 59:561-569./Merrifield R B, 1965, Nature, 207(996): 522-523/Merrifield R B., 1965, Science, 150(693): 178-185/Miele EA et al., 1983, J. Mol. Biol., 171: 281-295//Morton N. E., Am.J. Hum.Genet., 7:277-318, 1955/Muzyczka et al. (1992) Curr. Topics in Micro. and Immunol. 158:97-129./Nada S. et al. (1993) Cell 73:1125-1135./Nagy A. et al., 1993, Proc. Natl. Acad. Sci. USA, 90: 8424-8428./Narang S A, Hsiung H M, Brousseau R, Methods Enzymol 1979;68:90-98/Neda et al. (1991) J. Biol. Chem. 266:14143-14146./Newton et al. (1989) Nucleic Acids Res. 17:2503-2516./Nickerson D. A. et al. (1990) Proc. Natl. Acad. Sci. U.S.A. 87:8923-8927./Nicolau C. et al., 1987, Methods Enzymol., 149:157-76./Nicolau et al. (1982) Biochim. Biophys. Acta. 721:185-190./Nyren P, Pettersson B, Uhlen M, Anal Biochem 1993;208(1):171-175/O'Reilly et al. (1992) Baculovirus Expression Vectors: A Laboratory Manual. W. H. Freeman and Co., New York./Ohno et al. (1994) Science. 265:781-784./Oldenburg K. R. et al., 1992, Proc. Natl. Acad. Sci., 89:5393-5397/Orita et al. (1989) Proc. Natl. Acad. Sci. U.S.A.86: 2776-2770./Ott J., Analysis of Human Genetic Linkage, John Hopkins University Press, Baltimore, 1991/Ouchterlony, O. et al., Chap. 19 in: Handbook of Experimental Immunology D. Wier (ed) Blackwell (1973)/Ovyn C. et al., 1996, Mol. Cell. Probes, 10: 319-324/Parmley and Smith, Gene, 1988, 73:305-318/Pastinen et al., Genome Research 1997; 7:606-614/Pearson and Lipman, 1988, Proc. Natl. Acad. Sci. USA 85(8):2444-2448/Pease S. ans William R. S., 1990, Exp. Cell. Res., 190:209-211./Perlin et al. (1994) Am. J. Hum. Genet. 55:777-787./Peterson et al., 1993, Proc. Natl. Acad. Sci. USA, 90: 7593-7597/Pietu et al. Genome Research 6:492-503, 1996/Potter et al. (1984) Proc. Natl. Acad. Sci. U.S.A. 81(22):7161-7165/Porath J et al., 1975, Nature, 258(5536): 598-599./Ramunsen et al., 1997, Electrophoresis, 18: 588-598/Reid L. H. et al. (1990) Proc. Natl. Acad. Sci. U.S.A. 87:4299-4303./Reimann K A, et al., 1997, AIDS Res Hum Retroviruses. 13(11): 933-943/Ridder R, Schmitz R, Legay F, Gram H, 1995, Biotechnology (NY), 13(3):255-260/Risch, N. and Merikangas, K. (Science, 273:1516-1517, 1996/Robertson E., 1987, Embryo-derived stem cell lines. In: E. J. Robertson Ed. Teratocarcinomas and embrionic stem cells: a practical approach. IRL Press, Oxford, pp. 71./Ross C A et al., 1993, TINS, 16, 254-260/Rossi et al., Pharmacol. Ther. 50:245-254, (1991)/Roth J. A. et al. (1996) Nature Medicine. 2(9):985-991./Rougeot, C. et al.,. Eur. J. Biochem. 219 (3): 765-773, 1994/Roux et al. (1989) Proc. Natl. Acad. Sci. U.S.A. 86:9079-9083./Ruano et al. (1990) Proc. Natl. Acad. Sci. U.S.A. 87:6296-6300./Saiki R K et al., 1985, Science, 230: 1350-1354/Sambrook, J., Fritsch, E. F., and T. Maniatis. (1989) Molecular Cloning: A Laboratory Manual. 2ed. Cold Spring Harbor Laboratory, Cold Spring Harbor, N.Y./Samson M, et al. (1996) Nature, 382(6593):722-725./Samulski et al. (1989) J. Virol. 63:3822-3828./Sanchez-Pescador R. (1988) J. Clin. Microbiol. 26(10):1934-1938./Sandou et al., 1994, Science, 265: 1875-1878/Sarkar, G. and Sommer S. S. (1991) Biotechniques./Sauer B. et al. (1988) Proc. Natl. Acad. Sci. U.S.A. 85:5166-5170./Schaid D. J. et al., Genet. Epidemiol., 13:423-450, 1996/Schedl A. et al., 1993a, Nature, 362: 258-261./Schedl et al., 1993b, Nucleic Acids Res., 21: 4783-4787./Schena et al. Science 270:467-470, 1995/Schena et al., 1996, Proc Natl Acad Sci USA,.93(20):10614-10619/Schneider et al.(1997) Arlequin: A Software For Population Genetics Data Analysis. University of Geneva./Schwartz and Dayhoff, eds., 1978, Matrices for Detecting Distance Relationships: Atlas of Protein Sequence and Structure, Washington: National Biomedical Research Foundation/Sczakiel G. et al. (1995) Trends Microbiol. 3(6):213-217./Segev D. et al., 1992, Amplification of nucleic acid sequences by the “Repair Chain Reaction”. In: Non-radioactive labeling and detection of biomolecules. Kessler C. Springer-Verlag, Berlin, New York, pp. 142-147/Shay J. W. et al., 1991, Biochem. Biophys. Acta, 1072:1-7./Sheffield, V. C. et al. (1991) Proc. Natl. Acad. Sci. U.S.A. 49:699-706./Shizuya et al. (1992) Proc. Natl. Acad. Sci. U.S.A. 89:8794-8797./Shoemaker D D, et al., Nat Genet 1996;14(4):450-456/Smith (1957) Ann. Hum. Genet. 21:254-276./Smith et al. (1983) Mol. Cell. Biol. 3:2156-2165./Sosnowski R G, et al., Proc Natl Acad Sci USA 1997;94:1119-1123/Sowdhamini et al., Protein Engineering 10:207, 215 (1997)/Spargo C A et al., 1996, Mol. Cell. Probes, 10: 247-256/Spielmann S. and Ewens W. J., Am. J. Hum. Genet., 62:450-458,1998/Spielmann S. et al., Am. J. Hum. Genet., 52:506-516, 1993/Sternberg N. L. (1992) Trends Genet. 8:1-16./Sternberg N. L. (1994) Mamm. Genome. 5:397-404./Stone B B et al., 1996, Mol. Cell. Probes, 10: 359-370/Stryer, L., Biochemistry, 4th edition, 1995/Syvanen A C, Clin Chim Acta 1994;226(2):225-236/Szabo A. et al. Curr Opin Struct Biol 5, 699-705 (1995)/Tacson et al. (1996) Nature Medicine. 2(8):888-892./Te Riele et al. (1990) Nature. 348:649-651./Terwilliger J. D. and Ott J., Handbook of Human Genetic Linkage, John Hopkins University Press, London, 1994/Thomas K. R. et al. (1986) Cell. 44:419-428./Thomas K. R. et al. (1987) Cell. 51:503-512./Thompson et al., 1994, Nucleic Acids Res. 22(2):4673-4680/Tur-Kaspa et al. (1986) Mol. Cell. Biol. 6:716-718./Tyagi et al. (1998) Nature Biotechnology. 16:49-53./Urdea M. S. (1988) Nucleic Acids Research. 11:4937-4957./Urdea M. S. et al.(1991) Nucleic Acids Symp. Ser. 24:197-200./Vaitukaitis, J. et al. J. Clin. Endocrinol. Metab. 33:988-991 (1971)/Valadon P., et al., 1996, J. Mol. Biol., 261:11-22/Van der Lugt et al. (1991) Gene. 105:263-267./Vlasak R. et al. (1983) Eur. J. Biochem. 135:123-126./Wabiko et al. (1986) DNA.5(4):305-314./Walker et al. (1996) Clin. Chem. 42:9-13./Wang et al., 1997, Chromatographia, 44: 205-208/Weir, B. S. (1996) Genetic data Analysis II: Methods for Discrete population genetic Data, Sinauer Assoc., Inc., Sunderland, Mass., U.S.A./Westerink M. A. J., 1995, Proc. Natl. Acad. Sci., 92:4021-4025/White, M. B. et al. (1992) Genomics. 12:301-306./White, M. B. et al. (1997) Genomics. 12:301-306./Wong et al. (1980) Gene. 10:87-94./Wood S. A. et al., 1993, Proc. Natl. Acad. Sci. USA, 90: 4582-4585./Wu and Wu (1987) J. Biol. Chem. 262:4429-4432./Wu and Wu (1988) Biochemistry. 27:887-892./Wu et al. (1989) Proc. Natl. Acad. Sci. U.S.A. 86:2757./Yagi T. et al. (1990) Proc. Natl. Acad. Sci. U.S.A. 87:9918-9922./Zhao et al., Am. J. Hum. Genet., 63:225-240, 1998/Zou Y. R. et al. (1994) Curr. Biol. 4:1099-1103.

What is claimed:

1. An isolated, purified, or recombinant polynucleotide comprising a contiguous span of at least 12 nucleotides of SEQ ID Nos 1 to 4, 6 and 31, or the complements thereof, wherein said contiguous span comprises:
at least one of the following nucleotide positions of SEQ ID No 1: 1 to 3585 and 4644 to 5222; and/or
at least one of the following nucleotide positions of SEQ ID No 2: 1 to 16155 and 16331 to 21278; and/or
at least one of the following nucleotide positions of SEQ ID No 3: 1 to 5531 and 6355 to 21636; and/or
at least one of the following nucleotide positions of SEQ ID No 4: 1 to 519 and 2563 to 5566; and/or
at least one of the following nucleotide positions of SEQ ID No 6: 1 to 1791.

2. An isolated, purified, or recombinant polynucleotide comprising a contiguous span of at least 12 nucleotides of SEQ ID No 31, or the complements thereof, wherein said contiguous span comprises at least one of the following nucleotide positions of SEQ ID No 31: 1 to 480 and 717to 983.

3. An isolated, purified, or recombinant polynucleotide consisting essentially of a contiguous span of 8 to 50 nucleotides of anyone of SEQ ID Nos 1 to 3 and 32 to 69 or the complement thereof, wherein said span includes a G713 or 13q31-q33-related biallelic marker in said sequence.

4. A polynucleotide according to claim 3, wherein said G713 or 13q31-q33-related biallelic marker is selected from the group consisting of A1 to A49, and the complements thereof.

5. A polynucleotide according to claim 3, wherein said 13q31-q33-related biallelic marker is selected from the group consisting of A16 to A20 and the complements thereof.

6. A polynucleotide according to claim 3, wherein said contiguous span is 18 to 35 nucleotides in length and said biallelic marker is within 4 nucleotides of the center of said polynucleotide.

7. A polynucleotide according to claim 6, wherein said polynucleotide consists of said contiguous span and said contiguous span is 25 nucleotides in length and said biallelic marker is at the center of said polynucleotide.

8. A polynucleotide according to claim 6, wherein said polynucleotide consists essentially of a sequence selected from the following sequences: P1 to P49, and the complementary sequences thereto.

9. A polynucleotide according to any one of claims 1, 2 or 3, wherein the 3′ end of said contiguous span is present at the 3′ end of said polynucleotide.

10. A polynucleotide according to claim 3, wherein the 3′ end of said contiguous span is located at the 3′ end of said polynucleotide and said biallelic marker is present at the 3′ end of said polynucleotide.

11. An isolated, purified, or recombinant polynucleotide consisting essentially of a contiguous span of 8 to 50 nucleotides of anyone of SEQ ID Nos 1 to 3 and 32 to 69 or the complement thereof, wherein the 3′ end of said contiguous span is located at the 3′ end of said polynucleotide, and wherein the 3′ end of said polynucleotide is located within 20 nucleotides upstream of a G713 or 13q31-q33-related biallelic marker in said sequence.

12. A polynucleotide according to claim 11, wherein the 3′ end of said polynucleotide is located 1 nucleotide upstream of said G713 or 13q31-q33-related biallelic marker in said sequence.

13. A polynucleotide according to claim 12, wherein said polynucleotide consists essentially of a sequence selected from the following sequences: D1 to D49, and E1 to E49.

14. An isolated, purified, or recombinant polynucleotide consisting essentially of a sequence selected from the following sequences: B1 to B49 and C1 to C49.

15. An isolated, purified, or recombinant polynucleotide which encodes a polypeptide comprising a contiguous span of at least 6 amino acids of SEQ ID Nos 5 or 7.

16. A polynucleotide for use in a genotyping assay for determining the identity of the nucleotide at a G713- or 13q31-q33-related biallelic marker, or the complement thereof.

17. A polynucleotide according to claim 16, wherein the polynucleotide is used in an assay selected from the group consisting of a hybridization assay, a sequencing assay, a microsequencing assay and a mismatch detection assay.

18. A polynucleotide according to claim 16, wherein the polynucleotide is used in amplifying a segment of nucleotides comprising said biallelic marker.

19. A polynucleotide according to any one of claims 1, 2, 3, 11, 14, 15 or 16 attached to a solid support.

20. An array of polynucleotides comprising at least one polynucleotide according to claim 19.

21. An array according to claim 20, wherein said array is addressable.

22. A polynucleotide according to any one of claims 1, 2, 3, 11, 14, 15 or 16 further comprising a label.

23. A recombinant vector comprising a polynucleotide according to any one of claims 1, 2, or 15.

24. A host cell comprising a recombinant vector according to claim 23.

25. A non-human host animal or mammal comprising a recombinant vector according to claim 23.

26. A mammalian host cell comprising a G713 gene disrupted by homologous recombination with a knock out vector, comprising a polynucleotide according to any one of claims 1, 2, or 15.

27. A non-human host mammal comprising a G713 gene disrupted by homologous recombination with a knock out vector, comprising a polynucleotide according to any one of claims 1, 2, or 15.

28. A method of genotyping comprising determining the identity of a nucleotide at a G713- or 13q31-q33-related biallelic marker or the complement thereof in a biological sample.

29. A method according to claim 28, wherein said biological sample is derived from a single subject.

30. A method according to claim 29, wherein the identity of the nucleotides at said biallelic marker is determined for both copies of said biallelic marker present in said individual's genome.

31. A method according to claim 28, wherein said biological sample is derived from multiple subjects.

32. A method according to claim 28, further comprising amplifying a portion of said sequence comprising the biallelic marker prior to said determining step.

33. A method according to claim 32, wherein said amplifying is performed by PCR.

34. A method according to claim 28, wherein said determining is performed by an an assay selected from the group consisting of a hybridization assay, a sequencing assay, a microsequencing assay and an enzyme-based mismatch assay.

35. A method of estimating the frequency of an allele of a G713- or 13q31-q33-related biallelic marker in a population comprising:
a) genotyping individuals from said population for said biallelic marker according to the method of claim 28; and
b) determining the proportional representation of said biallelic marker in said population.

36. A method of detecting an association between a genotype and a trait, comprising the steps of:
a) determining the frequency of at least one G713- or 13q31-q33-related biallelic marker in trait positive population according to the method of claim 35;
b) determining the frequency of at least one G713- or 13q31-q33-related biallelic marker in a control population according to the method of claim 35; and
c) determining whether a statistically significant association exists between said genotype and said trait.

37. A method of estimating the frequency of a haplotype for a set of biallelic markers in a population, comprising:
a) genotyping at least one G713- or 13q31-q33-related biallelic marker according to claim 29 for each individual in said population;
b) genotyping a second biallelic marker by determining the identity of the nucleotides at said second biallelic marker for both copies of said second biallelic marker present in the genome of each individual in said population; and
c) applying a haplotype determination method to the identities of the nucleotides determined in steps a) and b) to obtain an estimate of said frequency.

38. A method according to claim 37, wherein said haplotype determination method is selected from the group consisting of asymmetric PCR amplification, double PCR amplification of specific alleles, the Clark algorithm, or an expectation-maximization algorithm.

39. A method of detecting an association between a haplotype and a trait, comprising the steps of:
a) estimating the frequency of at least one haplotype in a trait positive population according to the method of claim 37;
b) estimating the frequency of said haplotype in a control population according to the method of claim 37; and
c) determining whether a statistically significant association exists between said haplotype and said trait.

40. A method according to claim 36, wherein said genotyping steps a) and b) are performed on a single pooled biological sample derived from each of said populations.

41. A method according to claim 36, wherein said genotyping steps a) and b) performed separately on biological samples derived from each individual in said populations.

42. A method according to either claim 36 or 39, wherein said trait is schizophrenia.

43. A method according to either claim 36 or 39, wherein said control population is a trait negative population.

44. A method according to either claim 36 or 39, wherein said case control population is a random population.

45. An isolated, purified, or recombinant polypeptide comprising a contiguous span of at least 6 amino acids of SEQ ID Nos 5 or 7.

46. An isolated or purified antibody composition are capable of selectively binding to an epitope-containing fragment of a polypeptide according to claim 45, wherein said epitope comprises:
at least one of the amino acid positions 62 to 102 or 203 to 458 of SEQ ID No 5, and/or;
amino acid positions 1 to 467 of SEQ ID No 7.

47. A method of determining whether an individual is at risk of developing schizophrenia, comprising:
a) genotyping at least one 13q31-q33-related biallelic marker according to the method of claim 30; and
b) correlating the result of step a) with a risk of developing schizophrenia.

48. A method according to any one of claims 28, 35, 36, 37, 39 or 47 wherein said 13q31-q33-related biallelic marker is selected from the group consisting of A12 to A49 and the complements thereof.

49. A method according to claim 47, wherein said 13q31-q33-related biallelic marker is selected from the following list of biallelic markers: A16 to A20, and the complements thereof.

50. A diagnostic kit comprising a polynucleotide according to any one of claims 3, 8, 12, 13 or 14.

51. A computer readable medium having stored thereon a sequence selected from the group consisting of a nucleic acid code comprising one of the following:
a) a contiguous span of at least 12 nucleotides of SEQ ID Nos 1, 2 or 3, or the complements thereof, wherein said contiguous span comprises at least one of the following nucleotide positions:
1 to 3236, 3547 to 3585 and 4649 to 5222 of SEQ ID No 1, or a variant thereof or a sequence complementary thereto;
1 to 16155 and 16331 to 21278 of SEQ ID No 2 or a variant thereof or a sequence complementary thereto; and
1 to 5531, 6844 to 7237, 7798 to 8184, 8667 to 9074, and 9356 to 21636 of SEQ ID No 3, or a variant thereof or a sequence complementary thereto;
b) a contiguous span of at least 12 nucleotides of SEQ ID No 31 or the complements thereof, wherein said contiguous span comprises at least one of the following nucleotide positions: 1 to 480 and 717 to 983 of SEQ ID No 31;
c) a contiguous span of at least 12 nucleotides of SEQ ID No 4 or the complements thereof, wherein said contiguous span comprises at least one of the following nucleotide positions: 1 to 519 and 2563 to 5566 of SEQ ID No 4;
d) a contiguous span of at least 12 nucleotides of SEQ ID No 6 or the complements thereof;
e) a contiguous span of at least 12 nucleotides of at least one of SEQ ID Nos 32 to 69, or the complements thereof; and
f) a nucleotide sequence complementary to any one of the preceding nucleotide sequences.

52. A computer readable medium having stored thereon a sequence consisting of a polypeptide code comprising a contiguous span of at least 6 amino acids of SEQ ID Nos 5 or 7.

53. A computer system comprising a processor and a data storage device wherein said data storage device comprises a computer readable medium according to claim 51 or 52.

54. A computer system according to claim 53, further comprising a sequence comparer and a data storage device having reference sequences stored thereon.

55. A computer system of claim 54 wherein said sequence comparer comprises a computer program which indicates polymorphisms.

56. A computer system of claim 53 further comprising an identifier which identifies features in said sequence.

57. A method for comparing a first sequence to a reference sequence, comprising the steps of:
reading said first sequence and said reference sequence through use of a computer program which compares sequences; and
determining differences between said first sequence and said reference sequence with said computer program,
wherein said first sequence is selected from the group consisting of a nucleic acid code comprising one of the following:
a) a contiguous span of at least 12 nucleotides of SEQ ID Nos 1, 2 or 3, or the complements thereof, wherein said contiguous span comprises at least one of the following nucleotide positions:
1 to 3236, 3547 to 3585 and 4649 to 5222 of SEQ ID No 1, or a variant thereof or a sequence complementary thereto;
1 to 16155 and 16331 to 21278 of SEQ ID No 2 or a variant thereof or a sequence complementary thereto; and
1 to 5531, 6844 to 7237, 7798 to 8184, 8667 to 9074, and 9356 to 21636 of SEQ ID No 3, or a variant thereof or a sequence complementary thereto;
b) a contiguous span of at least 12 nucleotides of SEQ ID No 31 or the complements thereof, wherein said contiguous span comprises at least one of the following nucleotide positions: 1 to 480 and 717 to 983 of SEQ ID No 31;
c) a contiguous span of at least 12 nucleotides of SEQ ID No 4 or the complements thereof, wherein said contiguous span comprises at least one of the following nucleotide positions: 1 to 519 and 2563 to 5566 of SEQ ID No 4;
d) a contiguous span of at least 12 nucleotides of SEQ ID No 6 or the complements thereof;
e) a contiguous span of at least 12 nucleotides of at least one of SEQ ID Nos 32 to 69, or the complements thereof;
f) a nucleotide sequence complementary to any one of the preceding nucleotide sequences; and
a polypeptide code comprising a contiguous span of at least 6 amino acids of SEQ ID Nos 5 or 7.


SEQUENCE LISTING (71 sequences)

SEQ ID NO: 1 - DNA, 5222 bp, Homo sapiens
  misc_feature 1076..3075: 5′ regulatory region
  exon 3076..4643: exon 1
  allele 4872: 8-58-301 : polymorphic base C or T
  allele 3606: insertion of AGAG in SEQID4
  primer_bind 4572..4587: 8-58.pu
  primer_bind 4990..5005: 8-58.rp complement
  misc_binding 4849..4895: 8-58-301.probe
  primer_bind 4853..4871: 8-58-301.mis
  primer_bind 4873..4891: 8-58-301.mis complement
  misc_feature 148,686,902,1258,1322,2440,2794,2852,3018..3023,3052..3053,3064: n=a, g, c or t
caatatacaggtcacacacaacacagcccacttatctgtcagtagtcctaccagtgccctgggctccttcaaggtcccaagtccttgaaatttgctgtccttctgaattgtccttttctcctccctagaagacttctttctgctcccnttaaatgtccccaattcctatctatccctagtccctcttaaaatccaactggagtttactgctctactctgaagtctcaaaacatgctaatcttcacagattaccttgtattgtaattgttggcctagttagtattatctgccccttgactaagcttcaagaggccagacttggtcttttatctgtcttccaacagttcattctagaaccaacacacaacaaacaaaagtcaaattggttctcaacagtttactttcagtgggcttgcgtccttaaaattgtcttttataaaaatatcctaattcatgagaaacattagaggaaggcaaggcccagcttacttaaaaataattaactatagtctgttacataaagtaaaaatagattaaacactattattttaatgcagtatataagacttttactaagtatcactgaactaccttctgagacatgatttttactagatctgaatagtaataagataagtcacagagtcttcttttgcttcggttccttgtttgataattatgaaccntttaaatttatattgtttattttatttcccctagttttcgttctctttctgatttaatcacacatgcagagtaagacattggatgctaataatctcagccaccattaaaatgtttgtgtctgattaaaattaatgcattattgaagggaaatatattattatctaataagtgtggatgtgtattctatcatacttcaatccttcgaaaatgctggnactagtgaatatcacaaaacaggaaattagtaatacaaaataaaaatgttaggaagatgctgtaaaatgaaaatactgattatatctgggtgttgatatcaccatgactttttaaattgctcttctgatttttacaccttttttacaatgcacaaactgtacttgtataataatagttatgtcagcgagaatgtatgcactcgtatttagttaagaaatattgtggcatgtacagtacagtcctgattaataaacaatctggttaataaataataattttgtctgtattatgaggaaaaactgacattactcagaaattcaacacactgaaatcgaaagtactagaatgatctacnttacacaaatcgtcttttaaaacactacatgacacttgagtaaaccagccatcaaacaggacgncggcttttggatgatcattacctatagttggtaagaactgaaaatttttaggggtaaaatttagtgtatacagtaccttattatgcagcagaagaggctagtaggtcgaatcctgcccaagaggttttgcaattttctctttaaaaaacacctacttctcccgcatccccaacacccacagcgctccttaaaggaaaatggatacggatgccccatccctttggtttctcaatgcattttacttagaaagaggtcacctagtaagacccatgtggtgaatgtgcacccgggcggggcatgagggtccttggaaagcaccgcagagagggcgacaccgcaaaccgctgtcccacgcgacccccggggcgagccggggcagcgctgcactcggtgcgcaccggcagcgtctggcacccacgccacctgctgctcccgcgggtctggcaggggcaaggggcggagtgggcaggatcttcagatcaaaggagaccgatgcttgcccacggcatccagcgtaggagccggaatcgaggtgaccccagattttcccagagtgtctgtctcctttcccttctcagaactccgagagccaacgctaaggggcaccagaggctcagactgcggcggcgactcgcaggcacccagctcgcccgcggctgcggaggtcctcggaggagcccacccgggagggggtctggacgcaggcagcgggggcagagggagggtcgaagccgctccagagggtccaggtccgaggaggagccctcggagacgcggacggtgcgcgcggagggcagggctaggcagtgaccccgggtggcacagggcttactgtttcttcctaagactctatgaggccacttctaatcgtttactttagaataggaaagaaaacatagacaaatgggtggctgtcacccaggcagctacacggtcgatgcaggtgcacaggcgtgcgcgccattctcccgtgtcacacagcaggacggaataactgcacgcactgggagcgatggatctcgcggtccgggatccgagacgcattcagagaccaaatgtcacctcatccctcctctagccaattcgagcttcctccgcggacccccaccttccatccngaagtggctcggtccttagttgagaggactctgcccagaccctcgcaccagtagagacctctgcccgggcgcctgcaactcggacgcttcccggagccagagcgtgcgggcggcgcggccgcgaggagcctcaccggccatgccgtccgcagtctgacttttatttttctcttcagtgattaaaacaaaatatcgtggacagggggacagaaaggaaagagcaggcgcctgagcccggggagccgggcgggggcccccgggccccgcggccccgccgccgcccgcccccgcgcgccttctccctcctcccgggccgagccggcgccactgcgccaggatggagcgagctcggcngcgggaggagatgacgcaaaagccagcgctccccccaaaaaagtgtttctccaggacngaagatggcggcaacttagcccgcggactgaagatgactgtggctctcgggaggcccagcccaggcgattcggcccggagctccgcgggaggcggcgtcagcagtcggagccccggcggcggcgcccgcgggcggccctgcggcagcggcggcggcggcggcggcnnnnnnagcacttagagcgcggggtggggggtggnngggagggagcngcnccgccggaggcagcccaggctccggactctgggagagccagcgcggagcaggagcgggaggcggaggagaagaagaaggagaaggaaaggaaggaggaggaagaggaggagaagaagaggaggaggaggaggaggaggggaggcgctccgggcgccgtcagtgggcagcggaggcgcggcatgcccctggcaggggagagcgggctgggctccgcggggccatggggacccgcgcgctgacaatgcctgggtgaccggagccgcgccagccaccctgtcacctccaccatcgccccctgcacccagcctcgccgccggccaccagcaccagcctcctccgtctctctctctctctctctcnncattattgtttcccactctacagaggatggggtcggctgatgattaggaagcaagtcagnccttccttcaaagcctggtaatatttatgtaggcaaaagagacagagagagataanagagagggagagggagagagagagagaggagagagggagaggggaagagagagaaagagaggagagagaagaaacgggaggaggggataaggaaattaaaccctttaagtcaatgcatattgtggtgacaccggcacaggcgccctcacggtggagtcggccagggctgtgcgttcccaaaatatgaccaggggtgcttggatgtgtcggcagtatgacgacggcttaaaaatctggttggcagcaccccgagagaacgagaaaccgttcatcgattccgagagggctcagaaatggcgactgtctctggcatctctcttgtttttcacagtcctgctctctgatcacttgtggttctgcgccgaggccaagctgacccgggcccgggacaaggagcaccagcagcagcagcggcagcagcagcagcagcagcagcagcagaggcagcggcagcagcagcagcagcagcggcggcagcaggagccctcctggcccgcgctcctggcgagcatgggggagtcctcgcccgccgcccaggcacacagactcctctccgcctcctcgtcccccaccctgcccccctccccgggagacggcggcggcggcggcggcaagggcaaccgaggcaaagacgaccggggcaaggctctttttctaggaaactctgccaagcccgtgtggcgcctggagacttgttacccccagggcgcgtcctcgggccagtgcttcacggtggagaatgcggacgcggtgtgcgccaggaactggagtcggggggcggccgggggggacgggcaggaggtgaggagcaagcatcccactccgctctggaacttgtcggatttttacctttcgttttgtaattcctacacactttgggagttgttctcggggttgtccagtcccaacactttgaactgcagtctggatgtggtgctcaaggaaggcggcgagatgaccacttgcaggcagtgcgtcgaggcttaccaggactatgaccaccatgctcaggagaaatacgaagagtttgaaagcgtgctccacaaatatttacagtcggaggagtactcggtgaaatcctgtcctgaagactgtaaggtaggaaccctcgggggtttccccctttcctgcactttcctatctctgcttggttggggtgtcttctcaagtttattattattattttttggctatggttttgttactttgtgttttctgcggttattaagagcttggcttgctgtttgagatgagaatacctggtggattgctggtggactgactgatgccttcttctgggagtccttggaagatcagggaagtcccyggttatctgagataatcaagctggcactttaactgtttcttacattcatgcagttggctcaaactctgttacatcttttaagaaaaggaagttgaaaaagtcttgtcaggaaaatagcaatgagactgcacactgatgctcacaggtaaagtcagaagagttcgagacaggcatgtgtggtttgcaatatagatctgcacacaggaacgctctcatagtgacatcattaagttggttgagcaagagaagtggttgtagnttgtttcggtagttaaaaagaattacctgtttttacaggggtaattctggntgttcagaaaagtctgggggcctacctgaggatatttact

SEQ ID NO: 2 - DNA, 21278 bp, Homo sapiens
  exon 16158..16329: exon 2
  allele 3262: 99-16063-218 : polymorphic base A or G
  allele 6231: 99-16073-282 : polymorphic base A or G
  allele 9871: 99-16074-266 : polymorphic base C or T
  allele 14968: 99-13817-215 : polymorphic base C or T
  allele 20259: 99-16066-123 : polymorphic base C or T
  primer_bind 3045..3062: 99-16063.pu
  primer_bind 3547..3565: 99-16063.rp complement
  primer_bind 6058..6076: 99-16073.rp
  primer_bind 6493..6512: 99-16073.pu complement
  primer_bind 9661..9678: 99-16074.rp
  primer_bind 10119..10136: 99-16074.pu complement
  primer_bind 14754..14774: 99-13817.pu
  primer_bind 15183..15203: 99-13817.rp complement
  primer_bind 20137..20155: 99-16066.pu
  primer_bind 20569..20588: 99-16066.rp complement
  misc_binding 3239..3285: 99-16063-218.probe
  misc_binding 6208..6254: 99-16073-282.probe
  misc_binding 9848..9894: 99-16074-266.probe
  misc_binding 14945..14991: 99-13817-215.probe
  misc_binding 20236..20282: 99-16066-123.probe
  primer_bind 3243..3261: 99-16063-218.mis
  primer_bind 3263..3281: 99-16063-218.mis complement
  primer_bind 6212..6230: 99-16073-282.mis
  primer_bind 6232..6250: 99-16073-282.mis complement
  primer_bind 9852..9870: 99-16074-266.mis
  primer_bind 9872..9890: 99-16074-266.mis complement
  primer_bind 14949..14967: 99-13817-215.mis
  primer_bind 14969..14987: 99-13817-215.mis complement
  primer_bind 20240..20258: 99-16066-123.mis
  primer_bind 20260..20278: 99-16066-123.mis complement
  misc_feature 285,982,2264,2273,2823,11227,11232,13663,13681,13842..13843: n=a, g, c or t
gggcgcatcagttctgggagatgaactctggcttcacttcagtcagggacttaaactttctgggcctcgtattttcaaacccatggaatagaggaaaaatattactgtctacctcagaggtttgtgtaagaattaaatgagttactacatgtaagacctttatctagttgctggcatgcagtgagtgctatgccagcatcagtagttatcttctgcttttgtaaatatgttgcattggtgagatttgaaatcaccgtttcaaattcactatgatcatttatattngctgtgcgttgatagtggttataaaaagtcagaaacccgatgctaagtatgttttacattatagtctatgaaattcgagcaacaggattttatgttttgtcatgtactcattttgttccacagtgaataagaatctgtttcaaatgaacttttacatttataccatgtgaaagaaacagattattggataatttttcattgtaaaagaaacaatttccatctaagcatttacacattctaataaatgtatagttaacgttattttgtgcttacgctgaaaaatgtaaaagatattttctggcttgacctgattttaaataatagtgagtatcttaaattctaaggaataaaataaaaccgtatttttgccggggttttctgaccgcctctttccataacatacaattggcttccctttctgtctgccttgtccaaggtcttcatccaatgcagcctgctctctggaggctctgctgtttaggtagtggtgtgtgtttaaaccccaggaacataccaaggtcaatagttttccttccagctcactgactcttaagaaaacgacctttatgcaggggactaaattctgtaaagactattgaagccatagttctaaaaaaaagttagaaacgtccacgtgtgtggcaagtagaaagtttttgtttgttttaatgtgtaaggaatagctcaggtccagttnccaatgtaagggaaacaagcagaaggtgtttcattccttgataagctaacgacatctgacctaatgtatggaaagtgcgtctgtgcgcatgcgtgtgtgtgtgcgtgcgtgtgtgtgcatatggcgagagtatggcaatcagaattcaggggatgtgatgctcctttggtttgtgtgttcagtatctctatggaaagagcaggttgattgctaagtttactttgttatttgagctttcagagtgagcatttaaagatctattccatacacactgttgattgtctgggtcccaccccatccccatccccatcccccaccatttcgttggttgaaatttaagtttaaatgtcacctgaggccaggttctgtggttcacgcctgtaatgccagtactttgggaagctgatgcaggaggattacctgaggccaggaattcaagaccaccctgggcaacacagtgagatcctatctctattaaaaatttaaaaattagctaggcatggtggcatgcacctgttgtcccagttactcaggaggctgaggcagaaagatcttgagccagggaggctgaggctacagtgagaataaaaacaataacaaaaacaaaaaataaaaaataaggccaggcacagtaagtagctcacgcctgtaatcctagtactttgggaggccgaggcgggtggatcacctgaggtcaggagtccaagaccagcctggccaacatggtgaaaccccgtctctgcttaaaatacaaaaattagccaggtgtggtggtgcgtgcctgtaatcccagctacttgggaggctgaggcaggagaatcacttgagccctggaggcggaggttgcagtgagccgagattgtgccactgcactccagcctgggtgacaagagtgagactccatctcaaaaaacaaaacaaaataaaataaaataaaataaaaataaatgttacctgaatattcccacttaggtatgaaattgatgtgcttgctttgttttatttccatttgttctttctgttgttattgttgtttttcagtgaggaatcattaaaaatatcaaattattagctgtggagactctcatttacctaagaataatctaataactaatttcttttctaatttttaaaccaaagttcagtgtgagatttgtgtattaaaaagtgcaatttgtatcacaatcagtactaagccataaatctttatcattgttattgatttaaaattatctttgagccttgatatatttgcaattgaatgaaaataatttgttaaggngcttatttnaggatccatggcagtcagcacggcaaaacacatgctaggaaagtaaatggttagttttaaattttaagacattgctacggacataaaatttattagcttttttaaccagttaaccaattttttaccttttttaccaattgccaatttggggctgaaaatataaatatatgtaatttgtattatatttatattatatataatttgtattatatttatattatatacataattcaaaaatggaggtatcatttgtatatacaatgacatatcttattgatgaatgtaaacatgcattctcaagacagaaaattattaaaattaaatgaccagattttattacatgttttgcatcatgatatatgtgctttattaatttgatttttgcatatttcagtattattctacattttttgccaggctttactctgagcataaaactgggtgctgccatttaattccaaatatataatgatcaactgtctgctccaaagctgcaccttccaatgttaccaccaattttattattaaggattgttaattccattgattnatagttttcaataataatgaataatattattcattaatcaataatcaactgataatcaataattaattaattaatcaattaaacaataatgttatgattattcataatccattattgtatactaaaaacatatttctgatggtatgtttagctcaggataaagaagacatgggctaagataggccctctgtttctgccaaagtagcagagaaaagtcttctttcctagagttttgtgccctaagcacatttgcagggatgggttcaaagacttgcagagtgtgtacttatattcttaactctataagtaggaggatgctcatttttctctaagatacacacaacacacttagagaacagaaaatgcactgtattatactaacttcacaggggttagaccgcagcaagtcaatggaacacagggagcacccaacgagaarctgtccttgaaaatgttttagggaggaattctaaagtcttgcctttcaacttaactgtgtatcttcatttgaattatggaaccattgttcatgagtgaaatctatttttatgttctgaatcttcccatttaaagacatatgttttctcagcttttagacaataacatgatctaaaatgtcaaaataatacattctgctgaatctcagtatttactgaggaaatacagaaacatggcaaaagttaggaagaaaaatgtttcctttttctgctgtccacaaaagttggtggaacatttatgactgcaaaaaaaaatgagttattaacactgttttgattgaattcaacagatgctctgactttcttatatatatgtgtgtgtatgtatacatatatatttaacaactataaatatttatatgtaacaacactttcacaagatgccaataaatttagtatgagcatttacatatctaaatctttctttggctctagaaactgatgctatgccaaccagaacaaagaaatagtagctttcactactgtcactgctaataaaattcagcatcattacagcttatacaaaattgtacttattagcccaatttctattgcttgccttaaaaaaatgtatcggagaaaatggatgccattagtttagtttgtagattaaactgataataataatggcaaaaccttgatcaagtaaataaatcccactaaatagaacaggaatgtgttgctaagaccaaggccatttggattcaccctgagcttgtgtttagaggcattctttcattcattcattcattcattcaggcactaatggctgagtcatgccagaggctaggcccctcctgcggagagttcactgctgcctaaagacagaccaggttcttgcccttatgttaatattcgggtcttgcctagtcagttatataaagttcattaaaatttgcttacataattcaagcaagtgctcagttttaaatgtctgtgccacacaatttgacaatttacataatttatttcagtgctaaagaagttgacaatcgctgtttaacttcagtcgggcttctcctaagcggggcgaacacacacaggacatgcacaagataatctgttaaagtatgacaagaaaataataaattctacttgcagttataattttatcctaacctttgcaatcacttgtgctctgtgtgtgttgtataatgtacacattatataacagttcatgtgtatataaatgaatatttatgtaagggaaatatgctaaaaatcttaaatgggtggaataactactaaaaatgtttagcaagtacaattaaaacatatacaagttagtgtcacttctgtataatttaatagttaaatagttgtttctatggggaatagtaagccacagagagaaagaaggtcttaagaccgggaccagaaaaataagcttcttaattggaggcggcggttacttctagtgataggcgattgttttttccagatataattctggctccttcctaattaagattagtgttactcatgtcaccaatttgcagggtttcaagcttgtgttaataagatcacaaacattgtgagcctttgccatctttaaaatctttcactctaaagcaaaacgcacataacagttgccgaagtgccttctttgagcttctttgaaaacagccgcattttctttgagctatggagagcagcatagaacatgtttgaatccccaaagcttaaccccagaaaagatatttcttgcaagccacaaacaattctacacttacagtatcatgagttaatatttacctctgcataactggtatagaataaaacacagtttgatgtggggtggcggggtgtagggtaaaatgacctaaaagcccaccctgtctctgttacattttgctgtgggactttgtccatattgcttacattctctgagtctcggttttcatattcataaattagaataatatcactcacatcacagataccgtgcattgaaaacacctggcaaaataactgatagtaaatagttcttttttatttttttactttttattttttgagacagagtctcactctgtcacccaggctggagtgcaatggtgcagtcttggctccctgcagtttctgactcctgagttcaagtgattctcctgcctcagcctcccaagtagctggaattacaggcatgcaccgccacgcccagctaatttttgtaattttttagtagagacagagttttgccaggttggccaggctggtcttgaactcctgagctcaggtgatccacccacctcggcctcccaaagtactaggcttacaggcatgagccaccatgcctggccttgaattttttaaatttcaatagctttagtggtgcaagtggtttttggtcacatggatgagttgtataatggtgaagtctgtgattttagtgcaccggtcacctgagtagtgtacattgtaccctacaggtagttttctatctctcacccccttcacaaactcccccattctgagtctccactgtctgttgtatcaccctgtgtacctttgcataccccataggttagctcccacttataaatgagaatatgcagtatttggtttttcattcctgagttacttcaatgcaaacttgctgcaaaagaaattttgttcttttttttatggctgactagtattctatggtgtttatattatatacatttcattatctccttccttcccttgctgtcttcctcccatgctctgcccttcctccctctctttctcatatcagaagcacactaacctccaccccactcctctctctgacatgcccatctcactccctcacatgccgtcctcttctcttctccagaaaccctcagcccccattgacttctgctccctccacacggaggtgcactgacgggagcattcacagtgggattcatcttaccctggctcctcraattttacagcactatttctgatgccagggaaaatgtcttatgatgaaatttttaacctgccctacacctctgctccccaaggcacctatctactgtcagactccaaagttggctctcagtgaaaaatgtgggattaacagacagattggatggtacctaggcagccagtcctctgtttcatggtaacaggctacaagtctgtatttcaattagaatactgaagtgttcactctgactttttaaatatgtaaattctaatagaatcactggcatttttctgacagtaatgcagaccaggtttaaataggcaacatgtttaaagaaccacgtgacacaattctactccattcccagggtgccggttggctcttaatttacaaaaggggtttgcattcatacctttcccagcttctcactagcaggacacattaagataatgcagaaaacaacaaagatagtgtggtgttagctgctgtttcttgaggagcaaatgcaatgacaaaatcagaaatgctggcatctgtgggaaaaacaaagaatgcaaagagcctgctccactgaggaacagcagattttctttgagcaaaagcagattattcttgtttcaattataatttttgtgactgtgacattggtaaagtagggtggcaatcatgcactcttggtacacaagtgacctgacaatatggccaagaagtaaaacttttagatacataccaaaagcattatatattctagttctataatgtgcacagctatgtaaaagtatcagattcttagataactaactatagtttattatgtcacattttccatcctttactttggattatatgacaaagttatgcattaactttcttcagactcttttttttgcccatttctactaagtgaggctgttacagtaagttcctttctctttggttttatgaagaaacgaatacatctgcattttatagtgaatttactgattacttattatagctctactcagtgcgttggtattattggtattttctaaatagattgtagattatggaagataacattcatttattaatcatcaagtgctccatgaaatcttatatgagttctggaaaagaaggaaagcctaacacattctctcttctcctatccttggtgaatatattatccctttatatccttagattagtttcagatttcttatttaagactcaattccatttccctttctttagaagactgatttcccactttttaaacccttttcaacattctcaactcactcaagatgtggagcccaaaattgaatttatattgaagtgttcacttgctgcctttttagagatataaattttaataggatcactggcacttttgtgacagtccatacaggccaggttaaataagtggtatgtttagaggacatgtaatactctgcacttctaaggcattgcttgttggctgagagtccacaaaaggtgctttcatttactacgggcccattccgactctagataaggctattgagttatatccatccacggccaacactcagaagtagtcttatggagtgactttcttcttttttgatgcccacagcagagctcccaccatcttcgtcactaacatgataatccggtgaaggcagcttgttaaaagagatcttccttcacaccaaaggaaacgcttccttatttactgtgaagttaattcactggttattatcatcgttaatattcatttctggatatataatagaactgttctggaaaggtatgaattagtataacctctctgctcaacactttttgccacaatcccagccaaagtagtgtgtccgtttaaataacagctataaaatatactttgccctctgaagctctgtggccttccatgagctcagaacaataaaccacaccattccccatctttgtgtgacaatctttctcagcttgcaacccaatcccatattgtgctttaggacctgaaccatggggtctttttggaaggagaggttcacttcatgaagtggacttttaaaaatttatttctttagtttttgtttgtttgtttgttttgtttgtttttgttttcggccttcaataacggccactgggttggtcatatgaaaagggattcttaggatttaatatgttgggggctcatcagggttcctggtttgagtctctgctctgggcaggatctcagggccaggtggactggctctcacgctaaccaatcgcggggcatcattatgcaagcccaaaggccatgttactagtttccagtgggcattccactgacggtaaatttctgccagatacatccagggcactggggatacaagaaggcacaaagcttggccttcattccaagagcatagggtctgggaaggaggcagacgagaagacagcatcacatgtactgaactgccgaagaagagctcattgcagggtctagagggaatgcaggaaaggcttccaagtgaggtcacacaggatggacaggaattaataggctgagggattcgtgagggaggtggaaggaagagcctggtgcagagccaccgagggcacgctgcacatcgagtgacctgcttgtcactcctgtggccagagagcagagtgccgggtgggagtgggcagagatgagtctcgactgctcagcagggcaaggtcgcagagctgtgaatcggttaggagctttgaacattctcctgggtgttgctgtgggatggggtggggtgctgaattattaaaaatgaagtagtggaataatctggaataatcaaattaattttctatagattgctctaactgcagcatgaagcaggagttcacaactgaaggcacaagggtaggctccacggggtatatccaaggcagaaatccaagtgagagatgctgatggcgcgagcagcactggtgtcagtagaaatcaagagatttgggcagacatagagctattgaggaagcaggctcacagggcgtggtgattgaagagctgtggagcgtggaagaggcacccggatgacccccaggtttctcacctgcttccctccgaaaagaagaggccgaattccactttagagtttgtagtgcttgaggtattgaaacagagtgagagagggagagaaagagagagacagagaaagagagagagagatcagcattggtaatgaagatttaggcttcactattttaaagactacaattgaatcacgaggatggacggttgcccagaaagaatgtgtagaaacaagcttggaacagaagccggagggatactaacattttggggaatggcagggaaaacaaacaaacaacagcaacagcaaacaaatgaagatggagaaggaggggcagaggactaagagggacaccaggataaaacaagtcccagtggcatttccggaagctgttcaatcaaccatgatcagggctggcaagaggtcaaataagacaacaactgattttccagctggatttgtcagcagaggggccaaggagcttggaaagaacaatctcagtggagtggttcgagctgaggccaaatcatagtgagatgaagtgaaagataaaacaaggaagatattgactgttacatgagacgtttattaatgaggaagagagaaaaagagaaaactaagttgcytctgtgtcatagttaggacacgttttcctttgcataacgctggagacatattgacaaatgcttcttaggtttcccttttttcctaagccctgtgcagattatctctggatgggcacattctctatttgcgaaagcacagccgagttgatctgcttttcaccttctgtacaatcatatgtctgggcagctccgtggtgccataccctgattggccaactgggcttgcgttggccctgtgatgttcagactgattccctctcctgtataaatggagtctgggatgctggcgtggatgggcactgcaggagcctcattttggtgttgagaatggtttgggtggactgggaaatgtgggacagagccaacaagctcaggaggccctgaaatgcaagggaatagaccaagtgtgatgacaggattcaaaaagtgggtcaccaccagaaaacaggaagacatttgcgttttacttcaggaaatggagccaagacaaagccctagaaggcaaggggtgctgatgtcataagaccccccacaagggatcaagaagctcagagcgctgcttaccgcccatccttaagcttacttcacagactcagctcagcctcaagcagtggggcctttcttgtttccctttgagaatatatcacgatttccaatttctaggtgcaatgtgctttcaccagacactgacacagacatagcagaaagcagacattcagacataggaacgtgaaaatttattttgagtttcaccctcacttaaaactgtagcaacatgcgttatgcgtcaaagtgcattagcctgaggcggttgctgtcgcagactaatgcttacataaccaattatttcacccacacatacccaaaactcaggccgctagcggggttagagctggcagaaacggatctaataaaatgtatcaacaaatgctacaaatgtgaaaggaaagttagtccttactaataagtgggggaaaagggagagcagctgcttagcctcttgatttgaagctgacaaccatttttaatatctgacatttttctttgtgataaaatatatagccaacatgatattttttcagcaggctaacatatatttacattagcaatcttctaccttgctctaaaatagagagatggattttggagatgggggtaatatatatttaaaaagtaatgagaagctgacctgctggaaaaatattaatcatagggcattttttgaaaggttctgcacagattagctatcttcaaatggaatgcccagaaccaagagaggacatgaggacaccacacaagcacacctgccagccgtctatatggcaccattgttaaccatggtgatcctggtcccgngtagnatttctattcccaaatgcatgggctacttcaggattgtccagatttgcatgtaacgcatttctttctagagttcgtggcttcacatcactccccgtaatcaggaagcaaagtactccccctcggtctgtctccggaggctgtgccgttataaccaaagctttaaaaaaaaaaaatcttcattaagagtgtgaaaatgagtttgctagatgagctctcttgggttctccttggtctcttcagcctttgctctccctgatcacctcttcaaacaggcctttgtgaccaagggattctcagctgagaatccaaacacagggtagatgtggacgtaggtctggaggtagagggtgggggctcatacagcccactctgcagaactgcaagtgagtttcaaaatgaagtgacttgcaaaaaagaaaaaaagaaaaaggatatgttcggaatgaataatactcaagtgataccttccgaataacatggagtgtaatttcaacagccgcccaggggccctaccttgctcagctgtcatctgcatctttctagaccccacttccgacgttaacagaggctcagctaggtggtctctaaggctttccaccctggaatgtctcaccttagtgactccagagaccccgacgtatccaatgctcccttcactttctgtcctctcagaagagttcctgttacaagagttcccactgaaaaaaactatggactgggctgtggagagagttgaattccacttatttgatgcctaatttccctgtatacgtttccttatttgtaaaacatttcccaaacaagtctcaagggctttatgaggaaagatagcaagagagaaaatgcttcaggggaaaaagagaaaaatcgattcaagtattaagtaaaaatgatgctgtcatttggaccatttttctagtgtagtctgctcctcttgcagacagcaccttgagaacactgtatggtgggcagctgtccagcaaagattgtgacagtcaataagtcatttccagaggacacagtggaggagcgtgggggcggtgggccatttgaaataccctttcagagcataatttcctgacaaatcaagctgcttcttgggtagatggaggcactggccatcaaggggaagagtgggtggatgtcggcgagccattactctgtcaccagagacctcgcacacatttttattccttcccacagttattctttcgtttcatataattatttatactctgcagtgacacatagaaaagcaatttacctaatatttctctctataggagaaggccaagccattataaaaaagatttctagacttctttgtactccatagttcttatgaaataagacaaatatactcatttttcatgatgtgcttttgtttgaagttctttattacttttaagatttagtcaatttgttcttctagcccggtgccaccacaaaacatccttttcaaccccttcctgcaaaaacttctagaaagacgttgggaagtgtcttggagccatggccttctctcaccttcctaacataggaatcttcctcatcgtgttttgacaagaggtattgaaagtctattggaatttttctagctctagagaactcggcacatttgaagatagctcattgtatttctatgcatctcttctggcaggaaagctcgtctttatatcacgctggtagctacctgtttgttttttctcttgtctttgttttgtgattccacaatttattgtttaaatccatcgtcattggcacctggccgtttctcagacacgagataactgtaagaacctcacccctcgtttgctcttagccgcagtatattctcaggtattctccgttcttcacataacacagttttcaggtgtttactaacgtgctatttcacttttcaagtaatcattttcaagattctagaagaatttttgtttacttcccatgcgaacttttaccagtttattctgaaatatttttagcaaatgtgctgtatgctttgcctcttttattgtaaacacatctacacagagaagataaagcctttagttatgtaacatctgcttctcatccttaatctcttaagtagcaataaaatatttctcctccgtccttcggatttcgattcagctcacatggtttcccccacttgttttctgatgtatcttacccctagagctaaaattttagaaaatatttaatactcaaatgaggaatattcattgcacactcaaagcccaggaggtagctcataacggatgccaccgtggagcctggccggagaggtcgtcatagctcagtgtataaggtctgggattctgccggcagctttgcaaatgatgctcgatcacttttcttcataactaactactacctgatacttaaaaaaaaatgctgtgatctcttactagtttctttttcttttcnttttttttttttttttgnagttaaaaaaaaatctgcaaaggaaatatagcggaaatagaatttaaggagccagaataaaaatggatatgttcccatcgggcaatgctgaggaaagtggttgcaaagaggatggctagatctgaagagactccagtactggacgccccaccgtctgcganngagaggagacgttgtttcttaagactttatggctgcaggatgcgattccctattcagaacccattttaactggaataacggtggaaaggcaacgtgtgagggagcggtgggaatggggaccccaatttggccaatgaaaacatgagccactttctggcattacatagggcactccttaaagaaaaacaggcaactctagactttagaatcccagaaatgtgcggaaagcaagctgtcctcataatggaatgttattttttgaactgcaggtataagcctatcctgcttcctctagtacctgagaaaggttattaagcccaggaatcagaggatacatggctcattcctatactacagcttagtttacataataaatgtcatgtcatattacgtatcatgtcatattacacaaacgtcaatgtctatggcattttgacaaacgttttcaacttccccatggtttggttaagaaatcatttcactttttcttctgaagctggtgcttccagcctcttgctgccggcaccacctccaccttcacctgtccccctgaggcagcctcttgctgtcctcctggggcacacagccacagcccacctccgtttcccagttggcataagaattcttagaggtttcttcggctattgtaacgttttcttctagtgcagtttttcagtgtacactgttacgagaagactcattcaaaagacctataagaattctaattatttgtaggttttactaacaagttacatggtgattgtgagccattgatgactggcaaagaaaaattacttttggccatttaaaagtgtatctcactttgatatttatctcattttacctctaccaagtcaccttgatcacagaataaagcgtcattttgatcaacctaatgttatgaataaaaaaatacaatcctccttctaccaaatagatattgacccggggcatggaaggccatgctgattttaggttcatctgcaaactcccttgtgatttcataagtgctttaaaagtagttttcagattaaatataggataactattaggaacacagtctttggagccctacagaacatgggtttgaagtctggattttccatttccattacctaatagtaytattacttaatattattcctgttcaaaatgtgagtaatagttatggtcaaaagtgcatgctgagaatcacatggaaaaaaaagtttacagcttacttagaaaagagtataaagttgataaataccagctattattcttgttattattaggtatttataatctcttaggctacctttaactgctgcgcctccatttttctaggtaaaatataaccggcaaaatgttacttggataatattaaccctaaaaatagataagtaaaagccattagtgccaaggacctggagaataactgaatttcagtaacctgtactgcaaattggtacatctgccattgttgtatttgaatttaagatcagcttattccttcgtgtcagaaaaaggcaattttttcaatgtttgttttcccaaatagccaccaaaacaaagagatgaatgaagtcccagtcctttcacatttgatgaaattacataaatacgtagcaaacattgatgtggcttgcacatcatccagaaggttgaagctgtggctctgatgaggcttccctcctactcccaaccacaccacagcatatgatgtctgctctatgtcgggtgtttctgggctttcagacagtgagatttttaatgcatttggtgtgagtcatgaatgtaaatgtccatatctgcctgttctgtgcctctcaaagtccaagatctaaacagcagtttcccaacccctgctgggccatctttataaagtgcttcgcgccatctagcgtcctatggaggcatagacgtgggactccgtgtggagggaactggctcatcagccacgttgcaagtaactacagcacagaagagcaatagagtcaattatttattttactttagatatcgctgaattttgaaaaattaacttcctgcttaaatccactgattttcaaataagccacataaaattaaaggtcaaaataccttttacattctattagggatgcaatcaaatactgttttgcttttattgcggagactttcgttgctgtgtttttatcacttgcagctgtcgttgtgtaaatacgctttctcacaatgagggctctcttaggatccacttgaaaccacagcacgcttgcacgcttctcacagtgcctctatcactgtaactgttgtcacgcctctatatttttcattcttctctttttttcagattgtctacaaagcctggctctgttcccagtattttgaagtcacacagtttaactgcagaaagacaattccttgcaagcaatactgtttggaggttcagacgaggtgtccatttatattgcccgacaatgatgaagtcatctacggaggcctctccagtttcatctgtacaggtacagtgccgggtgggagtctgtacaccagtctccggtggttttgctttcttgtgtgatgtttgtttgctttgctgtttcctttcactgatataaccttgaagatgcggctggtagttttccgcactttcactaatagaagcaccgaaagctcccctcaacaggtctggaaatgattcagcttctgtaccacatggtctatctgtaactgagaccacagttattcactgtgtgaaagaagaaatctgatggcaaaatattagcaatcattagatatacttacatgtgacctagtataggttagattcagcctgcattgcctttgaattccataagaacaggagatgtgctaaggccagtgaaataatgcttttcaaattagtcagaagtttatgttctcttgcctattttcttttattttattttctgagaatttttatgttactttatctgtaaaaagtattaagagcattaaaaatatctgcctaaattgtacgtgtgtgtgtgtgtgtgtgtgtcnnntattagagccacacaacaagtctatccattaaacgatgtttacttctttcagcaaatgagtcagactgggcaaaacaccaatatcagcactccttttctggatatttataagcattacataatttagacagaataagatataaagagcaagagcctttttttaaaaaatanaaatgataattaaatggtgagaacaagatgctttgccagggtggtgtgataatccgagtggtggctctngcattctcttctataacaataatgctataaggcgtccctggaattacgtggctcaagctgtagcacgttttgttttcaagtgttttgataaaacaccatcatatgtgtagagacacatgaaaatgctcttctgacattagacttgccaaactcctctaaaacagagtagcatattaagatgctcaacatgagaatttatcaaattatttcagcatcgcttatttacatacgtattcattagccacagtgttctaagtgataagctatcaagagatagtctgccaaagctcgccctaagaactggatgagtatttctttctttttttttttttgagatggagtctcgctctgtgtcacccaggctggtgtgatctcggctcactttaagctccgcctccggggttcaagtgattctcatgcctcagcctcccgagtagctgagattacaggcgtgcaccaccacacctggctaatttttgtatttttagtagagacggtgtttcaccgtgttggccaggctggtctccaactcctgacctcaagtgatccaccctccttggcctcccaaaagtgttggaattacaggcgtgagccactgtgcccagcctggattagtatttcttatgctgcccaagcttgtggaaagaacacgtgactgaaggtcaggagcattggccttcatcctgtttctattgtacattctctcagaggcaccaccccttcactagtaaattagaaagtgcaatggccaaaccctcccccatgtggatatgctgcaaggattacagataagagagaagggcaagttatgtcattgaccagccatagccgttgcacacgagaaagtgaaatgcagaatgatttcacaggcctgtgtgttattctactgacttgatgctcaagtcattttttatgtagctcctctctgctggagggggaggaggggaaggaaggagggaagagggaaagaaggaataagggaaggagggaaggaggatatcaatatcttcttgctttgagtttgagacaaaattcnccagacacaggctattaattcatattgaattttgcatacaacattccacaaataaccatgaagataaggccttcattttttttttcttgtgggacaatgaggtgtctgtgacatgaggacttcagagaagctattcgatttttatgtacaggttgtgtttatgcatttacactcacattggcttttgtcctagagaagaacaatagtttttgaaccaagttctcaaagtagacaaaaaacccaaatgaagggcgaggtgtttgcgtttatcttctttttcctacatgtatctgtatttgtgaatctgaaatatatgtttgaatgttcacccacttcctccatacacattcagaatgccagggtagaattaaatctctcacttcacaacatgggcagagaatccagtctgcagttttaattgttttcgagtttggtaaaaacagcttcttcatagacatagctacgtcattaaggaaaagtggcaggaaaagagagatttatcagatccagagaaaagtgccatctccgtgagaggccccctaggataatctcatcactgaacatttggaaacctaagaacagtagttcttatgtcactttcagttgggttaatgttcattttatgtttaaattgctcgaaaatatatggaataaattctgaggttatttcaccctgaagttctgtaaaacaatctgacagaaaaagataataattttcatattgatcttcttttctaaggtgtgacctatgcctctttgaagaattctcattcatgttgataactagcagccatcatttcccatcatcggttacaggtgtcccttaagagtgcactgctgaggccagatgcagtggctcatgcctgtaatccacacaattttggtaggctgagataggagaattgcttgaagccaggggtttgaggacagtctggacaacgtagcaagaccctgcctctacaaaaaattaaaaataaaagaactagccagacacagttatgtgcacctgtagtcgcagctatttggaaggcagagacaggattgcttgagctcaggtgctggagattgcagtgagctgtgatcctgccactgcactcctgcctgggcagcatagcgagaccctgtctcaaatgaaaaagaaaatgaaagagtgcactgctggacctgagcaaggagatttctactcctgtccatcacagctgcctgcccgttgcttataagagtcccagacatgggccccagaatgggtttcatcttggatgatgttagaatagaggacagctcccagaggaccactggctaaactgtggagtggcttgtcatgcgtctaaggaaactagtaagacatatgtgcatataatttgttcccagattttaagttgcacatttttggatgtccgtctcctacaaatgtacacggatttttaaatagtttacattataaataagtttgcaagtaaatgtgagccatttacattctgttttttttctctgaaaacagaataataaaaagaccagtgtagttatagcatcatttaatctcatacaaaatattttaataaatatatcaaagtttaagggaaaaaagaaatgggaagggagatggtatgcccagaataaaataaaaaaatgagagtaggttatgaaaccaagtaaaatgcagtaacttcatcaaaaagaattcaccaagtaaggtgaccttcctcttgcctccagcaatttttctttttgaaaaaaatcatcaagcatttgatttccactctagatgttaaaatgcaatcatttctcactaagaattaaaggaataggtggagtttccttcaacttcaacagatttaattataatggttttcctactatttgaactgcatccaattcacagcttccagaaggacggagaattccctctaggatatcgaggattcttcacttacttttcttattgacctcaaaaaaaagtattctgtaagcacgtaactgcatatggcctgatgacagatactgtccatgtgagcatcagttttcttcaaggtgcttctccttagtgacaaaccagtggcatggtgattccttttcttagcgttctctgtacccagaatcttagagatctcaagttcttcctgtcattgtcytattggctatatactgatacctaggattcctattattcacaaaaaagggaatatcaccccttggaatcaatgaacaatttctgtaaatgatcaggagttatttaccattttggtcacacttactaaattcagtatctcgtgcatcatttctttatggtgctcacatcaccatgtaccaaatttgaaagttttaaatgtatatatttttgatcatctgctacctatgatcctttagtaagccaggaacttactttaaaaaatgaaatatctgagaatgcatgtccattactatagaatgcttttgattgaggatgttacagaaggtcatgaccaaatgttctaaccacggatcttgatgacaagcgcatccccgtgcatatgtttaattccaattctgtcttaggagataaaaggaaagacactttgcctgagatttctcttgaatttttttctattaaagaataaaactgacacgtgactaaagaaatgttttaaaaattaattggaaagaaattctgaggtgctgtcagggtgaaacatgtcatttaatgttaggcattattgagctaaaatttgtcaaggtgggttctgttgggtttgcaagctcagttcgcataatgcagtgctattttagaaactcagttgcttcatctttaaagtgagggggtggnaagtcgtttttggattctacgatgtcaacccngtcatcgtggtaattacgtgggcttccctctnccacactccctcgcctcattccagagtcccctggagaaatccttgattgcttacctttaacagtgaaccaagctgaagaaaacagaggcagcagaggagcacaggctgtgggtgctatggtaacaaacattaagaaaattatcatttgattgatagataaaatagaccagccatgatttactgctctcttacagagggacaattattgaaggnaataaaagtaaaagttggtctgctgggggttactgacaatggagataaaacatattttaagaattctatgt

SEQ ID NO: 3 - DNA, 21636 bp, Homo sapiens
  exon 5537..9359: exon 3
  misc_feature 16330..18329: 3′ regulatory region
  allele 8277: 99-13821-332 : polymorphic base C or T
  allele 15915: 99-15215-60 : polymorphic base A or G
  allele 16053: 99-13525-395 : polymorphic base A or G
  allele 17032: 99-13526-368 : polymorphic base A or G
  allele 19401: 99-15208-87 : polymorphic base A or G
  primer_bind 7946..7965: 99-13821.pu
  primer_bind 8454..8472: 99-13821.rp complement
  primer_bind 15475..15495: 99-15215.rp
  primer_bind 15943..15960: 99-13525.rp
  primer_bind 15954..15974: 99-15215.pu complement
  primer_bind 16430..16447: 99-13525.pu complement
  primer_bind 16950..16970: 99-13526.rp
  primer_bind 17381..17401: 99-13526.pu complement
  primer_bind 19315..19333: 99-15208.pu
  primer_bind 19797..19817: 99-15208.rp complement
  misc_binding 8254..8300: 99-13821-332.probe
  misc_binding 15892..15938: 99-15215-60.probe
  misc_binding 16030..16076: 99-13525-395.probe
  misc_binding 17009..17055: 99-13526-368.probe
  misc_binding 19378..19424: 99-15208-87.probe
  primer_bind 8258..8276: 99-13821-332.mis
  primer_bind 8278..8296: 99-13821-332.mis complement
  primer_bind 15896..15914: 99-15215-60.mis
  primer_bind 15916..15934: 99-15215-60.mis complement
  primer_bind 16034..16052: 99-13525-395.mis
  primer_bind 16054..16072: 99-13525-395.mis complement
  primer_bind 17013..17031: 99-13526-368.mis
  primer_bind 17033..17051: 99-13526-368.mis complement
  primer_bind 19382..19400: 99-15208-87.mis
  primer_bind 19402..19420: 99-15208-87.mis complement
  misc_feature 110,4287,4291,4453..4454,4506,4520..4521,4558,4580,5958,17188: n=a, g, c or t
aaaaaaaactctgtgaggttggtactgtaatgtcaaacagcaggtaatgattcactagagatttcaaatagaacagcttataagcaaaatatatcagaacaacatttcantacccggaaaatttgcttttgttttttcctaatgctaattagtgcaattgtacttgtttgtgcaaggaggttcttgtttgagaaattatttttgtttggaaatatgtttttataatttcttcaatgctgaggaaaaggtcatttctgtaggtaggctttcctaggaaatttgtcccagcaggttgtgtgcaggtggtttttgcgggagaactctcaggcatgctacctatgagggggtgaagaaggagaaatttggtagagggaaaaaaatctgaagctgggatggcccttgagagatgccttgacttgagtcaagtggacccagccttgggacctgtacatcgttaggaggctgcctagggcctgagaaataggtcaagactgctaacttcaaccaagggcagtgtccgggaggaatgcagggtgaaaccatctgtaggtagcactgccaactggaggagtctgtgcccccaggtcccaagggtgtcattgatctgcttatgtgacctactgactctatataaggcaaagaaataatggccttagaaggctgcatgtccttgcttattcagaaatgcatttgacagagaaaaaggcatctgtcagaattcaagtcagagaagaatgatgtagaaaaaagagctggaaaattttctaggcagtcccaaccaaaaagacattgttttatggcataaatccaaaagaatcaaagaataggtaaggagacaataatcaaaacctcttagaaattagaaagttgatgcatgctaacagatttgctgaaacttagaaaaagtaatatgaagtagcagagaggaaacagagtcagttcgatttacatccagagtcaggtattgacaacacgagatgcaatggaagaaagagcaaaataaggaagactgggtggaagtcagttttaagaggcagtcaggtccccaaatcccattccctactcccatcccagagactgggggcatatttcttcctggagggaactttttctttttgagatgataaagaaaaggttgggccgggcgcgatggctcacgcctgtaatcccagcactttgggaggccgaggcgggcagatcacgaggtcaggagatcgagaccatcctggctaacacggtgaaaccccgtctctactaaaaatataaaaaattagccgggcgtggcggcgggcgcctgtagtcccagctactcgggaggctgaggcaggagaatggcgtgaacccgggaggcggagcttgcagtgagccgagatcgcgccgctgcactccagcctgggcgacagagcgagactccgtctcaaaaaaaaaagaaaaagaaaaagaaaagattgttgatctgagcaacagtaggaggagttcagggcgggggaccactctgaccaccaggattccatgcacaactccaccctgaagctgagactcccccagccacatcctgcttctccctcctgctccctggccttctgcttcctggatggatattggatagacttttctgggaagtctgatcaacctaaagacagaatttcaaaagttcctccatcctggccgggcacggtggctcaagcctgtattcccagcactctgggaggccgaggcgggtggatcacgaggtcaagagatcaagaccatcctggccaacatggtgaaaccctgtctctactaaaaatacaaaaaattagccaggtgtgttggcgggagcctgtagttccagctactggggaggctgaggcaggtgaatcacttgaacccgggaggcagagcttgcagtgagctgagattgcaccaccgagctccagcctggcgacagagcgagactctgtctcaaaaataaataaataaataaaaataaaaataaaagtttctccattctaacagttcaggcatgtcatctcacgtgcagctcacaggccgcagccctccttaggtgcccagacttcctgtgcttgttattttgtgcagacaactaggaatgcatggtgtttccatgagaagaacaagggacaaacaaacacaaaagggtggcaacacagagaaaatggagggaatttattcagggagggaatgaaacacaacaaataagcgtaccccaaatatctcagagtgagaaaaacataaacatggtaaccataaaataagaacagttgcttcgaaaagcaacattcggataacacaaaggagctcttggaacttgatcctagggaaaaaagcaaaattcagtgaaattatgtcacgtgaaaactcaccaaattccctgaaagcagagttaagggatgaagtaatatgaaatattaacagcagaggtaagaaaattaaaggacaagttctgaaggtccaacagccaaacataggaccagtgcttgaataaatctgaagaacgatcatttctaaattagaattccatacccagccaaacaatcaattaaatatgaggtagaataaacacactttaaggcatatggcatcttggaaattcgccctcatgagcttttcctactgaagacactagaggttgtaaaccctcaaaaggagaaagaaggggatgaggcacaggatacagggaacaggggatccagcactgcagtgcacggagcagtggagactcatcaggcatgacggcaaccagtccagatggtgggaggctgcaggagatatttcttcagaaaagaactgatagaacacttcatgcagctgaaagaactggcaatgcatttgggttgaatgagttagaagagggaaaataacaagacaagtattatctgtagaaaaaaaaacgaagtggtatagggatagaaaattggactttactacatggttgagctaagaattacatttatgtcatccttatactgtaaatggaacagcaggtatcaaagttactatttatggaggaatgggtgagggtttggggggaagcatgtttgctggagggaggaggagaataaatgttttatctccacacttcctgaggggaatctacagataatgctaaaaatgaaaaatcaagaagagcataaaacatgttattctcagaaatgcagctaaatacaaagcagctgaaagatatgcaaatatttgcctttgagaaagaagaatttgagatgtgggaggtggaggattttttttgttattttgtaaaatactctgtggaattattttatcttaatatagatgcatgttattcataaatttgagtaaaaacttgttaaaaagatggacaggagagaaaaaagaaagaatattaattgtgccagacaaaaagaagggttcatttattggcctgaatgaactgcttccatttttttttctgcgatgactgtagttgtagatcagcaaaccagtttctgagcaagtttggaagagcttcttatttacctaacattgttaaatatattctgattattagcattctaaaatgttgatctaaaggacttctagatttctatttttataatcttattgtaaactccttttgttgtagaatgtagtattggaaacgtatatttcaataaatgattagtctagcagtgagatatcttatatgaaaaaagaaaatatcatacaataaaaatgtcagagctgaagacctcattaattcagtttttttttatattctacctaggcgaaggtgaagtttacctttgaatgcctaagaagaaatgaatattgtaagcatcattataaggatatcttgttcatttctttaaaagaaaatgaagattgaagtgtatattatttgagattatttttaattaaataaataggtatactgagtttattcatgcaataaatgcttattaagtattctgctcaggcattctgctacatattgggaatacaaaactaagaatgtatagtttatgcttttaaattgacagataagtaagaaaattgaaattaaaatgagcaattgtgattacaagaaaaataagaataattgctataattaagagttaagcaaaatgctaatagcccaggggtatgagtacaagcttcatacagggagtttcacttgaaccgcatttgaaggataaatgagaagtatgtagaggaattggtggaggacattttaggaagaaacagtagcttggcgagtgatacaggcttgggtgctctgggaagagccaatgcttcaggattgcgggaacattgagcagtgagcctggngagngaggagagacctttagattttattgtttaggttgaagcaatgagataccattgataactattgttattcacagcagcccaaattttcatgttatttctatgtgctaggcactgaaccaagcatgtgacatgaaaaatctcatgtaatccttacaacaactcnnactattatccccatttttaagtttttcatgcatgacattaatatgacaaaantttgtgtagaaagnnttcattctaacacccatggataaatggttcagtcaangtagaaaaaaaatacactatgnacttatatagcttgcataaaaatattctatgtaatacagcaaggtaaaggattggaaagattgtagatacacagttgctttattcttttcaccacttccaaagttttaaaatatgcatctgtcaattttaaaataagaacaatgtgtgttatcagaggaagaagacaaaaagggaaggaagagtttcagcaaggaggaagaaagtgaagagagaacaagagaagcggagagagacagacaattcctgataggcacaattggaggacagatcaaatgagaagatgaacagcagagagactccttctgtgatggtcccagcaaaagataatgaagcctcaactgaggcagacttctgtagaaagcaaatggagaaaatggagaaaaataagagaaatgtttagaaaataaatcaaaatgacccgatcacccactggacatatagatggttttgcccattctcccagagagggggagagaagacttgatgaggcttctgagaggaaacgatgataacatggttcctagagctatgacgttatgatctgttgcttatttcactcagcataactagctaatgtcagtttacgcagtgcatcaattggtcacattcctgtgaaagaagaaaggatgactgcatgtacttgatatccaagttgatccggaacctcaagttctgattttgacttaatgaaatcttctattctgtattcccaacaacccagaactttcatggtatgactggcaggaatataaaagaacaacttgaggaatctaacaatactggtttgagaccatgggtgactctttatccaattgcattgacttgattttcctttaaataaatgtctaattgcctgtaagattttgtgggttagaatgttaaagcaacagcttcactctactatgtcgcaaatgtatttccttcctgctgacactctacttccttcttcctacagggctttatgaaacctttctaaccaatgatgaaccagaatgctgtgacgtcaggagagaagaaaaatcaaataacccatccaaagggaccgtagagaaaagtggctcctgtcacaggacatcgctcacagtgtcatcagcaacaagactgtgcaacagcagactcaagctgtgtgttcttgtactgattctcttacacacagtgctcacagcctcggcagcacagaacacagccggactgagctttggaggcatcaacacgctggaagaaaactcaaccaatgaggagtaacggaaggacgagtgtcaccacagcagcagctggcccgccgtgaaaaatggcaactgctgtctcatgtaacagaaactgggtgcttttaccctcgaattacttattgcaaggcctttagggtaaaatttaaancagatgggcctgaatccaaacaaggacacaaccacagctttttattgactaaaaggctggaaagtgactttaaatttctcacaccattttatacactgtgttttaatgtttggaggttttatttgctttcgttttggtttgggtttatttgtttgtttattttttgcacttgttaatacaggatttattttgggggatggtttctcagaggtaaactaagtcttttcactgtctctatctctctatatatttctagtcattgtgtgtgttcatcagatagttctgtctttatgtcctgtcagcttctattagaggaatgattgctatgacctcatggtatagcaaaaaacaacaacaaaaaaagaataaaaaataaaaaagacaaaaaaaagaaaacaacaaaaaataaaaataaaaaaaatccctaagtctcccttctacccacagaaccaacaacacccttcccagcctttcctttccctcgccctcttctcgtcccctaagcaaacaacatccgcttgcttctgtctgtgtaaccacagtgaatgggtgtgcacgcttggtgggcctctgagcccctgttgcacaaaccagaaacagagcagagccaagggggcctgacaagagttcctttttagctgaacaaacaagtgctctccataataggtggaatcagacagttaacacatttttatgttgaaaacaaaataaaaggaaaaaattaaaaaaaactatcatgaactgtattgctccagttcccatccccaagtggcccagccctttcttgctggtccagctggacaggagcagctatctagaatcaggatgcggggagtgaggaagtttttcctttgacaatgaaggtgggctttcattgtgatttttgttctgttgcagtaatataggagcacattttggccattgtaattacagggaacaaagggattgcggacacatatctggacttcttttcctcccttattgttgtggaagagacactagaaatgctcaaacacctgcaatatacagaatatacacaattttattccagtatttccctaacatatggtttaaaattattccaggtatacagtgtatgcaattctgcattatcacagaggaacaacttcttttttaaaaaataaataggtcagccatttttattaacgtgcaaaaactttatcactctaacatgctctaggtagttgaggaaaagaggtctgatcactgtttgtattttattttctttgtgggaacatttcacctgctgagtgtacatgaatttgctttctataaaaggcttttatgagtttacagtagaatcagtggaaggaagagttaataagggctgtttttaaaaaaacaaacaaacaaacaaaacaaataattaaaaaaaaattttacattccttcctattctctaactacacttgggaagtgcacttcagataagtttgcagtgtgactgagagatgaaggaaatccatagaaaaggtcctcttagtgaacaaaatttagttattaactttatagctatgaaatttccccgggcatttgtttttgttcaaacagactttaacctctgcatcatacttaaccctgcgacatgcgtacagtatgcatattttgttttgaaaaaaaatgtttcgttccagtctgttaagaatattcaaaaataataaaggtattgcttaataaaattgctagaattgtttagcagtacatgcacaatattttactagattctttgttttaatagtgttttgttgagactgaaaatcttaaaatggtctgcgcaaatacaaaaaaaaagaaaacaccaaaaatgcaaaattctcccgtttttgttcctttttaaaaatttttttttctgcaaatgcaaatacattcacatgtggacagatacatacacataggcatcatattttagtgtatggaaacatggtgcttttctggggacacaagctgaggaagtgaatggctcgggggagacacattaattgcatggaattgttgactgtgaaacacttgtagaaatgagcactttggtatcccaaccaatggtggaatacagatttcaatggcgctggggggacgagaagcagaggaattgaggctgcaagtgattcctgaggaagagctcacagggtgattttattcagatgacctctaagggaatggctaggaaggagtctgttctgacctttatctacctctgctgtggtaaggccactccatgtgccatctcaccatctcttcagcgttcctcatgctgcttctccccgtgccataggcgtttccattcttgcaggaaaatgayaatgcaagtgcaaattccactcacttttattggtatgacaataaattattctttctaaacacttagcaaaatgacgaaaagcagtattccttaaaatataggttttcattttctcaatatattttgaactgactaaagttctttacaaagacaagaggatcagaaggttccacttacccgtgttttagcgtttgtttgaagataatgccaaccaaaaggtggaaaactaaaaccaccattgctttacataagaggtacacactgccccccacccccaaaaaaaaacacagctttgaaagttgggagaaacactgcagtcttcattgtaaatataaagtgataatttaggtgaggaaggaacggtaaggagggaaggggaatgagtcctttctccccatcaagtcagccagcattggaatagtctctaattctggcggaatattttctttgtcatgttcatctattcttattacaggagaatgatttcagtgcctagtatttattagtgtaagtgtgtaaccactttcccgcaatatttccactggacatgttgaagcaaagcagcttcttctggggtcaggcagaggatccaaataggcaacgactcctttcccaagctcagaaacaatcactgatgatgcagatttcactacatccgttgattctttgtagtagttttcctggcacacacttgagcatgcttaaggttgtgcatatgttcattttgaacaaataagttcagcccctgctcctttgtaatcatcatcttgtttagtttttcctgtagaggatgtctaccgtatactggctaatgaactacaatattaaaataattatttctgcaagcaagttgaaaaccaagaaaacaatatttgtttcataaatgtatggcatagatttccctgtttattattctttttatctgcaatagagaaatttcagtttctggaatatgttggtactgctaagctaaatggcaaataacatagagttcaatgtcattctttattgcatttacatttggtgatacgttatattgaacttttcttgcattaaaaagatagtatttttcccctctctcacaaggcagtaagtgaaagttgttcttttaaatggcatcattacaaaaagtaatttggtttcttaccaataaattggtaccttaggtactagaacctgtagtcattaaaacaaaagaaaataggcagatagcagaaacaaaagaatgacaggaaagaaggaaggaaagaggggagtgagggagggagggaagaagggagggatagagtcagggaatgagggggaaaggaggtttcatatgaatgaataagtacatacttttttccgggggaaaaaaacataaatgaaataagcaattggatgccagacccatgtgccaaatacctgaattggcatatggtatttgatatctgaacaacatcaaaaacgttaaccagtctgaagcatgatttacattctttctcagtaacaaagacatttacaattttcctgctactcaaagcattaaatcaacacagaactgaaagctgcattttgttttatttatttagaactgcagtcaggtatttaatttcttttctgtgtgtttgtttgttttttgagatggcatcttgctctgttgctcaagctggaatgcagcggcaagatctctgctcactgcaacctccacttcctgggttcaagcgattctcctgcctcaagcctctcgagtagctgggactacaggcttgtgccaccacgcctggctaatttgtgtatttttagtagagacgtggtgtcgccatgttggccaggctagtcttgaactcctgaccttgggagatccgccgattaggcctcccaaagtgttgggattacaggcgtgagataccacacccagccaggtatttaatttcataagtgtgcatatgcatatatgaggcgttagggttatatactatggcatatctttagagagatataaagatatatatacatacacacacacacacacacacacacacacacacacatcgcttgcaaataaaacttcaaccaaaacattatgtagtccataacagcacatgtataccataatgatgaatgatcctacttgtttgtcaaagttagagcagcatggtcatttaaaaaccatatctactttgacaagggaaacagactctcaaaatatctccattaatgaataaatcacaattttgaaacaggcagaatctccatcttccaagctttaacaatgtttataaaaaacctaatatagcaggtaggtattgggtgacaatgaataaaaagactttcagtgggggggaaggaaaataactaagtatagttttcaagcaatgtcttcatgacatttataagaactatcaaacagctacaaattctagaaattctccaaataatgagaaaatgtatagataatcttcttagccttggaataaaacttaaggcatgaaacaaaatatgaactatggtttttattcttgagatactgtacttcagggctatgtttaccacagacaagacattcagattaaactcagaagaaaatcacctggttattttgtcctgggtcacacaggtaggtaatgcaaatgatggaactcaagcatcttctgattacaaattacacaagctggctccgaggcacagtgggatccctaggtctcagacacacttgaagagttgaatggattttcttcatactccaagatatttgtagaatcactcaatcttaaatgaaaaaatcaaaatgatcaggaagatctagcgaatgtctcagagttcgtttgtcatgttacattttgttccatgactttgctgtgttattcaatattttggaggtaaaggagaagagggtctatgggaatattttaataaaatatagatttttaatttttatttttaatgtggagaaaaggcacaggatcttagggcaaagccatggtttagggatgagctttggctccaggtgacacagggaggtcaatgatgtatttgtacgtgacacctcatgcaagaagaatacttcgtggcaacattggaggcttaaaagagataagtaatataaatgtgcctggacaaaacaggtgctcagtagtagttagttatcatgactattcattcacaaagtgaaactggattggcttgctcatagggactgctcctggaagttgttcagagtttctgaatttctttggcttatctcttaattacattttttttttaacaacacctcacatccttaagctagcagactgaccatcaatagattttcaatacagtttgaaagtcttaacatatattacagaactaaagtgagctctgatgacttttaatataaacagcattggctattttcgatttccactcccaaaaggctactactgacaagcaaaagttaaaggcatctacacaaattgaagtggttctacaagcaagtgtaaattgatcaacagtggtagtagaaataatgatgcaacttcaaagtggttgtttaaggcactcatagatcaaaaaatatatataaatatttgatgcctctgaattccttaaaaaaactatagtaattttaaacatgaaaatgtttaaattggaacatttattatgaaaattaattaaactgaattaatatatattttaattaacactataattattatagaatgaaataacagtgaaaaagttagaaaacctgttcagtttttgataaagtatatggaaatagattaagcagggctcttgagtaaattatttcattgatttgtatctatttaggtaggtgagagttgcatagaatcagtggcgagaaggatcagtgaaataaatttacaacttcagtgaagaccaggtaatgagcattgatctatgaatacagactttcttcagtgactatttcccctaaaactcccagctcaccgcagcactgctgcttcaagattccagttattggcgtgctagccaagtagaaggtcatttttaccgaaacgcaaatttcaacttatggccattttctactgaccatttcactgcaaaatggataaaacaataggaaatataaactacagcaaaagttaaagctctatatggacgttaaaagaaaacctcttgctttctttttcttcctttttctcttgttttctttttctacctttttctgcatatggcatttattttcatatcatgataagcattctcatgtttgcacttaattttccttatttctatttttcttagatttgttgatgttgtgtggcatattaaaagtaggaaggactgtataaactcatgtattatttttaaattgtttttctgaatgtactcaagtacttttttatgtgaatatgaacacggaataaaagtactaaaaccagttaattatgagagactaagaatgtggaaaggaacattccggagtatattagtagctaaagaaatgccttggatcttaaatttcaacttctaaaattattaaaatacaaatattaaaaagatatagaggccactgctaaagtttgtttacgccttatttcttcggttgcctagattattcgttttaggtgaatttgctttttctgtcaccgtggttatgttaacccaactgattcatccaaaacggaattggctaaactcctttatgcacatatttatattagggctgattattttgctcattgagtttcaaaaaattatctagaggactgcgaagttttcattgctttacttaatttctttttcgcttgtgtgatttttatttcttactcttgactatatctctatacggtttgaagtaacagggcagaaagttaagctaataatttctgctgacttgataattcatcagtttgacaaaaagaccagtgggccaccattaggaactgaaggcaactgaaatgacatggcttactcttgctggaatcttagcttgaaagttgaacatctactccacaatgccacgggatccgttcatgatatcatttgctgaagcacttgccaagaatttcattatttagaaaaagcagaattaacatttctatctattatttttcttttctttctttaagctgaaaggcattcagaggctgtcagtaaataaaatctatcaggaacaaaatcattttcaaaatttttaaacatgatttattcttgatgcaaatcttattgttcttacaattctatgcaagtcaagttgagctaaagcatttttatttaattcaattttctttcttctgaaatgtgatacagtaaaatcaggttttgaatatgtacaaacacatcaggatgattattatacttacataagtaaaatatatgtaaaatattatcatatcttcaccatatttagagtctaatatttgttagattttttgtgtgtgcatttttcctttccccttcagaaaacgtagttttttatttaattcatgtggtttttctatagctttctgggtctcatcaaatttcacatgaaactatgcagagtcatatttatgttaaggaagacatgcaattaaaaccctacccatttgttttgttagtcccaacacgtatcaaacaaaaaacttatgttgttagatgtagatttgattatctccctttatttatttgggtctctctctctctctctctccccgccccaccgtctgtctcactctcactctctctctctcactttctctctgatctcttttcctttcaactcagcttgcaggaatggatctcatttgaaatgagctgcactggtgtcagcatggttgtttaaccatgtaaactatctcatgatgcttagaattgaagagaagcttaaacgcttgtgtcagaaaaatatcttacaatacacaaaacacattaaatatattattagaaaaagtttagttgtgcaacaaagacctttaatttcaactaaaaactgatgcagtaatgtattacaatgacccaaaaagaaatattcttttcaaaatgacttcgggaagctatcagagtttgtgggaaaaatgctaaggtaatactttcctaagaacaaatgtaaagctgcaaacaatgactttgaatttttcaagattagcagtttggatttacatgtattttaacaaaatataaaaaatttactttcaaatcttggctttaaaaaaaaatcgtgtggctcatgcctgtaatcccagcactttgggaggctgaggagggcggatcccaaggtcaggagactgagaccatcctggctaacacggtgaaaccccgtctttactaaaaatacaaaaaattagctgggcgtggtggcgggcgcctgtagtcccagctactcgggaggctgaggcaggagaatggcgtgagcctgggaggcagagcttgcagtgagccgagatcgcgccactgcactccagcctgggcaacagagcgagactacgtctcaaaaaaaaaaaaaaaaaatcatgtttcaactgaaatggatatttattatctgcaaaagtccattttgttgcatatattgtttaaaataattcttaagaaattgttacctttctatttctatggagaactaacaccttaatacaagtctagcccccttttccaagacacaacgtcccccacaatgccactgagtttgtgtttacctgaatactttcaaacgacagctgatccttgaataacatgggtttgaactgcatgagttctcttctatgaagattttctttacctctgccatccctgagacagcaaaaccaacccctcctcatcttcctccttcaaagcctactcaacatttgaagatgatgaagacgaaggccttcatgatgacttcatatgactttcccttaataaatagtaaatatacttttccttccttatgtttttttataccattttctcttctctagcttactttattgtgagaacacaatttataagcatataaaaggcagtatagcaagatcccatctctaaaaacaaatattaaaaaaaaaattagcttggcagaggggcaggcacttgtggtcccagctaacagagaggctgaggtgggaagattccttgagcccagaagtctgagattgcagtgatggcaccactgcactccagcctaggtgaccgtatgtcttcattgattatttctgttatcagtaaggcatctagtcaacagcaggctattagtggttaagttttgggggagtcaaaattatacatgaattttcaactgttcagaggtcagagcccctaactcccagatttttcaaggactatctgtatatacaattgtagcactgctgctataaatgtgcagatatgttttaaatgatctaaggctaacaaaaacatattcagcagtttttttaattttaagaataatctattcagaatgtgtatccctgtgcaattaattacaaatagctaaaatcttgcaaaatatttaatgtatcaaatcagcatgtgatttcatttatagaaataaaagactagttttctcccttcttttacccatgaacttggctaattaattttctatttttccctcaaacttttctcaaaggtgggtatgaagggaaaacttgtttttgtgacactagagagaaattgatgaagcctgggcacaacgtcatttgtctccctgtcacaaagttgcaggtragctggccattcatcacctggtacacagcacacctcatcttgaccaaagagcacctttcttcatcagagaagctgtgctattgagagcactgggaaccaggtctgaattggaacaagaggcagaaaagcagaatgatrtttctttctaaaatcatataataaacaccaataaaatttctgaatagtagtaacgtgtcttttgttattcaaagtagtattatatagcttttcctcttcattttattgaaagtttcaggaagggggaatgtactaaagcttaaaattaatatctaatgcttttcatcttgagaacagtatgtacatcttgcacaactatattttagtttgtataaaatagacatataaattacctgcattcatggctttcattcatttatctgaataaacttactgagcagcctctatgttcctgggactgggcaaggcattggagaaatagatacaaaagacctatttttggacttccaggggttcttagaccactgtggaaaaagggcatacgattacaatgcatgttatgtaaaaaatatggcaaagttagagtacactggaagcatttaactcctattgacagaagttagttgcacaaatagagtggcattttaaatttaataatcagatctaaattcagccagcaaaagacacagaaattttgtcatgtgcaaagatgggaaatatcaaaatagaaagttgggggaagagtcactttataaaacaaatgattacctttagattgaaagaaagtcatgacccctattgaaaacagacagacaaatgtcggtataggatctgttgaggaacagagggagtaacagtggggaatgcctcaatgctattttgcctttcagcagagatgggcagaagagatttgaaaaagggttcatttttatagatgtatgtaggtatttgtatacgtgtgtatgtgagttcacccacacatgtatgtatgtgtattaataacccactgacctcaaagtaagagagaaataggatttacccagtatttacccaagaatccccatcatccttatcccaggattctgcagtcagcaaaccaggctttttaacactctccaatcatgaggtatttaaagarctccatgatttgttttgtatttaacgctgcaataaccattctcagataaacatgtttgagggcttcttgagatacatttttctaaggaaagtttatgggttaatagagctttaattgttttgggtatatatttccaattttccctaccaacatttntgcaaaaggcacagagcaatccagctgtacacatgcatgcctgctgcattttcatagaaagttcttcctaccatgaaaacataccttttactcagttctctgaactgtggatttcacgtcaggagagtaaaaatgtggnngtagttatgttcttggttttctttgggagtgggttcttgaggaaagagatgacaaacaagaataggcaacttcaggtctccaaaccncagaattggcaaatatagctaaaccggaaggggacagtcattttcaagtgctgtgtttttttaaaatatcaggtaatttttaaaatctacgtttaatataactttatcagcaatcaaaaaaaaaactggtcaacttgaatgatcttagtcgtcttttctatgaggatctcttcttgatgtaaaatatccaaaggccctgttcacaccttaggacaccagttagcattcgtttctgttgatcaagaaatatatatacagatgctctttgacttacaatgaggcgccatcctgataaacccataataagttgaaaatattccaagtaaaacgtgcatttggtacaagtacacctaacctactgaacatcatagttagtcctgcctacctgaaacgtgcttggaacacttacattagcctacagttgagcaaaatcatctcacacaaaaccaatttttttttttgagatgtagtttcactcttgttgcccaggctagagtgcagnggcacaatctcagctccctgcaacctntgcctcctgagttcaagngattctcctgcctnaacctcccaaatagctgggattacaggcatgtgccaccacacccagctaattctgtatttttagtagagatggggtttcaccgtgtttgtcagacaaaacctattttatagtgaagtgtcgaatatctcatttaatttattgatgttctactgagtgtatatcacttttaaaccatcgaaaagtttaaaatcattaagtcaaacataatctgtcaaaccattatagtcgaggaccatctctaatctgnaatatgaaacattacattacatatttatgtatcatataatacttcaattatataaaatgaatttacatatgaatcgaaatagtatccacacatatttaacaatatatgtacatatatttcatttcacatagtgtagacccctagaaattaggtcattgtctgcctactccaaatatcatctaaataccatccaaaggttgaaaattagttcctcaattatgttaacatttcagtggaaacattaaaatggtagtaaccataagcttgccattaatccagtaatgacatcagatttttgaagatttatgttcaatttcagacttcatattttaaaagatatgagctatgagaacatgacaatgattaaaactttacaaaataaaatatctggggagaaaggaataatattgggatatattactgagaaggaaaagaaggtgaaagagagacaacttatttttcaggcatctgtcagagccaagaactttggataaattatttctggctctcacaacaaacttgcataaaaggttttttaatcctaaatttttggaggagatacaaatctctgagagagtatgaaaatgagtcagagtcccataaatcgggaacatctgttcttggtctcaaaccccatgccaggaccatgttctttcgataacaacacacttaatacagtcctgctctcctaatcaggaaagacacaagggtctatgaaatcacttaaaattcaagatttagccttaattagtaaaaagaaattgcaaatttaattgtcagaaatgtggaagaaatgcaataacattgtttaggtttatattcctcaaacctttggcctcagaataaagggttaggttccgtattggttaaggatgatcatatcacatttatataaaatcgctaacaaaatcagtcaatattaactgaaaaatggtgttcaggacaatggtcaagatgaataatagacactaactcctattttgacaagtttataaattagttaaggatacaggcacatggcttaaagacaattggcagcacacttttcatcccatgttctcattctgttgttggcaaataacaaattatctttgtgtccagtgatttagtgatgggaaatcattctcttaaacttgataaagaaaattactgttctccagaaaaaaatatttcaagrgagagaatatcagtgatgtaatgagaacttataaatgtaaggaatgcctcattataaacatagtattatatccagctcagtgtcaacaatggtaatttgtggaggaggttagcgattctccatgaactcgacccaatgtcataaacactcatttctcaagaaaacatttaatttaaggaaccctcagtctgctttttagcaagatatgtagtttgaataataatattctggtttgaactgttttaaacttaggcacactgggtagttggcctctccatgaccaactggctttaggcattgtgctcactttctgtgagatggcagtaagggataaaactgcatcctgccagcatacagaggattctggatatcatgggctatccntggcttttagcctgctttctggaaataagccaaaaataagtatctttataaggcctgctaggttttatgtgcagaaattctaaataattttcattatgaaatatagtaaggagaggccgggcatggtggctcacgactgtaatcccagcatttggggaggccaaggtgggtggatcatgtgaggtcaggagttcgagaccagcctggccaacatggcaaaaccccatcgctgctgaaaataaaaaaaaaaaattagctgaacacagtggcaggtgcctgtagttccagctgttcggganactgaggctggaaaattgcttgaaactgggaggtggaggttgcagtgagctganatggcatcgttgtactccagcctaggtgacagagtgagactctatctcaaaaatgaaaagagagagagagaaagaaagagaaggaaagaaagaaagaaagaaggaaggaaggaaggaaggaaggaaggaaggaaggaacaaagaagaaagaaagtgttgaataaacttgtatttagattgtctaggattatctatataaggtcaaactcaatttccagattaatattaatctattcccacttaataaaatgtctctatttctatatacacttaagagaccagggattttgctgatggtaatttaaatttatatagtgaggtttacattttgaaattaataacaattttttattagcattagagacttaattgtttaaagcaatcttacctacctactatatcagtagctaactgctgcctaccacagcagtcacacaacagcatgagcacaaaatgtttacagttaccatgtccctgcccagagggtatccaaaaattacattggcactacagctaaataaacccatgtaaaacatatactataaaagagtgtagacatatatcacatttttatggaaagtaaaacattgcctaattttagataacaaaaggaaaattgttatctacacaataaatgaattaaaattctcatttgtcataaaacagaaaggagagaaaatcaggtcacttgtttactagtcaaaattatcagaattcacccaaatatgtagtgtgatacttctaaatacaataatatagagagaaatatgagccaattacaatcatttttatttagagtactagttattttaatgaaatttatttattgataatggaaaactttttattttttattttatgttacttcaaaataattattaacttacggaatagcttcaagaacagtataatagactttttcctgttttcccaaaccatattataataaattgtcaacctgaagcctgatcattcagaatatcttaagtatacatttcataaaataaaagcattctcctttatatcgatgatacagtattaaaaaacagaaagttatactgatattactaccatctagtcctcagatcctatccaactttcactcattatcccaaaaaataaccttgagagcaaaagaacccaatttataattacatgttgcattgagttgccttgtctcttctgtctccttcagtctgaaacagttcctcagtgtgtattttactttaatagccttgaaactttggatggttacaggccagttacattttagaangcctcctgatctgncttgnctgatatttccatattattagcttgaggtnacacatctttgctaaantatcagaaatgtttaccctatcatgtaaaacattattttaccctgttccagtaatgatgaccttcactttaattgcatgaatgaagtggtagcctcatgcctggggataccacacacacacacacacacacacacacacacacacacacacacaca

SEQ ID NO: 4 - DNA, 5566 bp, Homo sapiens
  CDS 659..2032
  allele 4484: 99-13821-332 : polymorphic base C or T
  polyA_signal 2531..2536: potential
  polyA_signal 2538..2543: potential
  polyA_signal 2873..2878: potential
  polyA_signal 3307..3312: potential
  polyA_signal 3843..3848: potential
  polyA_signal 3859..3864: potential
  polyA_signal 4524..4529: potential
  polyA_signal 5536..5541: ATTAAA
  misc_feature 10,39,2574,2580,2978,4692,4696,5466: n=a, g, c or t
ggcagcccangctccggactctgggagagccagcgcggnagcaggagcgggaggcggaggagaagaagaaggagaaggaaaggaaggaggaggaagaggaggagaagaagaggaggaggaggaggaggaggggaggcgctccgggcgccgtcagtgggcagcggaggcgcggcatgcccctggcaggggagagcgggctgggctccgcggggccatggggacccgcgcgctgacaatgcctgggtggccggagccgcgccagccaccctgtcacctccaccatcgccccctgcacccagcctcgccgccggccaccagcaccagcctcctccgtctctctctctctctctctctccattattgtttcccactctacagaggatggggtcggctgatgattaggaagcaagtcagaccttccttcaaagcctggtaatatttatgtaggcaaaagagacagagagagataaaagagagggagagggagagagagagagaggagagagggagaggggaagagagagaaagagagagaggagagagaaaaaacgggaggaggggataaggaaattaaaccctttaagtcaatgcatattgtggtgacaccggcacaggcgccctcacggtggagtcggccagggctgtgcgttcccaaaatatgaccaggggtgcttggatgtgtcggcagtatgacgacggcttaaaaatctggttggcagcaccccgagagaacgagaaaccgttcatcgattccgagagggctcagaaatggcgactgtctctggcatctctcttgtttttcacagtcctgctctctgatcacttgtggttctgcgccgaggccaagctgacccgggcccgggacaaggagcaccagcagcagcagcggcagcagcagcagcagcagcagcagcagaggcagcggcagcagcagcagcagcagcggcggcagcaggagccctcctggcccgcgctcctggcgagcatgggggagtcctcgcccgccgcccaggcacacagactcctctccgcctcctcgtcccccaccctgcccccctccccgggagacggcggcggcggcggcggcaagggcaaccgaggcaaagacgaccggggcaaggctctttttctaggaaactctgccaagcccgtgtggcgcctggagacttgttacccccagggcgcgtcctcgggccagtgcttcacggtggagaatgcggacgcggtgtgcgccaggaactggagtcggggggcggccgggggggacgggcaggaggtgaggagcaagcatcccactccgctctggaacttgtcggatttttacctttcgttttgtaattcctacacactttgggagttgttctcggggttgtccagtcccaacactttgaactgcagtctggatgtggtgctcaaggaaggcggcgagatgaccacttgcaggcagtgcgtcgaggcttaccaggactatgaccaccatgctcaggagaaatacgaagagtttgaaagcgtgctccacaaatatttacagtcggaggagtactcggtgaaatcctgtcctgaagactgtaagattgtctacaaagcctggctctgttcccagtattttgaagtcacacagtttaactgcagaaagacaattccttgcaagcaatactgtttggaggttcagacgaggtgtccatttatattgcccgacaatgatgaagtcatctacggaggcctctccagtttcatctgtacagggctttatgaaacctttctaaccaatgatgaaccagaatgctgtgacgtcaggagagaagaaaaatcaaataacccatccaaagggaccgtagagaaaagtggctcctgtcacaggacatcgctcacagtgtcatcagcaacaagactgtgcaacagcagactcaagctgtgtgttcttgtactgattctcttacacacagtgctcacagcctcggcagcacagaacacagccggactgagctttggaggcatcaacacgctggaagaaaactcaaccaatgaggagtaacggaaggacgagtgtcaccacagcagcagctggcccgccgtgaaaaatggcaactgctgtctcatgtaacagaaactgggtgcttttaccctcgaattacttattgcaaggcctttagggtaaaatttaaacagatgggcctgaatccaaacaaggacacaaccacagctttttattgactaaaaggctggaaagtgactttaaatttctcacaccattttatacactgtgttttaatgtttggaggttttatttgctttcgttttggtttgggtttatttgtttgtttattttttgcacttgttaatacaggatttattttgggggatggtttctcagaggtaaactaagtcttttcactgtctctatctctctatatatttctagtcattgtgtgtgttcatcagatagttctgtctttatgtcctgtcagcttctattagaggaatgattgctatgacctcatggtatagcaaaaaacaacaacaaaaaaagaataaaaaataaaaaagacaaaaaaaagaaaacaacaaaaaatnaaaatnaaaaaaatccctaagtctcccttctacccacagaaccaacaacacccttcccagcctttcctttccctcgccctcttctcgtcccctaagcaaacaacatccgcttgcttctgtctgtgtaaccacagtgaatgggtgtgcacgcttggtgggcctctgagcccctgttgcacaaaccagaaacagagcagagccaagggggcctgacaagagttcctttttagctgaacaaacaagtgctctccataataggtggaatcagacagttaacacatttttatgttgaaaacaaaataaaaggaaaaaattaaaaaaaactatcatgaactgtattgctccagttcccatccccaagtggcccagccctttcttgctggtccagctggacaggagcagcnatctagaatcaggatgcggggagtgaggaagtttttcctttgacaatgaaggtgggctttcattgtgatttttgttctgttgcagtaatataggagcacattttggccattgtaattacagggaacaaagggattgcggacacatatctggacttcttttcctcccttattgttgtggaagagacactagaaatgctcaaacacctgcaatatacagaatatacacaattttattccagtatttccctaacatatggtttaaaattattccaggtatacagtgtatgcaattctgcattatcacagaggaacaacttcttttttaaaaaataaataggtcagccatttttattaacgtgcaaaaactttatcactctaacatgctctaggtagttgaggaaaagaggtctgatcactgtttgtattttattttctttgtgggaacatttcacctgctgagtgtacatgaatttgctttctataaaaggcttttatgagtttacagtagaatcagtggaaggaagagttaataagggctgtttttaaaaaaacaaacaaacaaacaaaacaaataattaaaaaaaaattttacattccttcctattctctaactacacttgggaagtgcacttcagataagtttgcagtgtgactgagagatgaaggaaatccatagaaaaggtcctcttagtgaacaaaatttagttattaactttatagctatgaaatttccccgggcatttgtttttgttcaaacagactttaacctctgcatcatacttaaccctgcgacatgcgtacagtatgcatattttgttttgaaaaaaaatgtttcgttccagtctgttaagaatattcaaaaataataaaggtattgcttaataaaattgctagaattgtttagcagtacatgcacaatattttactagattctttgttttaatagtgttttgttgagactgaaaatcttaaaatggtctgcgcaaatacaaaaaaaagaaaacaccaaaaatgcaaaattctcccgtttttgttcctttttaaaaatttttttttctgcaaatgcaaatacattcacatgtggacagatacatacacataggcatcatattttagtgtatggaaacatggtgcttttctggggacacaagctgaggaagtgaatggctcgggggagacacattaattgcatggaattgttgactgtgaaacacttgtagaaatgagcactttggtatcccaaccaatggtggaatacagatttcaatggcgctggggggacgagaagcagaggaattgaggctgcaagtgattcctgaggaagagctcacagggtgattttattcagatgacctctaagggaatggctaggaaggagtctgttctgacctttatctacctctgctgtggtaaggccactccatgtgccatctcaccatctcttcagcgttcctcatgctgcttctccccgtgccataggcgtttccattcttgcaggaaaatgayaatgcaagtgcaaattccactcacttttattggtatgacaataaattattctttctaaacacttagcaaaatgacgaaaagcagtattccttaaaatataggttttcattttctcaatatattttgaactgactaaagttctttacaaagacaagaggatcagaaggttccacttacccgtgttttagcgtttgtttgaagataatgncaancaaaaggtggaaaactaaaaccaccattgctttacataagaggtacacactgccccccacccccaaaaaaaaacacagctttgaaagttgggagaaacactgcagtcttcattgtaaatataaagtgataatttaggtgaggaaggaacggtaaggagggaaggggaatgagtcctttctccccatcaagtcagccagcattggaatagtctctaattctggcggaatattttctttgtcatgttcatctattcttattacaggagaatgatttcagtgcctagtatttattagtgtaagtgtgtaaccactttcccgcaatatttccactggacatgttgaagcaaagcagcttcttctggggtcaggcagaggatccaaataggcaacgactcctttcccaagctcagaaacaatcactgatgatgcagatttcactacatccgttgattctttgtagtagttttcctggcacacacttgagcatgcttaaggttgtgcatatgttcattttgaacaaataagttcagcccctgctcctttgtaatcatcatcttgtttagtttttcctgtagaggatgtctaccgtatactggctaatgaactacaatattaaaataattatttctgcaagcaagttgaaaaccaagaaaacaatatttgtttcataaatgtatggcatagatttccctgtttattattctttttatctgcaatagagaaatttcagtttctggaatatgttggtactgctaagctaaatggcaaanaacatagagttcaatgtcattctttattgcatttacatttggtgatacgttatattgaacttttcttgcattaaaaagatagtatttttcccctctctca

SEQ ID NO: 5 - protein, 458 aa, Homo sapiens
MTRGAWMCRQYDDGLKIWLAAPRENEKPFIDSERAQKWRLSLASLLFFTVLLSDHLWFCAEAKLTRARDKEHQQQQRQQQQQQQQQRQRQQQQQQRRQQEPSWPALLASMGESSPAAQAHRLLSASSSPTLPPSPGDGGGGGGKGNRGKDDRGKALFLGNSAKPVWRLETCYPQGASSGQCFTVENADAVCARNWSRGAAGGDGQEVRSKHPTPLWNLSDFYLSFCNSYTLWELFSGLSSPNTLNCSLDVVLKEGGEMTTCRQCVEAYQDYDHHAQEKYEEFESVLHKYLQSEEYSVKSCPEDCKIVYKAWLCSQYFEVTQFNCRKTIPCKQYCLEVQTRCPFILPDNDEVIYGGLSSFICTGLYETFLTNDEPECCDVRREEKSNNPSKGTVEKSGSCHRTSLTVSSATRLCNSRLKLCVLVLILLHTVLTASAAQNTAGLSFGGINTLEENSTNEE

SEQ ID NO: 6 - DNA, 1791 bp, mus musculus
agccctcacgggtggagtggagtcggccagggctgtgcgttcccaaaatatgaccaggggtgcttggatgtgtcggcagtatgacgacggcttaaaaatctggttggcagcaccccgggagaacgagaaaccgttcatcgattcagagcgggctcagaaatggcgactgtctctggcttctctcttgtttttcacagtcctgctctctgatcacttgtggttctgcgccgaggccaagctgacccggacccgggacaaagagcatcaccaacagcagcagcaacagcagcaacagcagcagcaacagcagcaacagcagcagcagcagcagcagcgacagcagcagcggcagcgacagcagcagaggcagcgacagcaggagccctcctggcccgcgctcctggccagcatgggggagtcctcgcccgccgcccaggcacacagactcctctccgcctcctcgtcccccaccctgcccccctccccgggaggcggcggcggcagcaagggcaaccgaggcaagaacaaccggagcagggctctttttctaggaaactctgccaagccggtgtggcgcctagagacttgttacccccagggcgcctcctccggccagtgcttcaccgtggagagcgcggacgctgtgtgcgccaggaactggagtcggggggcggccgcgggggaggagcagtcgtccaggggctctcggccaactccgctgtggaacttgtcggatttttacctttcattttgtaattcctacacactttgggagttgttttcggggctgtccagccccagtactttgaactgcagtctggacgtggtgctcacggagggcggtgagatgaccacgtgtagacagtgcatcgaggcttaccaggactacgaccaccacgctcaggagaagtacgaagagtttgaaagcgtgctgcataagtacttacagtcggatgagtactcggtgaagtcctgtcctgaggactgcaagattgtctacaaagcctggctctgctcccagtattttgaagtcacacagtttaactgcagaaagaccattccttgcaagcaatattgcttggaggtgcagacaaggtgtccattcatattgcccgacaatgacgaagtcatttacggaggcctctccagcttcatctgcacagggctctacgaaaccttcctaaccaatgatgaacccgaatgctgtgacatcaggagcgaggagcaaaccgcacccagacccaaaggaaccgtggacagaagagactcctgtcccaggacatcgctcacagtgtcctcggccactagactgtgccccggccggctgaagctgtgtgtactcgtcctcattctcctccacacagtgctcacggcctccgcagcgcagaactccacgggactgggcctgggtggcctccccacgctcgaggacaactccacccgggaggactgagcgcagccaggcgcgtgcgcagagcgcagggctgggcagggacacgcgcttggcacagagcagcagtgacccaccggggatgctcacctgctgcagcccgggaactgaacccacccgggtgctctacccttggacttctcgcaaggcctgtgggtaacattcaacaagatgggcccgatccccaacatggacacagccgcagctttttgccgactaaaaggctgcaagtgactcagtttctcacaccattttatacactgtgttttaacgtttggaggttttctttgctttcagttcggtttgggtttattttccgtttttaaacttttttttttttg

SEQ ID NO: 7 - protein, 467 aa, mus musculus
MTRGAWMCRQYDDGLKIWLAAPRENEKPFIDSERAQKWRLSLASLLFFTVLLSDHLWFCAEAKLTRTRDKEHHQQQQQQQQQQQQQQQQQQQQQQRQQQRQRQQQRQRQQEPSWPALLASMGESSPAAQAHRLLSASSSPTLPPSPGGGGGSKGNRGKNNRSRALFLGNSAKPVWRLETCYPQGASSGQCFTVESADAVCARNWSRGAAAGEEQSSRGSRPTPLWNLSDFYLSFCNSYTLWELFSGLSSPSTLNCSLDVVLTEGGEMTTCRQCIEAYQDYDHHAQEKYEEFESVLHKYLQSDEYSVKSCPEDCKIVYKAWLCSQYFEVTQFNCRKTIPCKQYCLEVQTRCPFILPDNDEVIYGGLSSFICTGLYETFLTNDEPECCDIRSEEQTAPRPKGTVDRRDSCPRTSLTVSSATRLCPGRLKLCVLVLILLHTVLTASAAQNSTGLGLGGLPTLEDNSTRED

SEQ ID NO: 8 - DNA, 23 bp, Artificial Sequence
  oligonucleotide g713LF1
cgcttgcttctgtctgtgtaacc

SEQ ID NO: 9 - DNA, 26 bp, Artificial Sequence
  oligonucleotide g713LR1
gtatttgcgcagaccattttaagatt

SEQ ID NO: 10 - DNA, 26 bp, Artificial Sequence
  oligonucleotide 713.LF1.5.1
actgtctgattccacctattatggag

SEQ ID NO: 11 - DNA, 25 bp, Artificial Sequence
  oligonucleotide g713.LF1.5.1n
tgattccacctattatggagagcac

SEQ ID NO: 12 - DNA, 20 bp, Artificial Sequence
  oligonucleotide g713RACE5R1
gggtagaagggagacttagg

SEQ ID NO: 13 - DNA, 17 bp, Artificial Sequence
  oligonucleotide g713RACE5R-49
gggcatagcaatcattc

SEQ ID NO: 14 - DNA, 20 bp, Artificial Sequence
  oligonucleotide g713CTGLF132
ggctgtgcgttcccaaaata

SEQ ID NO: 15 - DNA, 28 bp, Artificial Sequence
  oligonucleotide g713RACE3N
aaaaatgtttcgttccagtctgttaaga

SEQ ID NO: 16 - DNA, 29 bp, Artificial Sequence
  oligonucleotide g713RACE3Nn
attgctagaattgtttagcagtacatgca

SEQ ID NO: 17 - DNA, 20 bp, Artificial Sequence
  oligonucleotide SG1polyA
tttttttttttttgacagag

SEQ ID NO: 18 - DNA, 25 bp, Artificial Sequence
  oligonucleotide SG1LR100
tttgccatttagcttagcagtacca

SEQ ID NO: 19 - DNA, 22 bp, Artificial Sequence
  oligonucleotide g713.PU
aatattcttaacagactggaac

SEQ ID NO: 20 - DNA, 22 bp, Artificial Sequence
  oligonucleotide g713.RP
ctttatagctatgaaatttccc

SEQ ID NO: 21 - DNA, 24 bp, Artificial Sequence
  oligonucleotide g34301.PU
ctgatcacttgtggttctgcgccg

SEQ ID NO: 22 - DNA, 22 bp, Artificial Sequence
  oligonucleotide g34301.RP
aggactcccccatgctcgccag

SEQ ID NO: 23 - DNA, 23 bp, Artificial Sequence
  oligonucleotide SG1LR1102
aaaatactgggaacagagccagg

SEQ ID NO: 24 - DNA, 18 bp, Artificial Sequence
  oligonucleotide SG1LF790
gcacttagagcgcggggt

SEQ ID NO: 25 - DNA, 15 bp, Artificial Sequence
  oligonucleotide SG1LF834
gccggaggcagccca

SEQ ID NO: 26 - DNA, 17 bp, Artificial Sequence
  oligonucleotide moCTGR1511
tgtcctcgagcgtgggg

SEQ ID NO: 27 - DNA, 27 bp, Artificial Sequence
  oligonucleotide moCTGLR20
cggaggaggggatacggaaattaaacc

SEQ ID NO: 28 - DNA, 25 bp, Artificial Sequence
  oligonucleotide moCTG1440
tgggtcactgctgctctgtgccaag

SEQ ID NO: 29 - DNA, 20 bp, Artificial Sequence
  oligonucleotide moCTG5RACE1
tcacagtgtcctcggccact

SEQ ID NO: 30 - DNA, 20 bp, Artificial Sequence
  oligonucleotide moCTG5RACEn
tcctccacacagtgctcacg

SEQ ID NO: 31 - DNA, 983 bp, Homo sapiens
  misc_feature 14: n=a, g, c or t
aaaaaaaaaaaaanaacaaaaacaaaacatgaaacaggaagacaggaaagatccatttgacagagtacttgacacaggagagagaagaaatactcatgtatctgaaagtattcaaagggggagtgttaggagatgaattaatttaaaaaatgagtaagagtaaaatagtttaaagttagaccctgaggaactccagggaagacaaagtaacacaaggaacaagcaatgttagccactgcctaactttcctcagggtcatgtgtgcctcgccataattatgtaaacacttacattgttaaaacgaaattcggagaactagtttgagtaaaggggaaaagaaagtatgttattcatgtcggagttggaaatatgtgataggttgaaattctcaatttcctaattggaaatcattaagtcatactgaaacctgaaaattcaagaactgacaacacaattgatgttgagatatggaatttggtacctgatgaaagattagaaaattattaaaagcaatttcttctgggtggtgctacaagatggaagaagaaaggacagaaagctcttcataatcaggtagacgctttgactttttaagtggtatgcctatatgcctttaaaaaacaactcaatttaaaagaaaattaagagatgctaacagccgatttaaagaaaatttagtaaaatattcaattgtataaagatacacaaaatattggttatctacatgatagcaaagatgaattaagggatggggataaaactcttctcaataacaccaaaattaaaataaaacataattcatatatttagaaatatcattacagaaatatgttgaacttgtattaacagcctctcctcaaaggtagcatggagaatcatgcaaacttaatttggagatacaaaaaaaattgagaatgtgtagtgttgttctttaattctaactgtaatggctgaataatattttgatcatgattgtgatacta

SEQ ID NO: 32 - DNA, 450 bp, Homo sapiens
  allele 298: 99-15663-298 : polymorphic base C or T
  misc_binding 275..321: 99-15663-298.probe
  primer_bind 279..297: 99-15663-298.mis
  primer_bind 299..317: 99-15663-298.mis complement
  primer_bind 1..18: 99-15663.pu
  primer_bind 430..450: 99-15663.rp complement
tcccaccttcttctaaacgtgttgcttcaatacgttgataggtgaggacacttaaaaattagactttatagaaataggtttttttttgtttacatatatagttcttttggtatcatatatttagcctctttctaaaatttattttttgatactgaagggagaaatagggagttattaatcaacaggcattaattttagtcaagcaaaataaataagctgtagcgatctgctctgtaacattgtacctacagccaacaattatatgttgtccacttaaaaatgtgttagatctcatagyaactcttcttaccacaataaagtaaaaattctgaaacaataagtgaatacctaaataatacaaacaaatacaatattgtagttttgggcacttaataaatgacagcctcatttctcaattagagatcatcacaagttagacagatgacgatg

SEQ ID NO: 33 - DNA, 476 bp, Homo sapiens
  allele 398: 99-15665-398 : polymorphic base A or G
  misc_binding 375..421: 99-15665-398.probe
  primer_bind 379..397: 99-15665-398.mis
  primer_bind 399..417: 99-15665-398.mis complement
  primer_bind 1..20: 99-15665.pu
  primer_bind 458..476: 99-15665.rp complement
cgtaaatgtgaaaagcatagcctcttcttggaatgttaagtataaatatctgaaatactgggcttgatatgtcaacaggagattgatggataaaaatagaattttatataaaaaacaactggacatattagattgttaacttggaagaaagaccatattcaaagaagaaaacatagtgactaatttcaaacatttaaagtcttccctgtggaaacaaaggaatatctttgttctaacacttcaaagaacagggttaaaaaatagactcaccacagagtaaatgcacaattgacaatcgtgaatgaattaaaaaccaaacaaaatattttgtcagctttctatctatgaaactaagaaacaggcttcctactaaggtaatgaatgtaattcacagagarcattcacgtataagtttcattcatgtttcaaatttcattgatttgatcaatgggttattctaataccctcccttattt

SEQ ID NO: 34 - DNA, 547 bp, Homo sapiens
  allele 166: 99-15672-166 : polymorphic base C or T
  misc_binding 143..189: 99-15672-166.probe
  primer_bind 147..165: 99-15672-166.mis
  primer_bind 167..185: 99-15672-166.mis complement
  primer_bind 1..18: 99-15672.pu
  primer_bind 533..551: 99-15672.rp complement
ccaataccataactcctctataggacatggaagagtattatatatgacaaatgattgctatgattattattatcagtgttattattatcctaatcctaagtaatccaataaaagaaaaatacatctgtgcctgtgcgtatgtgcacgtgtgtgcagtcaaatacaygttgagtaaaggtaaagtctagctgtatttaatcaacctacctgaatcctcaggaaaaaattctaaacctagtttaaaacatgtaaactctaagctctctccttatagtcagttagtagcagcacatcttaaaatctggtgtgaatattctcttagttctacatgagtctaactaaacagaggattattcttaggtgtttgaaagagacatatgtgacactgctgttttgagaacaatttaagtgttgtcttgtcatgtacagaagttctcatattactttacataaatggttgcataattgttttatagtaaataatagactgtcaatatttctaggataactccaaaacaaaatttcctagammacattttgaaaaggg

SEQ ID NO: 35 - DNA, 502 bp, Homo sapiens
  allele 185: 99-15664-185 : polymorphic base G or T
  misc_binding 162..208: 99-15664-185.probe
  primer_bind 166..184: 99-15664-185.mis
  primer_bind 186..204: 99-15664-185.mis complement
  primer_bind 1..19: 99-15664.pu
  primer_bind 483..502: 99-15664.rp complement
  misc_feature 54: n=a, g, c or t
gtttaccattagcactgtcatatttgtgtgacttgtcattctctacagcggagnacgggctggcacggggcctgatgctgacttgcacaagggaagcctcctgtctctgacttccccaggataattcctggggaaagtgtgctccctagtgttaagagcggtttaatggctggagggtttcagckggctgaccaggcagagaaggagggtgaatcacctctcagcactctccacttagactttgtgtggtcgtcgggtggtcaaaccttctaactagttgtattgcagatttggcattccagtgcaaacaaaagacagaaacacaatgttcacatgctttccagagatcacctggatatcagatcatttgattttcaagtaagtcgaaaccttggtggaaatcattaactatcctgtttatgaccaaaaaataaaatcccaaatttcttctcttcatttcttacctgctttaaaattgtatccaaagcgtgraagtaaaaga

SEQ ID NO: 36 - DNA, 455 bp, Homo sapiens
  allele 205: 99-5919-215 : polymorphic base A or G
  misc_binding 182..228: 99-5919-215.probe
  primer_bind 186..204: 99-5919-215.mis
  primer_bind 206..224: 99-5919-215.mis complement
  primer_bind 1..19: 99-5919.pu
  primer_bind 435..455: 99-5919.rp complement
ctacagcaatgcagatttcaattctgccattgaattcccagacatattcgtcatccccattttcatcccccaccaccctgccattttcttcgtgttaacttgttttcctgactcacagaaatcaccttttcctgtatacatttttaggatgtcagactttattctaatgatttctcctagttgccccccaaaattgtattctacrgtgtgattttaaagctgaattttcaagatgatatttcatatctatattttcacaagcttttcttctatgaatgttattgtcagctgtcagggtgtgagatggtacttgatactacattctttccaagctgttgcctgaatcggtttaagacaaagtcattactaggctgtaaactgttgctctgcaaaattgagcagcacgtatttaaccactcatacttcttagctctccaacactttgagtcaataga

SEQ ID NO: 37 - DNA, 450 bp, Homo sapiens
  allele 157: 99-5862-167 : polymorphic base C or T
  misc_binding 134..180: 99-5862-167.probe
  primer_bind 138..156: 99-5862-167.mis
  primer_bind 158..176: 99-5862-167.mis complement
  primer_bind 1..20: 99-5862.pu
  primer_bind 430..450: 99-5862.rp complement
aatcaaggtagagatgtatgagaaatagccggttaaagaaacagcattactttcagactatcttttatttgaaatacacgtggggaaaccagaaggtgaaaccccttaggagatggatataggatactaaaatctgagttagaaaaatttgagcatyagcaccttacgtgtcatgctaagatagtgaatgagactgcacaggaattgcatgcagtttaacggaaaaagaagtcgaaagataaattcctagaacactaacaccgagttatgggaggagaaatatcctgcacaggtcactctgggagacatgtcaattgtttagccaatatccatttaactcatctttcttcctaatgaaaaccgaatttggagaagcaggtagtgcccctggctagaaatatgaaccttcccagcttctctcatgcactgaactgacaaagttcaggtctg

SEQ ID NO: 38 - DNA, 403 bp, Homo sapiens
  allele 292: 99-16032-292 : polymorphic base A or C
  misc_binding 269..315: 99-16032-292.probe
  primer_bind 273..291: 99-16032-292.mis
  primer_bind 293..311: 99-16032-292.mis complement
  primer_bind 1..19: 99-16032.pu
  primer_bind 384..403: 99-16032.rp complement
gttgttaccccacttcttccccccagctcccccttcctcacacagttcatgccacatgccactctcctggactactggaaatgcgtcagtccactctgggctcatcccatcatcccccatgctgcaacctgagagagagttgcaagttgcaaatctgatcttgtcaccaccactctccacactaaatccctctaatgcctccccctttctttttggataaattccttctgcttgcatagccacgtggttggcttctatagcatcacttcacactgtggtcacctgccttctmctcactcaggaacttctctccattgaagaagttcttcttccccatctccagggctttcccactgacagttgtatctcccccataccaagcccaggtggtcatctcatccca

SEQ ID NO: 39 - DNA, 476 bp, Homo sapiens
  allele 118: 99-16038-118 : polymorphic base A or G
  misc_binding 95..141: 99-16038-118.probe
  primer_bind 99..117: 99-16038-118.mis
  primer_bind 119..137: 99-16038-118.mis complement
  primer_bind 1..19: 99-16038.pu
  primer_bind 456..476: 99-16038.rp complement
gttgcttattctttctctcttctgcagggtataaaggaatctgaacacgactgatattttctttaatttttagatccagatatacattgggtaaaatctacttcataggttttcaaargagcattcttctgagcaaatctgaaaactctctaaactctattggtatgttactctttatctttatatgaatttaaattcttctagaagttagataaaactgtggtaaagctacataatacttttgacatattttcaagcgtagacaaacttcaattaatttgtaagatacaggaagaaaatttttccagttaaaatgtacctcttggtttctggagtgttagcaaccattcacacttacagttcaaacagtgcaaccttgtaaaacatatataacttatgaagagatcgatatctctttttataaagcaaacaagtaaatttttccctcaatccatgatttatttttgtgaagtggg

SEQ ID NO: 40 - DNA, 498 bp, Homo sapiens
  allele 133: 99-5897-143 : polymorphic base A or C
  misc_binding 110..156: 99-5897-143.probe
  primer_bind 114..132: 99-5897-143.mis
  primer_bind 134..152: 99-5897-143.mis complement
  primer_bind 1..18: 99-5897.pu
  primer_bind 475..492: 99-5897.rp complement
aaaagtgtttgccagtcctgtttcttacagagcacagaactcagatgctcttataaagatacaggataaatcacatcatttcctgctccatcatcagaatattattatatgatttagatcacttttttaaaamagaacatggacttagtacagaacaacagcaaaagcctggggaaggagaggagtgcaccatgaggagtcaatggggagcagaagccagtccatttgactgatttggttcgtgtgcaaaataattgctaaataattgcatatatgtgagactccgggtattttcaaaaccagctggcaaaattgtgttattctctaccctctgctggctttcacgggttctctgttctctctccttttcctccattctcctcttaccctaattcctgaccactgtaatccaataatctaaggttttaggatttggatgactaaggttacccatggaattgtttggaaatgtagacctgtaatggagaggggagaaaa

SEQ ID NO: 41 - DNA, 517 bp, Homo sapiens
  allele 360: 99-13601-360 : polymorphic base A or G
  misc_binding 337..383: 99-13601-360.probe
  primer_bind 341..359: 99-13601-360.mis
  primer_bind 361..379: 99-13601-360.mis complement
  primer_bind 1..19: 99-13601.pu
  primer_bind 500..517: 99-13601.rp complement
gttttacttgacagttaccaagaattgtttcgcatttaagaaaattatatctttgatggttccctcattaatggtgcctggatacccaatgcaacacacctacatcaaactgcatttgtaactgttggattcataatgattctacctaagatgcaagcatacggcatcattgtgccttgttgtatggatatgcttgagaagtcacatgctgaaatacatatattttaaatttgacagtatctcctacaatattttctttatattatagtaaggtattacattacagtttaaaacttatgactataagcaggtgatattatctatgaatttcatgtgaaattagcaaagggacagtctcaratgtttgctgtataaagtgtatttgaagcctgatagggttgagaaacactcagctacagtaagtaaaaacagctctcttagtggttgccttgttgagaagatcttgaaaacaaggttgaaaatacaaaagaaactgtgtggagtctacaaagatatt

SEQ ID NO: 42 - DNA, 533 bp, Homo sapiens
  allele 97: 99-13925-97 : polymorphic base A or G
  misc_binding 74..120: 99-13925-97.probe
  primer_bind 78..96: 99-13925-97.mis
  primer_bind 98..116: 99-13925-97.mis complement
  primer_bind 1..20: 99-13925.pu
  primer_bind 513..533: 99-13925.rp complement
catggaagtaaaagcatatcttcattataagacttctacacaaattatcacatctttacttacagcagctgaaacctggaaacaactctaatgcccrtcaacagaggaatggatggataaagaaactgtgatgcagtggaatacgactcaacgaagatgagactaaaaataattatactgagtaaaagaatccaaacaaaatagagcaaacactgtgccatcctgtttataccttactccagtaaatgcaaactaatacacaatgaaaaaaattacttatttgagaactggggagaggaaggagagggaaaggggtagataaagaaaagaggagagattaaaaggagcataagaaaacctcagagaataataggtttgtggtaaacattaccgtggtaatgtttttagggtatattcacatgtaaaaacttatccaattatacattttaaatatgtacagtttagtgtgtcagttatgcctctgtaaagttgattttaaaaaaagtcctattccaagtymacaatttcatttg

SEQ ID NO: 43 - DNA, 480 bp, Homo sapiens
  allele 201: 99-13929-201 : polymorphic base A or C
  misc_binding 178..224: 99-13929-201.probe
  primer_bind 182..200: 99-13929-201.mis
  primer_bind 202..220: 99-13929-201.mis complement
  primer_bind 1..19: 99-13929.pu
  primer_bind 460..480: 99-13929.rp complement
gggagaatactaataatggaagcattacttttattttttctataaattcctctggaaatatgtatttcttatgtcctaaggttattaacaaaaagagaaaataatttctgatttataattcactttccttcaaaaaataataactcagtgtctagtaaggtaaagcaaaaaaagttaaaagaacccataagtttattttamaatacctactcagaagcaaaactgactttctattaaaaattaaaaaaaaaagttttcttattattgttttgtttccttgtttttaggtgatgggattgtatttgcaactctctggtcagtaagtgataaaatgccatttctatgcacccacctggcctgtgtgactgggagaatctctctttttattaaatgtgcttcaagttttaacaactgacttttgttagtgatatgatttatctacccgtgactgtcaaacaacacagatgatttgcatatctc

SEQ ID NO: 44 - DNA, 477 bp, Homo sapiens
  allele 108: 99-14021-108 : polymorphic base A or G
  misc_binding 85..131: 99-14021-108.probe
  primer_bind 89..107: 99-14021-108.mis
  primer_bind 109..127: 99-14021-108.mis complement
  primer_bind 1..18: 99-14021.pu
  primer_bind 460..477: 99-14021.rp complement
tttgttgttaatcgccccttttctgcaacacttgtgggttagggaaaataattctaaagcaagagcaaagacagagttgggagatcaccagtgaggttcaattttccrtcacattcactctgctccacacctcagataatcatgtgcttaactgcgaaacttgcttgacaattacagaacactttctcacccattactaccttgatcctcacaattctgtggggtagtaggagcagatgctgaaattgccatacgcaaatcagtgaactgaagcttagagacctccagcaggggcagagggtcagcggaaactatcccagggttcagccaacaagaaagtatattggaatcagagtattaaaataagaataataaaaccaactaaaatttaccgtgctttttatttccactcagtgccaacaattcttaacagtgtcagtgatggatccctgtgccccaggggacagacttcttact

SEQ ID NO: 45 - DNA, 475 bp, Homo sapiens
  allele 314: 99-14359-314 : polymorphic base G or C
  misc_binding 291..337: 99-14359-314.probe
  primer_bind 295..313: 99-14359-314.mis
  primer_bind 315..333: 99-14359-314.mis complement
  primer_bind 1..18: 99-14359.pu
  primer_bind 457..475: 99-14359.rp complement
ataagggaatggtgtgaggtgggaccagaggaggctgcactgagaaagtgagaggggcaagacctcaggggaagaagggagggctgcacggatgtctcaggcagagcaggcagcaccggaaaaggtgggggacactccttttggaccagcatataatttggttaaagcctctcctgtttcacctaatatataagcacatttcaagataaaactactactttattgtcatcaaatataaaagtaattttttattcagggttttctaatactcatctataaaggcatttctttcccacatggcatgtgttacaggstgtttaacttaaagcaattgtaaaagaaaagcctgaagaaataagtctacaacgatttacatcgtgtttatttttgtgtcaaaatatatgttaaaatatacattagctatactaagggaatcaagagaagatcataattgctcttatgacttgggatttag

SEQ ID NO: 46 - DNA, 473 bp, Homo sapiens
  allele 316: 99-14364-415 : polymorphic base C or T
  misc_binding 293..339: 99-14364-415.probe
  primer_bind 297..315: 99-14364-415.mis
  primer_bind 317..335: 99-14364-415.mis complement
  primer_bind 1..19: 99-14364.pu
  primer_bind 453..473: 99-14364.rp complement
gtgttttaattcaacccagctataagatacgaaatgatagaattgctctagattctctattggttaaataaggagatatttgtgctattgccaataatacatgctgtacctggataaacccctttgggcaagttgtgatgcaaatactcaagaaaataggccacatagttacaacaggacttacctaattccccatggtcatttggctgattcagtcagttgctttcaagcctaggttcttggctcaatattattacataaactagaattttcctattactattaattttactttgtatttttctttataaacttygtacttattgcttgtcaaatttcagcagaagtacaactcctgagagaataatgctggctcagagttttgagatgataacccttgtctatgaaactgatgaagttggacttaacaacgaacactccccacagaactcctgatgctcaaatgtggctaa

SEQ ID NO: 47 - DNA, 502 bp, Homo sapiens
  allele 99: 99-15056-99 : polymorphic base C or T
  misc_binding 76..122: 99-15056-99.probe
  primer_bind 80..98: 99-15056-99.mis
  primer_bind 100..118: 99-15056-99.mis complement
  primer_bind 1..18: 99-15056.pu
  primer_bind 482..502: 99-15056.rp complement
caggaaactcacaagaagscagatttccttcgagcacctcctgaataaagaggcaaaggccttcttaactcttacaatttacaagtggctatgagtgcytttatagttcccataataatttctccacgtagacttcctaaataataatttctcctgttttatattctctgtgcttatgtttatatcaaacaagttaccacttaatcaaatgccgatttgcattgctcactatgtaactttaattttctttgcctcttatttttggatcttaattctaaaactagatgatcataaattcatttaggaataagcttgtgatctagccttcttttgaacccctttgtgctcctcacaatatttgtttcgatgaaacagtgagcaacatttgatctatgattgttaatagaaaaacaccaatgtctcaagttattgtaaacataggcataattgacctttggttctataaatatgtttggtgttccccaaaatacgtctccctttt

SEQ ID NO: 48 - DNA, 494 bp, Homo sapiens
  allele 412: 99-15229-412 : polymorphic base A or G
  misc_binding 389..435: 99-15229-412.probe
  primer_bind 393..411: 99-15229-412.mis
  primer_bind 413..431: 99-15229-412.mis complement
  primer_bind 1..20: 99-15229.pu
  primer_bind 476..494: 99-15229.rp complement
ctgtcattgagaaatgctaccaataatacttagagaatttgatacaactcagtctgaaaaagctaagattagcagaacagagctgtctccaaatatttgaagaactattttatttaagggattggacccatttttgtatgtagttccagaggagcagatggtgaccactgtccaggcagatgtgtctcaatgtaaggacaacatctgtaatattaataattagaatgtatcctgtaattttctctctacccttggaaaccagtcgagatccagagtctttcactgggaggcttaaagcctagagcagccttggtgctagaggcggacagggataatgaactaatcttgaaccaattcatccatagcaatctcaatgctttcgttagctcttataggtatttaatacggccavaggaatgaaggtagtcttgctggtttagaagccctgcctaccacaacccctacaccaccccatcccctgcatagtctgatgt

SEQ ID NO: 49 - DNA, 485 bp, Homo sapiens
  allele 291: 99-15232-291 : polymorphic base G or T
  misc_binding 268..314: 99-15232-291.probe
  primer_bind 272..290: 99-15232-291.mis
  primer_bind 292..310: 99-15232-291.mis complement
  primer_bind 1..18: 99-15232.pu
  primer_bind 467..485: 99-15232.rp complement
caatagaacaggctgctcctttataattattaatcatagtgtatattaattcatcatcacatacgtggctagaaaaaaatttagaacaaaaagatatgtgatatgtaaaggcctacgataattcagacttctttgaggagagcttttattttattgttattcttattttatctcttgtcaatataaattgagagaataaacagacaaacattacaaattagtgattaattgcatttaaagcctagttaagactatttaagactattatgcataatacaggaaaactacctktattatttatagtgggtgccttctgaaggatctgaaggagaatcagttctatgcctctctcctcattcccaggaggtgcctggcattccttggcttgtagacgcatcaccctaatctctacctctgccttcacatggtgtcccctgtgtgtgtgtttttgccccatgtgtctcctctttttatatggatgccag

SEQ ID NO: 50 - DNA, 464 bp, Homo sapiens
  allele 347: 99-15241-347 : polymorphic base A or G
  misc_binding 324..370: 99-15241-347.probe
  primer_bind 328..346: 99-15241-347.mis
  primer_bind 348..366: 99-15241-347.mis complement
  primer_bind 1..19: 99-15241.pu
  primer_bind 444..464: 99-15241.rp complement
gttatgggttgaaaatctctgagttcttgtacatacaaaaattttactgttgtcacagttgaatcttagtttagatgggtataggatttttattcaaaatgcttttactccataagtttaaaaatattgttacattttcctcaagtatctgatgttattgatgagaagtttaattctaatttgactcttgttcccttgtaggtactatttgttttccagtttgggaagcttacatttcttaaaattcacaacatataatttacatactacacaattctttttaaagtatacaattcaatgcatttagtatgttttagtacatataacttaaattatgtatatacaaratctctttataatatttgtagaatatgtagcatattcacaagattgttcaaccatcaccactctctatttccagaatcttttcctccaaaaagaaaccctgaacactatgatgaata

SEQ ID NO: 51 - DNA, 550 bp, Homo sapiens
  allele 196: 99-15244-196 : polymorphic base A or G
  misc_binding 173..219: 99-15244-196.probe
  primer_bind 177..195: 99-15244-196.mis
  primer_bind 197..215: 99-15244-196.mis complement
  primer_bind 1..20: 99-15244.pu
  primer_bind 532..550: 99-15244.rp complement
ctgcttctggttatgttttcctaattgccaaaatggtaaaaatgagaataatcattgaaagagaaagcataaagtagcaaaaatcctttccagattaaaaaacgaagcaaagcatgtttcccaagtaataatactctcatcttcctccctaatcctttaccccactaccagaagaagagtaaaatgtccggatatrtttgaaggtaaagatttctccttttaataaaattagtcaccttgtacacatcagtagatcttgagaatgaaaagcttttctagtacattcatttcaacctataaatgtttgacttttctctgtcattcatttacgacctgtgatcttttcattccctttcagttagaatatttttcaaatttttattgatattttctatttaacccataggttatttggaaatacattgtttaatttctaatatatttgcttttttttctacttatttctttttttcttaattccacactggtccaaatatattctgcatatgatttaatattttaagttctgtagagactaaccttgtgccct

SEQ ID NO: 52 - DNA, 452 bp, Homo sapiens
  allele 404: 99-15252-404 : polymorphic base C or T
  misc_binding 381..427: 99-15252-404.probe
  primer_bind 385..403: 99-15252-404.mis
  primer_bind 405..423: 99-15252-404.mis complement
  primer_bind 1..18: 99-15252.pu
  primer_bind 433..452: 99-15252.rp complement
atgggggcatatagcaaccctttagaaacaaaactacaaaaggtaagcttgtcttcttgcatttcctttctcttactacatttaacatgggaggttttctatgtctcacattcaaatattctcactcgggctgcctaatttttccctgattttccatcactctttatgaaggcttgctactttagaatacacattttcttaacagaagataataatcagaagatgtctcccaaatataagtccaaatctttcctatcatgctgtgttctttggctcttttgactttatttgaagtcagccttgaaggggatagagataggctgtatgaagtccacgctgagaagttttgccctgccctacttgtcctgtaatatttcatggatagcccagtggtgattaaaccygtgtgtacaggaataaccatgagaatttgttaaaaatataggctctgg

SEQ ID NO: 53 - DNA, 477 bp, Homo sapiens
  allele 382: 99-15253-382 : polymorphic base C or T
  misc_binding 359..405: 99-15253-382.probe
  primer_bind 363..381: 99-15253-382.mis
  primer_bind 383..401: 99-15253-382.mis complement
  primer_bind 1..19: 99-15253.pu
  primer_bind 459..477: 99-15253.rp complement
aaaatcaattccccaacactcattttgtacgctaattttgtaagatcctgaaaagtttcactattttatggtttcatgtgttacagatgaaaaaaaaactagaattcaaattttctgagtttttttttacaatattttatgattacaaagttagaagactaagaataaaatggcctaatttccataatgtgagtggtaaatgcagagcactggcctaaagaaaatatttcaaaaaattagtcatcttttccttaatttttttccaacctatgatctgttgaatgagcattttgcatatataaataaataaattactttgtaaataatcttgactggtttctgttgaccacagtaacccactgcacagcacagcctgtaattyctatgaacctagggaaatgtatttaagtttattttttgattacacaggtcctcattgtgtaactaaacattgcatagaatatgccagtgatgatg

SEQ ID NO: 54 - DNA, 456 bp, Homo sapiens
  allele 392: 99-15256-392 : polymorphic base C or T
  misc_binding 369..415: 99-15256-392.probe
  primer_bind 373..391: 99-15256-392.mis
  primer_bind 393..411: 99-15256-392.mis complement
  primer_bind 1..18: 99-15256.pu
  primer_bind 439..456: 99-15256.rp complement
cctctctatgatgcttcctattaagcaattggggaaatgtaataaacaagggttggtgagcatcttccttagtgagatgtttttggaagaattggataattgagtgaataatagtgagaaactcctgtgtctgatgttgctccatgttggaatgcttttatgttctcagagaatgagtcactgagagccaattgtgatgatacacaatggttttacccaggttggatatggtcctctgtactggtaccctttaagtcagtggcactaatcagtcagtcattgtcatgctttgtgttggtccatcatatggtatgccctcttagagaacatcctgattagtccttagacatcttttcaatttgaacactggggctcctcattcgggtaaaaaytatggacagtcagtgaaactgttgcaatggcccctcatagcagattggatctcaatgcactttg

SEQ ID NO: 55 - DNA, 501 bp, Homo sapiens
  allele 200: 99-15261-202 : polymorphic base A or G
  misc_binding 177..223: 99-15261-202.probe
  primer_bind 181..199: 99-15261-202.mis
  primer_bind 201..219: 99-15261-202.mis complement
  primer_bind 1..19: 99-15261.pu
  primer_bind 481..501: 99-15261.rp complement
cttctaatcctttgtttccacttattttatttcattcctcattttatcccttttttctaaattccattttattatacttaaggtgcttttaatatggttatcatactcctgatagtgttatttctttcttagtcttcttatataagcgctatacgttcacattccatctcctttggttatctttccatttcttcaccgarcctctttgctctctttttttatagctggttcactcaaaatgtcttactttgccatttttgaaatttattttcattcttttatgtactgaataaaatttaaaaatactttatcatggtgggaggtacccgtgatgtccaaataagtgtttatattaattgttggggtttttttgtttgtgtgttttttgaaaggttaagaaaatctcattcagaaagtaagttgtttaaaaattctggaccaaatttaccacacatcaagcagatacttaccaagttgtttggtagacattagcagtattta

SEQ ID NO: 56 - DNA, 541 bp, Homo sapiens
  allele 432: 99-15280-432 : polymorphic base C or T
  misc_binding 409..455: 99-15280-432.probe
  primer_bind 413..431: 99-15280-432.mis
  primer_bind 433..451: 99-15280-432.mis complement
  primer_bind 1..18: 99-15280.pu
  primer_bind 521..541: 99-15280.rp complement
atgtccatccatcttgcccagagagagtttctacaacacttcctctgcaagccctttccctacttgcctcacctattgctttcctctgttacgttgtattcccctcactgtttcttccaacatcttcccacctcagagcatggacacttgctgctctttctgtgtcatgatgctgctcacttgtccctttcttaatgtctcctccctgagccaatcttctccacccccacaacttacgcacacttacatgtcatattttccttcatagcctttaacaccatttgaaatgatatatatttgattgcttttaaaatttctctgtccccccactaaatataaacttcaggatggcaagaatgtagtccattatcttatttctccagcctccatacttttaagaaaataaattttggttgtataagccatccagtyagtggtacttggttatagcacccctagcaaaagaatacaaaaaaagggagaatgtttgcaatcatctgtttgaggctaggaattcccagagagggaaacaaggagtaat

SEQ ID NO: 57 - DNA, 514 bp, Homo sapiens
  allele 428: 99-15353-428 : polymorphic base C or T
  misc_binding 405..451: 99-15353-428.probe
  primer_bind 409..427: 99-15353-428.mis
  primer_bind 429..447: 99-15353-428.mis complement
  primer_bind 1..18: 99-15353.pu
  primer_bind 495..514: 99-15353.rp complement
tgggaatggaggtagtagacgatgaggtctccaccctctgactttgcagagatgggcaaggccaagtgttggaagggcttaaacacacaccggagtattctgtgagaaccagtggatttcagaggatggcaatgacaccacttgccttctgcctcaggaggataactgatggccgtgtgtgggatgcactggagagcaagagctggcttgcagggagaccagctggatgattttctttcatttattttattcattcaacacacattcatctggggttcactctgtgcccaacactgggcatttccaaatagtccagatggcagtaagcatggttgtggcagtaggaatgggaaggctgggaggggtatgagaggcattacaaacgggaagtgggagtggcaccccagaaaagtctagtttaaggtgcyagtggatgtgtgcatgtgtgcgcgggggtgtctagagggtggcgggcagctggaaattgaggtcaagtgcttaaagaacaactcgt

SEQ ID NO: 58 - DNA, 489 bp, Homo sapiens
  allele 150: 99-15355-150 : polymorphic base C or T
  misc_binding 127..173: 99-15355-150.probe
  primer_bind 131..149: 99-15355-150.mis
  primer_bind 151..169: 99-15355-150.mis complement
  primer_bind 1..18: 99-15355.pu
  primer_bind 471..489: 99-15355.rp complement
taacttctccgtctctccttcttagcccatatgtcaataatgactgaaagtattcatttccatctttaaactgcctattccagccacctcccacctccatctctttccttctaagttttcttcatcttctactttgggcaaaaggaaatygatgtgtcagacaggcctagttttgaattctggatctgctagcacttctctgtgtgtccttggttatatgatatagtcttaaaccttaatgttcttgcctgtaaaatggggataataaaaacctcttaacagtggttgtttcatgcagctttcattacaaacttcctcattcaaaatcttcaatgatttccatttttcacaaaatgaaattcaaaatttctgtagattattgagacaagtcccctactcttcacctaaatttatcttttatttattctctcatcattatcaacaactactaggctttgttgccttgactccagaggcaaaaatcttatc

SEQ ID NO: 59 - DNA, 468 bp, Homo sapiens
  allele 227: 99-15685-227 : polymorphic base A or G
  misc_binding 204..250: 99-15685-227.probe
  primer_bind 208..226: 99-15685-227.mis
  primer_bind 228..246: 99-15685-227.mis complement
  primer_bind 1..18: 99-15685.pu
  primer_bind 449..468: 99-15685.rp complement
aaacaaaggcacgcagaggataaggcatgagtccaaccagcagcatctccctcccgaatgagtacagaaatgatcaatactcgaagagaaaaagatgctttcagtgtgctttacctgaaaacttccttaagcagcttcactttattgtcaggatatcgctttgtgtttgtatcatctaagaaagctcgcgcatatgctagtgggccagcattgacctagacaaagarcaaagattttcagttccactaggaagaaaatcaccatgaccatctgctcagtttcagtttgcaggcactaaaaagcccgttcgcgtgagctactcacaatccctgccttccaggaacttaagcccaaaaagaaaccacaaagctcactctgttgcacaccacttgattccatgatctcagccatcttcagggcacttgtgatgatggtttactttatgtaagaagaaaccaatgcttggaa

SEQ ID NO: 60 - DNA, 500 bp, Homo sapiens
  allele 428: 99-15695-428 : polymorphic base C or T
  misc_binding 405..451: 99-15695-428.probe
  primer_bind 409..427: 99-15695-428.mis
  primer_bind 429..447: 99-15695-428.mis complement
  primer_bind 1..18: 99-15695.pu
  primer_bind 481..500: 99-15695.rp complement
atcagcctttgtgaggaggaggccctgcctgctctcctcctgagctgatgggtcagtcacaccaggacaaaggtctgcccggggctgtgtgggttcctccttcctgagctgcacaccagcatctgctgaacaccttctggagctcagctcagtgtctcgtccagagacactggttcccttggcttctcagcaactctcggatctgggcctgggtctaacctcagcggtggtcttgcccatttctagggcctcacaattcagcctcatgtcttcacctgtggctcttttgcaaggctcagaaagctctagggtcagttccagatgactcccaccagcatgccagtaggagccaccaccccctctcagccagcgccaccatattccaggcaaattccaactgacacagacttcaaggaacgattgtagcygttgttcttgcttcttccaaatggaagagtgcattattggggtcccttctagcacgcatttcattccccacc

SEQ ID NO: 61 - DNA, 472 bp, Homo sapiens
  allele 310: 99-15703-310 : polymorphic base C or T
  misc_binding 287..333: 99-15703-310.probe
  primer_bind 291..309: 99-15703-310.mis
  primer_bind 311..329: 99-15703-310.mis complement
  primer_bind 1..18: 99-15703.pu
  primer_bind 452..472: 99-15703.rp complement
agggctttgggttataggcgctgaatttcttctaaagctaacctgactctgatgctagaagagcccatttaaggaaagaaaaacacttttcattgctcgatcaaagttcatccattttggaaaagacatcaaaccaagtgtgtgacaccaggcacccatatccttcctctttcccaccaccccacccctgtcctcagggcagtgacagtgaagcctggtgcaggtcccgctgctgctttttgaagtggcacatgctttattttcttaaaaagaagtgagagacaacctatgctacaggaggctctgtgaygtttttctgaagtacaaccccttgctctgccagggcagctgtaaagggtctaaagagccctgagaaaggagagaggatttgggaagccgaggaggcagagggagaccacatagcacatggagttctgaaagggcccaagtggagacagaaaacgagtcatgt

SEQ ID NO: 62 - DNA, 470 bp, Homo sapiens
  allele 400: 99-15870-400 : polymorphic base A or G
  misc_binding 377..423: 99-15870-400.probe
  primer_bind 381..399: 99-15870-400.mis
  primer_bind 401..419: 99-15870-400.mis complement
  primer_bind 1..21: 99-15870.pu
  primer_bind 452..470: 99-15870.rp complement
gctcaaatgtatcaaacacagtttctgtggtcaagttcctctccttttctaaatttgcttagaggatctcataaaacgtaactcctctgacaagggaaccattttagcaccaacactgcaaaagcttctgtgttcctaagggaaagatcctttcctgaattaaatttaacctctttagtactcccatttagccacctgataaatccacttgagctatcttttgggaagagagaggtatctgggaacaataacacttcctttttgaacagtttaataaagctttgtgagatttcaagatgaaagataatgtgtaatgctgatagtgccctccaaggctctgcattcatggatccaattacgttttttgtcatggtaaaagccacagtggatatattaaatragagtgtggtttaagaatgaaggcccaggagtctggagatctggtttctaaggctgacttcacttctgct

SEQ ID NO: 63 - DNA, 469 bp, Homo sapiens
  allele 287: 99-16321-287 : polymorphic base A or C
  misc_binding 264..310: 99-16321-287.probe
  primer_bind 268..286: 99-16321-287.mis
  primer_bind 288..306: 99-16321-287.mis complement
  primer_bind 1..20: 99-16321.pu
  primer_bind 451..469: 99-16321.rp complement
ctttaggaatatcccttctgatttgaacaacattttgctatccaagttctgtctacttttttaacaagttcttgctccgtgtgtctccttttgcttgttctcaagtaagggagtaacagggataaactcccactccttggtaaatctttctatcatttttggaaatctcatccattgtagtaaatgctcttaaatcttcatcttcaggccgtgacttccatctagcctccattcacgtttccgggtttatgtctgcaatgagcattccgtggctctacatagatgcmccaccatacctagaacccatgtatcccaaactcaattctttctttcccaggacattacttcctgcacttccttagtctatcaatggcactgttattctcttgaccatctagacttgaaattttggggtttggactcctcctgctcccttgctttatatgtaatcagacatcaagtctcaatc

SEQ ID NO: 64 - DNA, 544 bp, Homo sapiens
  allele 194: 99-16333-194 : polymorphic base A or G
  misc_binding 171..217: 99-16333-194.probe
  primer_bind 175..193: 99-16333-194.mis
  primer_bind 195..213: 99-16333-194.mis complement
  primer_bind 1..19: 99-16333.pu
  primer_bind 524..544: 99-16333.rp complement
atttaccccgtctgccttgcaatttcaggatcagtatacatcaaatcaagtgaacaacccagggaattctgccgttaccttttagaaacagaataaatattaacagagctttacttctttccaccaaggaggactatatgttaatacagtaatttacactggaaaaaatataaatgaaagggtttagaacctcrtaactttaaaaataacataattcctcctagaacattcctttcacttgtgattctcaaagcactttgcatttcccagctattggcagggctggaattaggatcaaagtatcactaaatggtaggtgaaataaatgtgaagctgattttcaggagtacaggaatggagtcatcaggcgactttaaagttaagaatctgttggagcagctgccaataaatcaaggcccaaaggagaaagttctttggaaaccttgaaatattgtatacatttagataattattgttgttgtcaatgttaacgaaaaaagcaataaatcagggagatggcactgatgagtgaggagaaatagac

SEQ ID NO: 65 - DNA, 475 bp, Homo sapiens
  allele 149: 99-5873-159 : polymorphic base C or T
  misc_binding 126..172: 99-5873-159.probe
  primer_bind 130..148: 99-5873-159.mis
  primer_bind 150..168: 99-5873-159.mis complement
  primer_bind 1..18: 99-5873.pu
  primer_bind 457..475: 99-5873.rp complement
  misc_feature 409: n=a, g, c or t
gcgtaacaataagcagggttagtcgccacaaaacttgagataagaggaaaactaaaaaagtctaatgaaatcagtagtcttaaaaagatgacatgataggaagagaagtgttaaaaaagaaaaaaaataggtatgaaagagagtaacayataccggaaaagggataaaatacatcctttgaaagaacaaagagttattcaaattgaattcttaatgaattacttaaacagcagattagatattgttaaaaagaggaatagggaattaaatgatatatgtgatgatattacctagtgtaaccatcaaagatgtattgcaaatgataaagaaaaaaatgctgccatggcaatattaatatcataaaaatatactttaagaagtaaataaatgcaactaggaatagagaaansdvhatgaataataatatttaamaaavvgtataacaagtatacataagatgtaatatcctaaaccg

SEQ ID NO: 66 - DNA, 511 bp, Homo sapiens
  allele 49: 99-5912-49 : polymorphic base A or G
  misc_binding 26..72: 99-5912-49.probe
  primer_bind 30..48: 99-5912-49.mis
  primer_bind 50..68: 99-5912-49.mis complement
  primer_bind 11..31: 99-5912.pu
  primer_bind 494..511: 99-5912.rp complement
aaatataatagtcaaatcatgttaccattaggacacattaaaaatgtcraattaccttgggaccttatatgaacatattaagataataatgatagtgttcagtgcaatattcagatcaatagtttaaacccaaaatatttataccttcagattagatgtatgcaaatgcattgattcatgtgtcttttatctgttgtttacatttggagaaatatttgagaaatatttcaaaatggaatttatataaatttaaacacataatggttttatgtaaaaatattgctaaattacattttccccttaattcttatttcttggaaacgtgccttagtcgctgaaatattcatacattaacacaatgaaagaagtgaaccttactaggctttgactatcaggtttgctgttggtttttgactattgtgaaactatagcctgatttctaaatcaggaagaaacgtgtattgttgttaatatggacacatgacatatttgtctgcctgacttttgatcc

SEQ ID NO: 67 - DNA, 485 bp, Homo sapiens
  allele 210: 99-6012-220 : polymorphic base G or T
  misc_binding 187..233: 99-6012-220.probe
  primer_bind 191..209: 99-6012-220.mis
  primer_bind 211..229: 99-6012-220.mis complement
  primer_bind 1..19: 99-6012.pu
  primer_bind 467..485: 99-6012.rp complement
gtcttgacttgttttcctgagggtccaggttgatttgcatgctcttgaggaaatatacacgtcttctcagttttaataattgactgacagccctgtggtttctcaggacccagtgagctgctgctcccaggtcagtctgcaaaggatgctggttcccttgtggtctcatcaaggtgaggaatttcctgattttagagatttctttatcckaattttgaagactttctttcacatttctaggcataaaaaaatgtacagcactctactgcttgtttaacaaatggatagtgatatatctgccaacaaagaccacatggagtatttcattgactatcagagaagtttcctcgaaaggcaccatacttagtgttttatttccatgagtgaaggaaaattagttatttgaagtatttggctgtctttagttgtttctaaagtagtgctgattttatatgcccataatattcatatatacacccaggata

SEQ ID NO: 68 - DNA, 529 bp, Homo sapiens
  allele 89: 99-6080-99 : polymorphic base C or T
  misc_binding 66..112: 99-6080-99.probe
  primer_bind 70..88: 99-6080-99.mis
  primer_bind 90..108: 99-6080-99.mis complement
  primer_bind 1..18: 99-6080.pu
  primer_bind 509..529: 99-6080.rp complement
aaatgtgtccctgaaacccatgctatattcaactgaatattctaatgtctttgattacaaagccatctctagcaatttaatacaattaygaaatggaaaagttggcaaatgcaaaacaatagctcgtgttcaaggtatgtctttattaggggaagtttatcgaaacagatgtttatgctatttcctataaactagattctaaaatattttattctataaagatgtattgactttatatgaaaaaattattgaaaaatctacaagatggtgaaactctttagaactatatttctattacaagtttatttttaatttcaaaaatgtactgcataaatgcagcaaaacctttattgtcacatattaaaacatgtacattattgtgtgcaaattaaaatttcattaccttaaaccaaaaagtgagttggccagatagtaaataatttaggctctaaggctgaaaagcgcttgtattaattactcaactccaccactattttgccaaagcagtcacagacaatacgcattcaca

SEQ ID NO: 69 - DNA, 489 bp, Homo sapiens
  allele 156: 99-7308-157 : polymorphic base C or T
  misc_binding 133..179: 99-7308-157.probe
  primer_bind 137..155: 99-7308-157.mis
  primer_bind 157..175: 99-7308-157.mis complement
  primer_bind 1..18: 99-7308.pu
  primer_bind 469..489: 99-7308.rp complement
tgtggtctggatatggtgractgtccttcacacacagatgtgggaagccatgatcatcagttgcattattcctgaggggcaatgcattccagttacatagaaccagtttctacgtttcagggtatatgtattcatggtgacaaatttattcacatyttaagtaattttaagtaattcacattttaagtaattttcctgaatgtgcctcattggcttctgtgcctcttcagaaaagatgaactaaacactggcatatgtgttcagatttcaacattccgttgttttcattgtggataatttctgtcccatatttttgtgtaaagttagacaataaagtgttaatattctggcgtcggcacattttctttcctgataaataacaattcacatatctttttaaaatatcagagaatatagtaaccaatttccaattcttttttcaccatgtatctattggagttttaaaatgactaatactaaggcaactat

SEQ ID NO: 70 - DNA, 18 bp, Artificial Sequence
  sequencing oligonucleotide PrimerPU
tgtaaaacgacggccagt

SEQ ID NO: 71 - DNA, 18 bp, Artificial Sequence
  sequencing oligonucleotide PrimerRP
caggaaacagctatgacc